(19) United States
(12) Patent Application Publication
(10) Pub. No.: US 2023/0114207 A1
(43) Pub. Date: Apr. 13, 2023

(54) THE COMBINATION OF CYCLIN DEPENDENT KINASE 7 INHIBITOR AND IMMUNOTHERAPY FOR TREATMENT OF CANCER

(71) Applicants: Dana-Farber Cancer Institute, Inc., Boston, MA (US); New York University, New York, NY (US)

(72) Inventors: Nathanael S. Gray, Boston, MA (US); Kwok-kin Wong, Arlington, MA (US); Hua Zhang, New York, NY (US); Camilla Laulund Christensen Ross, Cambridge, MA (US)

(73) Assignees: Dana-Farber Cancer Institute, Inc., Boston, MA (US); New York University, New York, NY (US)

(21) Appl. No.: 17/789,047

(22) PCT Filed: Dec. 16, 2020
(86) PCT No.: PCT/US20/65267

§ 371 Date: Jun. 24, 2022

Related U.S. Application Data
(60) Provisional application No. 62/953,376, filed on Dec. 24, 2019.

(51) Int. Cl. A61K 31/4162; A61K 33/243; A61K 31/7048; A61K 39/395; A61P 35/00; A61P 11/00

(52) U.S. Cl. CPC A61K 31/4162; A61K 33/243; A61K 31/7048; A61K 39/3955; A61P 35/00; A61P 11/00

(57) ABSTRACT

The present disclosure provides methods and compositions related to combination therapy of a CDK7 inhibitor and immunotherapy. The combination of the CDK7 inhibitor and immunotherapy is useful in treating and/or preventing cancer in a subject. The combination therapy may further comprise chemotherapy (e.g., chemotherapeutic agents).

### CROSS REFERENCE TO RELATED APPLICATIONS

This application claims priority under 35 U.S.C. § 119(e) to U.S. Provisional Patent Application No. 62/953,376, filed Dec. 24, 2019, which is incorporated herein by reference in its entirety.

### STATEMENT OF GOVERNMENT INTEREST

This invention was made with government support under U01CA213333-03 and R01CA179483-01A1 awarded by the National Institutes of Health. The government has certain rights in the invention.

### BACKGROUND

Small cell lung cancer (SCLC) is one of the deadliest human cancers, accounting for about 15% of all lung cancer. It often arises in heavy smokers and is characterized by rapid growth and early metastasis. Although SCLC patients often initially respond to chemotherapy, tumors typically recur within 6 to 12 months, resulting in a 5-year survival rate of less than 7%. For the past few decades, first-line treatment regimens for SCLC have remained largely unchanged. Recent FDA approval of atezolizumab (anti-PD-L1 antibody) in combination with chemotherapeutic agents carboplatin and etoposide provided a new clinical option for the treatment of SCLC. However, this combination therapy only achieved a 2-month increase in overall survival. Thus, a need remains for new and effective treatments for lung cancers such as SCLC.

### SUMMARY

Targeting CDK7 disrupts cell cycle progression and causes DNA replicative stress and genome instability in tumor cells, leading to cellular responses including release of multiple pro-inflammatory cytokines/chemokines (including TNFα and Cxcl9/10). These tumor cell-intrinsic events provoke a robust immune surveillance, which leads to T-cell-mediated tumor control in vivo in mouse SCLC models. Combining selective CDK7 inhibitors (e.g., compounds of Formula (I)) with immunotherapies (e.g., immune checkpoint inhibitors) further promotes anti-tumor T cell immunity and confers remarkable survival benefit in this highly aggressive tumor. Accordingly, the present disclosure provides a rationale for new combination regimens comprising CDK7 inhibitors and immunotherapies for SCLC patients, as well as other types of cancer.

In one aspect, the present disclosure provides a method of treating cancer in a subject in need thereof, the method comprising administering a cyclin-dependent kinase 7 (CDK7) inhibitor and an immunotherapy.

In certain embodiments, the CDK7 inhibitor is a compound of Formula (I):

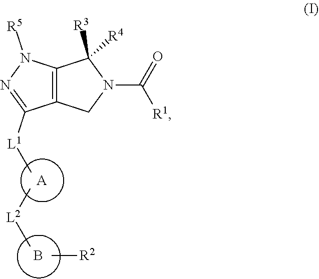

or a pharmaceutically acceptable salt, solvate, hydrate, polymorph, co-crystal, tautomer, stereoisomer, isotopically labeled derivative, or prodrug thereof, wherein:
R¹ is —NR^{a}R^{b}, —CHR^{a}R^{b} or —OR^{a}, wherein each of R^{a} and R^{b} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group when attached to a nitrogen atom, or an oxygen protecting group when attached to an oxygen atom, or R^{a} and R^{b} are joined to form an optionally substituted carbocyclic, optionally substituted heterocyclic, optionally substituted aryl, or optionally substituted heteroaryl ring;
each of R³ and R⁴ is independently hydrogen, halogen, optionally substituted C₁-C₆ alkyl, or optionally substituted aryl, or R³ and R⁴ are joined to form an optionally substituted C₃-C₆ carbocyclyl ring;
R⁵ is hydrogen, optionally substituted C₁-C₆ alkyl, or a nitrogen protecting group;
L¹ is —NR^{L1}—, —NR^{L1}C(═O)—, —C(═O)NR^{L1}—, —O—, or —S—, wherein R^{L1} is hydrogen, optionally substituted C₁-C₆ alkyl, or a nitrogen protecting group;
Ring A is optionally substituted carbocyclyl, optionally substituted heterocyclyl, optionally substituted aryl, or optionally substituted heteroaryl;
L² is a bond, —C(═O)—, —NR^{L2}—, —C(═O)NR^{L2}—, —NR^{L2}C(═O)—, —O—, or —S—, wherein R^{L2} is hydrogen, optionally substituted C₁-C₆ alkyl, or a nitrogen protection group;
Ring B is absent, optionally substituted carbocyclyl, optionally substituted heterocyclyl, optionally substituted aryl, or optionally substituted heteroaryl; and
R² is of the formula:

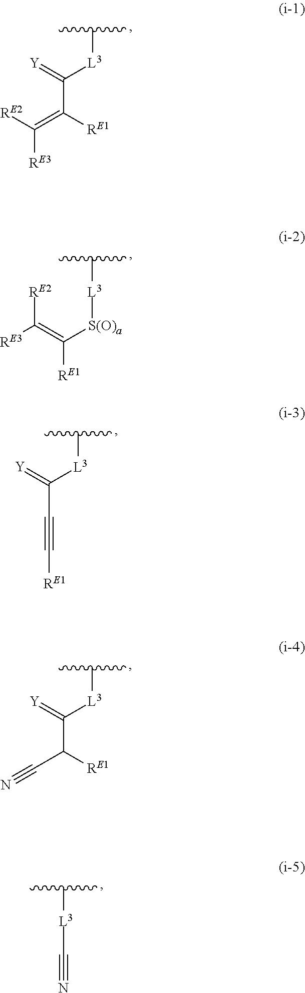

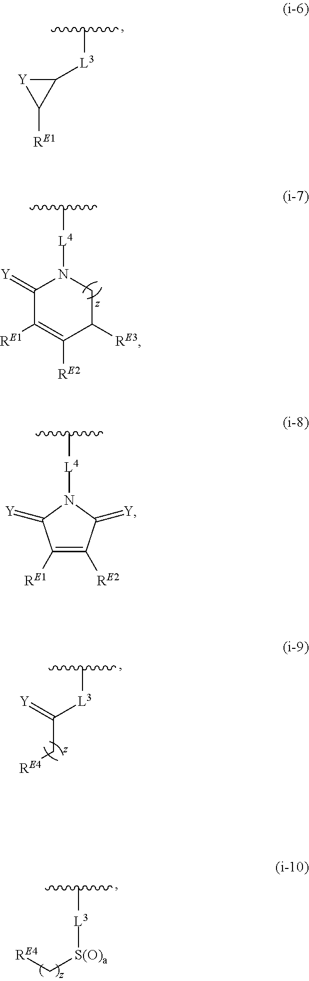

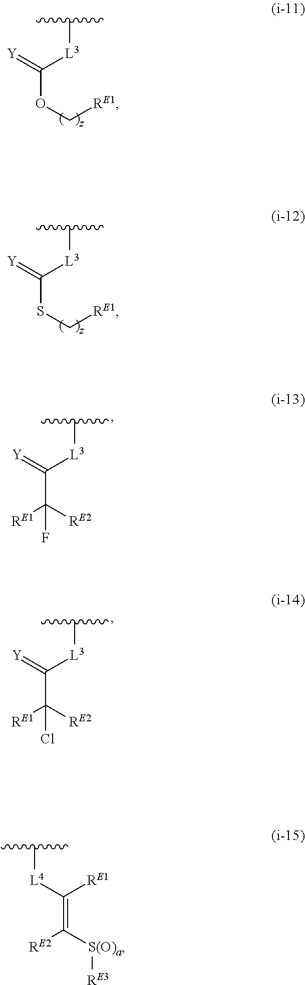

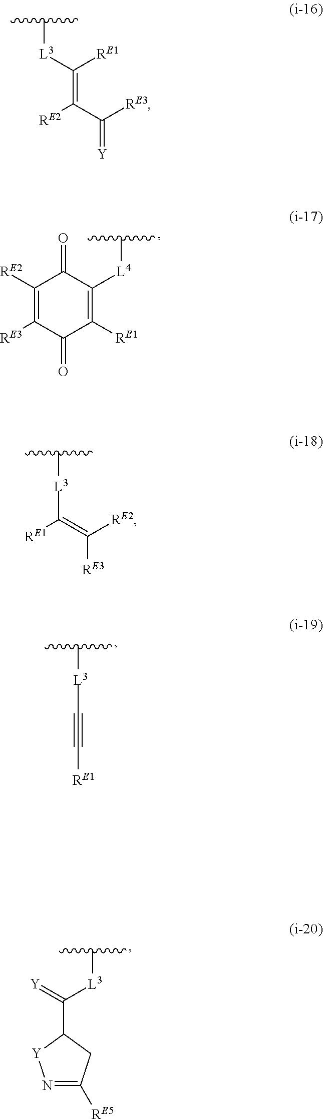

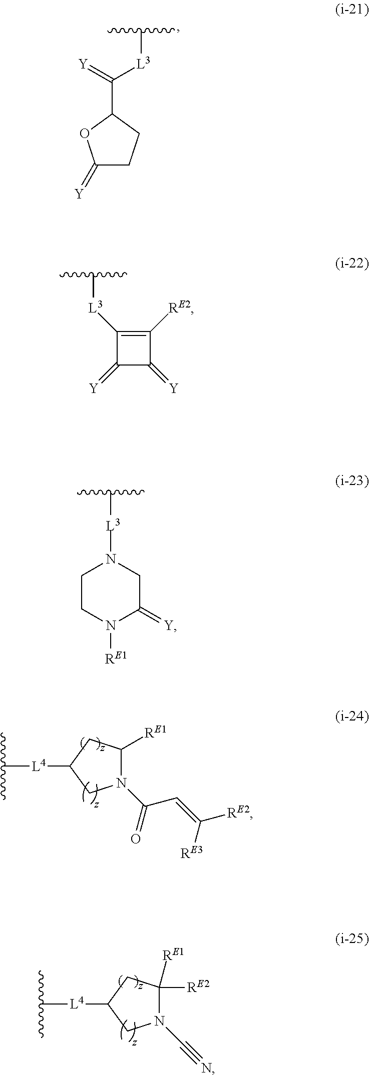

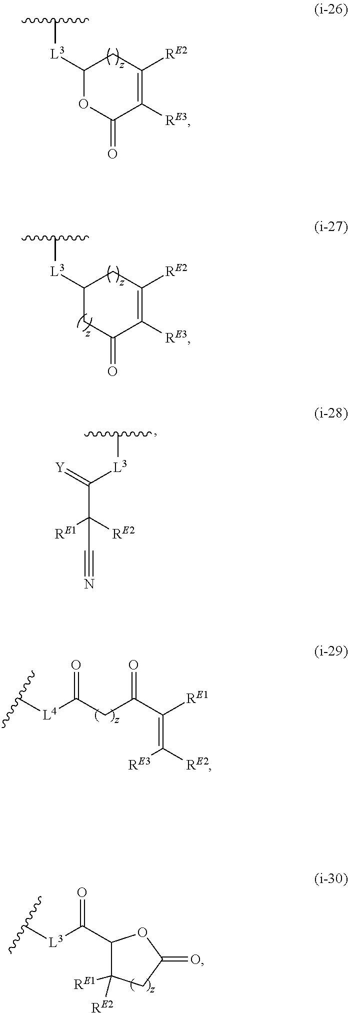

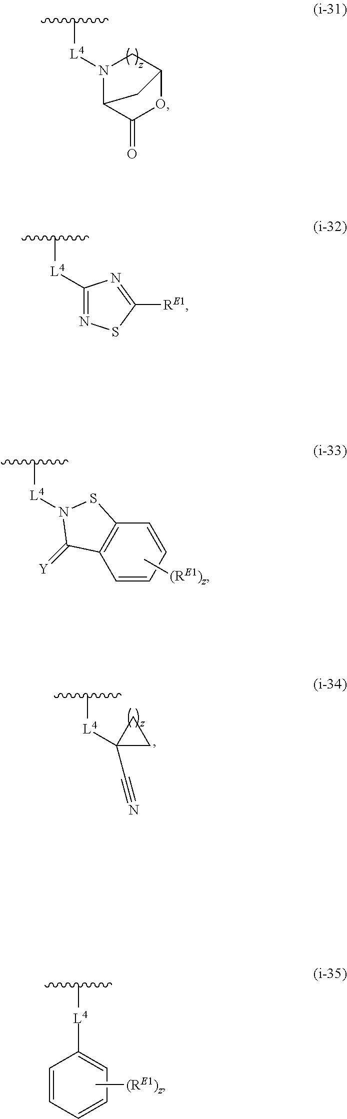

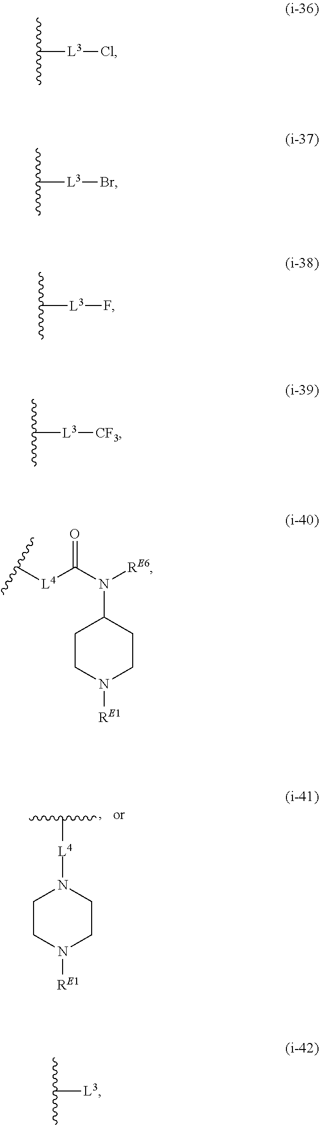

wherein:
L³ is a bond or an optionally substituted C₁₋₄ hydrocarbon chain, optionally wherein one or more carbon units of the hydrocarbon chain are independently replaced with —C═O—, —O—, —S—, —NR^{L3a}—, —NR^{L3a}C(═O)—, —C(═O)NR^{L3a}—, —SC(═O)—, —C(═O)S—, —OC(═O)—, —C(═O)O—, —NR^{L3a}C(═S)—, —C(═S)NR^{L3a}—, trans CR^{L3b}═CR^{L3b}—, cis-CR^{L3b}═CR^{L3b}—, —C≡C—, —S(═O)—, —S(═O)O—, —OS(═O)—, —S(═O)NR^{L3a}—, —NR^{L3a}S(═O)—, —S(═O)₂—, —S(═O)₂O—, —OS(═O)₂—, —S(═O)₂NR^{L3a}—, or —NR^{L3a}S(═O)₂—, wherein R^{L3a} is hydrogen, substituted or unsubstituted C₁₋₆ alkyl, or a nitrogen protecting group, and wherein each occurrence of R^{L3b} is independently hydrogen, halogen, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R^{L3b} groups are joined to form an optionally substituted carbocyclic or optionally substituted heterocyclic ring;
L⁴ is a bond or an optionally substituted, branched or unbranched C₁₋₆ hydrocarbon chain;
each of R^{E1}, R^{E2}, and R^{E3} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{EE}, —CH₂N(R^{EE})₂, —CH₂SR^{EE}, —OR^{EE}, —N(R^{EE})₂, —Si(R^{EE})₃, and —SR^{EE}, wherein each occurrence of R^{EE} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl, or two R^{EE} groups are joined to form an optionally substituted heterocyclic ring;
or R^{E1} and R^{E3}, or R^{E2} and R^{E3}, or R^{E1} and R^{E2} are joined to form an optionally substituted carbocyclic or optionally substituted heterocyclic ring;
R^{E4} is a leaving group;
R^{E5} is halogen;
R^{E6} is hydrogen, substituted or unsubstituted C₁₋₆ alkyl, or a nitrogen protecting group;
each instance of Y is independently O, S, or NR^{E7}, wherein R^{E7} is hydrogen, substituted or unsubstituted C₁₋₆ alkyl, or a nitrogen protecting group;
a is 1 or 2; and
each instance of z is independently 0, 1, 2, 3, 4, 5, or 6, as valency permits.

Examples of compounds of Formula (I) include

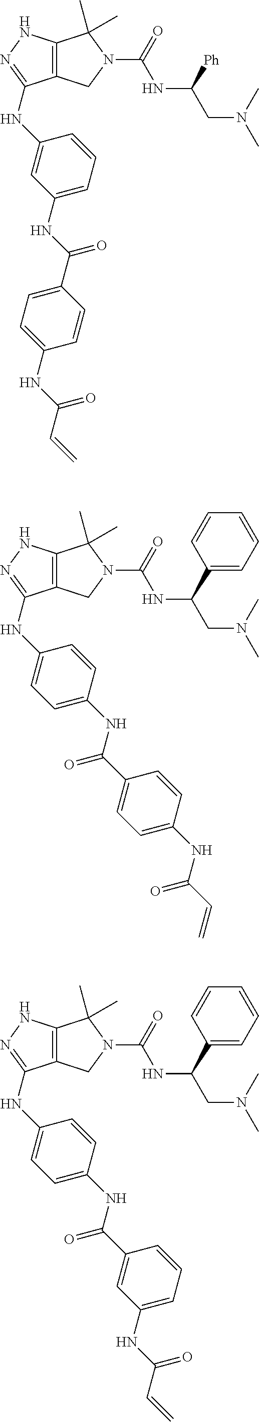 [SMILES: C=CC(=O)Nc1ccc(C(=O)Nc2cccc(Nc3n[nH]c4c3CN(C(=O)N[C@H](CN(C)C)c3ccccc3)C4(C)C)c2)cc1 ; C=CC(=O)Nc1ccc(C(=O)Nc2ccc(Nc3n[nH]c4c3CN(C(=O)N[C@H](CN(C)C)c3ccccc3)C4(C)C)cc2)cc1 ; C=CC(=O)Nc1cccc(C(=O)Nc2ccc(Nc3n[nH]c4c3CN(C(=O)N[C@H](CN(C)C)c3ccccc3)C4(C)C)cc2)c1]

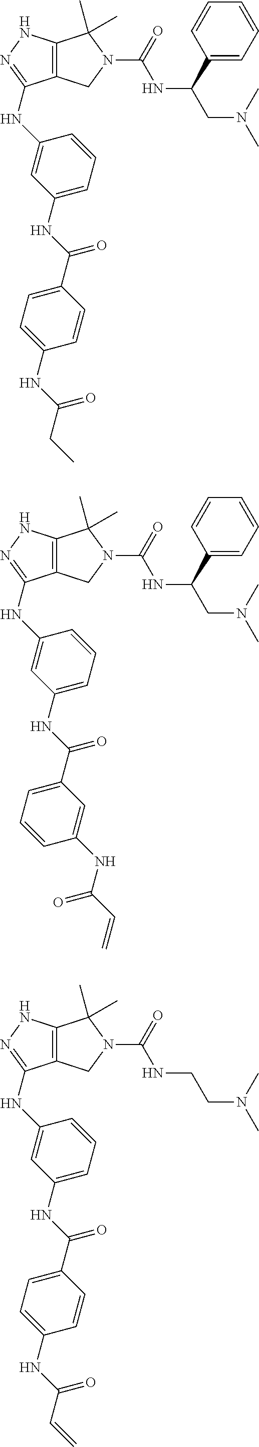

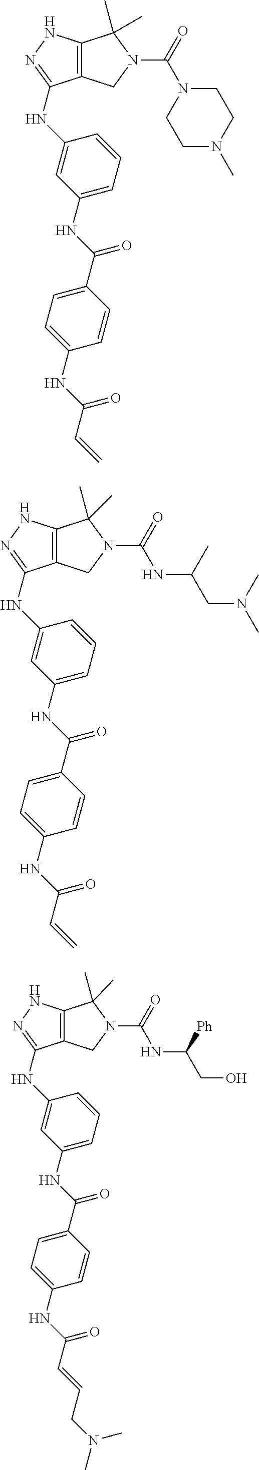

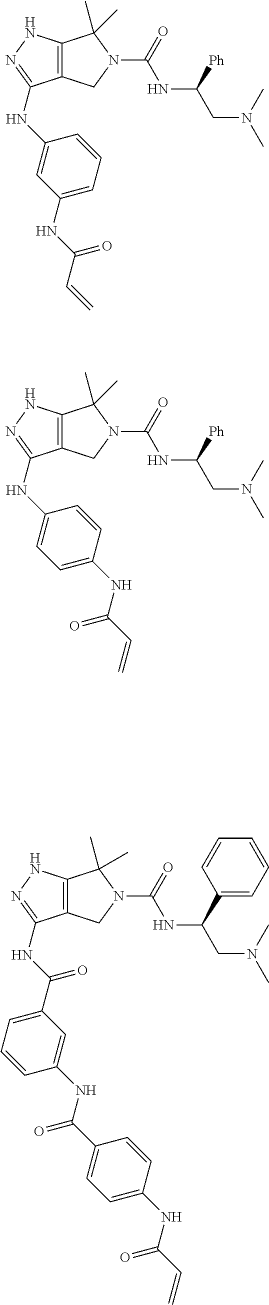 [SMILES: C=CC(=O)Nc1cccc(Nc2n[nH]c3c2CN(C(=O)N[C@H](CN(C)C)c2ccccc2)C3(C)C)c1 ; C=CC(=O)Nc1ccc(Nc2n[nH]c3c2CN(C(=O)N[C@H](CN(C)C)c2ccccc2)C3(C)C)cc1 ; C=CC(=O)Nc1ccc(C(=O)Nc2cccc(C(=O)Nc3n[nH]c4c3CN(C(=O)N[C@H](CN(C)C)c3ccccc3)C4(C)C)c2)cc1]

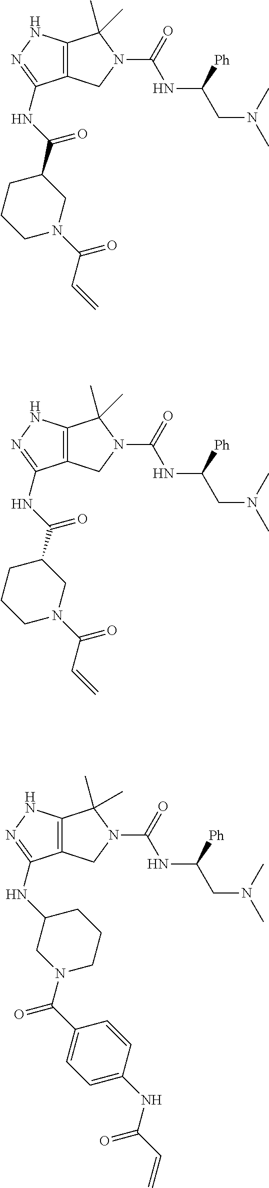 [SMILES: C=CC(=O)N1CCC[C@@H](C(=O)Nc2n[nH]c3c2CN(C(=O)N[C@H](CN(C)C)c2ccccc2)C3(C)C)C1 ; C=CC(=O)N1CCC[C@H](C(=O)Nc2n[nH]c3c2CN(C(=O)N[C@H](CN(C)C)c2ccccc2)C3(C)C)C1 ; C=CC(=O)Nc1ccc(C(=O)N2CCCC(Nc3n[nH]c4c3CN(C(=O)N[C@H](CN(C)C)c3ccccc3)C4(C)C)C2)cc1]

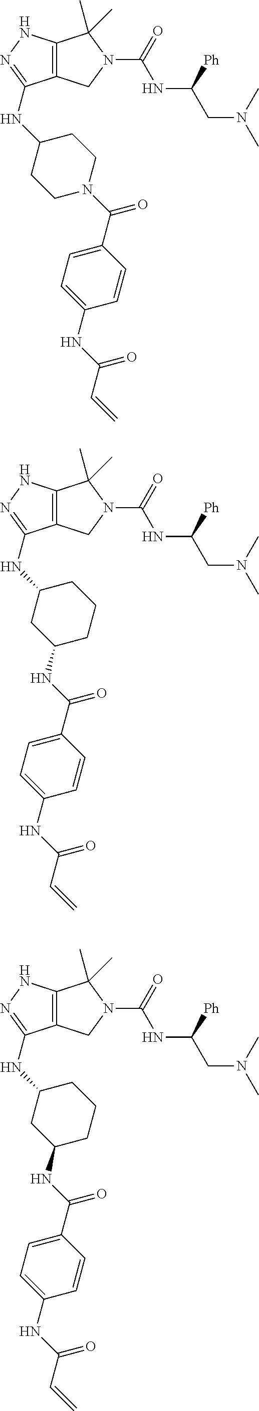

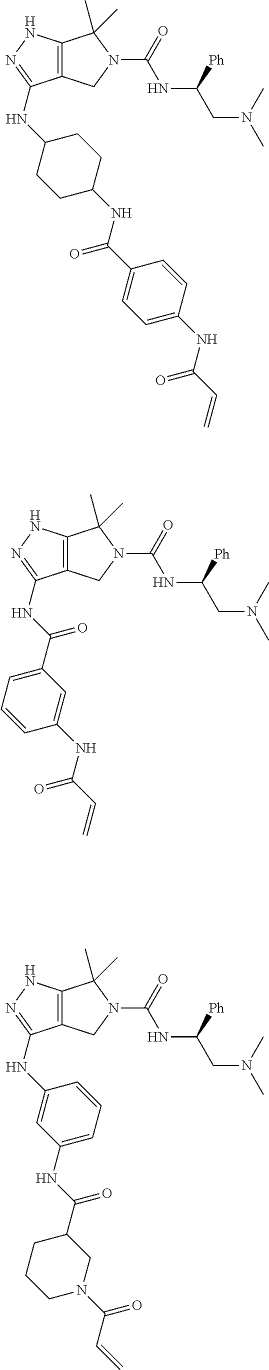 [SMILES: C=CC(=O)Nc1ccc(C(=O)NC2CCC(Nc3n[nH]c4c3CN(C(=O)N[C@H](CN(C)C)c3ccccc3)C4(C)C)CC2)cc1 ; C=CC(=O)Nc1cccc(C(=O)Nc2n[nH]c3c2CN(C(=O)N[C@H](CN(C)C)c2ccccc2)C3(C)C)c1 ; C=CC(=O)N1CCCC(C(=O)Nc2cccc(Nc3n[nH]c4c3CN(C(=O)N[C@H](CN(C)C)c3ccccc3)C4(C)C)c2)C1]

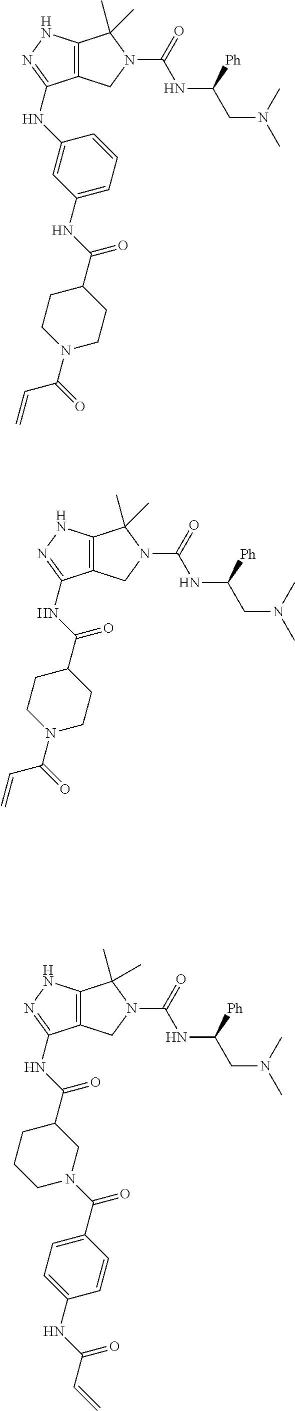

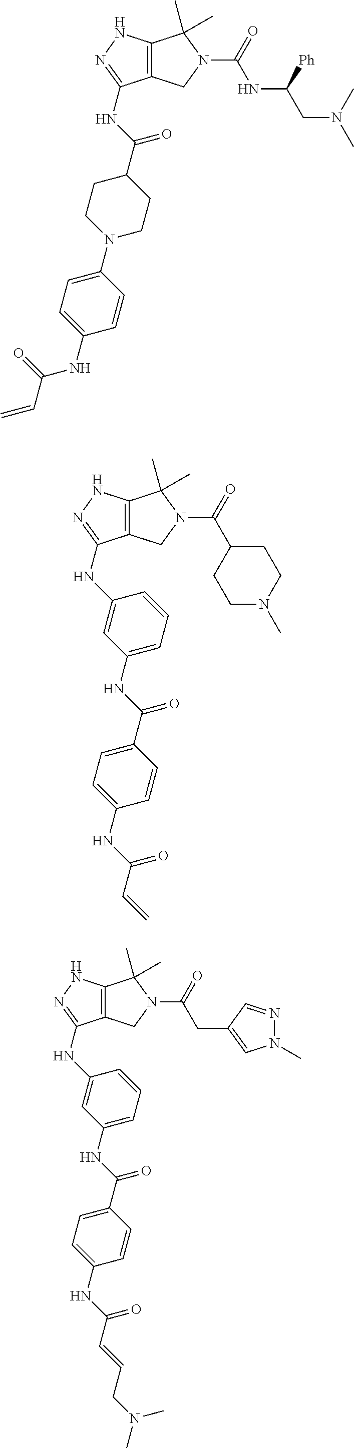

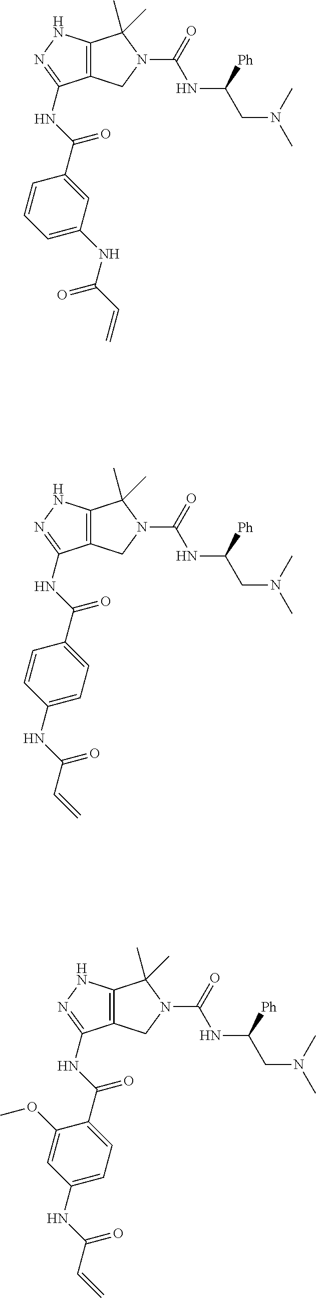

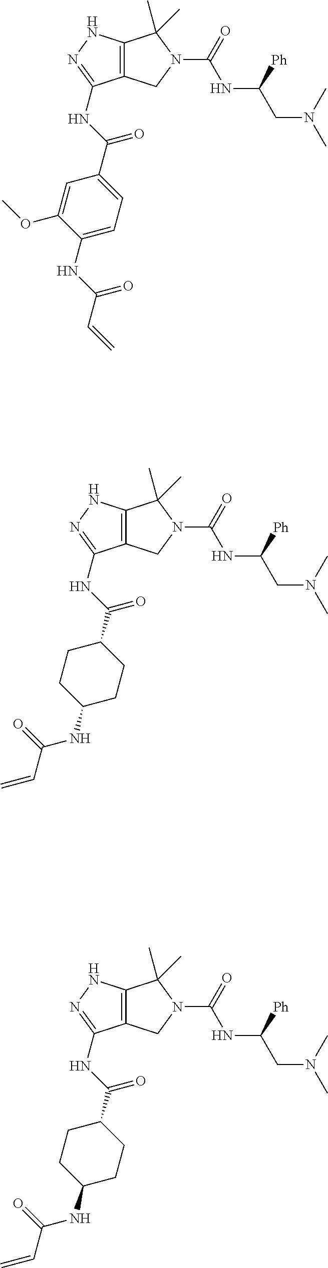

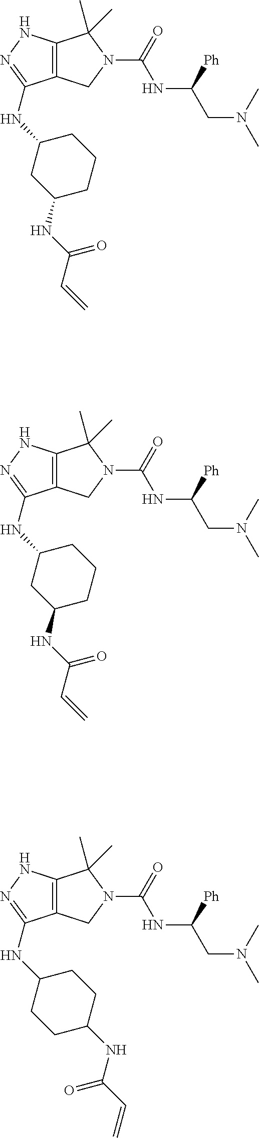 [SMILES: C=CC(=O)N[C@H]1CCC[C@@H](Nc2n[nH]c3c2CN(C(=O)N[C@H](CN(C)C)c2ccccc2)C3(C)C)C1 ; C=CC(=O)N[C@@H]1CCC[C@@H](Nc2n[nH]c3c2CN(C(=O)N[C@H](CN(C)C)c2ccccc2)C3(C)C)C1 ; C=CC(=O)NC1CCC(Nc2n[nH]c3c2CN(C(=O)N[C@H](CN(C)C)c2ccccc2)C3(C)C)CC1]

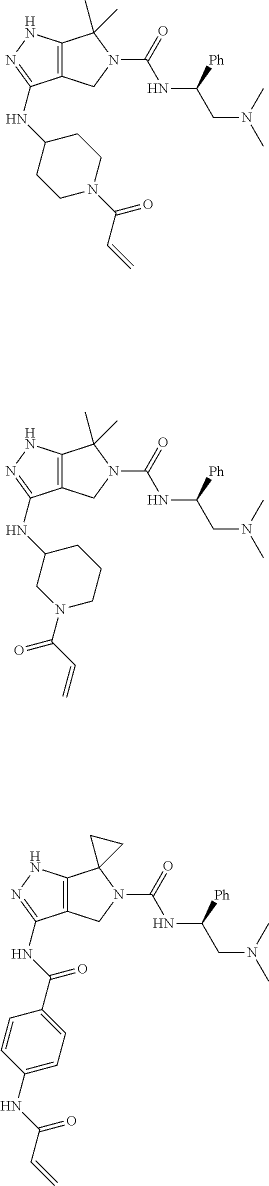 [SMILES: C=CC(=O)N1CCC(Nc2n[nH]c3c2CN(C(=O)N[C@H](CN(C)C)c2ccccc2)C3(C)C)CC1 ; C=CC(=O)N1CCCC(Nc2n[nH]c3c2CN(C(=O)N[C@H](CN(C)C)c2ccccc2)C3(C)C)C1 ; C=CC(=O)Nc1ccc(C(=O)Nc2n[nH]c3c2CN(C(=O)N[C@H](CN(C)C)c2ccccc2)C32CC2)cc1]

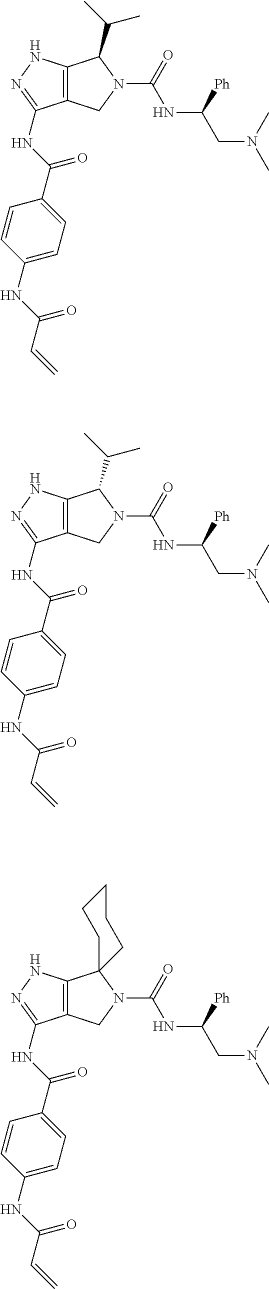 [SMILES: C=CC(=O)Nc1ccc(C(=O)Nc2n[nH]c3c2CN(C(=O)N[C@H](CN(C)C)c2ccccc2)[C@@H]3C(C)C)cc1 ; C=CC(=O)Nc1ccc(C(=O)Nc2n[nH]c3c2CN(C(=O)N[C@H](CN(C)C)c2ccccc2)[C@H]3C(C)C)cc1 ; C=CC(=O)Nc1ccc(C(=O)Nc2n[nH]c3c2CN(C(=O)N[C@H](CN(C)C)c2ccccc2)C32CCCCC2)cc1]

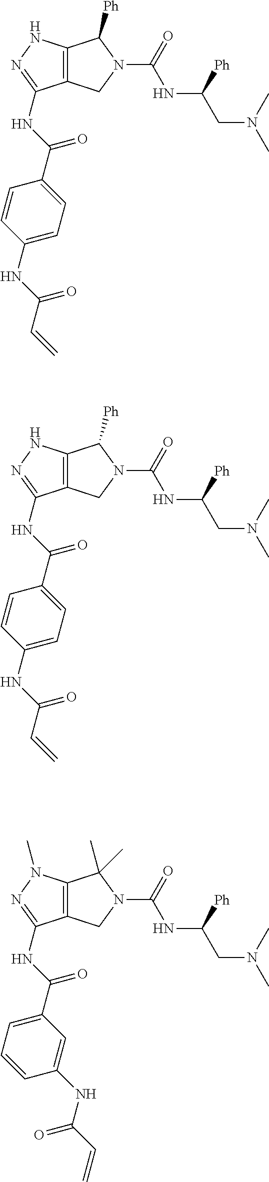 [SMILES: C=CC(=O)Nc1ccc(C(=O)Nc2n[nH]c3c2CN(C(=O)N[C@H](CN(C)C)c2ccccc2)[C@@H]3c2ccccc2)cc1 ; C=CC(=O)Nc1ccc(C(=O)Nc2n[nH]c3c2CN(C(=O)N[C@H](CN(C)C)c2ccccc2)[C@H]3c2ccccc2)cc1 ; C=CC(=O)Nc1cccc(C(=O)Nc2nn(C)c3c2CN(C(=O)N[C@H](CN(C)C)c2ccccc2)C3(C)C)c1]

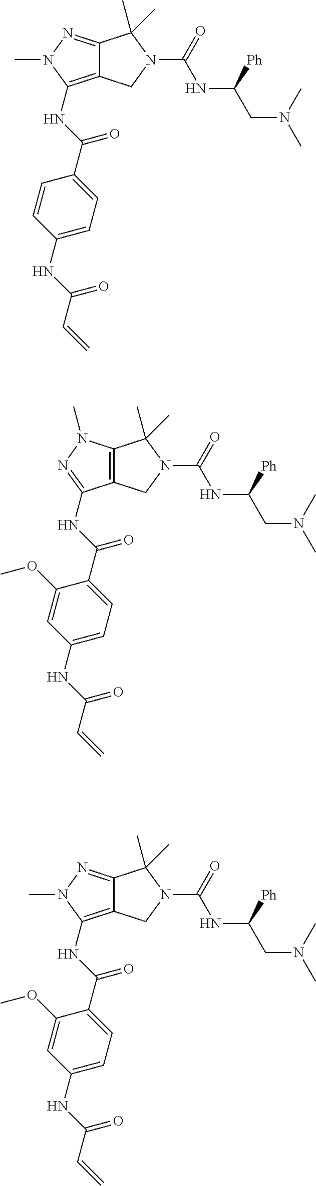

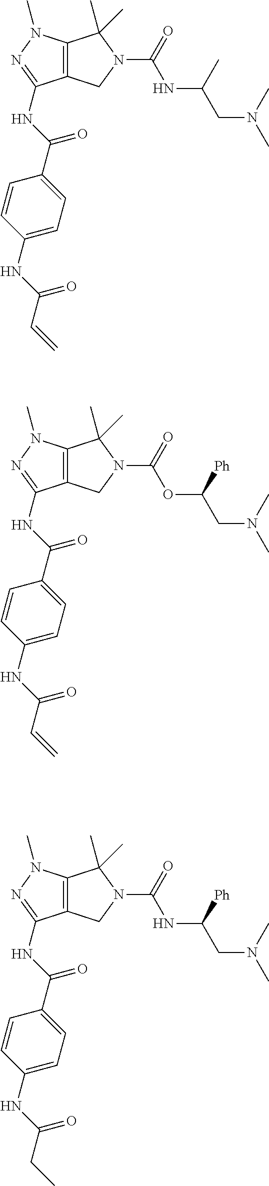 [SMILES: C=CC(=O)Nc1ccc(C(=O)Nc2nn(C)c3c2CN(C(=O)NC(C)CN(C)C)C3(C)C)cc1 ; C=CC(=O)Nc1ccc(C(=O)Nc2nn(C)c3c2CN(C(=O)O[C@H](CN(C)C)c2ccccc2)C3(C)C)cc1 ; CCC(=O)Nc1ccc(C(=O)Nc2nn(C)c3c2CN(C(=O)N[C@H](CN(C)C)c2ccccc2)C3(C)C)cc1]

and pharmaceutically acceptable salts, solvates, hydrates, polymorphs, co-crystals, tautomers, stereoisomers, isotopically labeled derivatives, and prodrugs thereof.

In certain embodiments, the immunotherapy is an immunotherapeutic agent. In certain embodiments, the immunotherapy is an activator of adaptive immune response. In certain embodiments, the immunotherapy is an immune checkpoint inhibitor. In certain embodiments, the immunotherapy is an inhibitor of PD-1, PD-L1, or CTLA-4. In certain embodiments, the immunotherapy is an inhibitor of PD-1. In certain embodiments, the immunotherapy is an anti-PD-1 antibody. In certain embodiments, the immunotherapy is ipilimumab, nivolumab, pembrolizumab, spartalizumab, atezolizumab, tislelizumab, tremelimumab, durvalumab, avelumab, or cemiplimab.

In certain embodiments, the method further comprises administering one or more chemotherapeutic agent (e.g., cisplatin, etoposide). In certain embodiments, the method further comprises administering one or more targeted agents.

In certain embodiments, the cancer is bladder cancer, esophageal cancer, stomach cancer, skin cancer, throat cancer, or lung cancer. In certain embodiments, the cancer is small cell lung cancer.

In another aspect, disclosed is a pharmaceutical composition comprising a CDK7 inhibitor and an immunotherapeutic agent, and optionally a pharmaceutically acceptable excipient. In certain embodiments, the pharmaceutical composition further comprises one or more chemotherapeutic agent (e.g., cisplatin or etoposide).

In another aspect, disclosed is a kit comprising a CDK7 inhibitor and an immunotherapeutic agent and instructions for using the kit.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

Compounds (e.g., CDK7 inhibitors and immunotherapeutic agents) described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The disclosure additionally encompasses compounds described herein as individual isomers substantially free of other isomers, and alternatively, as mixtures of various isomers.

When a range of values is listed, it is intended to encompass each value and sub-range within the range. For example “C₁₋₆” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

The term “aliphatic” includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, the term “alkyl” includes straight, branched and cyclic alkyl groups. An analogous convention applies to other generic terms such as “alkenyl”, “alkynyl”, and the like. Furthermore, the terms “alkyl”, “alkenyl”, “alkynyl”, and the like encompass both substituted and unsubstituted groups. In certain embodiments, “lower alkyl” is used to indicate those alkyl groups (cyclic, acyclic, substituted, unsubstituted, branched or unbranched) having 1-6 carbon atoms.

In certain embodiments, the alkyl, alkenyl, and alkynyl groups employed in the disclosure contain 1-20 aliphatic carbon atoms. In certain other embodiments, the alkyl, alkenyl, and alkynyl groups employed in the disclosure contain 1-10 aliphatic carbon atoms. In yet other embodiments, the alkyl, alkenyl, and alkynyl groups employed in the disclosure contain 1-8 aliphatic carbon atoms. In still other embodiments, the alkyl, alkenyl, and alkynyl groups employed in the disclosure contain 1-6 aliphatic carbon atoms. In yet other embodiments, the alkyl, alkenyl, and alkynyl groups employed in the disclosure contain 1-4 carbon atoms. Illustrative aliphatic groups thus include, but are not limited to, for example, methyl, ethyl, n-propyl, isopropyl, cyclopropyl, —CH₂-cyclopropyl, vinyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl, n-hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl, and the like.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

“Alkenyl” refers to a radical of a straight-chain or branched hydrocarbon group having from 2 to 20 carbon atoms, one or more carbon-carbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl. In an alkenyl group, a C═C double bond for which the stereochemistry is not specified (e.g., —CH═CHCH₃ or

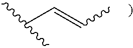

may be an (E)- or (Z)-double bond.

“Alkynyl” refers to a radical of a straight-chain or branched hydrocarbon group having from 2 to 20 carbon atoms, one or more carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

“Carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclic ring, as defined above, is fused with one or more aryl or heteroaryl groups wherein the point of attachment is on the carbocyclic ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system, such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclic ring, as defined above, is fused with one or more carbocyclyl groups wherein the point of attachment is either on the carbocyclyl or heterocyclic ring, or ring systems wherein the heterocyclic ring, as defined above, is fused with one or more aryl or heteroaryl groups, wherein the point of attachment is on the heterocyclic ring, and in such instances, the number of ring members continue to designate the number of ring members in the heterocyclic ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiiranyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Aralkyl” is a subset of alkyl and aryl and refers to an optionally substituted alkyl group substituted by an optionally substituted aryl group. In certain embodiments, the aralkyl is optionally substituted benzyl. In certain embodiments, the aralkyl is benzyl. In certain embodiments, the aralkyl is optionally substituted phenethyl. In certain embodiments, the aralkyl is phenethyl.

“Heteroaryl” refers to a radical of a 5-10 membered monocyclic or bicyclic 4n+2 aromatic ring system (e.g., having 6 or 10 pi electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl, and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Heteroaralkyl” is a subset of alkyl and heteroaryl and refers to an optionally substituted alkyl group substituted by an optionally substituted heteroaryl group.

“Unsaturated” or “partially unsaturated” refers to a group that includes at least one double or triple bond. A “partially unsaturated” ring system is further intended to encompass rings having multiple sites of unsaturation, but is not intended to include aromatic groups (e.g., aryl or heteroaryl groups). Likewise, “saturated” refers to a group that does not contain a double or triple bond, i.e., contains all single bonds.

Alkyl, alkenyl, alkynyl, carbocyclyl, heterocyclyl, aryl, and heteroaryl groups, which are divalent bridging groups, are further referred to using the suffix -ene, e.g., alkylene, alkenylene, alkynylene, carbocyclylene, heterocyclylene, arylene, and heteroarylene.

An atom, moiety, or group described herein may be unsubstituted or substituted, as valency permits, unless otherwise provided expressly. The term “optionally substituted” refers to substituted or unsubstituted.

A group is optionally substituted unless expressly provided otherwise. The term “optionally substituted” refers to being substituted or unsubstituted. In certain embodiments, alkyl, alkenyl, alkynyl, carbocyclyl, heterocyclyl, aryl, and heteroaryl groups are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. In certain embodiments, the substituent is a carbon atom substituent. In certain embodiments, the substituent is a nitrogen atom substituent. In certain embodiments, the substituent is an oxygen atom substituent. In certain embodiments, the substituent is a sulfur atom substituent.

Exemplary carbon atom substituents include, but are not limited to, halogen, —CN, —NO₂, —N₃, —SO₂H, —SO₃H, —OH, —OR—, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{bb}, —OCO₂R^{bb}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{cc})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁺, —P(OR^{cc})₃⁺X⁻, —P(R^{cc})₄, —P(OR^{cc})₄, —OP(R^{cc})₂, —OP(R^{aa})₃⁺X⁻, —OP(OR^{cc})₂, —OP(OR^{aa})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 Rad groups; wherein X⁻ is a counterion;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion.

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ee})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC¹⁻⁶ alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;

each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃—, —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆ alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

“Halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{aa})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R—, —NR^{bb}CO₂R^{aa}, —NR^{bb}C(═O)N(R^{bb})₂, —NR^{bb}C(═NR^{bb})N(R^{bb})₂, —NR^{bb}SO₂R—, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

“Acyl” refers to a moiety selected from the group consisting of —C(═O)R^{aa}, —CHO, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —C(═O)NR^{bb}SO₂R^{aa}, —C(═S)N(R^{bb})₂, —C(═O)SR^{aa}, or —C(═S)SR^{aa}, wherein R^{aa} and R^{bb} are as defined herein.

The term “carbonyl” refers a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on a nitrogen atom is a nitrogen protecting group (also referred to as an amino protecting group). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{aa}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Teroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{aa})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, t-butyloxycarbonyl (BOC or Boc), methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{aa})₃⁺X⁻, —P(OR^{cc})₂, —P(OR^{aa})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

A “leaving group” (LG) is an art-understood term referring to a molecular fragment that departs with a pair of electrons in heterolytic bond cleavage, wherein the molecular fragment is an anion or neutral molecule. As used herein, a leaving group can be an atom or a group capable of being displaced by a nucleophile. See, for example, Smith, March Advanced Organic Chemistry 6th ed. (501-502). Exemplary leaving groups include, but are not limited to, halo (e.g., chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{aa})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂ wherein R^{aa}, R^{bb}, and R^{cc} are as defined herein).

A “hydrocarbon chain” refers to a substituted or unsubstituted divalent alkyl, alkenyl, or alkynyl group. A hydrocarbon chain includes (1) one or more chains of carbon atoms immediately between the two radicals of the hydrocarbon chain; (2) optionally one or more hydrogen atoms on the chain(s) of carbon atoms; and (3) optionally one or more substituents (“non-chain substituents,” which are not hydrogen) on the chain(s) of carbon atoms. A chain of carbon atoms consists of consecutively connected carbon atoms (“chain atoms”) and does not include hydrogen atoms or heteroatoms. However, a non-chain substituent of a hydrocarbon chain may include any atoms, including hydrogen atoms, carbon atoms, and heteroatoms. For example, hydrocarbon chain —C^{A}H(C^{B}H₂C^{C}H₃)— includes one chain atom C^{A}, one hydrogen atom on C^{A}, and non-chain substituent—(C^{B}H₂C^{C}H₃). The term “Cₓ hydrocarbon chain,” wherein x is a positive integer, refers to a hydrocarbon chain that includes x number of chain atom(s) between the two radicals of the hydrocarbon chain. If there is more than one possible value of x, the smallest possible value of x is used for the definition of the hydrocarbon chain. For example, —CH(C₂H₅)— is a C₁ hydrocarbon chain, and

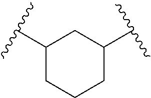 [SMILES: *C1CCCC(*)C1]

is a C₃ hydrocarbon chain. When a range of values is used, the meaning of the range is as described herein. For example, a C₃₋₁₀ hydrocarbon chain refers to a hydrocarbon chain where the number of chain atoms of the shortest chain of carbon atoms immediately between the two radicals of the hydrocarbon chain is 3, 4, 5, 6, 7, 8, 9, or 10. A hydrocarbon chain may be saturated (e.g., —(CH₂)₄—). A hydrocarbon chain may also be unsaturated and include one or more C═C and/or C≡C bonds anywhere in the hydrocarbon chain. For instance, —CH═CH—(CH₂)₂—, —CH₂—C≡C—CH₂—, and —C≡C—CH═CH— are all examples of a unsubstituted and unsaturated hydrocarbon chain. In certain embodiments, the hydrocarbon chain is unsubstituted (e.g., —C≡C— or —(CH₂)₄—). In certain embodiments, the hydrocarbon chain is substituted (e.g., —CH(C₂H₅)— and —CF₂—). Any two substituents on the hydrocarbon chain may be joined to form an optionally substituted carbocyclyl, optionally substituted heterocyclyl, optionally substituted aryl, or optionally substituted heteroaryl ring. For instance,

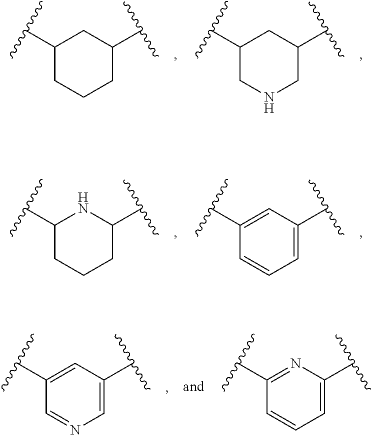 [SMILES: *C1CCCC(*)C1 ; *C1CNCC(*)C1 ; *C1CCCC(*)N1 ; *c1cccc(*)c1 ; *c1cncc(*)c1 ; *c1cccc(*)n1]

are all examples of a hydrocarbon chain. In contrast, in certain embodiments,

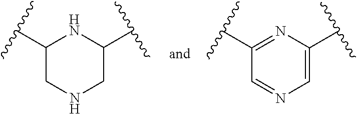 [SMILES: *C1CNCC(*)N1 ; *c1cncc(*)n1]

are not within the scope of the hydrocarbon chains described herein. When a chain atom of a Cₓ hydrocarbon chain is replaced with a heteroatom, the resulting group is referred to as a Cₓ hydrocarbon chain wherein a chain atom is replaced with a heteroatom, as opposed to a Cₓ₋₁ hydrocarbon chain. For example,

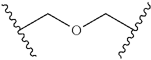 [SMILES: *COC*]

is a C₃ hydrocarbon chain wherein one chain atom is replaced with an oxygen atom.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated herein by reference. Pharmaceutically acceptable salts of the compounds described herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R.x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R.0.5 H₂O)), and polyhydrates (x is a number greater than 1, e.g., dihydrates (R.2 H₂O) and hexahydrates (R.6 H₂O)).

The term “tautomers” or “tautomeric” refers to two or more interconvertable compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction interconverting a tautomeric pair) may be catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorphs” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁-C₈ alkyl, C₂-C₈ alkenyl, C₂-C₈ alkynyl, aryl, C₇-C₁₂ substituted aryl, and C₇-C₁₂ arylalkyl esters of the compounds described herein may be preferred.

The term “small molecule” refers to molecules, whether naturally-occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention.

A “protein,” “peptide,” or “polypeptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

The term “inhibit” or “inhibition” in the context of modulating level (e.g., expression and/or activity) of a target (e.g., CDK7) is not limited to only total inhibition. Thus, in some embodiments, partial inhibition or relative reduction is included within the scope of the term “inhibition.” In some embodiments, the term refers to a reduction of the level (e.g., expression, and/or activity) of a target (e.g., CDK7) to a level that is reproducibly and/or statistically significantly lower than an initial or other appropriate reference level, which may, for example, be a baseline level of a target. In some embodiments, the term refers to a reduction of the level (e.g., expression and/or activity) of a target to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of a target.

As used herein, the term “inhibitor” refers to an agent whose presence or level correlates with decreased level or activity of a target to be modulated. In some embodiments, an inhibitor may act directly (in which case it exerts its influence directly upon its target, for example by binding to the target); in some embodiments, an inhibitor may act indirectly (in which case it exerts its influence by interacting with and/or otherwise altering a regulator of a target, so that level and/or activity of the target is reduced). In some embodiments, an inhibitor is one whose presence or level correlates with a target level or activity that is reduced relative to a particular reference level or activity (e.g., that observed under appropriate reference conditions, such as presence of a known inhibitor, or absence of the inhibitor as disclosed herein, etc.).

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. A “patient” refers to a human subject in need of treatment of a disease. The subject may also be a plant. In certain embodiments, the plant is a land plant. In certain embodiments, the plant is a non-vascular land plant. In certain embodiments, the plant is a vascular land plant. In certain embodiments, the plant is a seed plant. In certain embodiments, the plant is a cultivated plant. In certain embodiments, the plant is a dicot. In certain embodiments, the plant is a monocot. In certain embodiments, the plant is a flowering plant. In some embodiments, the plant is a cereal plant, e.g., maize, corn, wheat, rice, oat, barley, rye, or millet. In some embodiments, the plant is a legume, e.g., a bean plant, e.g., soybean plant. In some embodiments, the plant is a tree or shrub.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The terms “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay and/or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

A “prophylactically effective amount” of a compound described herein is an amount effective to prevent a condition, or one or more symptoms associated with the condition and/or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers include, but are not limited to, hematological malignancies. The term “hematological malignancy” refers to tumors that affect blood, bone marrow, and/or lymph nodes. Exemplary hematological malignancies include, but are not limited to, leukemia, such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma, such as Hodgkin lymphoma (HL) (e.g., B-cell HL, T-cell HL) and non-Hodgkin lymphoma (NHL) (e.g., B-cell NHL, such as diffuse large cell lymphoma (DLCL) (e.g., diffuse large B-cell lymphoma (DLBCL, e.g., activated B-cell (ABC) DLBCL (ABC-DLBCL))), follicular lymphoma, chronic lymphocytic leukemia/small lymphocytic lymphoma (CLL/SLL), mantle cell lymphoma (MCL), marginal zone B-cell lymphoma (e.g., mucosa-associated lymphoid tissue (MALT) lymphoma, nodal marginal zone B-cell lymphoma, splenic marginal zone B-cell lymphoma), primary mediastinal B-cell lymphoma, Burkitt lymphoma, Waldenstram's macroglobulinemia (WM, lymphoplasmacytic lymphoma), hairy cell leukemia (HCL), immunoblastic large cell lymphoma, precursor B-lymphoblastic lymphoma, central nervous system (CNS) lymphoma (e.g., primary CNS lymphoma and secondary CNS lymphoma); and T-cell NHL, such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); lymphoma of an immune privileged site (e.g., cerebral lymphoma, ocular lymphoma, lymphoma of the placenta, lymphoma of the fetus, testicular lymphoma); a mixture of one or more leukemia/lymphoma as described above; myelodysplasia; and multiple myeloma (MM). Additional exemplary cancers include, but are not limited to, lung cancer (e.g., bronchogenic carcinoma, small cell lung cancer (SCLC), non-small cell lung cancer (NSCLC), adenocarcinoma of the lung); kidney cancer (e.g., nephroblastoma, a.k.a. Wilms' tumor, renal cell carcinoma); acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); heavy chain disease (e.g., alpha chain disease, gamma chain disease, mu chain disease; hemangioblastoma; hypopharynx cancer; inflammatory myofibroblastic tumors; immunocytic amyloidosis; liver cancer (e.g., hepatocellular cancer (HCC), malignant hepatoma); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “immunotherapy” refers to a treatment of disease by inducing, enhancing, or suppressing an immune response. Immunotherapies designed to elicit or amplify an immune response are classified as activation immunotherapies, while immunotherapies that reduce or suppress an immune response are classified as suppression immunotherapies. Immunotherapy may encompass treatment with a molecular entity (e.g., immunotherapeutic agent) and/or a non-molecular entity (e.g., adoptive cell transfer)

The term “immunotherapeutic agent” refers to a molecular entity that induces, enhances, or suppresses an immune response. Immunotherapeutic agents include, but are not limited to, monoclonal antibodies, cytokines, chemokines, vaccines, small molecule inhibitors, and small molecule agonists. For example, useful immunotherapeutic agents may include, but are not limited to, inducers of type I interferon, interferons, stimulator of interferon genes (STING) agonists, TLR7/8 agonists, IL-15 superagonists, anti-PD-1 antibodies, anti-CD137 antibodies, and anti-CTLA-4 antibodies.

The term “immune checkpoint inhibitor” refers to an agent that blocks certain proteins made by some types of immune system cells, such as T cells, and some cancer cells. These proteins help keep immune responses in check and can keep T cells from killing cancer cells. When these proteins are blocked, immune system function is restored and the immune system is released enabling T cells to kill cancer cells. Examples of checkpoint proteins found on T cells or cancer cells include CD27, CD28, CD40, CD122, CD137, OX40, GITR, ICOS (CD278), A2AR, B7-H₃, B7-H₄, BTLA, CTLA-4, IDO, KIR, LAG3, NOX2, PD-1, PD-L1, TIM-3, VISTA, and SIGLEC7. Some immune checkpoint inhibitors are useful in treating cancer.

The terms “biologic,” “biologic drug,” and “biological product” refer to a wide range of products such as vaccines, blood and blood components, allergenics, somatic cells, gene therapy, tissues, nucleic acids, and proteins. Biologics may include sugars, proteins, or nucleic acids, or complex combinations of these substances, or may be living entities such as cells and tissues. Biologics may be isolated from a variety of natural sources (e.g., human, animal, microorganism) and/or may be produced by biotechnological methods and/or other technologies.

The term “antibody” refers to a functional component of serum and is often referred to either as a collection of molecules (antibodies or immunoglobulins) or as one molecule (the antibody molecule or immunoglobulin molecule). An antibody is capable of binding to or reacting with a specific antigenic determinant (the antigen or the antigenic epitope), which in turn may lead to induction of immunological effector mechanisms. An individual antibody is usually regarded as monospecific, and a composition of antibodies may be monoclonal (i.e., consisting of identical antibody molecules) or polyclonal (i.e., consisting of two or more different antibodies reacting with the same or different epitopes on the same antigen or even on distinct, different antigens). Each antibody has a unique structure that enables it to bind specifically to its corresponding antigen, and all natural antibodies have the same overall basic structure of two identical light chains and two identical heavy chains. Antibodies are also known collectively as immunoglobulins. An antibody may be of human or non-human (for example, rodent such as murine, dog, camel, etc) origin (e.g., may have a sequence originally developed in a human or non-human cell or organism), or may be or comprise a chimeric, humanized, reshaped, or reformatted antibody based, e.g., on a such a human or non-human antibody (or, in some embodiments, on an antigen-binding portion thereof).

In some embodiments, as will be clear from context, the term “antibody” as used herein encompasses formats that include epitope-binding sequences of an antibody, which such formats include, for example chimeric and/or single chain antibodies (e.g., a nanobody or Fcab), as well as binding fragments of antibodies, such as Fab, Fv fragments or single chain Fv (scFv) fragments, as well as multimeric forms such as dimeric IgA molecules or pentavalent IgM molecules. Also included are bispecific antibodies, bispecific T cell engagers (BiTEs), immune mobilixing monoclonal T cell receptors against cancer (ImmTACs), dual-affinity re-targeting (DART); alternative scaffolds or antibody mimetics (e.g., anticalins, FN3 monobodies, DARPins, Affibodies, Affilins, Affimers, Affitins, Alphabodies, Avimers, Fynomers, Im7, VLR, VNAR, Trimab, CrossMab, Trident); nanobodies, binanobodies, F(ab′)₂, Fab′, di-sdFv, single domain antibodies, trifunctional antibodies, diabodies, and minibodies.

The term “therapeutic agent” refers to an agent having one or more therapeutic properties that produce a desired, usually beneficial, effect. For example, a therapeutic agent may treat, ameliorate, and/or prevent disease. In some embodiments, a therapeutic agent may be or comprise a biologic, a small molecule, or a combination thereof.

The term “chemotherapeutic agent” refers to a therapeutic agent known to be of use in chemotherapy for cancer.

The term “targeted agent” refers to an anticancer agent that blocks the growth and spread of cancer by interfering with specific molecules (“molecular targets”) that are involved in the growth, progression, and spread of cancer. Targeted agents are sometimes called “targeted cancer therapies,” “molecularly targeted drugs,” “molecularly targeted therapies,” or “precision medicines.” Targeted agents differ from standard chemotherapy in that targeted agents act on specific molecular targets that are associated with cancer, whereas many chemotherapeutic agents act on all rapidly dividing cells (e.g., whether or not the cells are cancerous). Targeted agents are deliberately chosen or designed to interact with their target, whereas many standard chemotherapies are identified because they kill cells.

The term “antagonist” refers to an agent that (i) decreases or suppresses one or more effects of another agent; and/or (ii) decreases or suppresses one or more biological events. In some embodiments, an antagonist may reduce level and/or activity or one or more agents that it targets. In various embodiments, antagonists may be or include agents of various chemical class including, for example, small molecules, polypeptides, nucleic acids, carbohydrates, lipids, metals, and/or other entity that shows the relevant antagonistic activity. An antagonist may be direct (in which case it exerts its influence directly upon its target) or indirect (in which case it exerts its influence by other than binding to its target; e.g., by interacting with a regulator of the target, for example so that level or activity of the target is altered). In some embodiments, an antagonist may be a receptor antagonist, e.g., a receptor ligand or drug that blocks or dampens a biological response by binding to and blocking a receptor rather than activating it like an agonist.

The term “agonist” refers to an agent that (i) increases or induces one or more effects of another agent; and/or (ii) increases or induces one or more biological events. In some embodiments, an agonist may increase level and/or activity or one or more agents that it targets. In various embodiments, agonists may be or include agents of various chemical class including, for example, small molecules, polypeptides, nucleic acids, carbohydrates, lipids, metals, and/or other entity that shows the relevant agonistic activity. An agonist may be direct (in which case it exerts its influence directly upon its target) or indirect (in which case it exerts its influence by other than binding to its target; e.g., by interacting with a regulator of the target, for example so that level or activity of the target is altered). A partial agonist can act as a competitive antagonist in the presence of a full agonist, as it competes with the full agonist to interact with its target and/or a regulator thereof, thereby producing (i) a decrease in one or more effects of another agent, and/or (ii) a decrease in one or more biological events, as compared to that observed with the full agonist alone.

The term “activator of innate immune response” refers to an agent that activates the innate immune system. Such activation can stimulate the expression of molecules that initiate an inflammatory response and/or help to induce adaptive immune responses, leading to the development of antigen-specific acquired immunity. Activation of the innate immune system can lead to cytokine production, proliferation, and survival as well as improved T cell priming by enhancing presentation of antigens and expression of co-stimulatory molecules by antigen-presenting cells.

The term “activator of adaptive immune response” refers to an agent that activates the adaptive immune system. Such activation can restore antitumor function by neutralizing inhibitory immune checkpoints or by triggering co-stimulatory receptors, ultimately generating helper and/or effector T cell responses against immunogenic antigens expressed by cancer cells and producing memory B cell and/or T cell populations. In certain embodiments, the activator of adaptive immune response involves modulation of adaptive immune response and/or leukocyte trafficking.

The term “CDK” refers to a cyclin-dependent kinase. A CDK binds a cyclin (e.g., Cyclin H), which is a regulatory protein. CDKs phosphorylate their substrates at serines and threonines. The consensus sequence for the phosphorylation site in the amino acid sequence of a CDK substrate is [S/T*]PX[K/R], where S/T* is the phosphorylated serine or threonine, P is proline, X is any amino acid, K is lysine, and R is arginine. CDKs include CDK1, CDK2, CDK3, CDK4, CDK5, CDK6, CDK7, CDK8, CDK9, CDK10, CDK11, CDK12, CDK13, CDK14, CDK15, CDK16, CDK17, CDK18, CDK19 and CDK20.

CDK7, cyclin-dependent kinase 7, is a CDK, wherein the substrate is Cyclin H, MAT1 (e.g., MNAT1), or Cyclin H and MAT1. CDK7 is alternatively referred to as CAK1, HCAK, M015, STK1, CDKN7, and p39MO15. Non-limiting examples of the nucleotide and protein sequences for human CDK7 are described in GenBank Accession Number NP_001790, incorporated herein by reference. The amino acid sequence of this CDK7 is as follows:

MALDVKSRAKRYEKLDFLGEGQFATVYKARDKNT
NQIVAIKKIKLGHRSEAKDGINRTALREIKLLQE
LSHPNIIGLLDAFGHKSNISLVFDFMETDLEVII
KDNSLVLTPSHIKAYMLMTLQGLEYLHQHWILHR
DLKPNNLLLDENGVLKLADFGLAKSFGSPNRAYT
HQVVTRWYRAPELLFGARMYGVGVDMWAVGCILA
ELLLRVPFLPGDSDLDQLTRIFETLGTPTEEQWP
DMCSLPDYVTFKSFPGIPLHHIFSAAGDDLLDLI
QGLFLFNPCARITATQALKMKYFSNRPGPTPGCQ
LPRPNCPVETLKEQSNPALAIKRKRTEALEQGGL
PKKLIF

### BRIEF DESCRIPTION OF THE DRAWINGS

FIGS. 1A to 1F: YKL-5-124 specifically targets CDK7 and disrupts cell-cycle progression through inhibition of CDK7 CAK activity. FIG. 1A shows results of a competitive pull-down assay in mouse SCLC (mSCLC) RPP631 cells treated with YKL-5-124 at indicated concentrations for 6 hours. Western blotting showing the pull-down (PD) or input of Cyclin H and Cyclin K. FIG. 1B shows western blotting of RNA Pol II total (RNAP II), RNA Pol II p-Ser 2 and 5, CDK1, CDK2, pCDK1 (Thrl61), pCDK2 (Thr160), and Tubulin in RPP631 and human SCLC (hSCLC) DMS79 cells after treatment with YKL-5-124 at indicated concentrations for 24 hours. FIG. 1C shows cell viability measured at indicated time points (normalized to day 0) upon treatment with DMSO or increasing concentrations of YKL-5-124 in RPP631 and DMS79. FIG. 1D is a bar graph showing the cell distribution in G₁, S, and G₂/M phases quantified by flow-cytometry analysis of propidium iodide (PI) staining in RPP631 and DMS79 after YKL-5-124 treatment for 72 hours. FIG. 1E shows western blotting analysis of Cyclin E and Tubulin levels in RPP631 and DMS79 after treatment with YKL-5-124 at indicated concentrations for 24 hours. FIG. 1F shows qRT-PCR analysis of CCNE1, CCNB1, CCND1, and CCNA1 gene expression in RPP631 and DMS79 after treatment for 24 hours. The data are presented as fold changes compared with the vehicle (DMSO). *p<0.05 (unpaired two-tailed t test). In FIG. 1C, FIG. 1D, and FIG. 1F, data are shown as means±SD of three independent experiments run in triplicates.

FIGS. 2A to 2L: CDK7 inhibition impairs DNA synthesis and MCM2 complex and causes DNA damage and micronuclei formation. FIG. 2A shows flow-cytometry analysis of BrdU and 7-AAD co-staining in DMS79 after 24 and 48 hour treatment with DMSO or 100 nM YKL-5-124. FIG. 2B is a bar graph showing the cell distribution in G₁, S, and G₂/M phases. FIGS. 2C-2E show quantification of DNA synthesis indicated by EdU incorporation per nucleus as well as within each replication focus using STORM imaging of fluorescently labeled EdU in RPP631 cells treated with vehicle or 100 nM YKL-5-124 after 72 hours. FIG. 2C shows representative images of nuclei with EdU signal are shown in vehicle or YKL-5-124. Scale bars, 2,000 nm. FIG. 2D shows quantifications of EdU nuclear density (nm⁻²) per nucleus and FIG. 2E shows EdU content per focus are plotted. FIGS. 2F-2H show quantification of MCM2 complex per nucleus as well as within each replication focus in RPP631 cells. FIG. 2F shows representative images of nuclei with MCM2 content are shown. Scale bars, 2,000 nm. Dashed-line circle indicates nuclei. FIG. 2G shows quantification of MCM2 nuclear density (nm⁻²) per nucleus and FIG. 2H shows MCM2 content per focus are plotted. FIGS. 2I and 2J show quantification of γH2AX foci upon YKL-5-124 exposure by immunofluorescence (IF) microscopy in RPP631. FIG. 2I shows representative images of DAPI-stained nuclei in blue and γH2AX foci in red. FIG. 2J shows the percentages of γH2AX foci in cells are plotted. At least ten field images were counted (≥100 cells). FIG. 2K and FIG. 2L show quantification of micronuclei upon YKL-5-124 exposure in RPP631 and GLC16 by IF. FIG. 2K shows representative images of DAPI-stained nuclei. FIG. 2L shows the percentages of micronuclei in cells are plotted. At least ten field images were counted (≥100 cells). In FIG. 2B, FIG. 2D, FIG. 2E, FIG. 2G, FIG. 2H, FIG. 2J, and FIG. 2L, data are shown as means±SD of two to three independent experiments run in triplicates. In FIG. 2D, FIG. 2E, FIG. 2G, FIG. 2H, FIG. 2J, and FIG. 2L, *p<0.05, **p<0.01, ***p<0.001, and ****p<0.0001 (unpaired two-tailed t test).

FIGS. 3A to 3L: YKL-5-124 triggers immune-response signaling and induces pro-inflammatory cytokine and chemokine production. FIGS. 3A-3C show GSEA of the differentially expressed genes induced by YKL-5-124 in RPP631. Shown are three of the top five most positively regulated “hallmark” signatures: interferon-7 response (FIG. 3A), TNF-α signaling (FIG. 3B), and inflammatory response (FIG. 3C). Gene list was ranked with signed (from log₂ fold change [log₂FC]) likelihood ratio from YKL-5-124 versus vehicle comparison. FIGS. 3D-3F show heatmaps for differential expression of transcripts from three top positively regulated pathways (colors are log 2FC). FIGS. 3G-3I show a qRT-PCR analysis of Tnf (FIG. 3G), Cxcl10 (FIG. 3H), and Cxcl9 (FIG. 3I) levels in RPP631. The data are presented as fold changes compared with the vehicle. Data shown as means±SD of three independent experiments run in triplicates. **p<0.01, ****p<0.0001 (unpaired two-tailed t test). FIGS. 3J-3L show profiling of OT-IT cells activation markers CD69 (FIG. 3J), TNF-α (FIG. 3K), and IFN-γ (FIG. 3L) by flow cytometry after treatment with DMSO- or YKL-5-124-conditioned medium. Data are shown as means±SEM of three independent experiments run in ten replicates. *p<0.05, **p<0.01 (unpaired two-tailed t test).

FIGS. 4A to 4G: YKL-5-124 inhibits SCLC tumor growth in vivo and enhances tumor response to anti-PD-1 immunotherapy. FIG. 4A shows quantification of baseline tumor volumes of RPP orthotopic model. Combined vehicle and isotype immunoglobulin G (Control, n=13), anti-PD-1 (n=13), YKL-5-124 (n=17), anti-PD-1+YKL-5-124 (Combo, n=25), chemotherapy (Chemo)+anti-PD-1 (n=18), and Chem+Combo (n=17). Each dot represents one mouse. FIG. 4B show quantification of tumor volume changes of RPP orthotopic model after treatment. Waterfall plot shows tumor volume response after week 3. Each column represents one mouse, in comparison with baseline MRI measurement. FIG. 4C shows representative MR images show lung tumors of RPP orthotopic model before and after the treatment at indicated time points. Circled areas, heart. FIG. 4D shows quantification of baseline tumor volumes of RP orthotopic model: Control (n=9), anti-PD-1 (n=10), YKL-5-124 (n=12), and Combo (n=12). Each dot represents one mouse. FIG. 4E show quantification of tumor volume changes of RP orthotopic model after treatment. Waterfall plot shows tumor volume response after week 2. Each column represents one mouse, in comparison with baseline MRI measurement. FIG. 4F and FIG. 4G show a Kaplan-Meier survival curve of RPP (FIG. 4F) or RP (FIG. 4G) orthotopic model after indicated treatment. *p<0.05, **p<0.01, ***p<0.001, ****p<0.0001 (log-rank test). In FIG. 4A and FIG. 4D, data are shown as means±SEM. In FIG. 4A, FIG. 4B, FIG. 4D, and FIG. 4E, **p<0.01, ***p<0.001, and ****p<0.0001; NS, not significant (unpaired two-tailed t test).

FIGS. 5A to 5K: YKL-5-124 provokes a robust anti-tumor immune program, which is further enhanced by anti-PD-1 immunotherapy. FIG. 5A and FIG. 5B show tumor-infiltrating lymphocytes from RPP orthotopic model were analyzed at day 7 after treatment (n=5). Frequencies of infiltrating CD4⁺ T cells (FIG. 5A) and CD8⁺ T cells (FIG. 5B) are presented. FIGS. 5C-5F show the expression of CD44 (FIG. 5C), CD62L (FIG. 5D), and Ki67 (FIG. 5E) in CD4⁺ T cells; FIG. 5F shows frequencies of ICOS⁺ CD4⁺ T cells were analyzed (n=5). FIG. 5G shows frequencies of GzmB+CD8⁺ T cells were analyzed (n=5). FIG. 5H shows frequencies of CD11c⁺ CD103⁺ dendritic cells were analyzed (n=5). FIGS. 5I-5K show bronchoalveolar lavage fluid (BALF) was collected from mouse lung and secretion of TNF-α (FIG. 5I), CXCL9 (FIG. 5J), and CXCL10 (FIG. 5K) was measured by Luminex (pg/ml) (n=4). Data are shown as means±SD. *p<0.05, **p<0.01, ***p<0.001, ****p<0.0001; NS, not significant (unpaired two-tailed t test).

FIGS. 6A to 6J: Single-cell analysis identifies intratumoral cell populations and confirms connection of CDK7 inhibition in tumor-intrinsic signaling to immunity. FIG. 6A is a UMAP plot showing identified cell populations within whole tumor from all groups merged. FIG. 6B is a cluster dendrogram showing the lineage hierarchy of identified cell populations in FIG. 6A. FIG. 6C is a UMAP plot of cancer and infiltrating cells displaying marker gene expression. FIG. 6D is a UMAP plot showing the cell distribution within identified cell populations upon treatment. FIG. 6E shows a distribution fraction of cancer, immune, and stromal compartments in response to indicated treatment. FIG. 6F shows inferred dynamic phases of cell-cycle progression from scRNA-seq analysis. FIG. 6G is a bar plot showing cellular distribution within the cell-cycle progression states indicated in FIG. 6F. FIG. 6H and FIG. 6I show GSEA of the differentially expressed genes induced by YKL-5-124 in vivo. Shown are two of the top most negatively (FIG. 6H) and positively (FIG. 6I) regulated “hallmark” signatures. FIG. 6J shows a heatmap for most differentially expressed genes from the top positively regulated pathways (colors are log₂FC).

FIGS. 7A to 7O: Combinatorial therapy reinvigorates anti-tumor immunity. FIG. 7A is a UMAP plot of the identified intratumoral infiltrating immune cells. FIG. 7B shows a percentage of different intratumoral infiltrating immune populations identified in FIG. 7A. FIG. 7C is a UMAP plot highlighting the whole population of T cells identified in FIG. 7A in purple (left) and UMAP plot showing the subpopulations identified within the T cells (right). FIGS. 7D-7H are UMAP plots of T cells displaying select marker gene expression. FIG. 7I depicts UMAP density plots showing distribution of annotated clusters in FIG. 7C within intratumoral T cells upon treatment. FIG. 7J shows the percentage of cells in individual CD4⁺ T clusters annotated in FIG. 7C by treatment. FIG. 7K shows a heatmap displaying expression of select genes in CD4⁺ T cell clusters (colors are log₂FC). FIG. 7L shows the percentage of cells in individual CD8+ T cell clusters annotated in FIG. 7C by treatment. FIG. 7M shows a heatmap displaying expression of select genes in CD8⁺ T cell clusters (colors are log ₂ FC). FIG. 7N shows percentages of tumor volume after 2 weeks of treatment combining anti-PD-1 and YKL-5-124 (Combo) with or without αCD4 (400 g/mouse) or aCD8 (400 μg/mouse) antibodies (n=8). **p<0.01, ***p<0.001 (unpaired two-tailed t test). FIG. 7O is a waterfall plot shows tumor response after week 2. Each column represents one mouse, in comparison with baseline measurements (n=8). **p<0.01, ***p<0.001 (unpaired two-tailed t test).

FIGS. 8A to 8J: YKL-5-124 specifically targets CDK7, disrupts cell cycle progression through inhibition of CDK7 CAK activity, and impairs DNA synthesis and MCM2 complex. FIG. 8A shows competitive pulldown assay in DMS79 treated with YKL-5-124 at indicated concentrations for 6 hr. Western blotting showing the pulldown (PD) or input (total lysate) of Cyclin H. FIG. 8B shows western blotting analysis of RNA Pol II total, RNA Pol II p-Ser 2, RNA Pol II p-Ser 5, CDK1 total, CDK2 total, pCDK1 (Thrl61), pCDK2 (Thr160) and Tubulin expression in NCI-H69 and GLC16 after treatment with YKL-5-124 at indicated concentrations for 24 hr. FIG. 8C shows western blotting analysis of RNA Pol II total, RNA Pol II CTD-Ser2, RNA Pol II CTD-Ser5, CDK1 total, CDK2 total, pCDK1 (Thrl61), pCDK2 (Thr160) and Tubulin expression in RPP631 and DMS79 after treatment with YKL-5-124, THZ531 or THZ1 at indicated concentrations for 24 hr. FIG. 8D shows RT-qPCR analysis of INSM1, ASCL1, NFIB and MYC in DMS79 and GLC16 after indicated treatment for 24 hr. Gene expression was normalized to GUSB. The RT-qPCR data were presented as fold changes of gene expression in the test sample compared to the vehicle. FIG. 8E shows cell viability was measured at indicated time points (normalized to day 0) in GLC16, NCI-H69 and NCI-H82 upon treatment with DMSO (0) or increasing concentrations of YKL-5-124 (nM). FIG. 8F shows flow cytometry analysis of Propidium Iodide (PI)-staining in DMS79 cells after treatment with DMSO or increasing concentrations of YKL-5-124 for 72 hr. FIG. 8G and FIG. 8H show quantification of DNA synthesis indicated by EdU incorporation per nucleus as well as within each replication focus in RPP631 cells treated with vehicle or 100 nM YKL-5-124 after 48 hr. FIG. 8G shows quantification of EdU nuclear density (nm⁻²) per nucleus and FIG. 8H shows EdU content per focus are plotted. FIG. 8I and FIG. 8J) Quantification MCM2 complex per nucleus as well as within each replication focus in RPP631 cells treated with vehicle or 100 nM YKL-5-124 after 48 hr. FIG. 8I shows quantification of MCM2 nuclear density (nm⁻²) per nuclei and FIG. 8J shows relative MCM2 content per focus are plotted. In FIGS. 8D, 8E, 8G, 8H, 8I, and 8J, data is shown as means±SD of three independent experiments run in triplicates. FIGS. 8G, 8H, 8I, and 8J, show an unpaired two-tailed t-test. **p<0.01, ****p<0.0001.

FIGS. 9A to 9L: YKL-5-124 disrupts cell cycle and induces pro-inflammatory cytokine/chemokine production. FIG. 9A and FIG. 9B show GSEA analysis of the differentially expressed genes induced by YKL-5-124 in RPP631. Here shown are two of the top most negatively regulated ‘Hallmarks’ signatures. Gene list was ranked with signed (from log₂ fold change (FC)) likelihood ratio from YKL-5-124 versus vehicle comparison. FIG. 9C and FIG. 9D show heatmaps for differential expression of transcripts from two top negatively regulated pathways in GSEA analysis between vehicle and YKL-5-124 treated cells (colors are log 2FC). FIG. 9E and FIG. 9F show GSEA analysis of expressed genes associated with super enhancers (SEs) induced by THZ1 (FIG. 9E) or YKL-5-124 (FIG. 9F) in SCLC. Ranking of SE-genes enrichment was compared to all GO Biological Processes gene sets in each dataset. FIG. 9G shows RT-qPCR analysis of TNF and CXCL10 in HAP1 CDK7 WT and HAP1 CDK7 C312S cells after treatment with DMSO or 100 nM YKL-5-124 for 48 hr. The RT-qPCR data were presented as fold changes of gene expression compared to the vehicle. FIG. 9H shows western blotting analysis of the STING-TBK1-IRF3 pathway in YKL-5-124-treated DMS79 and RPP631. FIG. 9I shows RT-qPCR analysis of Tnf and Cxcl10 levels upon 48 hr YKL-5-124 treatment in the CRISPR/Cas9 cGAS knockout (KO) RPP631 cells. The RT-qPCR data were presented as fold changes of gene expression compared to the vehicle. FIGS. 9J-9L show profiling of OT-I CD8⁺ T cells by flow cytometry analysis after treatment with DMSO or YKL-5-124. T cell activity markers CD69 (FIG. 9J), TNFα (FIG. 9K) and IFNγ (FIG. 9L) were examined. FIGS. 9G and 9I, data is shown as means±SD of three independent experiments run in triplicates. FIGS. 9J, 9K, and 9L, data is shown as means±SEM of three independent experiments run in ten replicates. Unpaired two-tailed t-test. NS, not significant.

FIGS. 10A to 10C.: Histopathology of murine SCLC models and establishment of syngeneic mouse models for studying oncoimmunology in SCLC. FIG. 10A shows representative images of H & E and immunohistochemistry (IHC) staining for ASCL1 of lung tumors from RPP GEMM, RPP and RP orthotopic models as indicated. FIG. 10B shows representative images of H & E and IHC staining for MYC of lung tumors from RPP-MYC orthotopic model. FIG. 10C shows experimental approach of establishing RPP orthotopic model. Using in vivo CRISPR/Cas9-mediated loss-of-function gene editing, single-guide RNAs (sgRNAs) designed to inactivate tumor suppressors Rb1, p53 and p130 were delivered through intratracheal induction into mouse lung to generate a murine SCLC model. After 9 months, lung tumor nodules were harvested for in vitro culture. Established RPP631 cells were injected back to C57BL/6 background mice orthotopically. Tumor burden was monitored by MRI and histopathology of SCLC was confirmed by rodent pathologist.

FIGS. 11A to 11E: Immune profiling gating strategy and comparison of the TME in RPP orthotopic syngeneic model versus GEMM model FIG. 11A shows a representation of an example of gating strategy. Single cells were first gated and followed by aqua live/dead staining selection for live cells. Total immune cells were identified by CD45 staining. A sequential gating strategy was then used to identify various populations using specific markers: total T cells (CD3⁺), CD4⁺ T cells, CD8⁺ T cells, myeloid cells (CD11b⁺), and dendritic cells (CD11c⁺ CD103⁺). In addition, the expression of CD44, CD62L and Ki67 in CD4⁺ T cells was analyzed. FIGS. 11B-11E show tumor infiltrating lymphocytes from tumor-bearing lung in the RPP orthotopic syngeneic model and RPP GEMM were analyzed by flow cytometry when tumor sizes were comparable. Total T cells (CD45⁺ CD3⁺) (FIG. 11B), CD4⁺ T cells (FIG. 11C), CD8⁺ T cells (FIG. 11D) and myeloid cells (CD11b⁺) (FIG. 11E) were quantified and compared. Data is shown as means±SD. Unpaired two-tailed t-test. NS, not significant.

FIGS. 12A to 12E: In vivo evaluation of YKL-5-124 treatment toxicity and target engagement. FIGS. 12A-12D show results of in vivo examination of YKL-5-124 treatment toxicities. FIG. 12A shows body weight and blood cell counts including platelet (FIG. 12B), white blood cells (WBC) (FIG. 12C) and red blood cells (RBC) (FIG. 12D) were monitored and measured on a weekly basis. Data is shown as means±SD. Unpaired two-tailed t-test. *p<0.01. FIG. 12E shows results of in vivo target engagement of YKL-5-124 by competitive pulldown assay. Tumor-bearing mice were administered with three dosages of YKL-5-124 (10 mg/kg) and tumor tissues were harvested after the last dose at indicated time points. Western blotting showing the pulldown (PD) or input (total lysate) of Cyclin H and Cyclin K.

FIGS. 13A to 13P: YKL-5-124 inhibits tumor growth and enhances tumor response to anti-PD-1 immunotherapy in multiple murine SCLC models. FIG. 13A depicts representative MRI images show mouse lung tumors before and after the treatment at indicated time points in the RP orthotopic model. Circled areas, heart. FIG. 13B shows the quantification of baseline tumor volumes by MRI scan of RPP-MYC mice. Control group (n=5), YKL-5-124 (n=5), Combo (n=7). FIG. 13C shows the quantification of tumor volume changes by MRI scan of RPP-MYC mice. Each dot represents one mouse, comparing to baseline MRI measurement. FIG. 13D depicts representative MRI images show mouse lung tumors before and after the treatment at indicated time points in the RPP-MYC model. Circled areas, heart. FIG. 13E shows the quantification of baseline tumor volumes by MRI scan of RPP GEMMs. Control group (n=8), anti-PD-1 (n=6), YKL-5-124 (n=6), Combo (n=9). Each dot represents one mouse. NS, not significant. FIG. 13F shows the quantification of tumor volume changes by MRI scan of RPP GEMMs after treatment with Control, anti-PD-1, YKL-5-124 and Combo. Waterfall plot shows tumor volumes response to the treatment after week 3. Each column represents one mouse, comparing to baseline MRI measurement. FIG. 13G shows representative MRI images show mouse lung tumors before and after the treatment at indicated time points in the RPP GEMMs. Circled areas, heart. FIG. 13H shows a Kaplan-Meier survival curve of RPP GEMMs after indicated treatment. Log-rank test *p<0.05, **p<0.01. FIGS. 13I-13L show results of in vivo examination of Combo and Chemo+Combo treatment toxicities (n=5). FIG. 13I shows the relative body weight and blood cell counts including platelet (FIG. 13J), WBC (FIG. 13K), RBC (FIG. 13L) were monitored as shown. FIGS. 13M-13P show that the expression of CD44, CD62L, Ki67 and ICOS in infiltrating CD4⁺ T cells after indicated treatment in the RPP GEMMs was analyzed by flow cytometry. MFI of CD44 (FIG. 13M), CD62L (FIG. 13N) and frequency (FIG. 13O) of ICOS⁺ CD4⁺ T cells were plotted. FIG. 13P shows the expression of GzmB in infiltrating CD8⁺ T cells after indicated treatment was analyzed. Frequencies of GzmB⁺ CD8⁺ T cells were presented. In FIGS. 13B, 13C, and 13E, data is shown as means±SEM. In FIGS. 13I, 13J, 13K, 13L, 13M, 13N, 13O, and 13P, data is shown as means±SD. FIGS. 13B, 13C, 13E, 13F, 13M, 13N, 13O, 13P show an unpaired two-tailed t-test. *p<0.05, **p<0.01, ***p<0.001, ****p<0.0001. NS, not significant.

FIGS. 14A to 14D: Single-cell analysis identifies intratumoral cell populations and confirms connection of CDK7 inhibition in tumor intrinsic signaling to immunity. FIG. 14A depicts violin plots showing the gene expression distribution of representative markers in each identified cell type. FIG. 14B depicts a 3D cell cycle trajectory showing eight clusters of cells at different cell-cycle state based on calculated scores for genes specific to the G1, S and G2/M phase. FIG. 14C and FIG. 14D show results of flow cytometry that was performed using splenocytes from mice under αCD4 or αCD8 treatment. FIG. 14C is a representative density plot showing CD4⁺ T cell population in control (left) and αCD4 (right) group. FIG. 14D is a representative density plot showing CD8⁺ T cell population in control (left) and αCD8 (right) group.

### DETAILED DESCRIPTION OF CERTAIN EMBODIMENTS

CDK7 is a master regulator of cell cycle progression. CDK7 functions as the catalytic core of the CDK-activating kinase (CAK) complex and becomes activated by binding to Cyclin H and Matl. The trimeric CAK complex activates several central cell cycle CDKs by phosphorylation. Temporal activation of these CDKs by the CAK complex ensures orderly progression within the cell cycle. In addition, CAK is a component of the general transcription factor TFIIH, a protein complex important for RNA polymerase II (RNA pol II)-mediated transcription. Compounds of Formula (I), highly selective covalent CDK7 inhibitors with no off-target effect on CDK12/13, provide confirmation of the role of CDK7 in regulating cell cycle progression, suggesting potential redundancies in its control of gene transcription.

The present disclosure demonstrates that CDK7 inhibition results in cell cycle disruption and genomic instability while activating immune response signaling in SCLC. This tumor cell-intrinsic effect potentiates activation of infiltrating immune cells, supporting a role of CDK7 in regulating anti-tumor immunity. In certain embodiments, the CDK7 inhibitors (e.g., compounds of Formula (I)) increase the susceptibility of cancer cells (e.g., SCLC) to immunotherapy (e.g., anti-PD-1), and provide improved survival in multiple SCLC models. Thus, combining CDK7 inhibitors with immunotherapy may improve treatment efficacy and survival benefit in patients with SCLC.

### Methods of Treatment

One aspect of the present disclosure relates to methods of treating cancer in a subject in need thereof. The methods include administering a CDK7 inhibitor and an immunotherapy. In certain embodiments, the methods further include administering one or more chemotherapeutic agents. In other embodiments, the methods further include administering one or more targeted agents.

In another aspect, the present disclosure provides methods of treating a cancer in a subject in need thereof, the methods comprising administering to the subject an effective amount (e.g., therapeutically effective amount) of (1) a CDK7 inhibitor and an immunotherapy described herein, or (2) a pharmaceutical composition described herein. In certain embodiments, the CDK7 inhibitor and immunotherapy are synergistic in treating the cancer, compared to the CDK7 inhibitor and/or immunotherapy alone.

In another aspect, the present disclosure provides methods of preventing a cancer in a subject in need thereof, the methods comprising administering to the subject an effective amount (e.g., prophylactically effective amount) of (1) a CDK7 inhibitor and an immunotherapy described herein, or (2) a pharmaceutical composition described herein. In certain embodiments, the CDK7 inhibitor and immunotherapy are synergistic in preventing the cancer, compared to the CDK7 inhibitor and/or immunotherapy alone.

In another aspect, the present disclosure provides methods of reducing, delaying, and/or preventing in a subject in need thereof the resistance of a cancer to a CDK7 inhibitor and/or immunotherapy, the methods comprising administering to the subject an effective amount of (1) a CDK7 inhibitor and an immunotherapy described herein, or (2) a pharmaceutical composition described herein. In certain embodiments, the CDK7 inhibitor and immunotherapy are synergistic in reducing, delaying, and/or preventing the resistance of the cancer to the CDK7 inhibitor and/or immunotherapy, compared to the CDK7 inhibitor and/or immunotherapy alone.

In certain embodiments, the CDK7 inhibitor and immunotherapy are administered to the subject at the same time. In certain embodiments, the CDK7 inhibitor and immunotherapy are administered to the subject at different times.

In another aspect, the present disclosure provides methods of inhibiting the proliferation of a cell, the methods comprising contacting the cell with an effective amount of (1) a CDK7 inhibitor and an immunotherapy described herein, or (2) a pharmaceutical composition described herein. In certain embodiments, the CDK7 inhibitor and immunotherapy are synergistic in inhibiting the proliferation of the cell, compared to the CDK7 inhibitor and/or immunotherapy alone.

In another aspect, the present disclosure provides methods of reducing, delaying, and/or preventing the resistance of a cell to a CDK7 inhibitor and/or an immunotherapy, the methods comprising contacting the cell with an effective amount of (1) a CDK7 inhibitor and an immunotherapy described herein, or (2) a pharmaceutical composition described herein. In certain embodiments, the CDK7 inhibitor and immunotherapy are synergistic in reducing, delaying, and/or preventing the resistance of the cell to the CDK7 inhibitor and/or immunotherapy, compared to the CDK7 inhibitor and/or immunotherapy alone.

In another aspect, the present disclosure provides the CDK7 inhibitors and immunotherapies described herein for use in a method described herein (e.g., a method of treating cancer in a subject in need thereof, a method of preventing a cancer in a subject in need thereof, a method of reducing, delaying, and/or preventing in a subject in need thereof the resistance of a cancer to a CDK7 inhibitor and/or immunotherapy, a method of inhibiting the proliferation of a cell, or a method of reducing, delaying, and/or preventing the resistance of a cell to a CDK7 inhibitor and/or immunotherapy). In certain embodiments, the present disclosure provides the CDK7 inhibitors and immunotherapies for use in treating cancer in a subject in need thereof. In certain embodiments, the present disclosure provides a combination of the CDK7 inhibitors and immunotherapies for use in treating a cancer in a subject in need thereof.

In still another aspect, the present disclosure provides the pharmaceutical compositions described herein for use in a method described herein (e.g., a method of treating cancer in a subject in need thereof, a method of preventing a cancer in a subject in need thereof, a method of reducing, delaying, and/or preventing in a subject in need thereof the resistance of a cancer to a CDK7 inhibitor and/or immunotherapy, a method of inhibiting the proliferation of a cell, or a method of reducing, delaying, and/or preventing the resistance of a cell to a CDK7 inhibitor and/or immunotherapy). In certain embodiments, the present disclosure provides the pharmaceutical compositions for use in treating cancer in a subject in need thereof.

In certain embodiments, the CDK7 inhibitors and immunotherapies, or pharmaceutical compositions thereof, can be administered in combination with an anti-cancer therapy including, but not limited to, surgery, radiation therapy, transplantation (e.g., stem cell transplantation, bone marrow transplantation), and chemotherapy. In certain embodiments the CDK7 inhibitors and immunotherapies, or pharmaceutical compositions thereof, can be administered in combination with chemotherapy (i.e., one or more chemotherapeutic agents). In certain embodiments the CDK7 inhibitors and immunotherapies, or pharmaceutical compositions thereof, can be administered in combination with one or more chemotherapeutic agents.

In certain embodiments, the combination of administering CDK7 inhibitors and immunotherapies, or pharmaceutical compositions thereof, and one or more chemotherapeutic agents is synergistic in treating a cancer, compared to treatment with CDK7 inhibitors and/or immunotherapies, or pharmaceutical compositions thereof, alone, or compared to treatment with chemotherapy alone. The combination of CDK7 inhibitor, immunotherapy, and chemotherapy may be useful in treating cancers that are resistant to a CDK7 inhibitor alone, immunotherapy alone, and/or chemotherapy alone. The combination of CDK7 inhibitor, immunotherapy, and chemotherapy may be useful in treating a subject with a cancer that has failed therapy of the cancer with CDK7 inhibitor alone, immunotherapy alone, and/or chemotherapy alone. The CDK7 inhibitors, immunotherapy, and chemotherapy may be administered at the same time or administered separately at different times in any order. In some embodiments, the CDK7 inhibitor and immunotherapy are administered before chemotherapy. In some embodiments, the CDK7 inhibitor and immunotherapy are administered after chemotherapy. In some embodiments, the CDK7 inhibitor and immunotherapy are administered concurrently with chemotherapy, e.g., on the same day.

The methods described herein may be used to treat any cancer. In certain embodiments, the cancer is a cancer that is commonly treated with chemotherapy. In certain embodiments, the cancer is a cancer that is commonly treated with immunotherapy. In some embodiments, the cancer is a leukemia; a lymphoma; myelodysplasia; multiple myeloma; lung cancer (e.g., bronchogenic carcinoma, small cell lung cancer (SCLC), non-small cell lung cancer (NSCLC), adenocarcinoma of the lung); kidney cancer; acoustic neuroma; adenocarcinoma; adrenal gland cancer; anal cancer; angiosarcoma; appendix cancer; benign monoclonal gammopathy; biliary cancer; bladder cancer; breast cancer; brain cancer; bronchus cancer; carcinoid tumor; cervical cancer; choriocarcinoma; chordoma; craniopharyngioma; colorectal cancer; connective tissue cancer; epithelial carcinoma; ependymoma; endotheliosarcoma; endometrial cancer; esophageal cancer; Ewing's sarcoma; ocular cancer; familiar hypereosinophilia; gall bladder cancer; gastric cancer; gastrointestinal stromal tumor (GIST); germ cell cancer; head and neck cancer; heavy chain disease; leiomyosarcoma (LMS); mastocytosis; muscle cancer; myelodysplastic syndrome (MDS); mesothelioma; myeloproliferative disorder (MPD); neuroblastoma; neurofibroma; neuroendocrine cancer; osteosarcoma; ovarian cancer; papillary adenocarcinoma; pancreatic cancer; penile cancer; pinealoma; primitive neuroectodermal tumor (PNT); plasma cell neoplasia; paraneoplastic syndromes; intraepithelial neoplasms; prostate cancer; rectal cancer; rhabdomyosarcoma; salivary gland cancer; skin cancer; small bowel cancer; soft tissue sarcoma; sebaceous gland carcinoma; small intestine cancer; sweat gland carcinoma; synovioma; testicular cancer; thyroid cancer; urethral cancer; vaginal cancer; or vulvar cancer.

In some embodiments, the cancer is a lung cancer. In some embodiments, the cancer is small cell lung cancer (SCLC).

### CDK7 Inhibitor

In certain embodiments, the CDK7 inhibitor is any CDK7 inhibitor. In certain embodiments, the CDK7 inhibitor is any CDK7 inhibitor disclosed in U.S. Ser. No. 15/538,763, the entire content of which is incorporated herein by reference. In certain embodiments, the CDK7 inhibitor is a compound of Formula (I):

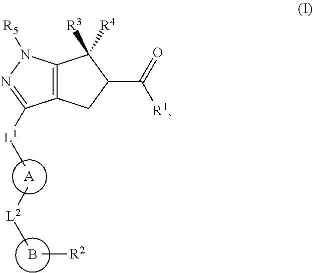

or a pharmaceutically acceptable salt, solvate, hydrate, polymorph, co-crystal, tautomer, stereoisomer, isotopically labeled derivative, or prodrug thereof, wherein:
R¹ is —NR^{a}R^{b}, —CHR^{a}R^{b} or —OR^{a}, wherein each of R^{a} and R^{b} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group when attached to a nitrogen atom, or an oxygen protecting group when attached to an oxygen atom, or R^{a} and R^{b} are joined to form an optionally substituted carbocyclic, optionally substituted heterocyclic, optionally substituted aryl, or optionally substituted heteroaryl ring;
each of R³ and R⁴ is independently hydrogen, halogen, optionally substituted C₁-C₆ alkyl, or optionally substituted aryl, or R³ and R⁴ are joined to form an optionally substituted C₃-C₆ carbocyclyl ring;
R⁵ is independently hydrogen, optionally substituted C₁-C₆ alkyl, or a nitrogen protecting group;
L¹ is —NR^{L1}—, —NR^{L}C(═O)—, —C(═O)NR^{L1}—, —O—, or —S—, wherein R^{L1} is hydrogen, optionally substituted C₁-C₆ alkyl, or a nitrogen protecting group;
Ring A is optionally substituted carbocyclyl, optionally substituted heterocyclyl, optionally substituted aryl, or optionally substituted heteroaryl;
L² is a bond, —C(═O)—, —NR^{L2}—, —C(═O)NR^{L2}—, —NR^{L2}C(═O)—, —O—, or —S—, wherein R^{L2} is hydrogen, optionally substituted C₁-C₆ alkyl, or a nitrogen protecting group;
Ring B is absent, optionally substituted carbocyclyl, optionally substituted heterocyclyl, optionally substituted aryl, or optionally substituted heteroaryl; and
R² is any of Formulae (i-1)-(i-42) as defined herein:

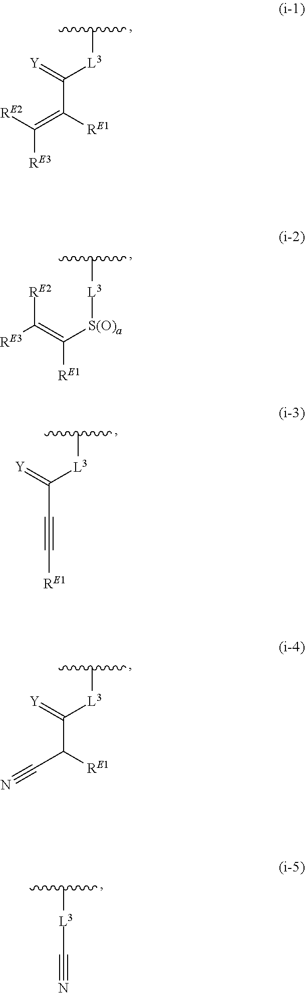

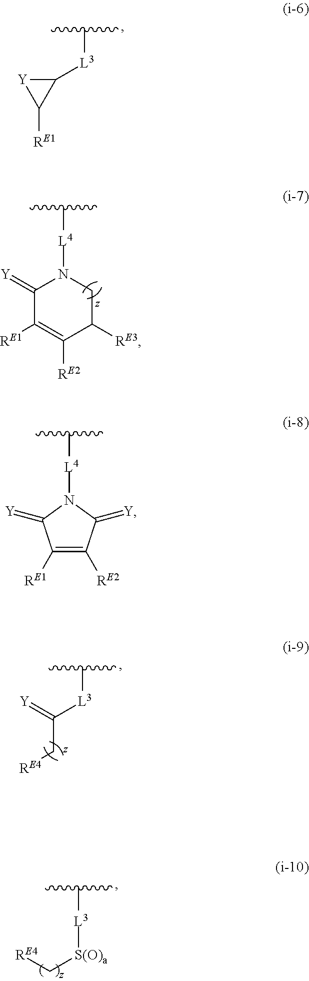

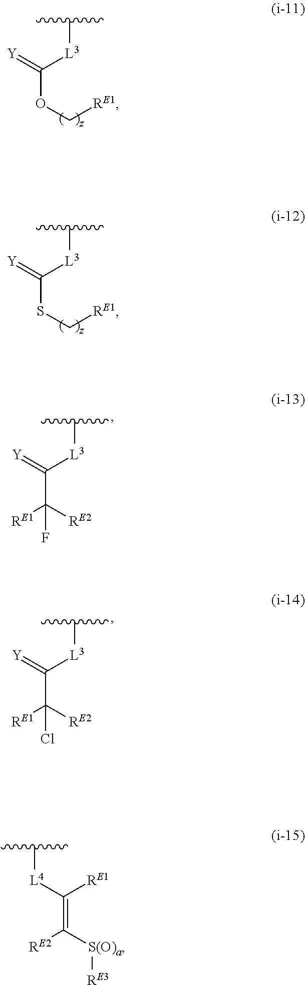

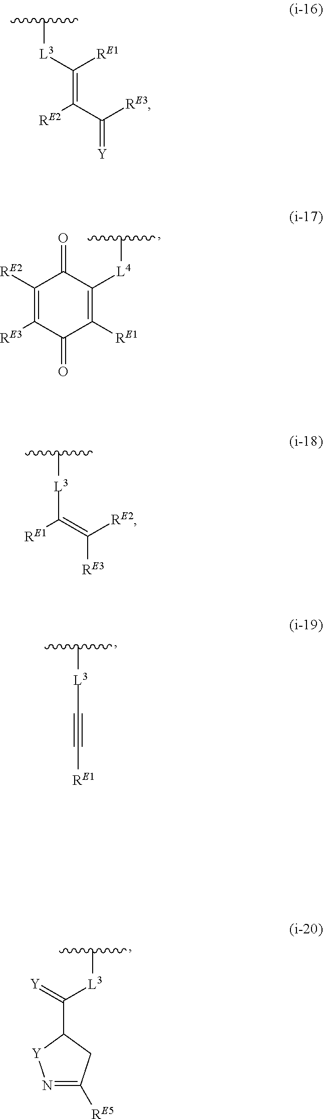

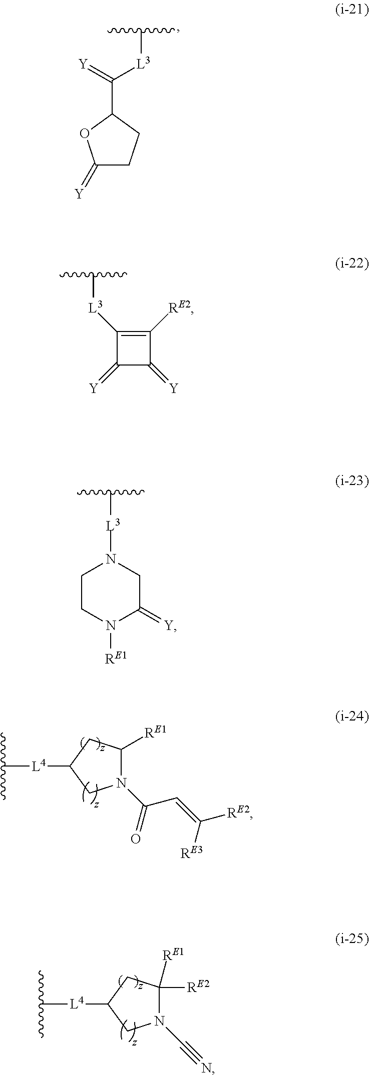

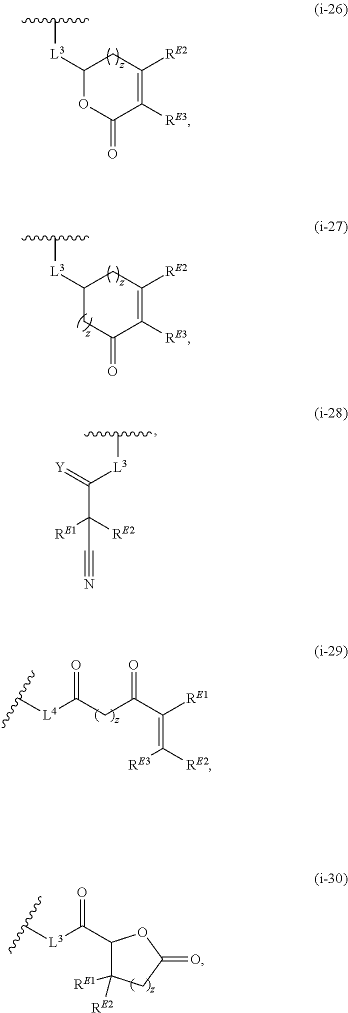

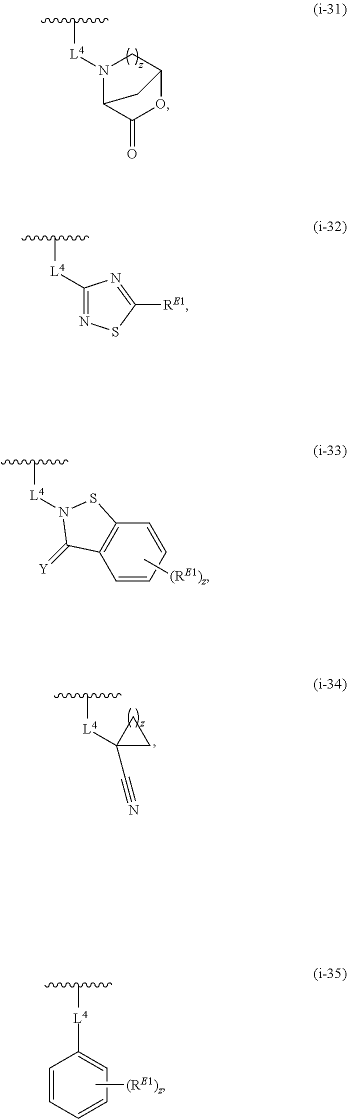

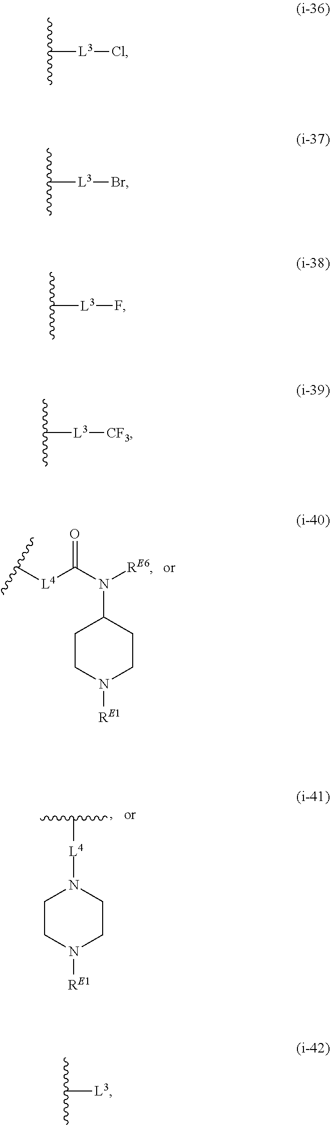

wherein
L³ is a bond or an optionally substituted C₁₋₄ hydrocarbon chain, optionally wherein one or more carbon units of the hydrocarbon chain are independently replaced with —C(═O)—, —O—, —S—, —NR^{L3a}—, —NR^{L3a}C(═O)—, —C(═O)NR^{L3a}—, —SC(═O)—, —C(═O)S—, —OC(═O)—, —C(═O)O—, —NR^{L3a}C(═S)—, —C(═S)NR^{L3a}—, trans-CR^{L3b}═CR^{L3b}—, cis-CR^{L3b}═CR^{L3b}—, —C≡C—, —S(═O)—, —S(═O)O—, —OS(═O)—, —S(═O)NR^{L3a}—, NR^{L3a}S(═O)—S(═O)₂—, —S(═O)₂O—, —OS(═O)₂—, —S(═O)₂NR^{L3a}—, or —NR^{L3a}S(═O)₂—, wherein R^{L3a} is hydrogen, substituted or unsubstituted C₁₋₆ alkyl, or a nitrogen protecting group, and wherein each occurrence of R^{L3b} is independently hydrogen, halogen, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R^{L3b} groups are joined to form an optionally substituted carbocyclic or optionally substituted heterocyclic ring;
L⁴ is a bond or an optionally substituted, branched or unbranched C₁₋₆ hydrocarbon chain;
each of R^{E1}, R^{E2}, and R^{E3} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{EE}, —CH₂N(R^{EE})₂, —CH₂SR^{EE}, —OR^{EE}, —N(R^{EE})₂, —Si(R^{EE})₃, and —SR^{EE}, wherein each occurrence of R^{EE} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl, or two R^{EE} groups are joined to form an optionally substituted heterocyclic ring; or R^{E1} and R^{E3}, or R^{E2} and R^{E3}, or R^{E1} and R^{E2} are joined to form an optionally substituted carbocyclic or optionally substituted heterocyclic ring;
R^{E4} is a leaving group;
R^{E5} is halogen;
R^{E6} is hydrogen, substituted or unsubstituted C₁₋₆ alkyl, or a nitrogen protecting group; each instance of Y is independently O, S, or NR^{E7}, wherein R^{E7} is hydrogen, substituted or unsubstituted C₁₋₆ alkyl, or a nitrogen protecting group;
a is 1 or 2; and
each instance of z is independently 0, 1, 2, 3, 4, 5, or 6, as valency permits.

In certain embodiments of the compound of Formula (I),
R¹ is —NR^{a}R^{b}, —CHR^{a}R^{b} or —OR^{a}, wherein each of R^{a} and R^{b} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group when attached to a nitrogen atom, or an oxygen protecting group when attached to an oxygen atom, or R^{a} and R^{b} are joined to form an optionally substituted carbocyclic, optionally substituted heterocyclic, optionally substituted aryl, or optionally substituted heteroaryl ring;
each of R³ and R⁴ is independently hydrogen, halogen, or optionally substituted C₁-C₆ alkyl, or R³ and R⁴ are joined to form an optionally substituted C₃-C₆ carbocyclyl ring;
R⁵ is hydrogen, optionally substituted C₁-C₆ alkyl, or a nitrogen protecting group;
L¹ is —NR^{L1}—, —NR^{L1}C(═O)—, —C(═O)NR^{L1}—, —O—, or —S—, wherein R^{L1} is hydrogen, optionally substituted C₁-C₆ alkyl, or a nitrogen protecting group;
Ring A is optionally substituted carbocyclyl, optionally substituted heterocyclyl, optionally substituted aryl, or optionally substituted heteroaryl;
L² is a bond, —C(═O)—, —NR^{L2}—, —C(═O)NR^{L2}—, —NR^{L2}C(═O)—, —O—, or —S—, wherein R^{L2} is hydrogen, optionally substituted C₁-C₆ alkyl, or a nitrogen protecting group;
Ring B is absent, optionally substituted carbocyclyl, optionally substituted heterocyclyl, optionally substituted aryl, or optionally substituted heteroaryl; and
R² is any of Formulae (i-1)-(i-41) as defined herein:

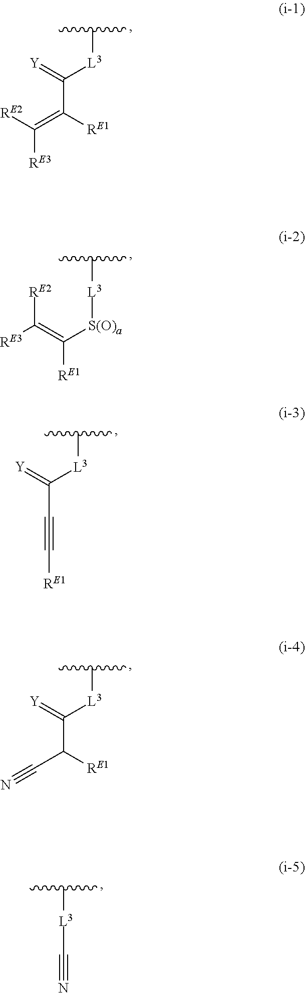

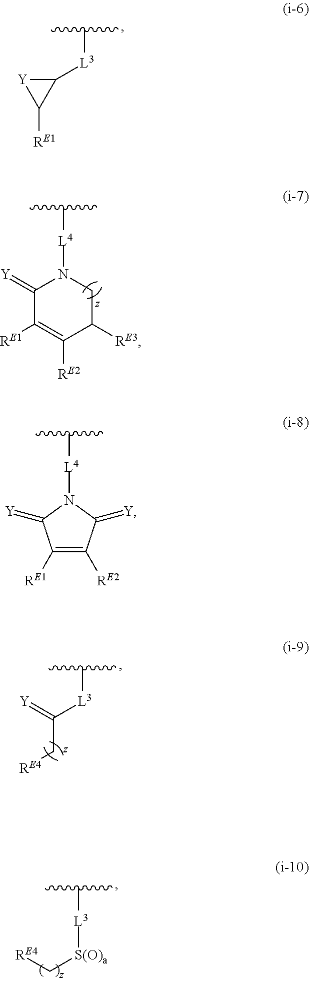

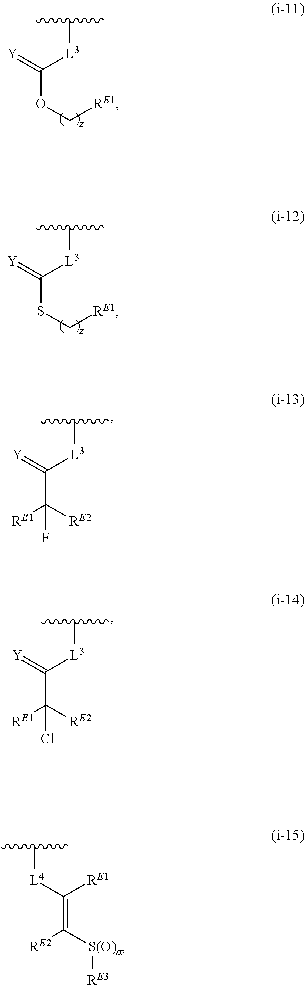

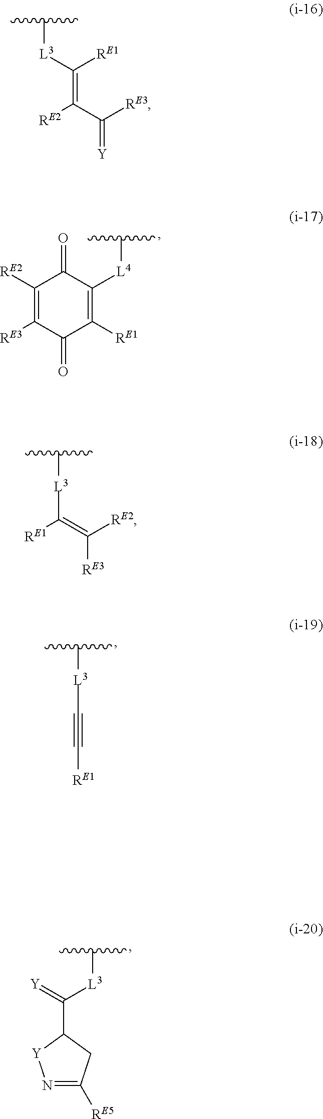

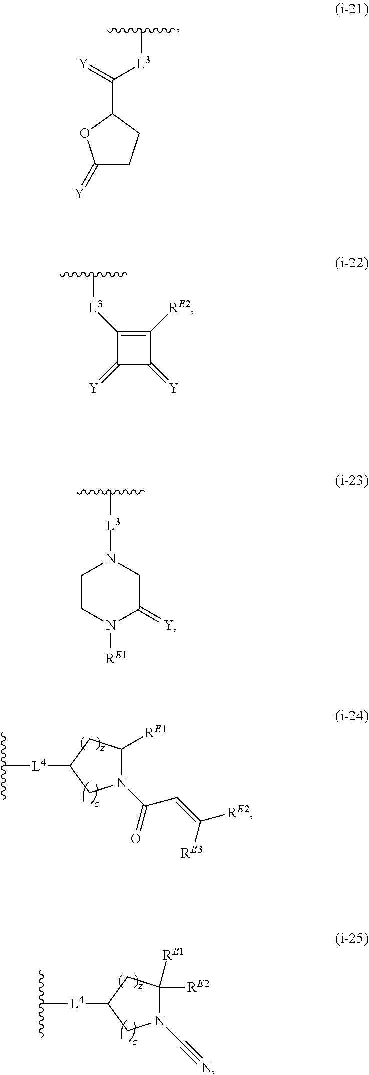

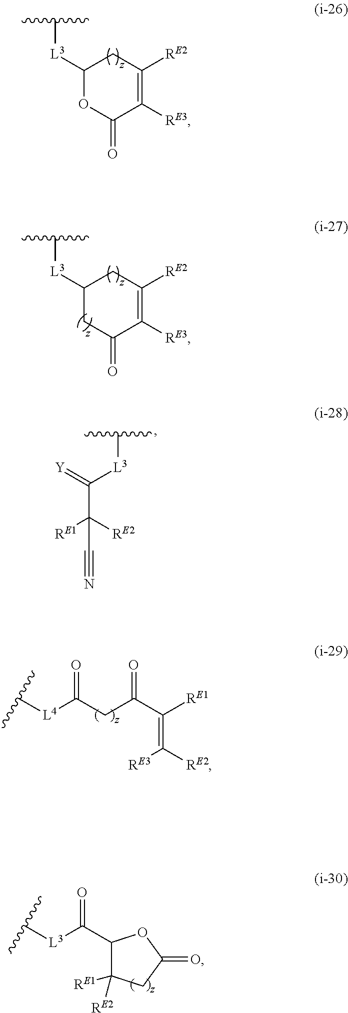

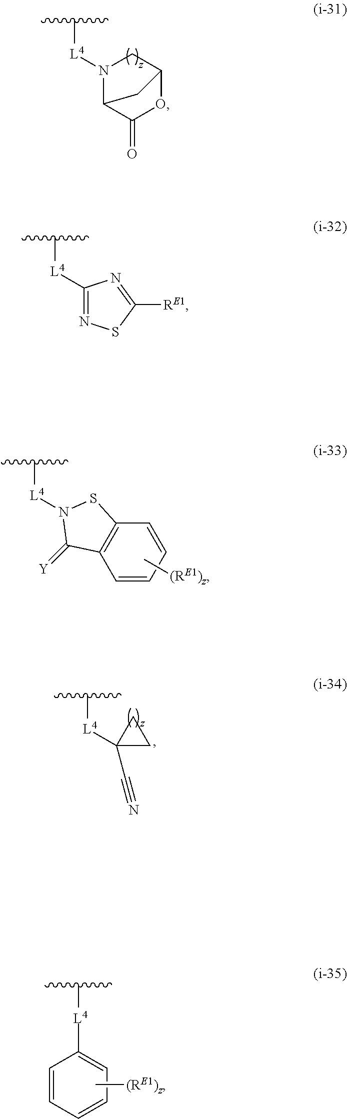

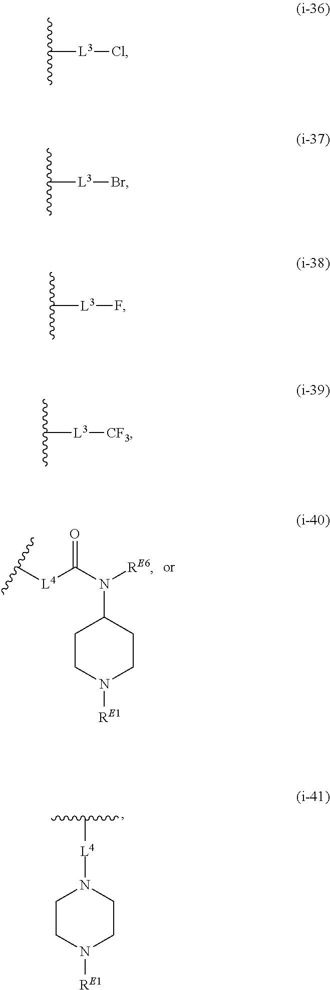

wherein:
L³ is a bond or an optionally substituted C₁₋₄ hydrocarbon chain, optionally wherein one or more carbon units of the hydrocarbon chain are independently replaced with —C(═O)—, —O—, —S—, —NR^{L3a}—, —NR^{L3a}C(═O)—, —C(═O)NR^{L3a}—, —SC(═O)—, —C(═O)S—, —OC(═O)—, —C(═O)O—, —NR^{L3a}C(═S)—, —C(═S)NR^{L3a}—, trans-CR^{L3b}═CR^{L3b}—, cis-CR^{L3b}═CR^{L3b}—, —C≡C—, —S(═O)—, —S(═O)O—, —OS(═O)—, —S(═O)NR^{L3a}—, NR^{L3a}S(O)—, —S(═O)₂—, —S(═O)₂O—, —OS(═O)₂—, —S(═O)₂NR^{L3a}—, or —NR^{L3a}S(═O)₂—, wherein R^{L3a} is hydrogen, substituted or unsubstituted C₁₋₆ alkyl, or a nitrogen protecting group, and wherein each occurrence of R^{L3b} is independently hydrogen, halogen, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R^{L3b} groups are joined to form an optionally substituted carbocyclic or optionally substituted heterocyclic ring;
L⁴ is a bond or an optionally substituted, branched or unbranched C₁₋₆ hydrocarbon chain;
each of R^{E1}, R^{E2}, and R^{E3} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{EE}, —CH₂N(R^{EE})₂, —CH₂SR^{EE}, —OR^{EE}, —N(R^{EE})₂, —Si(R^{EE})₃, and —SR^{EE}, wherein each occurrence of R^{EE} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl, or two R^{EE} groups are joined to form an optionally substituted heterocyclic ring; or R^{E1} and R^{E3}, or R^{E2} and R^{E3}, or R^{E1} and R^{E2} are joined to form an optionally substituted carbocyclic or optionally substituted heterocyclic ring;
R^{E4} is a leaving group;
R^{E5} is halogen;
R^{E6} is hydrogen, substituted or unsubstituted C₁₋₆ alkyl, or a nitrogen protecting group; each instance of Y is independently O, S, or NR^{E7}, wherein R^{E7} is hydrogen, substituted or unsubstituted C₁₋₆ alkyl, or a nitrogen protecting group;
a is 1 or 2; and
each instance of z is independently 0, 1, 2, 3, 4, 5, or 6, as valency permits.

Compounds of Formula (I) include R¹ attached to the carbonyl substituent of the pyrrolopyrazole bicyclic ring. R¹ may be —NR^{a}R^{b}, —CHR^{a}R^{b} or —OR^{a}, wherein each of R^{a} and R^{b} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group when attached to a nitrogen atom, or an oxygen protecting group when attached to an oxygen atom, or R^{a} and R^{b} are joined to form an optionally substituted carbocyclic, optionally substituted heterocyclic, optionally substituted aryl, or optionally substituted heteroaryl ring. In certain embodiments, R¹ is —NR^{a}R^{b}. In certain embodiments, R¹ is —CHR^{a}R^{b}. In certain embodiments, R¹ is —OR^{a}.

In certain embodiments, R¹ is

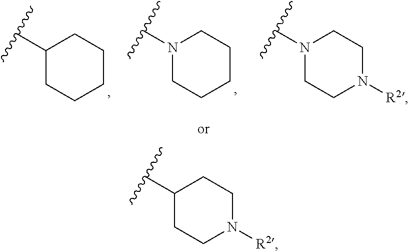

wherein R^{2′} is hydrogen, optionally substituted C₁-C₆ alkyl, or a nitrogen protecting group, and each ring atom is optionally substituted. In certain embodiments, R¹ is

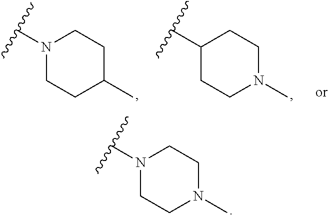

In certain embodiments, R¹ is of Formula (ii-1):

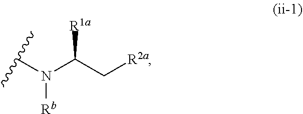

wherein:
R^{b} is hydrogen, optionally substituted C₁-C₆ alkyl, or a nitrogen protecting group;
R^{1a} is hydrogen, C₁-C₆ alkyl, or optionally substituted aryl; and
R^{2a} is hydrogen, —OR^{1N}, or —NR^{1N}R^{2N}, wherein each of R^{1N} and R^{2N} is independently hydrogen, C₁-C₆ alkyl, a nitrogen protecting group when attached to a nitrogen atom, or an oxygen protecting group when attached to an oxygen atom.

In certain embodiments, R¹ is of Formula (ii-2):

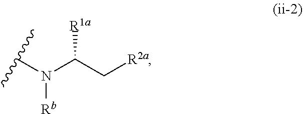

wherein:
R^{b} is hydrogen, optionally substituted C₁-C₆ alkyl, or a nitrogen protecting group;
R^{1a} is hydrogen, C₁-C₆ alkyl, or optionally substituted aryl; and
R^{2a} is hydrogen, —OR^{1N}, or —NR^{1N}R^{2N}, wherein each of R^{1N} and R^{2N} is independently hydrogen, C₁-C₆ alkyl, a nitrogen protecting group when attached to a nitrogen atom, or an oxygen protecting group when attached to an oxygen atom.

In certain embodiments, R^{b} is hydrogen. In certain embodiments, R^{b} is optionally substituted C₁-C₆ alkyl. In certain embodiments, R^{b} is unsubstituted C₁-C₆ alkyl. In certain embodiments, R^{b} is a nitrogen protecting group. In certain embodiments, R^{b} is Bn, BOC, Cbz, Fmoc, trifluoroacetyl, triphenylmethyl, acetyl, or Ts.

In certain embodiments, R^{1a} is hydrogen. In certain embodiments, R^{1a} is methyl. In certain embodiments, R^{1a} is ethyl. In certain embodiments, R^{1a} is propyl. In certain embodiments, R^{1a} is optionally substituted phenyl. In certain embodiments, R^{1a} is phenyl.

In certain embodiments, R^{2a} is hydrogen. In certain embodiments, R^{2a} is —OR^{1N}, wherein R^{1N} is hydrogen, C₁-C₆ alkyl, or an oxygen protecting group. In certain embodiments, R^{2a} is —OH. In certain embodiments, R^{2a} is —NR^{1N}R^{2N}, wherein each of R^{1N} and R^{2N} is independently hydrogen, C₁-C₆ alkyl, or a nitrogen protecting group. In certain embodiments, R^{1N} and R^{2N} are the same. In certain embodiments, R^{1N} and R^{2N} are distinct. In certain embodiments, R^{1N} and R^{2N} are both methyl. In certain embodiments, R^{1N} and R^{2N} are both ethyl. In certain embodiments, R^{1N} and R^{2N} are both propyl. In certain embodiments, R^{1N} and R^{2N} are both hydrogen. In certain embodiments, R^{1N} and R^{2N} are both nitrogen protecting groups. In certain embodiments, at least one of R^{1N} and R^{2N} is methyl. In certain embodiments, at least one of R^{1N} and R^{2N} is ethyl. In certain embodiments, at least one of R^{1N} and R^{2N} is propyl. In certain embodiments, at least one of R^{1N} and R^{2N} is hydrogen. In certain embodiments, at least one of R^{1N} and R^{2N} is a nitrogen protecting group. In certain embodiments, R^{1N} is methyl, and R^{2N} is hydrogen. In certain embodiments, R^{1N} is ethyl, and R^{2N} is hydrogen. In certain embodiments, R^{1N} is propyl, and R^{2N} is hydrogen. In certain embodiments, R^{1N} is a nitrogen protecting group, and R^{2N} is hydrogen. In certain embodiments, R^{1N} is methyl, and R^{2N} is a nitrogen protecting group. In certain embodiments, R^{1N} is ethyl, and R^{2N} is a nitrogen protecting group. In certain embodiments, R^{1N} is propyl, and R^{2N} is a nitrogen protecting group.

In certain embodiments, R¹ is of Formula (ii-1a):

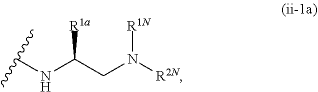

wherein:
R^{1a} is hydrogen, C₁-C₆ alkyl, or optionally substituted aryl; and
each of R^{1N} and R^{2N} is independently hydrogen, C₁-C₆ alkyl, or a nitrogen protecting group.

In certain embodiments, R¹ is of Formula (ii-2a):

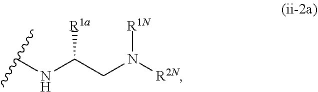

R^{1a} is hydrogen, C₁-C₆ alkyl, or optionally substituted aryl; and
each of R^{1N} and R^{2N} is independently hydrogen, C₁-C₆ alkyl, or a nitrogen protecting group.

In certain embodiments, R^{1a} is hydrogen. In certain embodiments, R^{1a} is methyl. In certain embodiments, R^{1a} is ethyl. In certain embodiments, R^{1a} is propyl. In certain embodiments, R^{1a} is optionally substituted phenyl. In certain embodiments, R^{1a} is phenyl. In certain embodiments, R^{1N} and R^{2N} are the same.

In certain embodiments, R^{1N} and R^{2N} are distinct. In certain embodiments, R^{1N} and R^{2N} are both methyl. In certain embodiments, R^{1N} and R^{2N} are both ethyl. In certain embodiments, R^{1N} and R^{2N} are both propyl. In certain embodiments, R^{1N} and R^{2N} are both hydrogen. In certain embodiments, R^{1N} and R^{2N} are both nitrogen protecting groups. In certain embodiments, at least one of R^{1N} and R^{2N} is methyl. In certain embodiments, at least one of R^{1N} and R^{2N} is ethyl. In certain embodiments, at least one of R^{1N} and R^{2N} is propyl. In certain embodiments, at least one of R^{1N} and R^{2N} is hydrogen. In certain embodiments, at least one of R^{1N} and R^{2N} is a nitrogen protecting group. In certain embodiments, R^{1N} is methyl, and R^{2N} is hydrogen. In certain embodiments, R^{1N} is ethyl, and R^{2N} is hydrogen. In certain embodiments, R^{1N} is propyl, and R^{2N} is hydrogen. In certain embodiments, R^{1N} is a nitrogen protecting group, and R^{2N} is hydrogen. In certain embodiments, R^{1N} is methyl, and R^{2N} is a nitrogen protecting group. In certain embodiments, R^{1N} is ethyl, and R^{2N} is a nitrogen protecting group. In certain embodiments, R^{1N} is propyl, and R^{2N} is a nitrogen protecting group.

In certain embodiments, R¹ is of Formula (ii-1b):

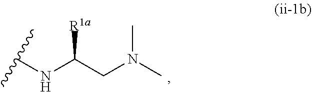

wherein R^{1a} is hydrogen, C₁-C₆ alkyl, or optionally substituted aryl.

In certain embodiments, R¹ is of Formula (ii-2b):

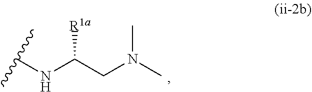

wherein R^{1a} is hydrogen, C₁-C₆ alkyl, or optionally substituted aryl.

In certain embodiments, R^{1a} is hydrogen. In certain embodiments, R^{1a} is methyl. In certain embodiments, R^{1a} is ethyl. In certain embodiments, R^{1a} is propyl. In certain embodiments, R^{1a} is optionally substituted phenyl. In certain embodiments, R^{1a} is phenyl.

In certain embodiments, R¹ is:

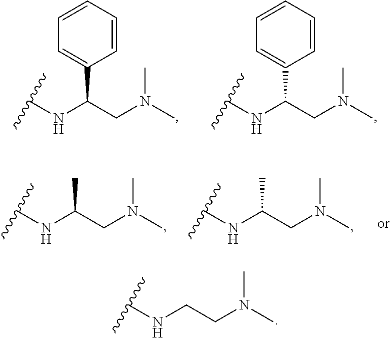

In certain embodiments, R¹ is:

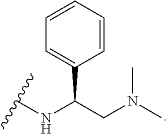 [SMILES: *N[C@H](CN(C)C)c1ccccc1]

In certain embodiments, R¹ is:

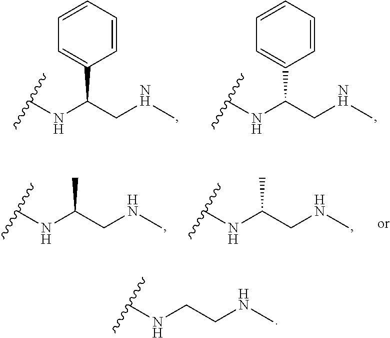

In certain embodiments, R¹ is:

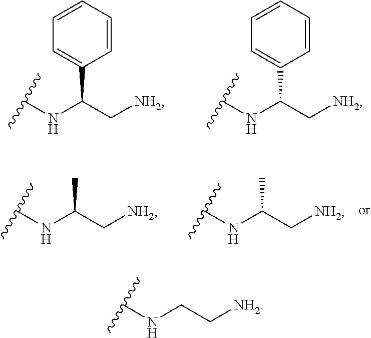

In certain embodiments, R¹ is:

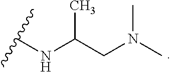 [SMILES: *NC(C)CN(C)C]

In certain embodiments, R¹ is of Formula (ii-1c):

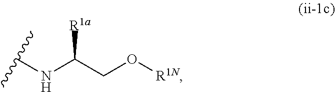

wherein:
R^{1a} is hydrogen, C₁-C₆ alkyl, or optionally substituted aryl; and
each of R^{1N} and R^{2N} is independently hydrogen, C₁-C₆ alkyl, or an oxygen protecting group.

In certain embodiments, R¹ is of Formula (ii-2c):

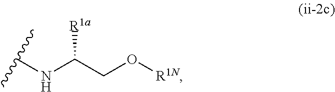

wherein:
R^{1a} is hydrogen, C₁-C₆ alkyl, or optionally substituted aryl; and
each of R^{1N} and R^{2N} is independently hydrogen, C₁-C₆ alkyl, or an oxygen protecting group.

In certain embodiments, R^{1a} is hydrogen. In certain embodiments, R^{1a} is methyl. In certain embodiments, R^{1a} is ethyl. In certain embodiments, R^{1a} is propyl. In certain embodiments, R^{1a} is optionally substituted phenyl. In certain embodiments, R^{1a} is phenyl. In certain embodiments, R^{1N} is hydrogen. In certain embodiments, R^{1N} is methyl. In certain embodiments, R^{1N} is ethyl. In certain embodiments, R^{1N} is propyl. In certain embodiments, R^{1N} is an oxygen protecting group.

In certain embodiments, R¹ is of Formula (ii-1d):

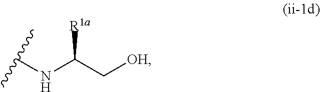

wherein R^{1a} is hydrogen, C₁-C₆ alkyl, or optionally substituted aryl.

In certain embodiments, R¹ is of Formula (ii-2d):

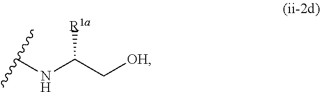

wherein R^{1a} is hydrogen, C₁-C₆ alkyl, or optionally substituted aryl.

In certain embodiments, R^{1a} is hydrogen. In certain embodiments, R^{1a} is methyl. In certain embodiments, R^{1a} is ethyl. In certain embodiments, R^{1a} is propyl. In certain embodiments, R^{1a} is optionally substituted phenyl. In certain embodiments, R^{1a} is phenyl.

In certain embodiments, R¹ is

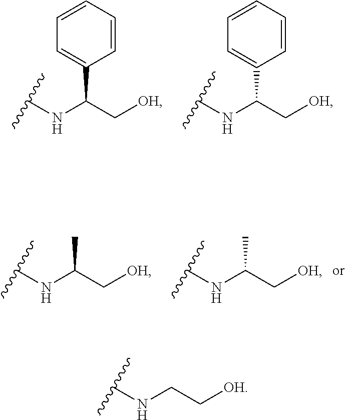

In certain embodiments, R¹ is —NR^{a}R^{b}, wherein R^{a} is optionally substituted aryl, and R^{b} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group. In certain embodiments, R¹ is —NR^{a}R^{b}, wherein R^{a} is optionally substituted heteroaryl, and R^{b} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group. In certain embodiments, R¹ is —NR^{a}R^{b}, wherein R^{a} is optionally substituted heterocyclyl, and R^{b} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group. In certain embodiments, R¹ is —NR^{a}R^{b}, wherein R^{a} is optionally substituted carbocyclyl, and R^{b} is selected from hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group. In certain embodiments, R¹ is —NR^{a}R^{b}, wherein R^{a} is optionally substituted aryl, and R^{b} is hydrogen. In certain embodiments, R¹ is —NR^{a}R^{b}, wherein R^{a} is optionally substituted heteroaryl, and R^{b} is hydrogen. In certain embodiments, R¹ is —NR^{a}R^{b}, wherein R^{a} is optionally substituted heterocyclyl, and R^{b} is hydrogen. In certain embodiments, R¹ is —NR^{a}R^{b}, wherein R^{a} is optionally substituted carbocyclyl, and R^{b} is hydrogen. In certain embodiments, R¹ is —NR^{a}R^{b}, wherein R^{a} is optionally substituted pyrazolyl, and R^{b} is hydrogen. In certain embodiments, R¹ is —NR^{a}R^{b}, wherein R^{a} is 1-methyl-1H-pyrazol-4-yl, and R^{b} is hydrogen. In certain embodiments, R¹ is

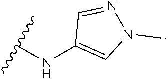 [SMILES: *Nc1cnn(C)c1]

Compounds of Formula (I) include linker L¹ joining the pyrrolopyrazole bicyclic ring and Ring A. Linker L¹ may be —NR^{L1}—, —NR^{L1}C(═O)—, —C(═O)NR^{L1}—, —O—, or —S—, wherein R^{L1} is hydrogen, optionally substituted C₁-C₆ alkyl, or a nitrogen protecting group. In certain embodiments, L¹ is —NR^{L1}—, wherein R^{L1} is hydrogen, optionally substituted C₁-C₆ alkyl, or a nitrogen protecting group. In certain embodiments, L¹ is —NR^{L1}C(═O)—, wherein R^{L1} is hydrogen, optionally substituted C₁-C₆ alkyl, or a nitrogen protecting group. In certain embodiments, L¹ is —C(═O)NR^{L1}—, wherein R^{L1} is hydrogen, optionally substituted C₁-C₆ alkyl, or a nitrogen protecting group. In certain embodiments, R^{L1} is hydrogen. In certain embodiments, L¹ is —NH—. In certain embodiments, L¹ is —NH(C═O)—. In certain embodiments, L¹ is —(C═O)NH—. In certain embodiments, L¹ is —O—. In certain embodiments, L¹ is —S—.

Compounds of Formula (I) include R³ and R⁴ attached to the pyrrolopyrazole bicyclic ring. Each of R³ and R⁴ is independently hydrogen, halogen, optionally substituted aryl, or optionally substituted C₁-C₆ alkyl, or R³ and R⁴ are joined to form an optionally substituted C₃-C₆ carbocyclyl ring. In certain embodiments, R³ is a substituted or unsubstituted aryl (e.g., substituted or unsubstituted phenyl). In certain embodiments, R⁴ is a substituted or unsubstituted aryl (e.g., substituted or unsubstituted phenyl). In certain embodiments, R³ and R⁴ are joined to form an optionally substituted C₃-C₆ carbocyclyl. In certain embodiments, R³ and R⁴ are joined to form an optionally substituted cyclopropane. In certain embodiments, R³ and R⁴ are joined to form an unsubstituted cyclopropane. In certain embodiments, R³ and R⁴ are joined to form an optionally substituted cyclohexane. In certain embodiments, R³ and R⁴ are joined to form an unsubstituted cyclohexane. In certain embodiments, R³ and R⁴ are the same. In certain embodiments, R³ and R⁴ are distinct. In certain embodiments, R³ and R⁴ are optionally substituted C₁-C₆ alkyl. In certain embodiments, R³ and R⁴ are unsubstituted C₁-C₆ alkyl. In certain embodiments, R³ and R⁴ are both methyl. In certain embodiments, R³ and R⁴ are both ethyl. In certain embodiments, R³ and R⁴ are both propyl. In certain embodiments, R³ and R⁴ are both hydrogen. In certain embodiments, R³ and R⁴ are both halogen. In certain embodiments, each of R³ and R⁴ is independently —Cl, —Br, or —I. In certain embodiments, R³ and R⁴ are both —F. In certain embodiments, R³ and R⁴ are joined as —CH₂CH₂—.

In certain embodiments, R³ is optionally substituted C₁-C₆ alkyl (e.g., isopropyl). In certain embodiments, R³ is unsubstituted C₁-C₆ alkyl. In certain embodiments, R³ is methyl. In certain embodiments, R³ is ethyl. In certain embodiments, R³ is propyl. In certain embodiments, R³ is hydrogen. In certain embodiments, R³ is halogen. In certain embodiment, R³ is —Cl, —Br, or —I. In certain embodiment, R³ is —F. In certain embodiments, R⁴ is optionally substituted C₁-C₆ alkyl (e.g., isopropyl). In certain embodiments, R⁴ is unsubstituted C₁-C₆ alkyl. In certain embodiments, R⁴ is methyl. In certain embodiments, R⁴ is ethyl. In certain embodiments, R⁴ is propyl. In certain embodiments, R⁴ is hydrogen. In certain embodiments, R⁴ is halogen. In certain embodiment, R⁴ is —Cl, —Br, or —I. In certain embodiment, R⁴ is —F.

In certain embodiments, R³ is hydrogen, and R⁴ is methyl. In certain embodiments, R³ is methyl, and R⁴ is hydrogen. In certain embodiments, R³ is hydrogen, and R⁴ is ethyl. In certain embodiments, R³ is ethyl, and R⁴ is hydrogen. In certain embodiments, R³ is hydrogen, and R⁴ is propyl. In certain embodiments, R³ is propyl, and R⁴ is hydrogen. In certain embodiments, R³ is hydrogen, and R⁴ is —Cl, —Br, or —I. In certain embodiments, R³ is —Cl, Br, or —I, and R⁴ is hydrogen. In certain embodiments, R³ is hydrogen, and R⁴ is —F. In certain embodiments, R³ is —F, and R⁴ is hydrogen. In certain embodiments, R³ is methyl, and R⁴ is —F. In certain embodiments, R³ is —F, and R⁴ is methyl. In certain embodiments, R³ is ethyl, and R⁴ is —F. In certain embodiments, R³ is —F, and R⁴ is ethyl. In certain embodiments, R³ is propyl, and R⁴ is —F. In certain embodiments, R³ is —F, and R⁴ is propyl. In certain embodiments, R³ is methyl, and R⁴ is —Cl, —Br, or —I. In certain embodiments, R³ is —Cl, —Br, or —I, and R⁴ is methyl. In certain embodiments, R³ is ethyl, and R⁴ is —Cl, —Br, or —I. In certain embodiments, R³ is —Cl, —Br, or —I, and R⁴ is ethyl. In certain embodiments, R³ is propyl, and R⁴ is —Cl, —Br, or —I. In certain embodiments, R³ is —Cl, —Br, or —I, and R⁴ is propyl.

Compounds of Formula (I) include R⁵ attached to a pyrazole nitrogen. R⁵ may be hydrogen, optionally substituted C₁-C₆ alkyl, or a nitrogen protecting group. In certain embodiments, R⁵ is optionally substituted C₁-C₆ alkyl. In certain embodiments, R⁵ is unsubstituted C₁-C₆ alkyl. In certain embodiments, R⁵ is substituted methyl. In certain embodiments, R⁵ is unsubstituted methyl. In certain embodiments, R⁵ is hydrogen. In certain embodiments, R⁵ is a nitrogen protecting group. In certain embodiments, R⁵ is Bn, BOC, Cbz, Fmoc, trifluoroacetyl, triphenylmethyl, acetyl, or Ts.

Compounds of Formula (I) may exist as tautomers or mixtures thereof of Formulae (I-a) and (I-b):

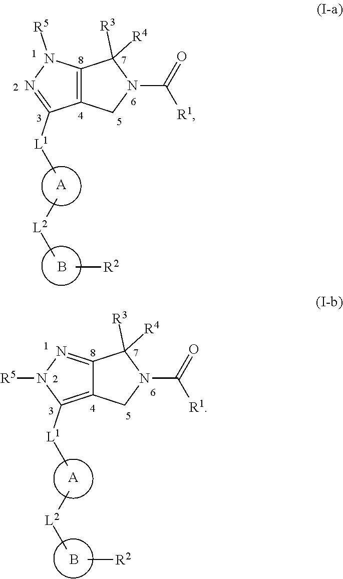

In each tautomer, R⁵ is attached to different pyrazole nitrogens in compounds of each formula. In certain embodiments, R⁵ is attached to the nitrogen at the position labeled 1, as in Formula (I-a). In certain embodiments, R⁵ is attached to the nitrogen at the position labeled 2, as in Formula (I-b). In certain embodiments, compounds of Formula (I) may exist as a mixture of compounds of Formulae (I-a) and (I-b), in which case R⁵ is attached to the nitrogen at the position labeled 1 for components of the mixture corresponding to Formula (I-a), and R⁵ is the nitrogen at the position labeled 2 for components of the mixture corresponding to Formula (I-b).

Compounds of Formula (I) include Ring A between linker L¹ and linker L². Ring A may be optionally substituted carbocyclyl, optionally substituted heterocyclyl, optionally substituted aryl, or optionally substituted heteroaryl. In certain embodiments, Ring A is optionally substituted carbocyclyl. In certain embodiments, Ring A is optionally substituted heterocyclyl. In certain embodiments, Ring A is optionally substituted aryl. In certain embodiments, Ring A is optionally substituted heteroaryl. In certain embodiments, Ring A is optionally substituted phenyl. In certain embodiments, Ring A is phenyl substituted with only L¹ and L2. In certain embodiments, Ring A is optionally substituted cyclohexyl. In certain embodiments, Ring A is optionally substituted piperidinyl. In certain embodiments, Ring A is optionally substituted piperizinyl. In certain embodiments, Ring A is optionally substituted pyridinyl. In certain embodiments, Ring A is optionally substituted pyrimidinyl.

In certain embodiments, linkers L¹ and L² are attached “ortho” or 1,2 to Ring A. In certain embodiments, linkers L¹ and L² are attached “meta” or 1,3 to Ring A. In certain embodiments, linkers L¹ and L² are attached “para” or 1,4 to ring A.

In certain embodiments, Ring A is

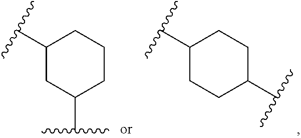 [SMILES: *C1CCCC(*)C1 ; *C1CCC(*)CC1]

wherein each ring atom is optionally substituted. In certain embodiments, Ring A is

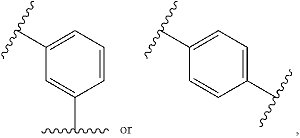 [SMILES: *c1cccc(*)c1 ; *c1ccc(*)cc1]

wherein each ring atom is optionally substituted. In certain embodiments, Ring A is

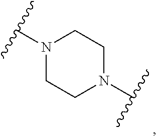 [SMILES: *N1CCN(*)CC1]

wherein each ring atom is optionally substituted. In certain embodiments, Ring A is

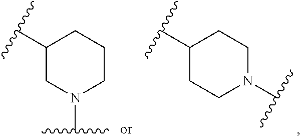 [SMILES: *C1CCCN(*)C1 ; *C1CCN(*)CC1]

wherein each ring atom is optionally substituted, and L¹ and L² may attach to ring A at either indicated position. In certain embodiments, Ring A is

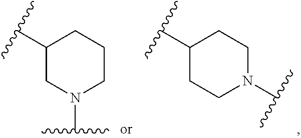 [SMILES: *C1CCCN(*)C1 ; *C1CCN(*)CC1]

wherein each ring atom is optionally substituted, and L¹ and L² may attach to ring A at either indicated position. In certain embodiments, Ring A is

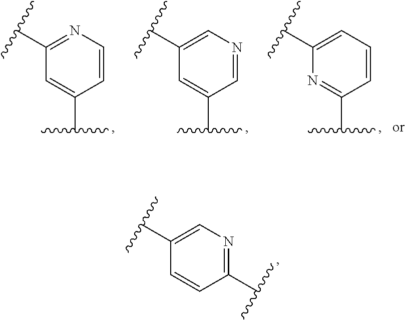 [SMILES: *c1ccnc(*)c1 ; *c1cncc(*)c1 ; *c1cccc(*)n1 ; *c1ccc(*)nc1]

wherein each ring atom is optionally substituted, and L¹ and L² may attach to ring A at either indicated position. In certain embodiments, Ring A is

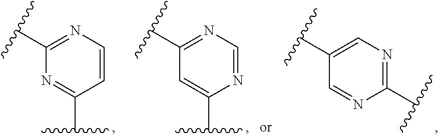 [SMILES: *c1ccnc(*)n1 ; *c1cc(*)ncn1 ; *c1cnc(*)nc1]

wherein each ring atom is optionally substituted, and L¹ and L² may attach to ring A at either indicated position.

In certain embodiments, Ring A is

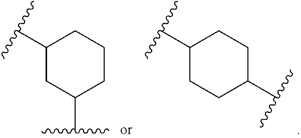 [SMILES: *C1CCCC(*)C1 ; *C1CCC(*)CC1]

In certain embodiments, Ring A is

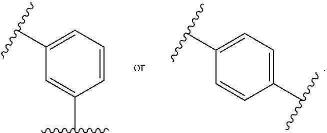 [SMILES: *c1cccc(*)c1 ; *c1ccc(*)cc1]

In certain embodiments, Ring A is

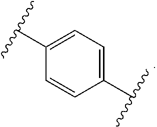 [SMILES: *c1ccc(*)cc1]

In certain embodiments, Ring A is

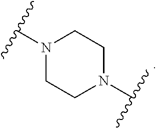 [SMILES: *N1CCN(*)CC1]

In certain embodiments, Ring A is

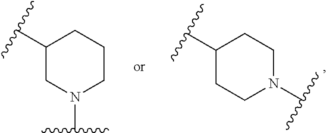 [SMILES: *C1CCCN(*)C1 ; *C1CCN(*)CC1]

L¹ and L² may attach to ring A at either indicated position. In certain embodiments, Ring A is

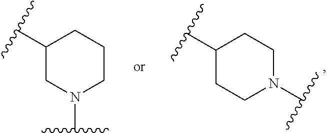

wherein L¹ and L² may attach to ring A at either indicated position. In certain embodiments, Ring A is

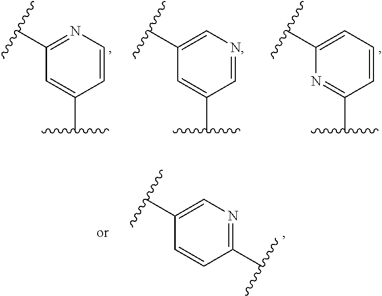

wherein L¹ and L² may attach to ring A at either indicated position. In certain embodiments, Ring A is

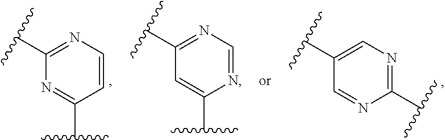

wherein L¹ and L² may attach to ring A at either indicated position.

Compounds of Formula (I) include linker L² joining Ring A to Ring B. Linker L² may be a bond, —C(═O)—, —NR^{L2}—, —C(═O)NR^{L2}—, —NR^{L2}C(═O)—, —O—, or —S— wherein R^{L2} is hydrogen, optionally substituted C₁-C₆ alkyl, or a nitrogen protection group. In certain embodiments, L² is a bond, such that Ring B or R² is directly attached to Ring A. In certain embodiments, L² is —NR^{L2}—, wherein R^{L2} is hydrogen, optionally substituted C₁-C₆ alkyl, or a nitrogen protection group. In certain embodiments, L² is —C(═O)NR^{L2}—, wherein R^{L2} is hydrogen, optionally substituted C₁-C₆ alkyl, or a nitrogen protection group. In certain embodiments, L² is —NR^{L2}C(═O)—, wherein R^{L2} is hydrogen, optionally substituted C₁-C₆ alkyl, or a nitrogen protection group. In certain embodiments, L² is —O—. In certain embodiments, L² is —S—. In certain embodiments, R^{L2} is hydrogen. In certain embodiments, L² is —C(═O)—. In certain embodiments, L² is —NH—. In certain embodiments, L² is —NHC(═O)—. In certain embodiments, L² is —C(═O)NH—. In certain embodiments, L² is —O—. In certain embodiments, L² is —S—.

Compounds of Formula (I) include Ring B between linker L² and group R². In certain embodiments, linker L² is a bond, such that Ring B is directly attached to Ring A. Ring B may absent, optionally substituted carbocyclyl, optionally substituted heterocyclyl, optionally substituted aryl, or optionally substituted heteroaryl. In certain embodiments, Ring B is absent, such that L² is directly attached to R². In certain embodiments, Ring B is absent and linker L² is a bond, such that Ring A is directly attached to R². In certain embodiments, Ring B is optionally substituted carbocyclyl. In certain embodiments, Ring B is optionally substituted heterocyclyl. In certain embodiments, Ring B is optionally substituted aryl. In certain embodiments, Ring B is optionally substituted heteroaryl. In certain embodiments, Ring B is optionally substituted phenyl. In certain embodiments, Ring B is optionally substituted cyclohexyl. In certain embodiments, Ring B is optionally substituted piperidinyl. In certain embodiments, Ring B is optionally substituted piperizinyl. In certain embodiments, Ring B is optionally substituted pyridinyl. In certain embodiments, Ring B is optionally substituted pyrimidinyl.

In certain embodiments, linker L² and group R² are attached “ortho” or 1,2 to each other on Ring B. In certain embodiments, linkers L² and group R² are attached “meta” or 1,2 to each other on Ring B. In certain embodiments, linkers L² and R² are attached “para” or 1,4 to each other on Ring B.

In certain embodiments, Ring B is

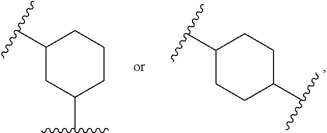 [SMILES: *C1CCCC(*)C1 ; *C1CCC(*)CC1]

wherein each ring atom is optionally substituted. In certain embodiments, Ring B is

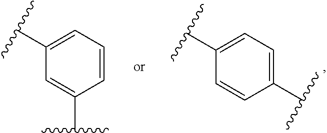 [SMILES: *c1cccc(*)c1 ; *c1ccc(*)cc1]

wherein each ring atom is optionally substituted. In certain embodiments, Ring B is

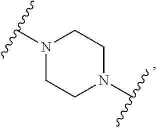 [SMILES: *N1CCN(*)CC1]

wherein each ring atom is optionally substituted. In certain embodiments, Ring B is

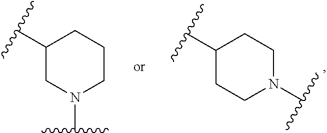 [SMILES: *C1CCCN(*)C1 ; *C1CCN(*)CC1]

wherein each ring atom is optionally substituted, and L² and R² may attach to Ring B at either indicated position. In certain embodiments, Ring B is

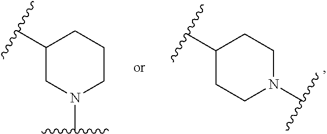 [SMILES: *C1CCCN(*)C1 ; *C1CCN(*)CC1]

wherein each ring atom is optionally substituted, and L² and R² may attach to Ring B at either indicated position. In certain embodiments, Ring B is

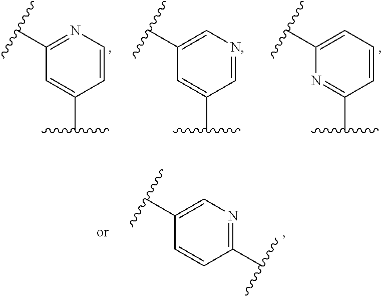 [SMILES: *c1ccnc(*)c1 ; *c1cncc(*)c1 ; *c1cccc(*)n1 ; *c1ccc(*)nc1]

wherein each ring atom is optionally substituted, and L² and R² may attach to Ring B at either indicated position. In certain embodiments, Ring B is

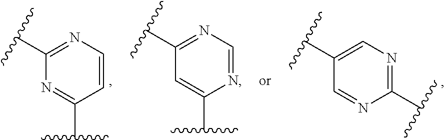 [SMILES: *c1ccnc(*)n1 ; *c1cc(*)ncn1 ; *c1cnc(*)nc1]

wherein each ring atom is optionally substituted, and L² and R² may attach to Ring B at either indicated position.

In certain embodiments, Ring B is

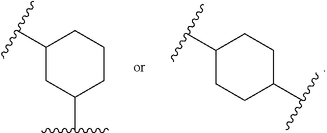 [SMILES: *C1CCCC(*)C1 ; *C1CCC(*)CC1]

In certain embodiments, Ring B is

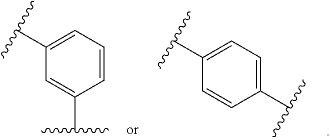 [SMILES: *c1cccc(*)c1 ; *c1ccc(*)cc1]

In certain embodiments, Ring B is

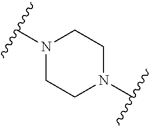 [SMILES: *N1CCN(*)CC1]

In certain embodiments, Ring B is

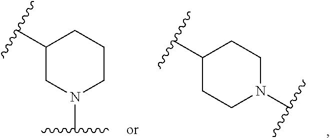 [SMILES: *C1CCCN(*)C1 ; *C1CCN(*)CC1]

L¹ and L² may attach to Ring B at either indicated position. In certain embodiments, Ring B is

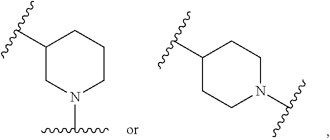

wherein L² and R² may attach to Ring B at either indicated position. In certain embodiments, Ring B is

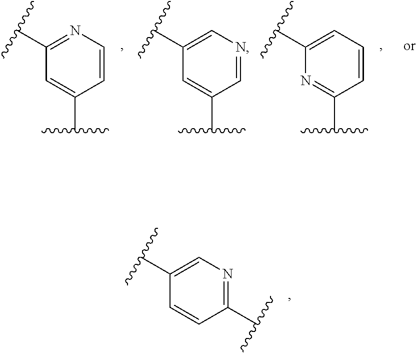

wherein L² and R² may attach to Ring B at either indicated position. In certain embodiments, Ring B is

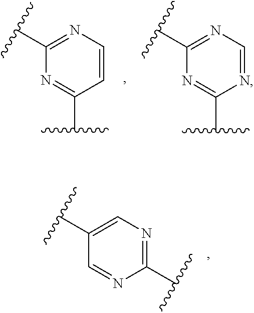

wherein L² and R² may attach to Ring B at either indicated position.

Compounds of Formula (I) include R² attached to Ring B. In certain embodiments, Ring B is absent, such that R² is directly attached to linker L². In certain embodiments, Ring B is absent and L² is a bond, such that R² is directly attached to Ring A. In certain embodiments, R² comprises an electrophilic moiety. In certain embodiments, R² comprises a Michael acceptor moiety. The electrophilic moiety (e.g., Michael acceptor moiety) may react with a cysteine residue of a kinase (e.g., CDK (e.g., CDK7)) to allow for covalent attachment of the compound to the kinase. In certain embodiments, the electrophilic moiety (e.g., Michael acceptor moiety) may react with a cysteine residue of a kinase (e.g., CDK (e.g., CDK7)). In certain embodiments, the electrophilic moiety (e.g., Michael acceptor moiety) may react with the Cys312 residue of CDK7. In certain embodiments, the covalent attachment is irreversible. In certain embodiments, the covalent attachment is reversible.

R² may be any one of Formulae (i-1)-(i-42). In certain embodiments, R² is of Formula (i-1):

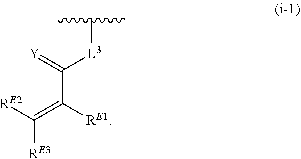

In certain embodiments, R² is of Formula (i-2):

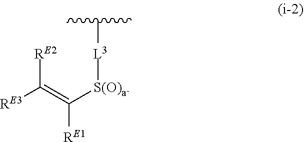

In certain embodiments, R² is of Formula (i-3):

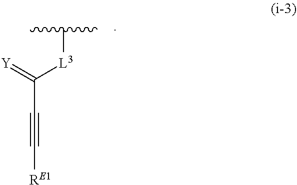

In certain embodiments, R² is of Formula (i-4):

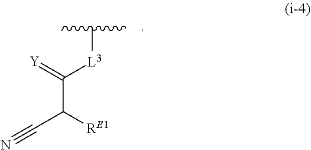

In certain embodiments, R² is of Formula (i-5):

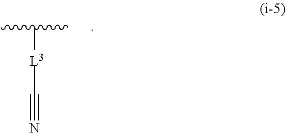

In certain embodiments, R² is of Formula (i-6):

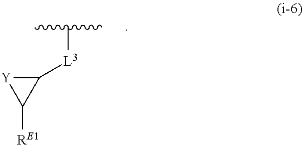

In certain embodiments, R² is of Formula (i-7):

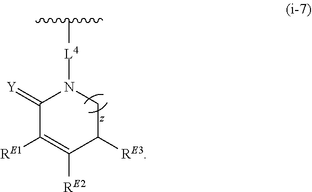

In certain embodiments, R² is of Formula (i-8):

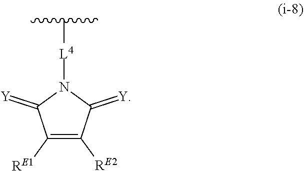

In certain embodiments, R² is of Formula (i-9):

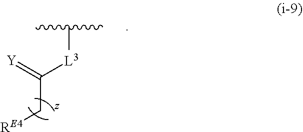

In certain embodiments, R² is of Formula (i-10):

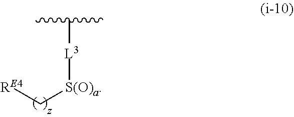

In certain embodiments, R² is of Formula (i-11):

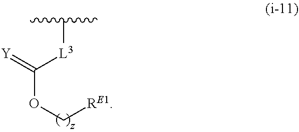

In certain embodiments, R² is of Formula (i-12):

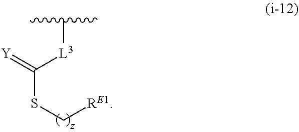

In certain embodiments, R² is of Formula (i-13):

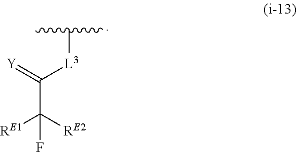

In certain embodiments, R² is of Formula (i-14):

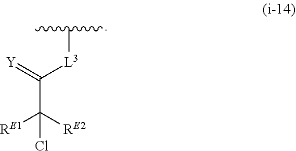

In certain embodiments, R² is of Formula (i-15):

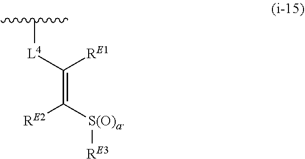

In certain embodiments, R² is of Formula (i-16):

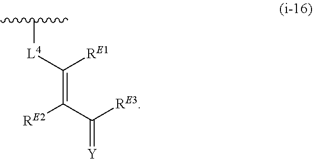

In certain embodiments, R² is of Formula (i-17):

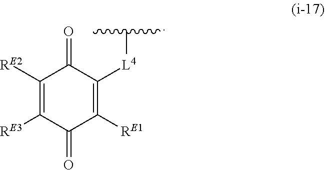

In certain embodiments, R² is of Formula (i-18):

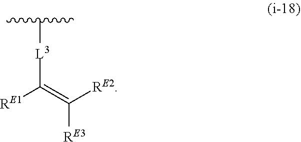

In certain embodiments, R² is of Formula (i-19):

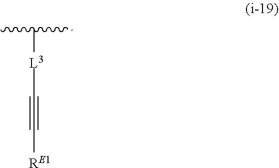

In certain embodiments, R² is of Formula (i-20):

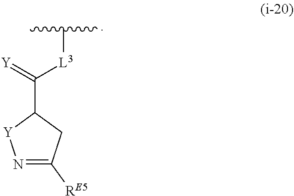

In certain embodiments, R² is of Formula (i-21):

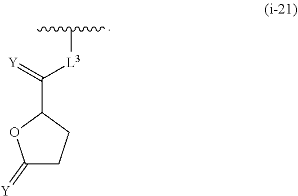

In certain embodiments, R² is of Formula (i-22):

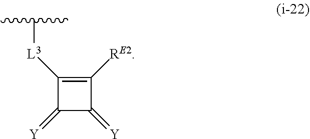

In certain embodiments, R² is of Formula (i-23):

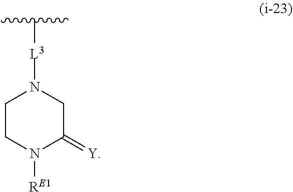

In certain embodiments, R² is of Formula (i-24):

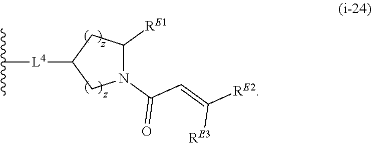

In certain embodiments, R² is of Formula (i-25):

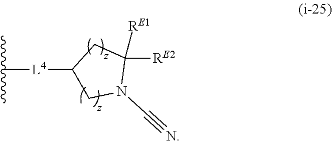

In certain embodiments, R² is of Formula (i-26):

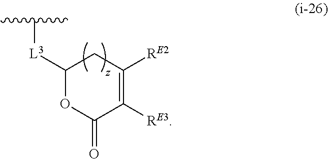

In certain embodiments, R² is of Formula (i-27):

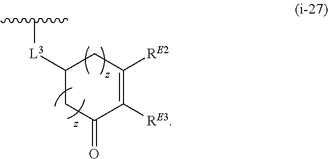

In certain embodiments, R² is of Formula (i-28):

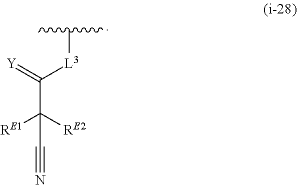

In certain embodiments, R² is of Formula (i-29):

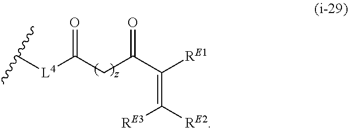

In certain embodiments, R² is of Formula (i-30):

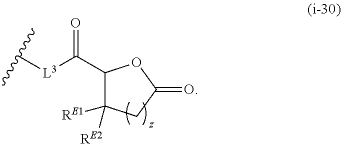

In certain embodiments, R² is of Formula (i-31):

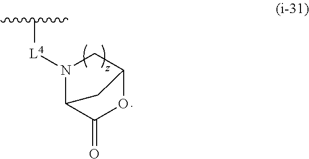

In certain embodiments, R² is of Formula (i-32):

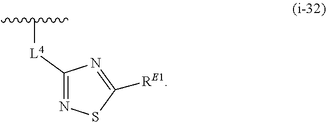

In certain embodiments, R² is of Formula (i-33):

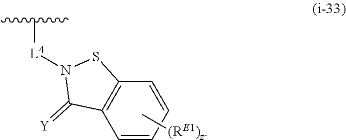

In certain embodiments, R² is of Formula (i-34):

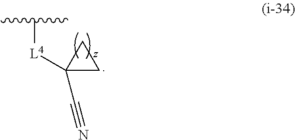

In certain embodiments, R² is of Formula (i-35):

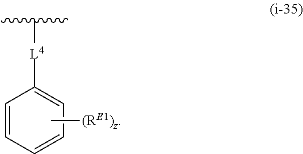

In certain embodiments, R² is of Formula (i-36):

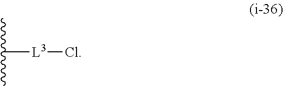

In certain embodiments, R² is of Formula (i-37):

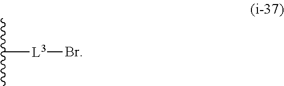

In certain embodiments, R² is of Formula (i-38):

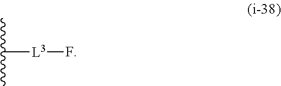

In certain embodiments, R² is of Formula (i-39):

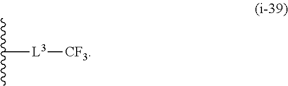

In certain embodiments, R² is of Formula (i-40):

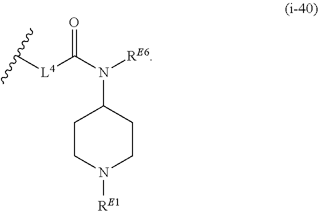

In certain embodiments, R² is of Formula (i-41):

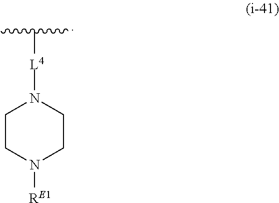

In certain embodiments, R² is of Formula (i-42):

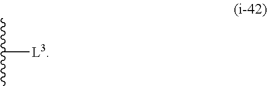

In certain embodiments, R² is of Formula (i-1a):

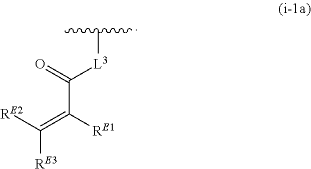

In certain embodiments, R² is of Formula (i-1b):

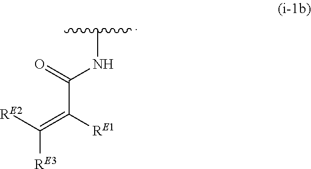

In certain embodiments, R² is of Formula (i-1c):

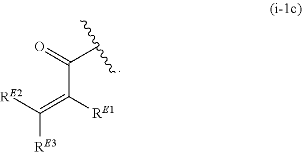

In certain embodiments, R² is of Formula (i-1d):

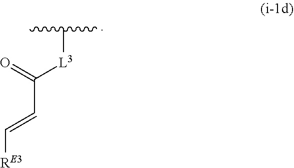

In certain embodiments, R² is of Formula (i-1e):

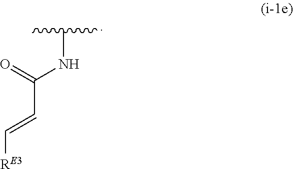

In certain embodiments, R² is of Formula (i-1f):

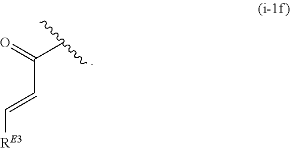

In certain embodiments, R² is of Formula (i-1g):

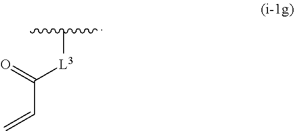

In certain embodiments, R² is of Formula (i-1g):

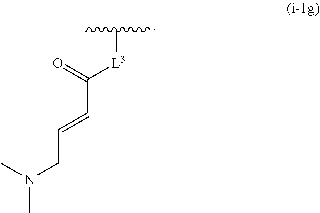

In certain embodiments, R² is

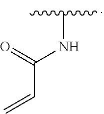 [SMILES: *NC(=O)C=C]

In certain embodiments, R² is

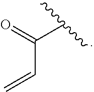 [SMILES: *C(=O)C=C]

In certain embodiments, R² is

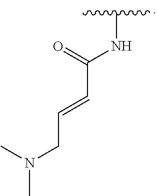 [SMILES: *NC(=O)/C=C/CN(C)C]

In certain embodiments, R² is

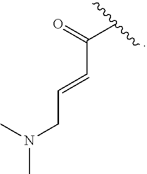 [SMILES: *C(=O)/C=C/CN(C)C]

In certain embodiments, R² is of Formula (i-1a):

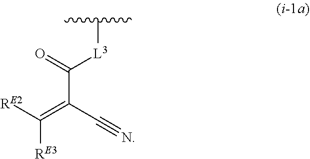

In certain embodiments, R² is of Formula (i-1b):

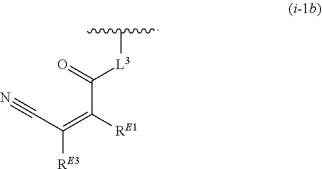

In certain embodiments, R² is of Formula (i-1c):

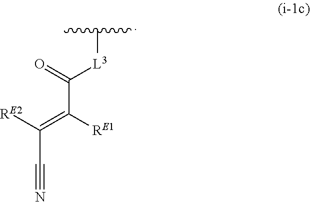

In certain embodiments, R² is of Formula (i-18a):

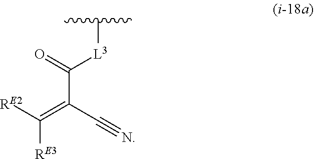

In certain embodiments, R² is of Formula (i-18b):

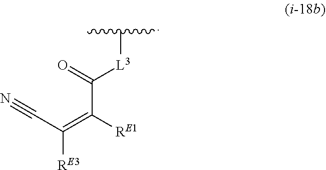

In certain embodiments, R² is of Formula (i-18c):

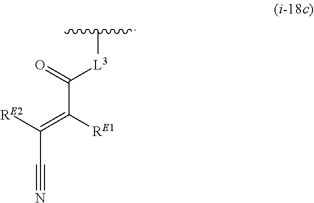

In certain embodiments, R² is of Formula (i-15a):

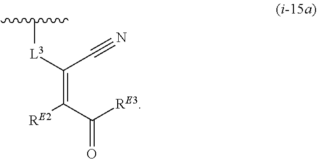

In certain embodiments, R² is of Formula (i-15b):

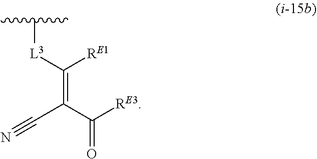

In certain embodiments, R² is of Formula (i-15c):

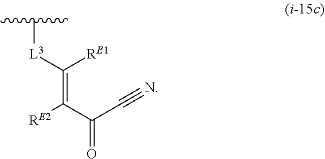

R² may contain linker L³ or L⁴. In certain embodiments, L³ is a bond. L³ is an optionally substituted C₁₋₄ hydrocarbon chain. In certain embodiments, L³ is optionally substituted ethyl. In certain embodiments, L³ is optionally substituted alkenyl. In certain embodiments, L³ is an optionally substituted C₁₋₄ hydrocarbon chain, wherein one or more carbon units of the hydrocarbon chain are independently replaced with —C(═O)—, —O—, —S—, —NR^{L3a}—, NR^{L3a}C(═O)—, —C(═O)NR^{L3a}—, —SC(═O)—, —C(═O)S—, —OC(═O)—, —C(═O)O—, —NR^{L3a}C(═S)—, —C(═S)NR^{L3a}—, trans-CR^{L3b}═CR^{L3b}—, cis-CR^{L3b}═CR^{L3b}—, —C≡C—, —S(═O)—, —S(═O)O—, —OS(═O)—, —S(═O)NR^{L3a}—, NR^{L3a}S(═O)—S(═O)₂—, —S(═O)₂O—, —OS(═O)₂—, —S(═O)₂NR^{L3a}—, or —NR^{L3a}S(═O)₂—. In certain embodiments, L³ is an optionally substituted C₁₋₄ hydrocarbon chain, wherein one carbon unit of the hydrocarbon chain is replaced with —NR^{L3a}— (e.g., —NH—). In certain embodiments, L³ is of the formula: —(CH₂)₁₋₄—NR^{L3a}— (e.g., —(CH₂)₁₋₄—NH—) or —NR^{L3a}—CH₂)₁₋₄— (e.g., —NH—CH₂)₁₋₄—). In certain embodiments, L³ is —NR^{L3a}—. In certain embodiments, L³ is —NR^{L3a}(C═O)—. In certain embodiments, L³ is —(C═O)NR^{L3a} In certain embodiments, L³ is —NH—. In certain embodiments, L³ is —(C═O)—. In certain embodiments, L³ is —NH(C═O)—. In certain embodiments, L³ is —(C═O)NH—. In certain embodiments, L³ is —O—. In certain embodiments, L³ is —S—. In certain embodiments, L⁴ is a bond. In certain embodiments, L⁴ is an optionally substituted C₁₋₄ hydrocarbon chain.

Linker L³ may contain groups R^{L3a} or R^{L3b} In certain embodiments, R^{L3a} is hydrogen. In certain embodiments, at least one instance of R^{L3b} is hydrogen. In certain embodiments, each instance of R^{L3b} is hydrogen. In certain embodiments, at least one instance of R^{L3b} is —Cl, —Br, or —I. In certain embodiments, each instance of R^{L3b} is —Cl, —Br, or —I. In certain embodiments, at least one instance of R^{L3b} is —F. In certain embodiments, each instance of R^{L3b} is —F. In certain embodiments, at least one instance of R^{L3b} is optionally substituted alkyl, optionally substituted alkenyl, optionally substituted alkynyl, optionally substituted carbocyclyl, optionally substituted heterocyclyl, optionally substituted aryl, or optionally substituted heteroaryl. In certain embodiments, two R^{L3b} groups are joined to form an optionally substituted carbocyclic or optionally substituted heterocyclic ring.

R² may contain groups R^{E1}, R^{E2}, and/or R^{E3}. In certain embodiments, R^{E1} is hydrogen. In certain embodiments, R^{E2} is hydrogen. In certain embodiments, R^{E3} is hydrogen. In certain embodiments, R^{E1} is —Cl, —Br, or —I. In certain embodiments, R^{E2} is —Cl, —Br, or —I. In certain embodiments, R^{E3} is —Cl, —Br, or —I. In certain embodiments, R^{E1} is —F. In certain embodiments, R^{E2} is —F. In certain embodiments, R^{E3} is —F. In certain embodiments, R^{E1} is optionally substituted alkyl (e.g., substituted or unsubstituted C₁₋₆ alkyl). In certain embodiments, R^{E2} is optionally substituted alkyl (e.g., substituted or unsubstituted C₁₋₆ alkyl). In certain embodiments, R^{E3} is optionally substituted alkyl (e.g., substituted or unsubstituted C₁₋₆ alkyl). In certain embodiments, R^{E1} is optionally substituted alkenyl, optionally substituted alkynyl, optionally substituted carbocyclyl, optionally substituted heterocyclyl, optionally substituted aryl, optionally substituted heteroaryl, —CN, —CH₂OR^{EE}, —CH₂N(R^{EE})₂, —CH₂SR^{EE}, —OR^{EE}, —N(R^{EE})₂, —Si(R^{EE})₃, or —SR^{EE}. In certain embodiments, R^{E2} is optionally substituted alkenyl, optionally substituted alkynyl, optionally substituted carbocyclyl, optionally substituted heterocyclyl, optionally substituted aryl, optionally substituted heteroaryl, —CN, —CH₂OR^{EE}, —CH₂N(R^{EE})₂, —CH₂SR^{EE}, —OR^{EE}, —N(R^{EE})₂, —Si(R^{EE})₃, or —SR^{EE}. In certain embodiments, R^{E3} is optionally substituted alkenyl, optionally substituted alkynyl, optionally substituted carbocyclyl, optionally substituted heterocyclyl, optionally substituted aryl, optionally substituted heteroaryl, —CN, —CH₂OR^{EE}, —CH₂N(R^{EE})₂, —CH₂SR^{EE}, —OR^{EE}. —N(R^{EE})₂, —Si(R^{EE})₃, or —SR^{EE}. In certain embodiments, R^{E1} is —N(R^{EE})₂. In certain embodiments, R^{E2} is —N(R^{EE})₂. In certain embodiments, R^{E3} is —N(R^{EE})₂. In certain embodiments, R^{E1} is —N(CH₃)₂. In certain embodiments, R^{E2} is —N(CH₃)₂. In certain embodiments, R^{E3} is —N(CH₃)₂. In certain embodiments, R^{E1} is —CH₂N(R^{EE})₂. In certain embodiments, R^{E2} is —CH₂N(R^{EE})₂. In certain embodiments, R^{E3} is —CH₂N(R^{EE})₂. In certain embodiments, R^{E1} is —CH₂N(CH₃)₂. In certain embodiments, R^{E2} is —CH₂N(CH₃)₂. In certain embodiments, R^{E3} is —CH₂N(CH₃)₂. In certain embodiments, R^{E1} is —CN. In certain embodiments, R^{E2} is —CN. In certain embodiments, R^{E3} is —CN.

In certain embodiments, R^{E1} and R^{E3} are joined to form an optionally substituted carbocyclic ring. In certain embodiments, R^{E1} and R^{E3} are joined to form an optionally substituted heterocyclic ring. In certain embodiments, R^{E2} and R^{E3} are joined to form an optionally substituted carbocyclic ring. In certain embodiments, R^{E2} and R^{E3} are joined to form an optionally substituted heterocyclic ring. In certain embodiments, R^{E1} and R^{E2} are joined to form an optionally substituted carbocyclic ring. In certain embodiments, R^{E1} and R^{E2} are joined to form an optionally substituted heterocyclic ring.

R² may contain group R^{E4}, where R^{E4} is a leaving group. In certain embodiments, R^{E4} is —Cl, —Br, or —I. In certain embodiments, R^{E4} is —F. In certain embodiments, R^{E4} is —OS(═O)R^{E4a} or —OS(═O)₂R^{E4a}, wherein R^{E4a} is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl. In certain embodiments, R^{E4} is —OR^{E4a}. In certain embodiments, R^{E4} is —OMs, —OTf, —OTs, —OBs, or 2-nitrobenzenesulfonyloxy. In certain embodiments, R^{E4} is —OR^{E4a}. In certain embodiments, R^{E4} is —OMe, —OCF₃, or —OPh. In certain embodiments, R^{E4} is —OC(═O)R^{E4a}. In certain embodiments, R^{E4} is —OC(═O)Me, —OC(═O)CF₃, —OC(═O)Ph, or —OC(═O)Cl. In certain embodiments, R^{E4} is —OC(═O)OR^{E4a}. In certain embodiments, R^{E4} is —OC(═O)OMe or —OC(═O)O(t-Bu).

R² may contain group R^{E5}, where R^{E5} is a halogen. In certain embodiments, R^{E5} is —Cl, —Br, or —I. In certain embodiments, R^{E5} is —F.

R² may contain group R^{E6}. In certain embodiments, R^{E6} is hydrogen. In certain embodiments, R^{E6} is substituted or unsubstituted C₁-C₆ alkyl. In certain embodiments, R^{E6} is a nitrogen protecting group.

In certain embodiments, a is 1. In certain embodiments, a is 2.

In certain embodiments, z is 0. In certain embodiments, z is 1. In certain embodiments, z is 2. In certain embodiments, z is 3, 4, 5, or 6.

R² may contain group Y. In certain embodiments, Y is O. In certain embodiments, Y is S. In certain embodiments, Y is NR^{E7}. In certain embodiments, Y is NH.

In certain embodiments, the compound of Formula (I) is a compound of Formula (II):

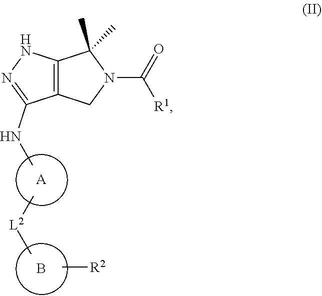

or a pharmaceutically acceptable salt, solvate, hydrate, polymorph, co-crystal, tautomer, stereoisomer, isotopically labeled derivative, or prodrug thereof, wherein R¹, R², linker L², Ring A, and Ring B are as defined for Formula (I).

In certain embodiments, the compound of Formula (I) is of Formula (III):

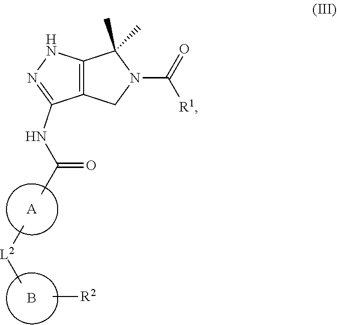

or a pharmaceutically acceptable salt, solvate, hydrate, polymorph, co-crystal, tautomer, stereoisomer, isotopically labeled derivative, or prodrug thereof, wherein R¹, R², linker L², Ring A, and Ring B are as defined for Formula (I).

In certain embodiments, the compound of Formula (I) is of Formula (IV-a):

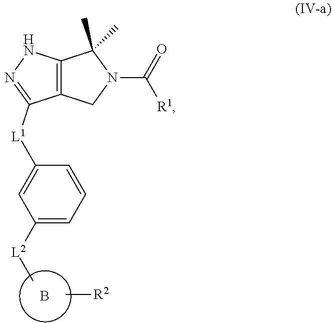

or a pharmaceutically acceptable salt, solvate, hydrate, polymorph, co-crystal, tautomer, stereoisomer, isotopically labeled derivative, or prodrug thereof, wherein R¹, R², linker L¹, linker L², and Ring B are as defined for Formula (I).

In certain embodiments, the compound of Formula (I) is of Formula (IV-b):

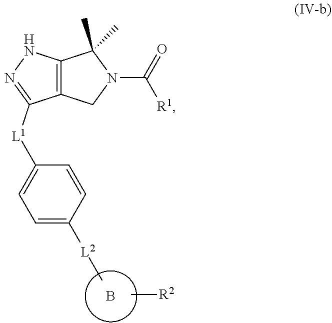

or a pharmaceutically acceptable salt, solvate, hydrate, polymorph, co-crystal, tautomer, stereoisomer, isotopically labeled derivative, or prodrug thereof, wherein R¹, R², linker L¹, linker L², and Ring B are as defined for Formula (I).

In certain embodiments, the compound Formula (I) is of Formula (IV-c):

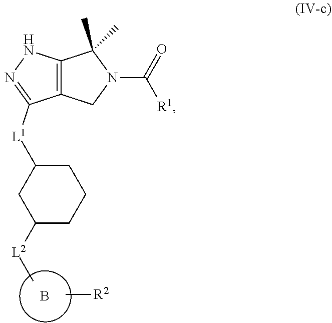

or a pharmaceutically acceptable salt, solvate, hydrate, polymorph, co-crystal, tautomer, stereoisomer, isotopically labeled derivative, or prodrug thereof, wherein R¹, R², linker L¹, linker L², and Ring B are as defined for Formula (I).

In certain embodiments, the compound Formula (I) is of Formula (IV-d):

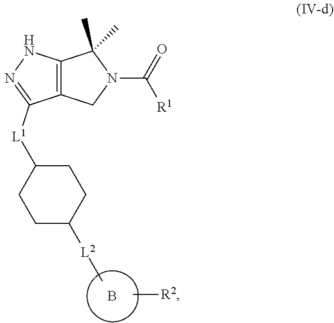

or a pharmaceutically acceptable salt, solvate, hydrate, polymorph, co-crystal, tautomer, stereoisomer, isotopically labeled derivative, or prodrug thereof, wherein R¹, R², linker L¹, linker L², and Ring B are as defined for Formula (I).

In certain embodiments, the compound Formula (I) is of Formula (IV-e):

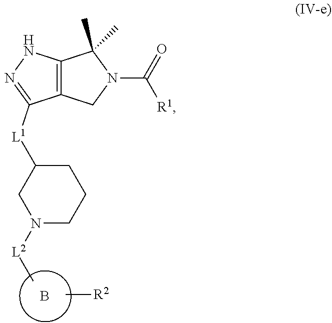

or a pharmaceutically acceptable salt, solvate, hydrate, polymorph, co-crystal, tautomer, stereoisomer, isotopically labeled derivative, or prodrug thereof, wherein R¹, R², linker L¹, linker L², and Ring B are as defined for Formula (I).

In certain embodiments, the compound of Formula (I) is of Formula (IV-f):

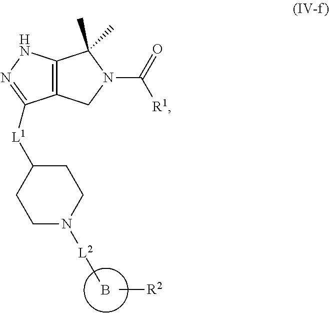

or a pharmaceutically acceptable salt, solvate, hydrate, polymorph, co-crystal, tautomer, stereoisomer, isotopically labeled derivative, or prodrug thereof, wherein R¹, R², linker L¹, linker L², and Ring B are as defined for Formula (I).

In certain embodiments, the compound of Formula (I) is of Formula (V-a):

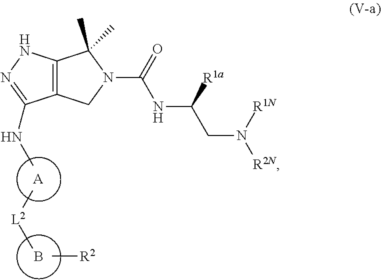

or a pharmaceutically acceptable salt, solvate, hydrate, polymorph, co-crystal, tautomer, stereoisomer, isotopically labeled derivative, or prodrug thereof, wherein:
R², linker L², Ring A, and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (V-b):

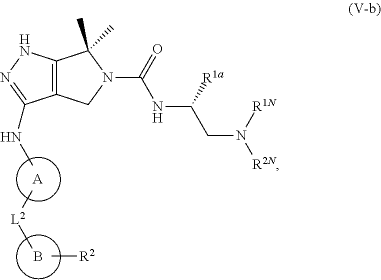

or a pharmaceutically acceptable salt, solvate, hydrate, polymorph, co-crystal, tautomer, stereoisomer, isotopically labeled derivative, or prodrug thereof, wherein:
R², linker L², Ring A, and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (V-c):

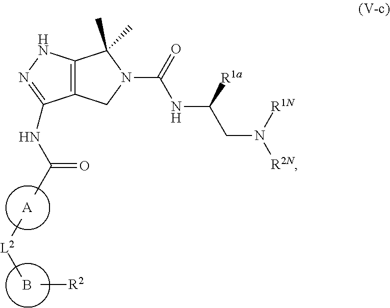

or a pharmaceutically acceptable salt, solvate, hydrate, polymorph, co-crystal, tautomer, stereoisomer, isotopically labeled derivative, or prodrug thereof, wherein:
R², linker L², Ring A, and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (V-d):

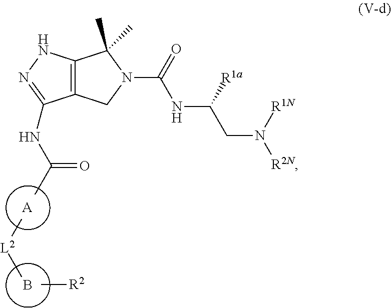

or a pharmaceutically acceptable salt, solvate, hydrate, polymorph, co-crystal, tautomer, stereoisomer, isotopically labeled derivative, or prodrug thereof, wherein:
R², linker L², Ring A, and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (VI-a):

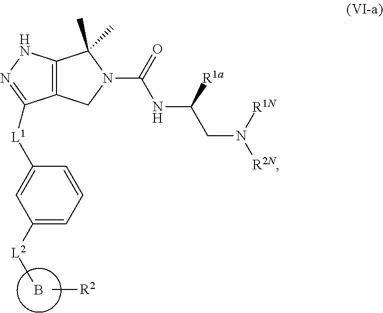

or a pharmaceutically acceptable salt, solvate, hydrate, polymorph, co-crystal, tautomer, stereoisomer, isotopically labeled derivative, or prodrug thereof, wherein:
R², linker L¹, linker L², and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (VI-b):

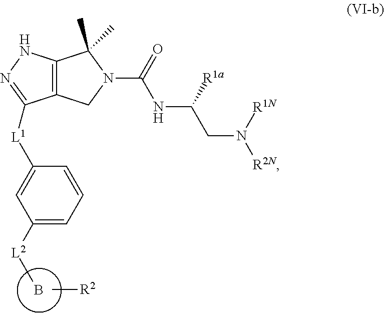

or a pharmaceutically acceptable salt, solvate, hydrate, polymorph, co-crystal, tautomer, stereoisomer, isotopically labeled derivative, or prodrug thereof, wherein:
R², linker L¹, linker L², and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (VI-c):

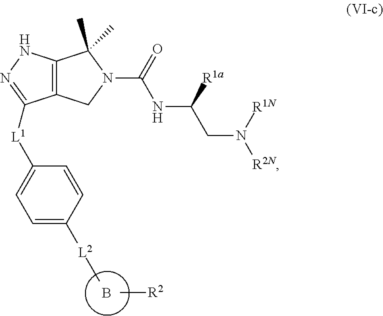

or a pharmaceutically acceptable salt, solvate, hydrate, polymorph, co-crystal, tautomer, stereoisomer, isotopically labeled derivative, or prodrug thereof, wherein:
R², linker L¹, linker L², and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (VI-d):

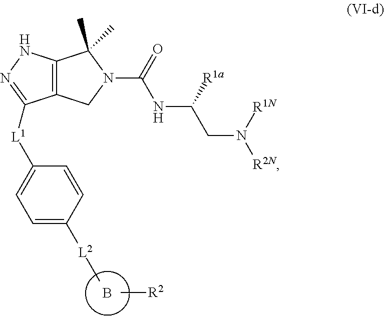

or a pharmaceutically acceptable salt, solvate, hydrate, polymorph, co-crystal, tautomer, stereoisomer, isotopically labeled derivative, or prodrug thereof, wherein:
R², linker L¹, linker L², and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (VI-e):

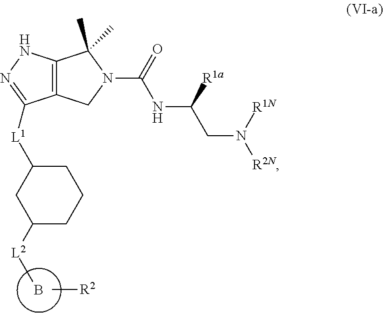

or a pharmaceutically acceptable salt, solvate, hydrate, polymorph, co-crystal, tautomer, stereoisomer, isotopically labeled derivative, or prodrug thereof, wherein:
R², linker L¹, linker L², and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (VI-f):

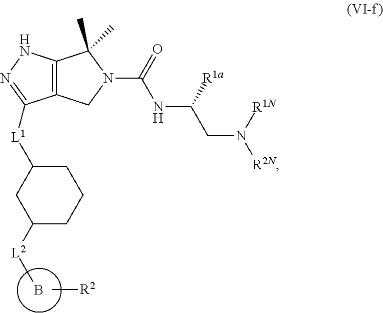

or a pharmaceutically acceptable salt, solvate, hydrate, polymorph, co-crystal, tautomer, stereoisomer, isotopically labeled derivative, or prodrug thereof, wherein:
R², linker L¹, linker L², and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (VI-g):

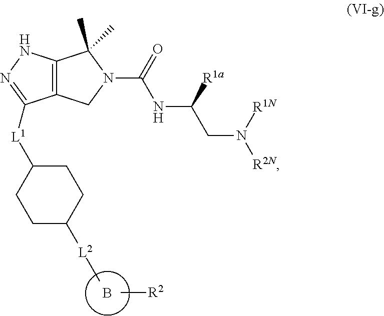

or a pharmaceutically acceptable salt, solvate, hydrate, polymorph, co-crystal, tautomer, stereoisomer, isotopically labeled derivative, or prodrug thereof, wherein:
R², linker L¹, linker L², and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (VI-h):

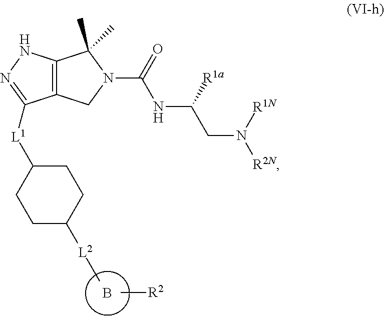

or a pharmaceutically acceptable salt, solvate, hydrate, polymorph, co-crystal, tautomer, stereoisomer, isotopically labeled derivative, or prodrug thereof, wherein:
R², linker L¹, linker L², and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (VI-i):

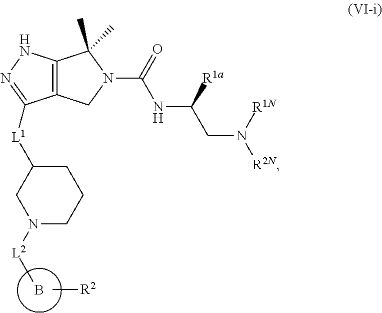

or a pharmaceutically acceptable salt, solvate, hydrate, polymorph, co-crystal, tautomer, stereoisomer, isotopically labeled derivative, or prodrug thereof, wherein:
R², linker L¹, linker L², and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (VI-j):

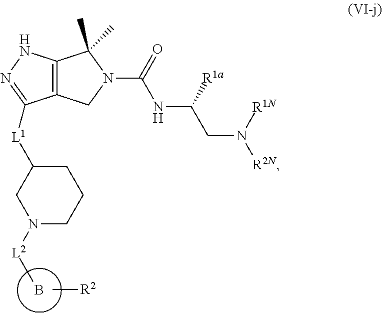

or a pharmaceutically acceptable salt, solvate, hydrate, polymorph, co-crystal, tautomer, stereoisomer, isotopically labeled derivative, or prodrug thereof, wherein:
R², linker L¹, linker L², and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (VI-k):

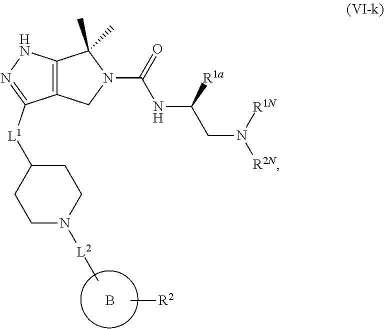

or a pharmaceutically acceptable salt, solvate, hydrate, polymorph, co-crystal, tautomer, stereoisomer, isotopically labeled derivative, or prodrug thereof, wherein:
R², linker L¹, linker L², and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (VI-1):

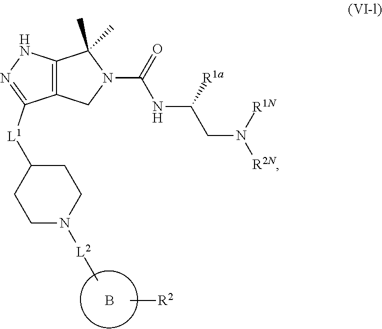

or a pharmaceutically acceptable salt, solvate, hydrate, polymorph, co-crystal, tautomer, stereoisomer, isotopically labeled derivative, or prodrug thereof, wherein:
R², linker L¹, linker L², and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (VII-a):

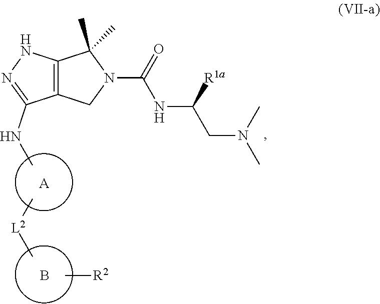

or a pharmaceutically acceptable salt, solvate, hydrate, polymorph, co-crystal, tautomer, stereoisomer, isotopically labeled derivative, or prodrug thereof, wherein:
R², linker L², Ring A, and Ring B are as defined for Formula (I); and
R^{1a} is hydrogen, C₁-C₆ alkyl, or optionally substituted aryl.

In certain embodiments, the compound of Formula (I) is of Formula (VII-b):

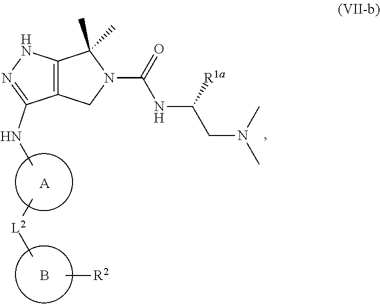

or a pharmaceutically acceptable salt, solvate, hydrate, polymorph, co-crystal, tautomer, stereoisomer, isotopically labeled derivative, or prodrug thereof, wherein:
R², linker L², Ring A, and Ring B are as defined for Formula (I); and
R^{1a} is hydrogen, C₁-C₆ alkyl, or optionally substituted aryl.

In certain embodiments, the compound of Formula (I) is of Formula (VII-c):

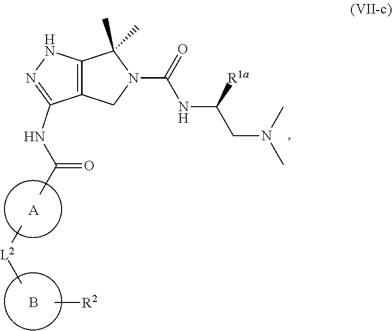

or a pharmaceutically acceptable salt, solvate, hydrate, polymorph, co-crystal, tautomer, stereoisomer, isotopically labeled derivative, or prodrug thereof, wherein:
R², linker L², Ring A, and Ring B are as defined for Formula (I); and
R^{1a} is hydrogen, C₁-C₆ alkyl, or optionally substituted aryl.

In certain embodiments, the compound of Formula (I) is of Formula (VII-d):

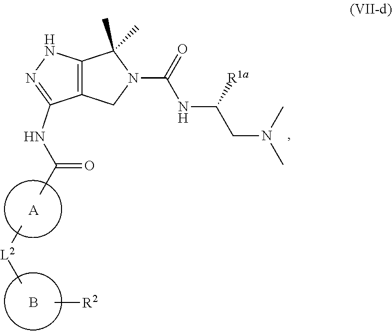

or a pharmaceutically acceptable salt, solvate, hydrate, polymorph, co-crystal, tautomer, stereoisomer, isotopically labeled derivative, or prodrug thereof, wherein:
R², linker L², Ring A, and Ring B are as defined for Formula (I); and
R^{1a} is hydrogen, C₁-C₆ alkyl, or optionally substituted aryl.

In certain embodiments, the compound of Formula (I) is of Formula (VIII-a):

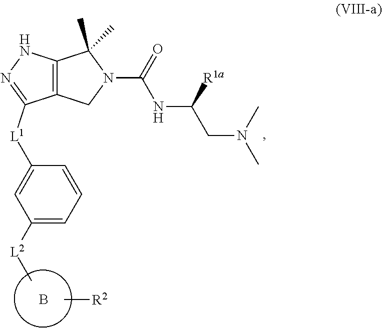

or a pharmaceutically acceptable salt, solvate, hydrate, polymorph, co-crystal, tautomer, stereoisomer, isotopically labeled derivative, or prodrug thereof, wherein:
R², linker Lt, linker L², and Ring B are as defined for Formula (I); and
R^{1a} is hydrogen, C₁-C₆ alkyl, or optionally substituted aryl.

In certain embodiments, the compound of Formula (I) is of Formula (VIII-b):

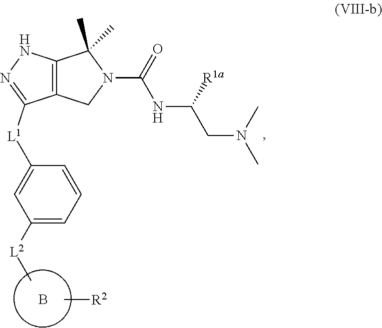

or a pharmaceutically acceptable salt, solvate, hydrate, polymorph, co-crystal, tautomer, stereoisomer, isotopically labeled derivative, or prodrug thereof, wherein:
R², linker L¹, linker L², and Ring B are as defined for Formula (I); and
R^{1a} is hydrogen, C₁-C₆ alkyl, or optionally substituted aryl.

In certain embodiments, the compound of Formula (I) is of Formula (VIII-c):

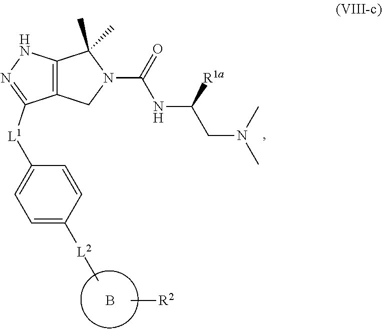

or a pharmaceutically acceptable salt, solvate, hydrate, polymorph, co-crystal, tautomer, stereoisomer, isotopically labeled derivative, or prodrug thereof, wherein:
R², linker L¹, linker L², and Ring B are as defined for Formula (I); and
R^{1a} is hydrogen, C₁-C₆ alkyl, or optionally substituted aryl.

In certain embodiments, the compound of Formula (I) is of Formula (VIII-d):

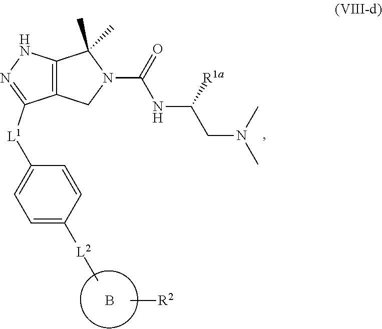

or a pharmaceutically acceptable salt, solvate, hydrate, polymorph, co-crystal, tautomer, stereoisomer, isotopically labeled derivative, or prodrug thereof, wherein:
R², linker L¹, linker L², and Ring B are as defined for Formula (I); and
R^{1a} is hydrogen, C₁-C₆ alkyl, or optionally substituted aryl.

In certain embodiments, the compound of Formula (I) is of Formula (VIII-e):

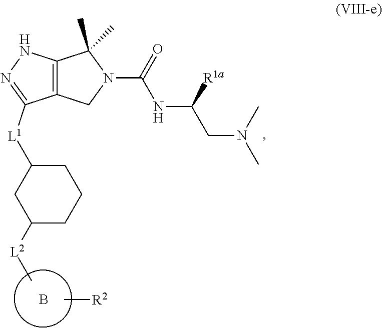

or a pharmaceutically acceptable salt, solvate, hydrate, polymorph, co-crystal, tautomer, stereoisomer, isotopically labeled derivative, or prodrug thereof wherein:
R², linker L¹, linker L², and Ring B are as defined for Formula (I); and
R^{1a} is hydrogen, C₁-C₆ alkyl, or optionally substituted aryl.

In certain embodiments, the compound of Formula (I) is of Formula (VIII-f):

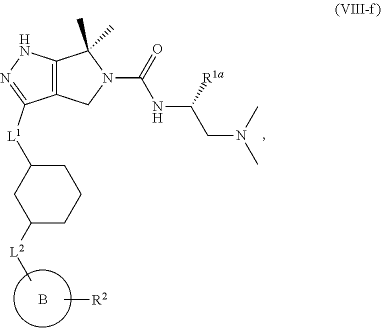

or a pharmaceutically acceptable salt, solvate, hydrate, polymorph, co-crystal, tautomer, stereoisomer, isotopically labeled derivative, or prodrug thereof wherein:
R², linker L¹, linker L², and Ring B are as defined for Formula (I); and
R^{1a} is hydrogen, C₁-C₆ alkyl, or optionally substituted aryl.

In certain embodiments, the compound of Formula (I) is of Formula (VIII-g):

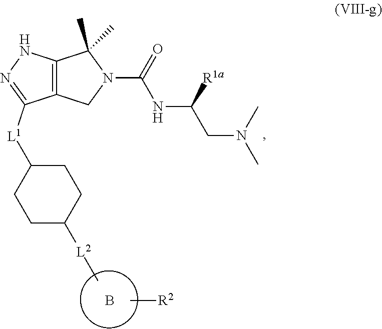

or a pharmaceutically acceptable salt, solvate, hydrate, polymorph, co-crystal, tautomer, stereoisomer, isotopically labeled derivative, or prodrug thereof wherein:
R², linker L¹, linker L², and Ring B are as defined for Formula (I); and
R^{1a} is hydrogen, C₁-C₆ alkyl, or optionally substituted aryl.

In certain embodiments, the compound of Formula (I) is of Formula (VIII-h):

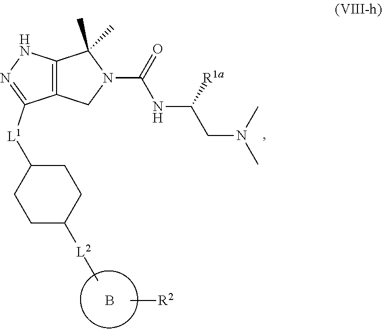

or a pharmaceutically acceptable salt, solvate, hydrate, polymorph, co-crystal, tautomer, stereoisomer, isotopically labeled derivative, or prodrug thereof wherein:
R², linker L¹, linker L², and Ring B are as defined for Formula (I); and
R^{1a} is hydrogen, C₁-C₆ alkyl, or optionally substituted aryl.

In certain embodiments, the compound of Formula (I) is of Formula (VIII-i):

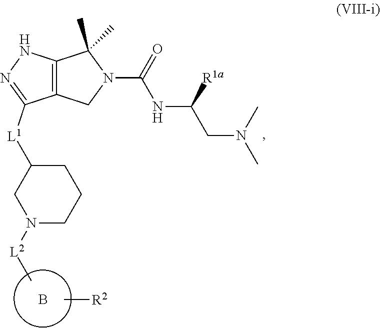

or a pharmaceutically acceptable salt, solvate, hydrate, polymorph, co-crystal, tautomer, stereoisomer, isotopically labeled derivative, or prodrug thereof wherein:
R², linker L¹, linker L², and Ring B are as defined for Formula (I); and
R^{1a} is hydrogen, C₁-C₆ alkyl, or optionally substituted aryl.

In certain embodiments, the compound of Formula (I) is of Formula (VIII-j):

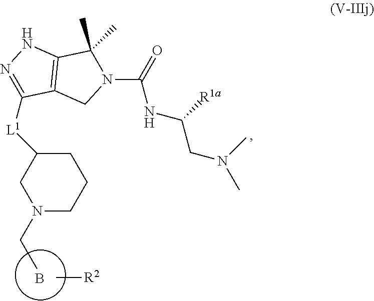

or a pharmaceutically acceptable salt, solvate, hydrate, polymorph, co-crystal, tautomer, stereoisomer, isotopically labeled derivative, or prodrug thereof wherein:
R², linker L¹, linker L², and Ring B are as defined for Formula (I); and
R^{1a} is hydrogen, C₁-C₆ alkyl, or optionally substituted aryl.

In certain embodiments, the compound of Formula (I) is of Formula (VIII-k):

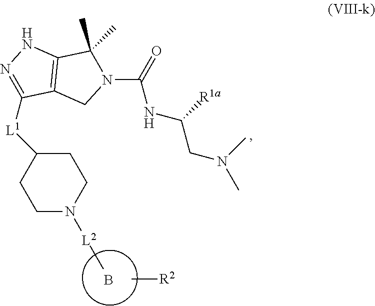

or a pharmaceutically acceptable salt, solvate, hydrate, polymorph, co-crystal, tautomer, stereoisomer, isotopically labeled derivative, or prodrug thereof wherein:
R², linker L¹, linker L², and Ring B are as defined for Formula (I); and
R^{1a} is hydrogen, C₁-C₆ alkyl, or optionally substituted aryl.

In certain embodiments, the compound of Formula (I) is of Formula (VIII-1):

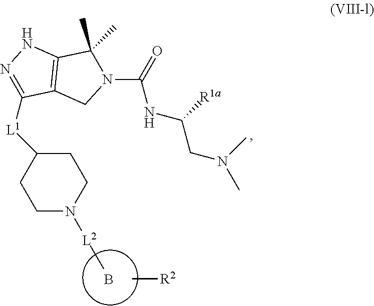

or a pharmaceutically acceptable salt, solvate, hydrate, polymorph, co-crystal, tautomer, stereoisomer, isotopically labeled derivative, or prodrug thereof wherein:
R², linker L¹, linker L², and Ring B are as defined for Formula (I); and
R^{1a} is hydrogen, C₁-C₆ alkyl, or optionally substituted aryl.

In certain embodiments, the compound of Formula (I) is of Formula (IX-a):

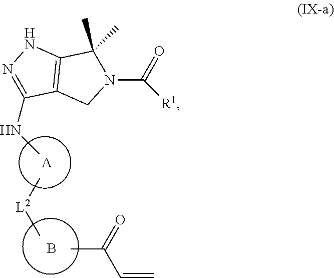

or a pharmaceutically acceptable salt, solvate, hydrate, polymorph, co-crystal, tautomer, stereoisomer, isotopically labeled derivative, or prodrug thereof, wherein R¹, linker L², Ring A, and Ring B are as defined for Formula (I).

In certain embodiments, the compound of Formula (I) is of Formula (IX-b):

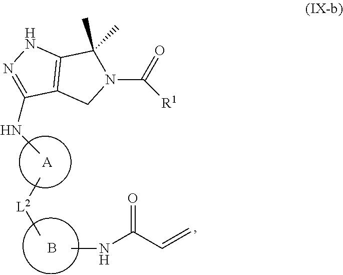

or a pharmaceutically acceptable salt, solvate, hydrate, polymorph, co-crystal, tautomer, stereoisomer, isotopically labeled derivative, or prodrug thereof, wherein R¹, linker L², Ring A, and Ring B are as defined for Formula (I).

In certain embodiments, the compound of Formula (I) is of Formula (IX-c):

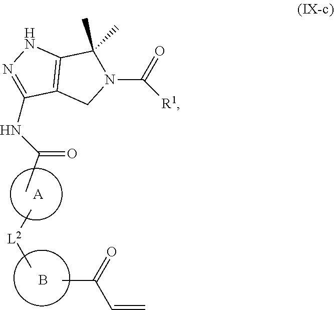

or a pharmaceutically acceptable salt, solvate, hydrate, polymorph, co-crystal, tautomer, stereoisomer, isotopically labeled derivative, or prodrug thereof, wherein R¹, linker L², Ring A, and Ring B are as defined for Formula (I).

In certain embodiments, the compound of Formula (I) is of Formula (IX-d):

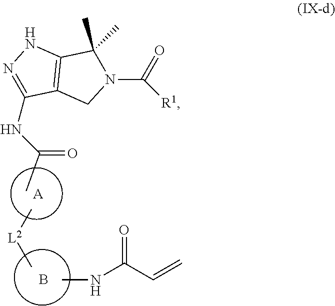

or a pharmaceutically acceptable salt, solvate, hydrate, polymorph, co-crystal, tautomer, stereoisomer, isotopically labeled derivative, or prodrug thereof, wherein R¹, linker L², Ring A, and Ring B are as defined for Formula (I).

In certain embodiments, the compound of Formula (I) is of Formula (X-a):

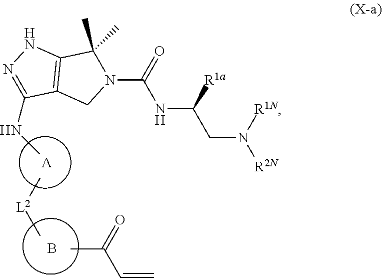

or a pharmaceutically acceptable salt, solvate, hydrate, polymorph, co-crystal, tautomer, stereoisomer, isotopically labeled derivative, or prodrug thereof, wherein:
linker L², Ring A, and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (X-b):

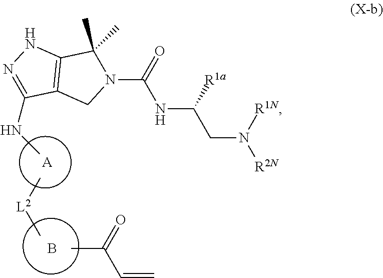

or a pharmaceutically acceptable salt, solvate, hydrate, polymorph, co-crystal, tautomer, stereoisomer, isotopically labeled derivative, or prodrug thereof, wherein:
linker L², Ring A, and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (X-c):

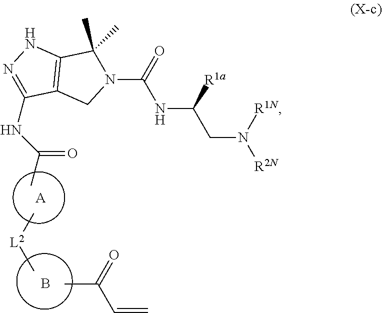

or a pharmaceutically acceptable salt, solvate, hydrate, polymorph, co-crystal, tautomer, stereoisomer, isotopically labeled derivative, or prodrug thereof, wherein:
linker L², Ring A, and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (X-d):

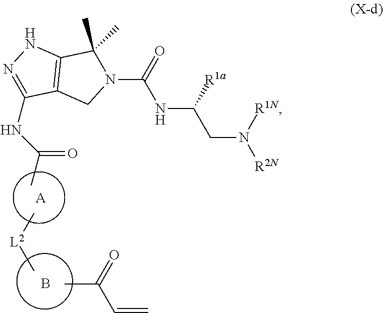

or a pharmaceutically acceptable salt, solvate, hydrate, polymorph, co-crystal, tautomer, stereoisomer, isotopically labeled derivative, or prodrug thereof, wherein:
linker L², Ring A, and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (XI-a):

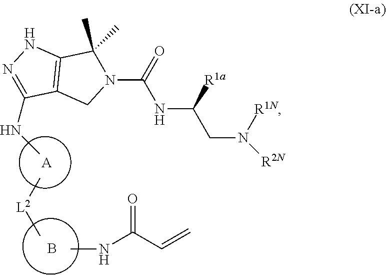

or a pharmaceutically acceptable salt, solvate, hydrate, polymorph, co-crystal, tautomer, stereoisomer, isotopically labeled derivative, or prodrug thereof, wherein:
linker L², Ring A, and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (XI-b):

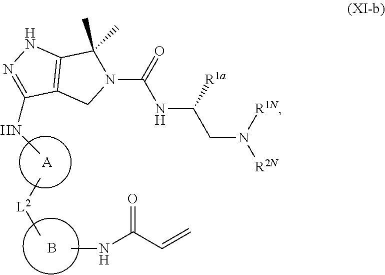

or a pharmaceutically acceptable salt, solvate, hydrate, polymorph, co-crystal, tautomer, stereoisomer, isotopically labeled derivative, or prodrug thereof, wherein:
linker L², Ring A, and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (XI-c):

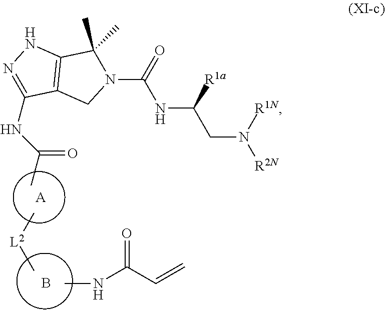

or a pharmaceutically acceptable salt, solvate, hydrate, polymorph, co-crystal, tautomer, stereoisomer, isotopically labeled derivative, or prodrug thereof, wherein:
linker L², Ring A, and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (XI-d):

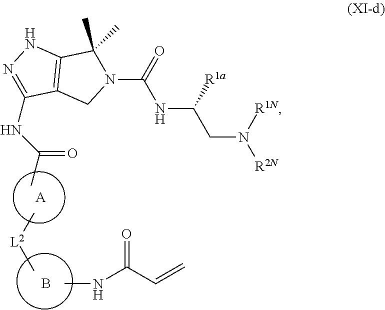

or a pharmaceutically acceptable salt, solvate, hydrate, polymorph, co-crystal, tautomer, stereoisomer, isotopically labeled derivative, or prodrug thereof, wherein:
linker L², Ring A, and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (XII-a):

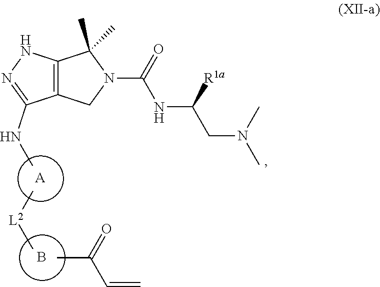

or a pharmaceutically acceptable salt, solvate, hydrate, polymorph, co-crystal, tautomer, stereoisomer, isotopically labeled derivative, or prodrug thereof, wherein:
linker L², Ring A, and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (XII-b):

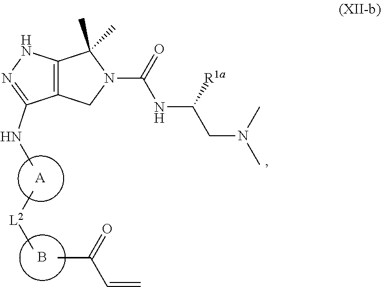

or a pharmaceutically acceptable salt, solvate, hydrate, polymorph, co-crystal, tautomer, stereoisomer, isotopically labeled derivative, or prodrug thereof, wherein:
linker L², Ring A, and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (XII-c):

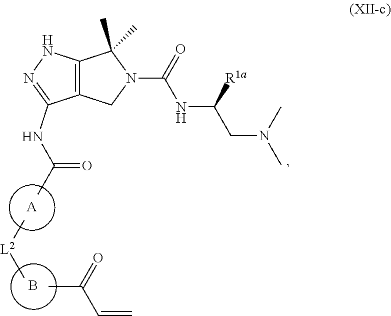

or a pharmaceutically acceptable salt, solvate, hydrate, polymorph, co-crystal, tautomer, stereoisomer, isotopically labeled derivative, or prodrug thereof, wherein:
linker L², Ring A, and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (XII-d):

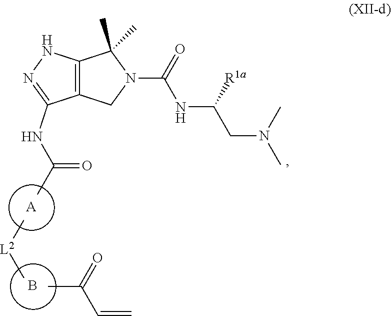

or a pharmaceutically acceptable salt, solvate, hydrate, polymorph, co-crystal, tautomer, stereoisomer, isotopically labeled derivative, or prodrug thereof, wherein:
linker L², Ring A, and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (XIII-a):

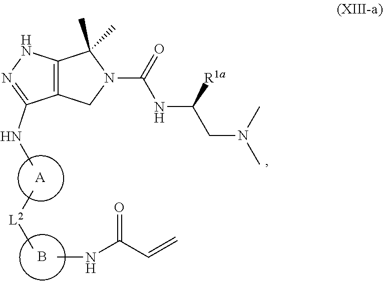

or a pharmaceutically acceptable salt, solvate, hydrate, polymorph, co-crystal, tautomer, stereoisomer, isotopically labeled derivative, or prodrug thereof, wherein:
linker L², Ring A, and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (XIII-b):

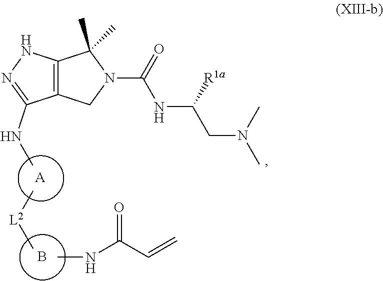

or a pharmaceutically acceptable salt, solvate, hydrate, polymorph, co-crystal, tautomer, stereoisomer, isotopically labeled derivative, or prodrug thereof, wherein:
linker L², Ring A, and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (XIII-c):

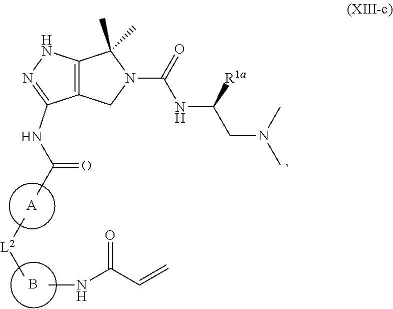

or a pharmaceutically acceptable salt, solvate, hydrate, polymorph, co-crystal, tautomer, stereoisomer, isotopically labeled derivative, or prodrug thereof, wherein:
linker L², Ring A, and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound of Formula (I) is of Formula (XIII-d):

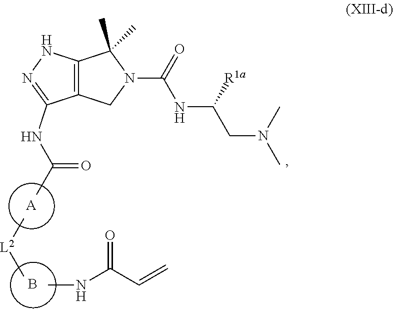

or a pharmaceutically acceptable salt, solvate, hydrate, polymorph, co-crystal, tautomer, stereoisomer, isotopically labeled derivative, or prodrug thereof, wherein:
linker L², Ring A, and Ring B are as defined for Formula (I);
R^{1a} is hydrogen, C₁-C₆ alkyl, or optionally substituted aryl; and
each of R^{1N} and R^{2N} is independently hydrogen, C₁-C₆ alkyl, or a nitrogen protecting group, or R^{1N} and R^{2N} are joined to form an optionally substituted carbocyclic, optionally substituted heterocyclic, optionally substituted aryl, or optionally substituted heteroaryl ring.

In certain embodiments, the compound according to Formula (I) is a compound listed in Table 1.

TABLE 1
Exemplary Compounds of Formula (I).

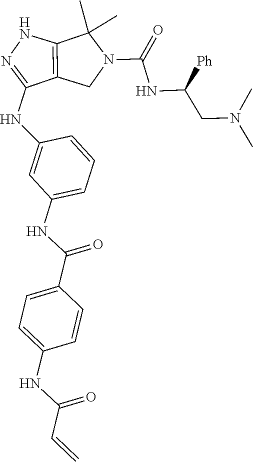 [SMILES: C=CC(=O)Nc1ccc(C(=O)Nc2cccc(Nc3n[nH]c4c3CN(C(=O)N[C@H](CN(C)C)c3ccccc3)C4(C)C)c2)cc1]

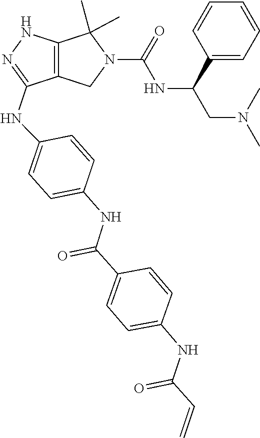 [SMILES: C=CC(=O)Nc1ccc(C(=O)Nc2ccc(Nc3n[nH]c4c3CN(C(=O)N[C@H](CN(C)C)c3ccccc3)C4(C)C)cc2)cc1]

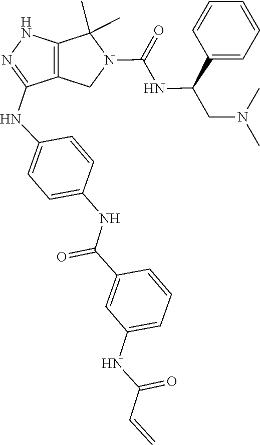 [SMILES: C=CC(=O)Nc1cccc(C(=O)Nc2ccc(Nc3n[nH]c4c3CN(C(=O)N[C@H](CN(C)C)c3ccccc3)C4(C)C)cc2)c1]

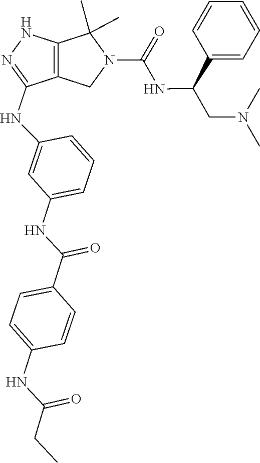 [SMILES: CCC(=O)Nc1ccc(C(=O)Nc2cccc(Nc3n[nH]c4c3CN(C(=O)N[C@H](CN(C)C)c3ccccc3)C4(C)C)c2)cc1]

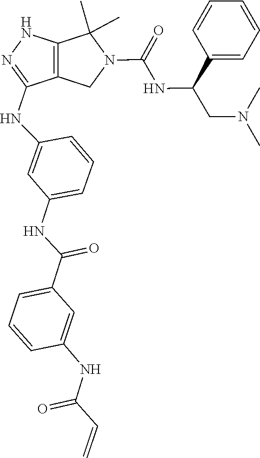 [SMILES: C=CC(=O)Nc1cccc(C(=O)Nc2cccc(Nc3n[nH]c4c3CN(C(=O)N[C@H](CN(C)C)c3ccccc3)C4(C)C)c2)c1]

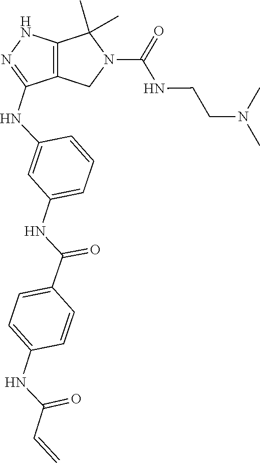 [SMILES: C=CC(=O)Nc1ccc(C(=O)Nc2cccc(Nc3n[nH]c4c3CN(C(=O)NCCN(C)C)C4(C)C)c2)cc1]

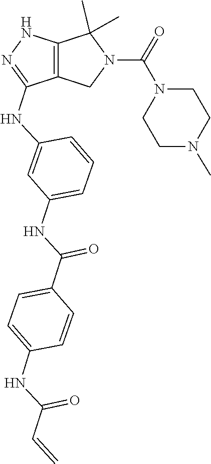 [SMILES: C=CC(=O)Nc1ccc(C(=O)Nc2cccc(Nc3n[nH]c4c3CN(C(=O)N3CCN(C)CC3)C4(C)C)c2)cc1]

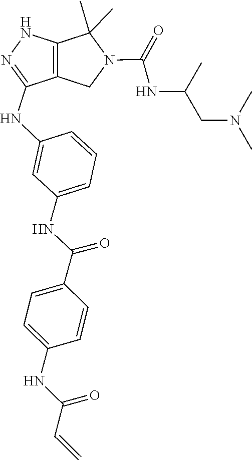 [SMILES: C=CC(=O)Nc1ccc(C(=O)Nc2cccc(Nc3n[nH]c4c3CN(C(=O)NC(C)CN(C)C)C4(C)C)c2)cc1]

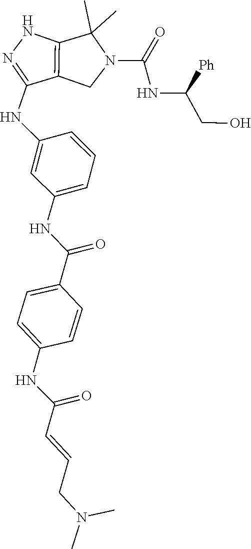 [SMILES: CN(C)C/C=C/C(=O)Nc1ccc(C(=O)Nc2cccc(Nc3n[nH]c4c3CN(C(=O)N[C@H](CO)c3ccccc3)C4(C)C)c2)cc1]

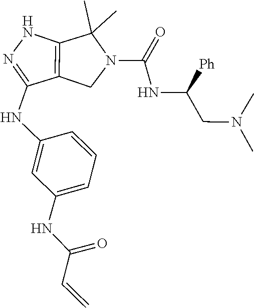 [SMILES: C=CC(=O)Nc1cccc(Nc2n[nH]c3c2CN(C(=O)N[C@H](CN(C)C)c2ccccc2)C3(C)C)c1]

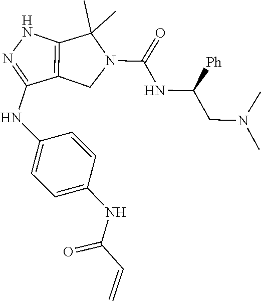 [SMILES: C=CC(=O)Nc1ccc(Nc2n[nH]c3c2CN(C(=O)N[C@H](CN(C)C)c2ccccc2)C3(C)C)cc1]

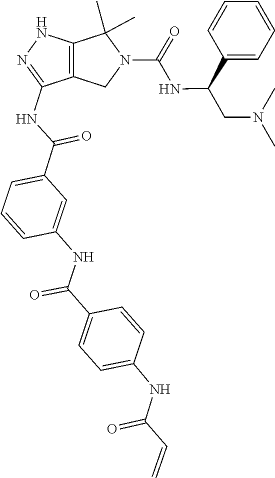 [SMILES: C=CC(=O)Nc1ccc(C(=O)Nc2cccc(C(=O)Nc3n[nH]c4c3CN(C(=O)N[C@H](CN(C)C)c3ccccc3)C4(C)C)c2)cc1]

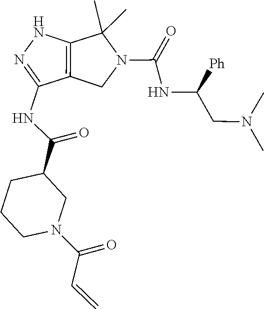 [SMILES: C=CC(=O)N1CCC[C@@H](C(=O)Nc2n[nH]c3c2CN(C(=O)N[C@H](CN(C)C)c2ccccc2)C3(C)C)C1]

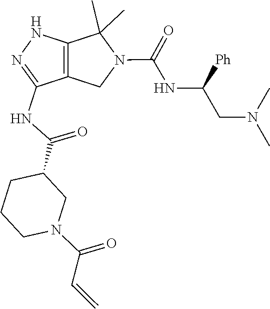 [SMILES: C=CC(=O)N1CCC[C@H](C(=O)Nc2n[nH]c3c2CN(C(=O)N[C@H](CN(C)C)c2ccccc2)C3(C)C)C1]

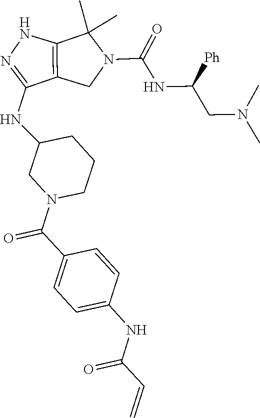 [SMILES: C=CC(=O)Nc1ccc(C(=O)N2CCCC(Nc3n[nH]c4c3CN(C(=O)N[C@H](CN(C)C)c3ccccc3)C4(C)C)C2)cc1]

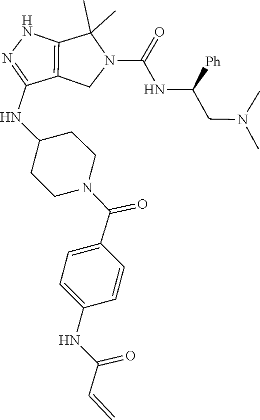 [SMILES: C=CC(=O)Nc1ccc(C(=O)N2CCC(Nc3n[nH]c4c3CN(C(=O)N[C@H](CN(C)C)c3ccccc3)C4(C)C)CC2)cc1]

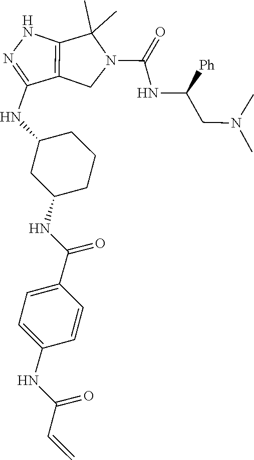 [SMILES: C=CC(=O)Nc1ccc(C(=O)N[C@H]2CCC[C@@H](Nc3n[nH]c4c3CN(C(=O)N[C@H](CN(C)C)c3ccccc3)C4(C)C)C2)cc1]

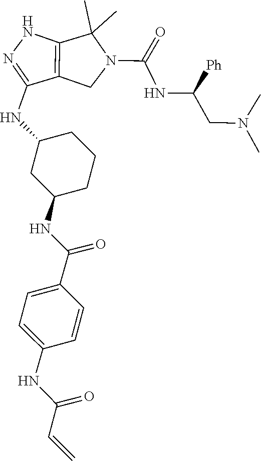 [SMILES: C=CC(=O)Nc1ccc(C(=O)N[C@@H]2CCC[C@@H](Nc3n[nH]c4c3CN(C(=O)N[C@H](CN(C)C)c3ccccc3)C4(C)C)C2)cc1]

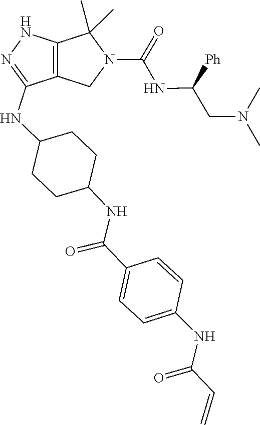 [SMILES: C=CC(=O)Nc1ccc(C(=O)NC2CCC(Nc3n[nH]c4c3CN(C(=O)N[C@H](CN(C)C)c3ccccc3)C4(C)C)CC2)cc1]

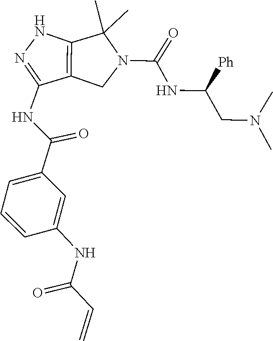 [SMILES: C=CC(=O)Nc1cccc(C(=O)Nc2n[nH]c3c2CN(C(=O)N[C@H](CN(C)C)c2ccccc2)C3(C)C)c1]

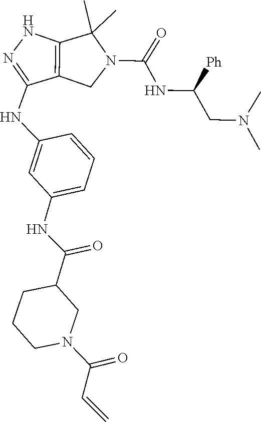 [SMILES: C=CC(=O)N1CCCC(C(=O)Nc2cccc(Nc3n[nH]c4c3CN(C(=O)N[C@H](CN(C)C)c3ccccc3)C4(C)C)c2)C1]

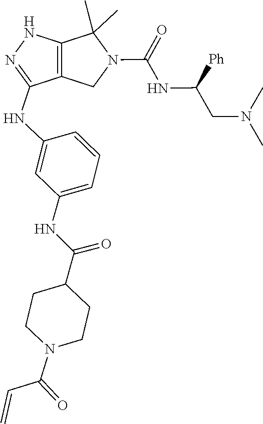 [SMILES: C=CC(=O)N1CCC(C(=O)Nc2cccc(Nc3n[nH]c4c3CN(C(=O)N[C@H](CN(C)C)c3ccccc3)C4(C)C)c2)CC1]

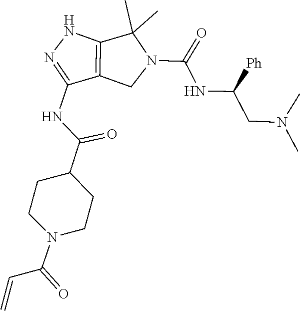 [SMILES: C=CC(=O)N1CCC(C(=O)Nc2n[nH]c3c2CN(C(=O)N[C@H](CN(C)C)c2ccccc2)C3(C)C)CC1]

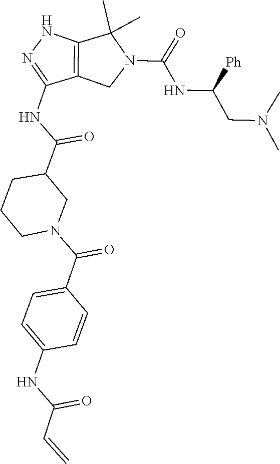 [SMILES: C=CC(=O)Nc1ccc(C(=O)N2CCCC(C(=O)Nc3n[nH]c4c3CN(C(=O)N[C@H](CN(C)C)c3ccccc3)C4(C)C)C2)cc1]

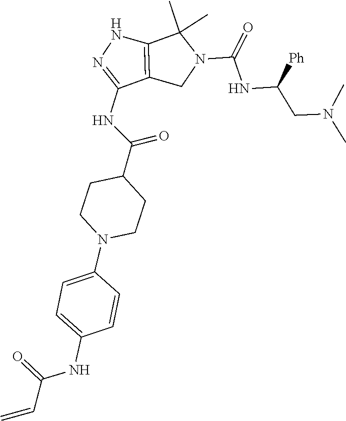 [SMILES: C=CC(=O)Nc1ccc(N2CCC(C(=O)Nc3n[nH]c4c3CN(C(=O)N[C@H](CN(C)C)c3ccccc3)C4(C)C)CC2)cc1]

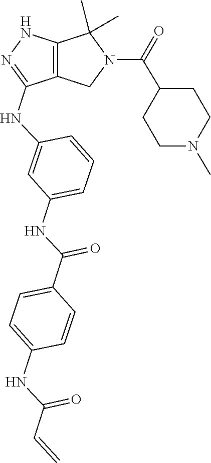 [SMILES: C=CC(=O)Nc1ccc(C(=O)Nc2cccc(Nc3n[nH]c4c3CN(C(=O)C3CCN(C)CC3)C4(C)C)c2)cc1]

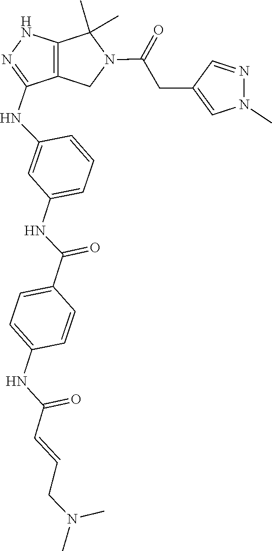 [SMILES: CN(C)C/C=C/C(=O)Nc1ccc(C(=O)Nc2cccc(Nc3n[nH]c4c3CN(C(=O)Cc3cnn(C)c3)C4(C)C)c2)cc1]

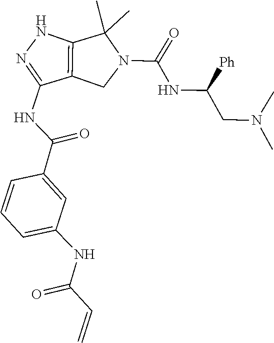 [SMILES: C=CC(=O)Nc1cccc(C(=O)Nc2n[nH]c3c2CN(C(=O)N[C@H](CN(C)C)c2ccccc2)C3(C)C)c1]

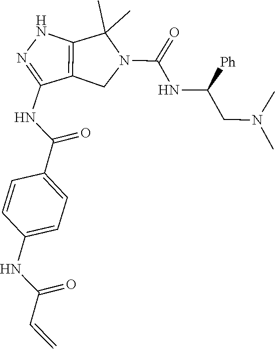 [SMILES: C=CC(=O)Nc1ccc(C(=O)Nc2n[nH]c3c2CN(C(=O)N[C@H](CN(C)C)c2ccccc2)C3(C)C)cc1]

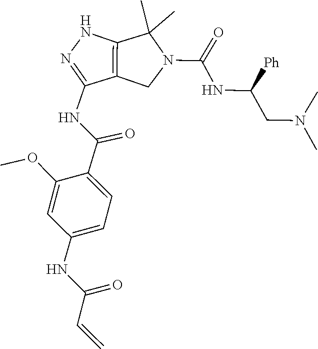

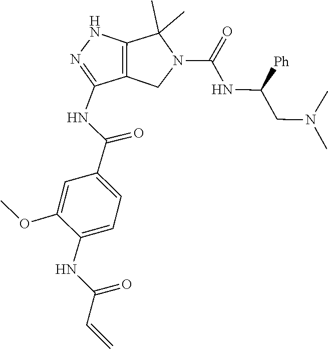

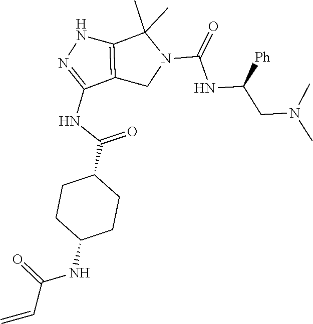 [SMILES: C=CC(=O)N[C@H]1CC[C@@H](C(=O)Nc2n[nH]c3c2CN(C(=O)N[C@H](CN(C)C)c2ccccc2)C3(C)C)CC1]

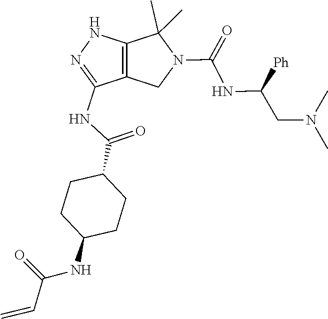 [SMILES: C=CC(=O)N[C@H]1CC[C@H](C(=O)Nc2n[nH]c3c2CN(C(=O)N[C@H](CN(C)C)c2ccccc2)C3(C)C)CC1]

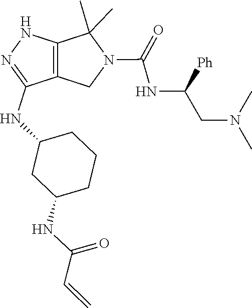 [SMILES: C=CC(=O)N[C@H]1CCC[C@@H](Nc2n[nH]c3c2CN(C(=O)N[C@H](CN(C)C)c2ccccc2)C3(C)C)C1]

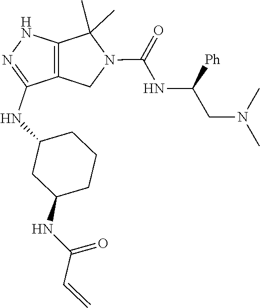 [SMILES: C=CC(=O)N[C@@H]1CCC[C@@H](Nc2n[nH]c3c2CN(C(=O)N[C@H](CN(C)C)c2ccccc2)C3(C)C)C1]

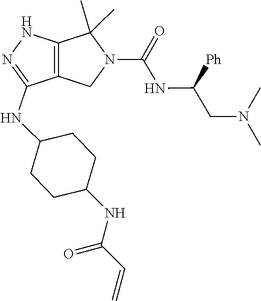 [SMILES: C=CC(=O)NC1CCC(Nc2n[nH]c3c2CN(C(=O)N[C@H](CN(C)C)c2ccccc2)C3(C)C)CC1]

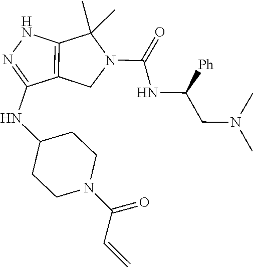 [SMILES: C=CC(=O)N1CCC(Nc2n[nH]c3c2CN(C(=O)N[C@H](CN(C)C)c2ccccc2)C3(C)C)CC1]

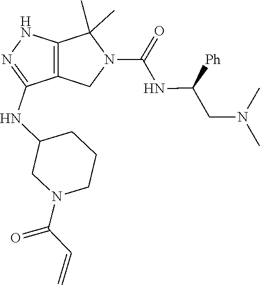 [SMILES: C=CC(=O)N1CCCC(Nc2n[nH]c3c2CN(C(=O)N[C@H](CN(C)C)c2ccccc2)C3(C)C)C1]

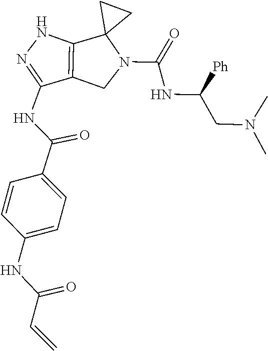 [SMILES: C=CC(=O)Nc1ccc(C(=O)Nc2n[nH]c3c2CN(C(=O)N[C@H](CN(C)C)c2ccccc2)C32CC2)cc1]

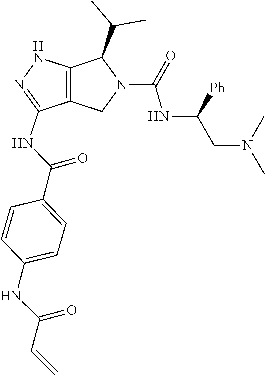 [SMILES: C=CC(=O)Nc1ccc(C(=O)Nc2n[nH]c3c2CN(C(=O)N[C@H](CN(C)C)c2ccccc2)[C@@H]3C(C)C)cc1]

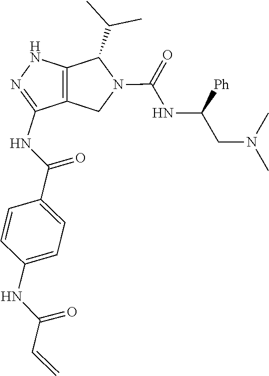 [SMILES: C=CC(=O)Nc1ccc(C(=O)Nc2n[nH]c3c2CN(C(=O)N[C@H](CN(C)C)c2ccccc2)[C@H]3C(C)C)cc1]

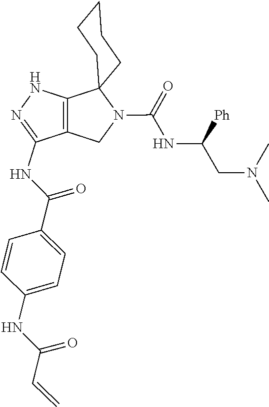 [SMILES: C=CC(=O)Nc1ccc(C(=O)Nc2n[nH]c3c2CN(C(=O)N[C@H](CN(C)C)c2ccccc2)C32CCCCC2)cc1]

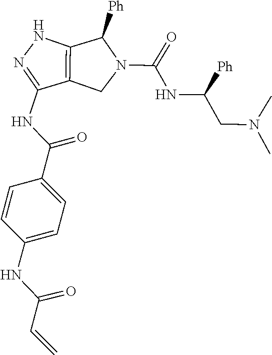 [SMILES: C=CC(=O)Nc1ccc(C(=O)Nc2n[nH]c3c2CN(C(=O)N[C@H](CN(C)C)c2ccccc2)[C@@H]3c2ccccc2)cc1]

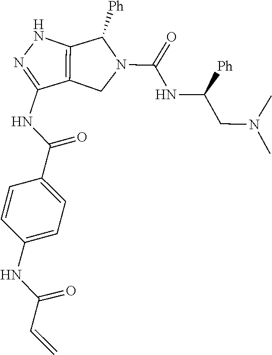 [SMILES: C=CC(=O)Nc1ccc(C(=O)Nc2n[nH]c3c2CN(C(=O)N[C@H](CN(C)C)c2ccccc2)[C@H]3c2ccccc2)cc1]

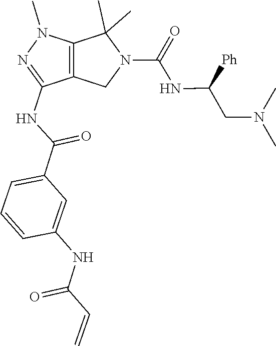 [SMILES: C=CC(=O)Nc1cccc(C(=O)Nc2nn(C)c3c2CN(C(=O)N[C@H](CN(C)C)c2ccccc2)C3(C)C)c1]

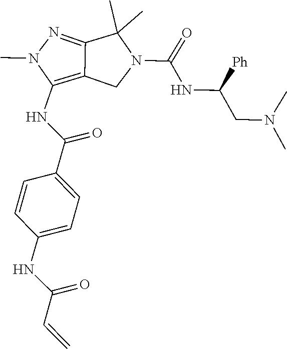

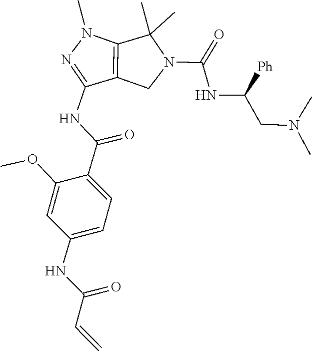

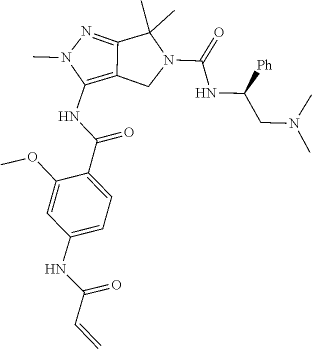

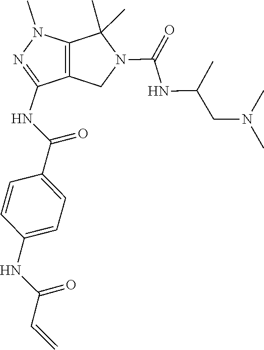 [SMILES: C=CC(=O)Nc1ccc(C(=O)Nc2nn(C)c3c2CN(C(=O)NC(C)CN(C)C)C3(C)C)cc1]

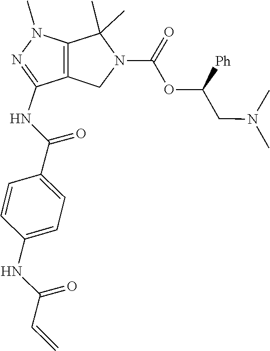 [SMILES: C=CC(=O)Nc1ccc(C(=O)Nc2nn(C)c3c2CN(C(=O)O[C@H](CN(C)C)c2ccccc2)C3(C)C)cc1]

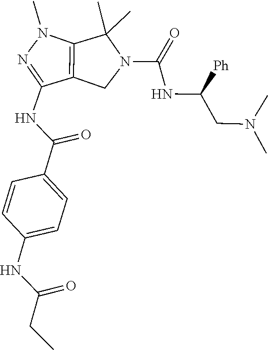 [SMILES: CCC(=O)Nc1ccc(C(=O)Nc2nn(C)c3c2CN(C(=O)N[C@H](CN(C)C)c2ccccc2)C3(C)C)cc1]

In certain embodiments, the compound of Formula (I) is of formula:

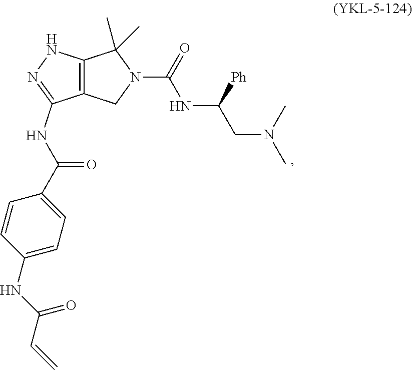 [SMILES: C=CC(=O)Nc1ccc(C(=O)Nc2n[nH]c3c2CN(C(=O)N[C@H](CN(C)C)c2ccccc2)C3(C)C)cc1]

or a pharmaceutically acceptable salt, solvate, hydrate, polymorph, co-crystal, stereoisomer, tautomer, isotopically labeled derivative, or prodrug thereof.

In certain embodiments, the CDK7 inhibitor is a compound of Formula (XIV):

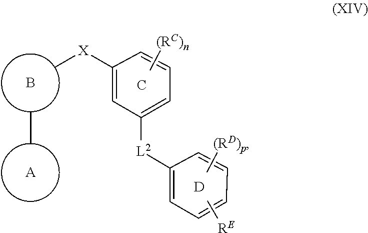

or a pharmaceutically acceptable salt, solvate, hydrate, polymorph, co-crystal, tautomer, stereoisomer, isotopically labeled derivative, or prodrug thereof;
wherein:

Ring A is an optionally substituted heteroaryl ring of any one of the Formulae (i-1)-(i-5):

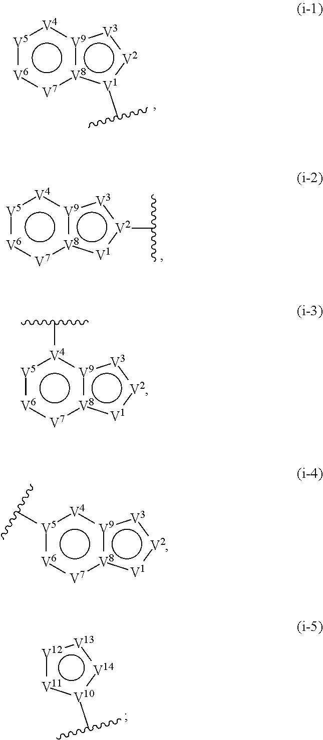

each instance of V¹, V², V³, V⁴, V⁵, V⁶, V⁷, V⁸, V⁹, V¹⁰, V¹¹, V¹², V¹³, and V¹⁴ is independently O, S, N, NR^{A1}, C, or CR^{A2};

each instance of R^{A1} is independently selected from the group consisting of hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, and a nitrogen protecting group;

each instance of R^{A2} is independently selected from the group consisting of hydrogen, halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{A2a}, —N(R^{A2a})₂, and —SR^{A2a}, wherein each occurrence of R^{A2a} is independently selected from the group consisting of hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, and a sulfur protecting group when attached to a sulfur atom, or two R^{A2a} groups are joined to form an optionally substituted heterocyclic ring;

optionally any two of R^{A1}, R^{A2}, and R^{A2a} groups are joined to form an optionally substituted carbocyclic, optionally substituted heterocyclic, optionally substituted aryl, or optionally substituted heteroaryl ring;

Ring B is of the formula:

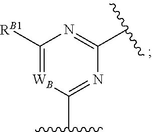

R^{B1} is selected from the group consisting of hydrogen, halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{B1a}, —N(R^{B1a})₂, and —SR^{B1a}, wherein each occurrence of R^{B1a} is independently selected from the group consisting of hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, and a sulfur protecting group when attached to a sulfur atom, or two R^{B1a} groups are joined to form an optionally substituted heterocyclic ring;

W_{B} is N or CR^{B2}, wherein R^{B2} is selected from the group consisting of hydrogen, halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{B2a}, —N(R^{B2a})₂, and —SR^{B2a}, wherein each occurrence of R^{B2a} is independently selected from the group consisting of hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, and a sulfur protecting group when attached to a sulfur atom, or two R^{B2a} groups are joined to form an optionally substituted heterocyclic ring;

optionally R^{B1} and R^{B2} are joined to form an optionally substituted carbocyclyl, optionally substituted heterocyclyl, optionally substituted heteroaryl, or optionally substituted aryl ring;

X is an optionally substituted C₁₋₄ hydrocarbon chain, optionally wherein one or more carbon units of the hydrocarbon chain is replaced with —O—, —S—, or —NR^{X}—, wherein R^{X} is hydrogen, C₁₋₆ alkyl, or a nitrogen protecting group;

L² is a bond, —O—, —S—, —NR^{L2a}—, —NR^{L2a}C(═O)—, —C(═O)NR^{L2a}—, —SC(═O)—, —C(═O)S—, —OC(═O)—, —C(═O)O—, —NR^{L2a}C(═S)—, —C(═S)NR^{L2a}—, trans-CR^{L2b}═CR^{L2b}—, cis-CR^{L2b}═CR^{L2b}—, —C≡C—, —OC(R^{L2b})₂—, —C(R^{L2b})₂O—, —NR^{L2a}C(R^{L2b})₂—, —C(R^{L2b})₂NR^{L2a}—, —SC(R^{L2b})₂—, —C(R^{L2b})₂S—, —S(═O)₂O—, —OS(═O)₂—, —S(═O)₂NR^{L2a}, —NR^{L2a}S(═O)₂—, or an optionally substituted C₁₋₄ hydrocarbon chain, optionally wherein one or more carbon units of the hydrocarbon chain is replaced with —O—, —S—, —NR^{L2a}—, —NR^{L2a}C(═O)—, —C(═O)NR^{L2a}—, —SC(═O)—, —C(═O)S—, —OC(═O)—, —C(═O)O—, —NR^{L2a}C(═S)—, —C(═S)NR^{L2a}—, trans-CR^{L2b}═CR^{L2b}—, cis-CR^{L2b}═CR^{L2b}—, —C≡C—, —S(═O)₂O—, —OS(═O)₂—, —S(═O)₂NR^{L2a}—, or —NR^{L2a}S(═O)₂—, wherein R^{L2a} is hydrogen, C₁₋₆ alkyl, or a nitrogen protecting group, and wherein each occurrence of R^{L2b} is independently selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{L2b} groups are joined to form an optionally substituted carbocyclic or optionally substituted heterocyclic ring;

each instance of R^{C} is independently selected from the group consisting of hydrogen, halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{C1}, —N(R^{C1})₂, and —SR^{C1}, wherein each occurrence of R^{C1} is independently selected from the group consisting of hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, and a sulfur protecting group when attached to a sulfur atom, or two R^{C1} groups are joined to form an optionally substituted heterocyclic ring;

n is 0, 1, 2, 3, or 4;

each instance of R^{D} is independently selected from the group consisting of hydrogen, halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{D1}, —N(R^{D1})₂, and —SR^{D1}, wherein each occurrence of R^{D1} is independently selected from the group consisting of hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, and a sulfur protecting group when attached to a sulfur atom, or two R^{D1} groups are joined to form an optionally substituted heterocyclic ring;

p is 0, 1, 2, 3, or 4;

R^{E} is any one of the Formulae (ii-1)-(ii-17):

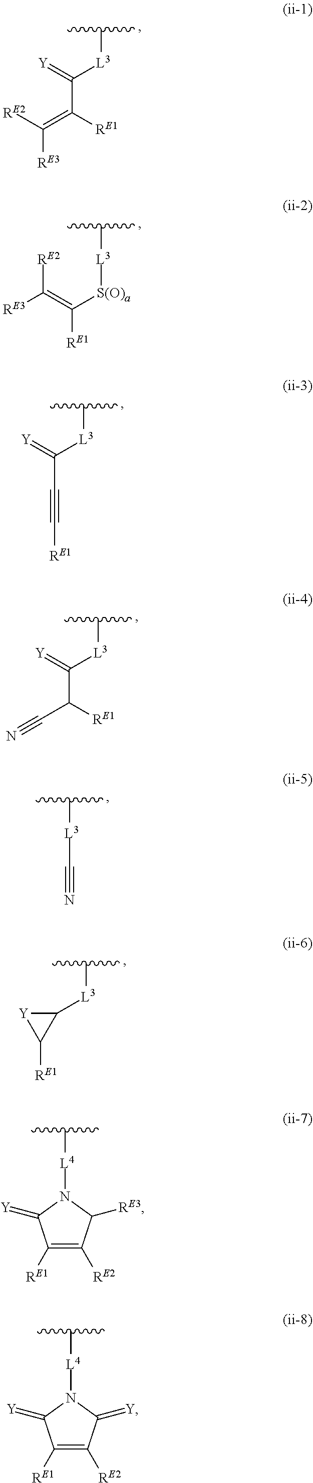

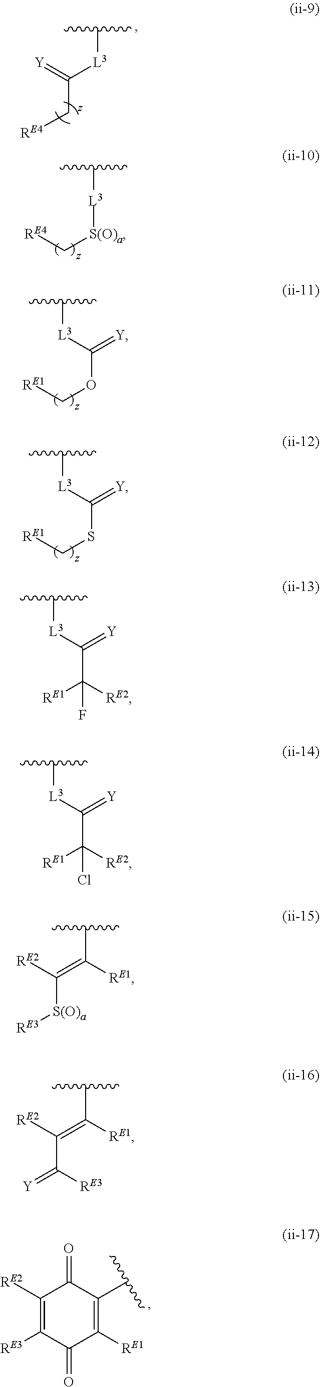

R^{E} and L² are para or meta to each other;

L³ is a bond, —O—, —S—, —NR^{L3a}—, or an optionally substituted C₁₋₄ hydrocarbon chain, optionally wherein one or more carbon units of the hydrocarbon chain is replaced with —O—, —S—, —NR^{L3a}—, —NR^{L3a}C(═O)—, —C(═O)NR^{L3a}—, —SC(═O)—, —C(═O)—, —C(═O)S—, —OC(═O)—, —C(═O)O—, —NR^{L3a}C(═S)—, —C(═S)NR^{L3a}—, trans-CR^{L3b}═CR^{L3b}—, cis-CR^{L3b}═CR^{L3b}—, —C≡C—, —S(═O)₂O—, —OS(═O)₂—, —S(═O)₂NR^{L3a}—, or —NR^{L3a}S(═O)₂—, wherein R^{L3a} is hydrogen, C₁₋₆ alkyl, or a nitrogen protecting group, and wherein each occurrence of R^{L3b} is independently selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{L3b} groups are joined to form an optionally substituted carbocyclic or optionally substituted heterocyclic ring;

L⁴ is a bond or an optionally substituted C₁₋₄ hydrocarbon chain;

R^{E1} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E1a}, —CH₂N(R^{E1a})₂, —CH₂SR^{E1a}, —OR^{E1a}, —N(R^{E1a})₂, and —SR^{E1a}, wherein each occurrence of R^{E1a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E1a} groups are joined to form an optionally substituted heterocyclic ring;

R^{E2} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E2a}, —CH₂N(R^{E2a})₂, —CH₂SR^{E2a}, —OR^{E2a}, —N(R^{E2a})₂, and —SR^{E2a}, wherein each occurrence of R^{E2a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E2a} groups are joined to form an optionally substituted heterocyclic ring;

R^{E3} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E3a}, —CH₂N(R^{E3a})₂, —CH₂SR^{E3a}, —OR^{E3a}, —N(R^{E3a})₂, and —SR^{E3a}, wherein each occurrence of R^{E3a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E3a} groups are joined to form an optionally substituted heterocyclic ring;

optionally R^{E1} and R^{E3}, or R^{E2} and R^{E3}, or R^{E1} and R^{E2} are joined to form an optionally substituted carbocyclic or optionally substituted heterocyclic ring;

R^{E4} is a leaving group;

Y is O, S, or NR^{E5}, wherein R^{E5} is hydrogen, C₁₋₆ alkyl, or a nitrogen protecting group;

a is 1 or 2; and

z is 0, 1, 2, 3, 4, 5, or 6.

In certain embodiments, the compound of Formula (XIV) is of formula:

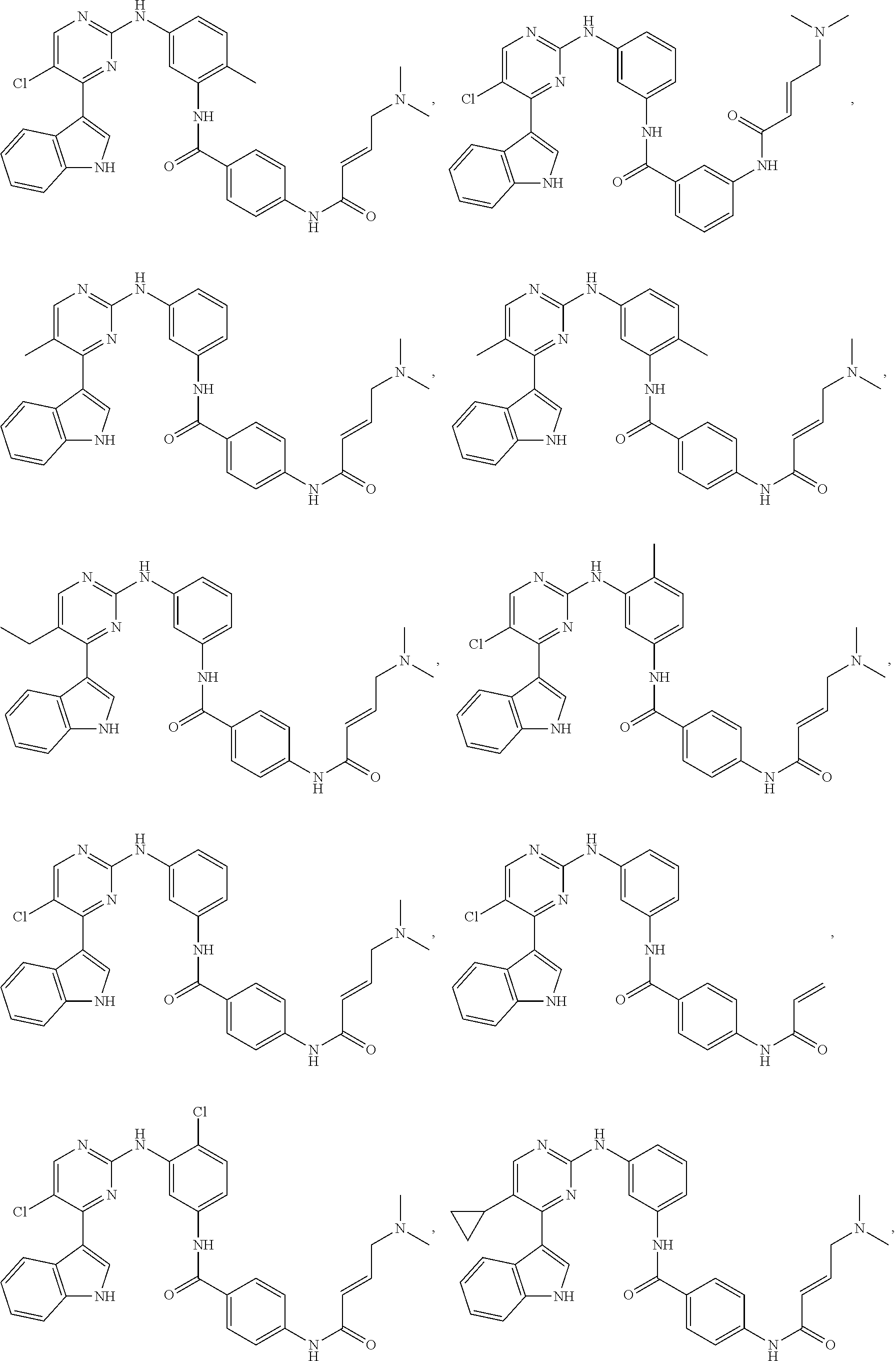

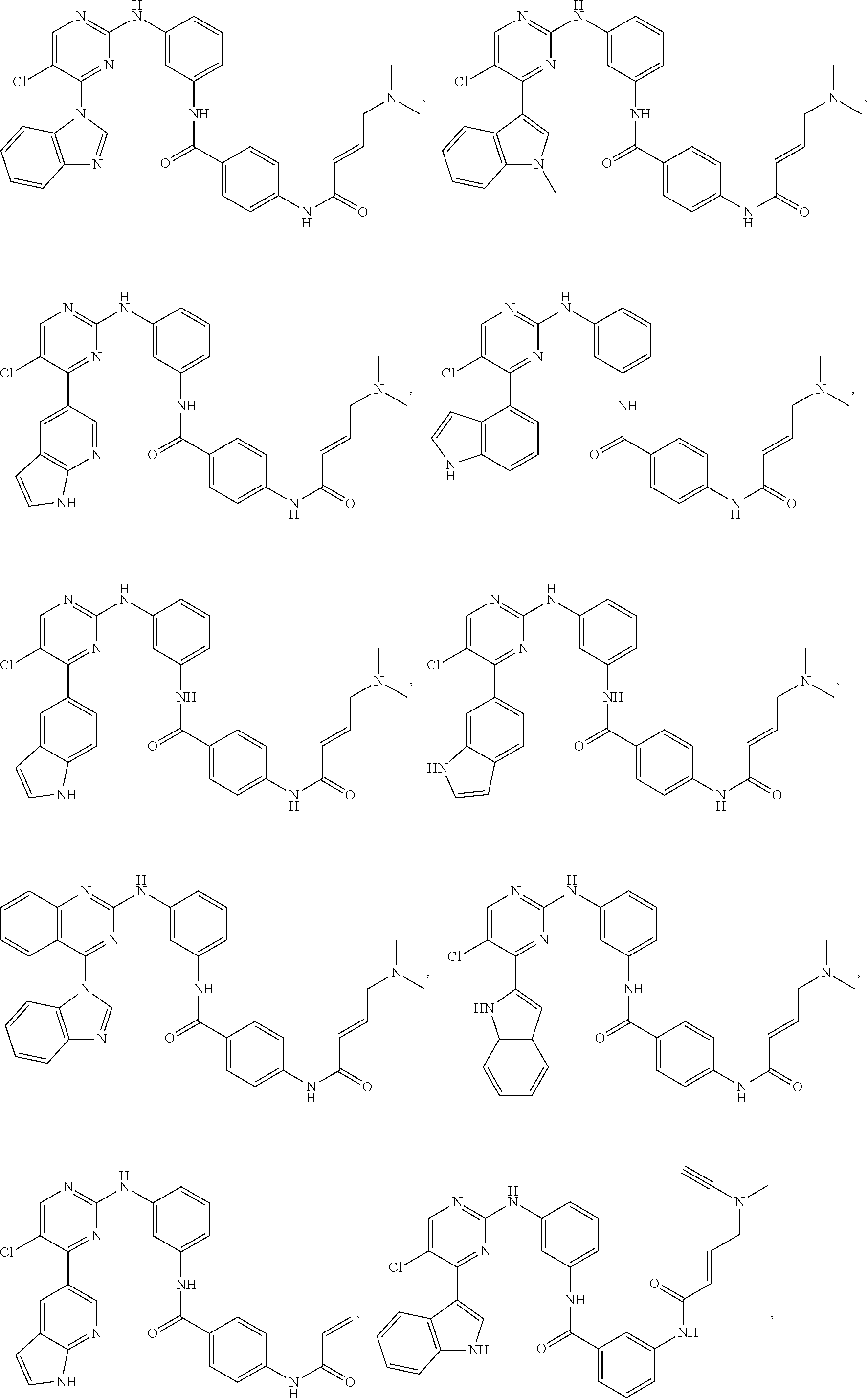

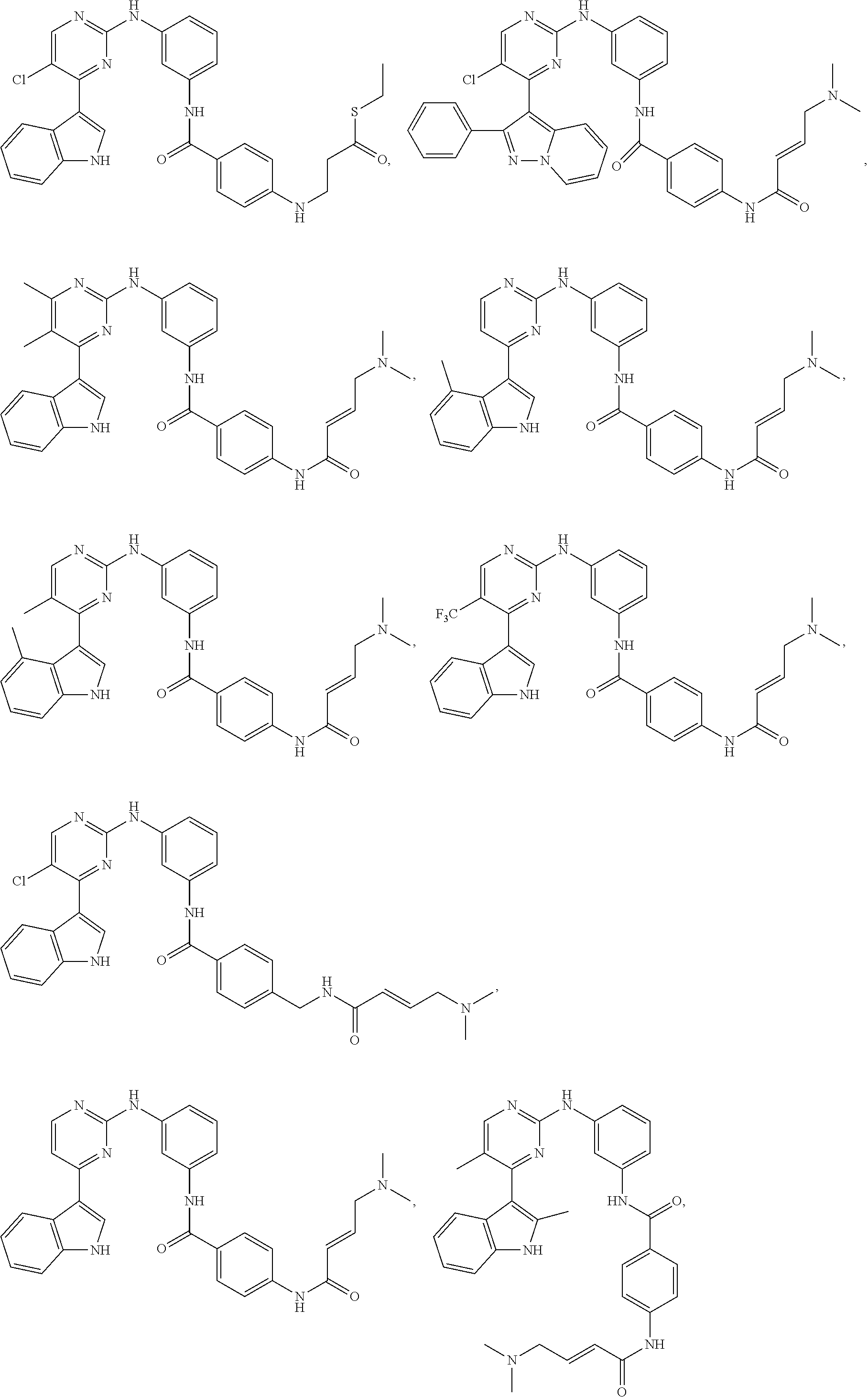

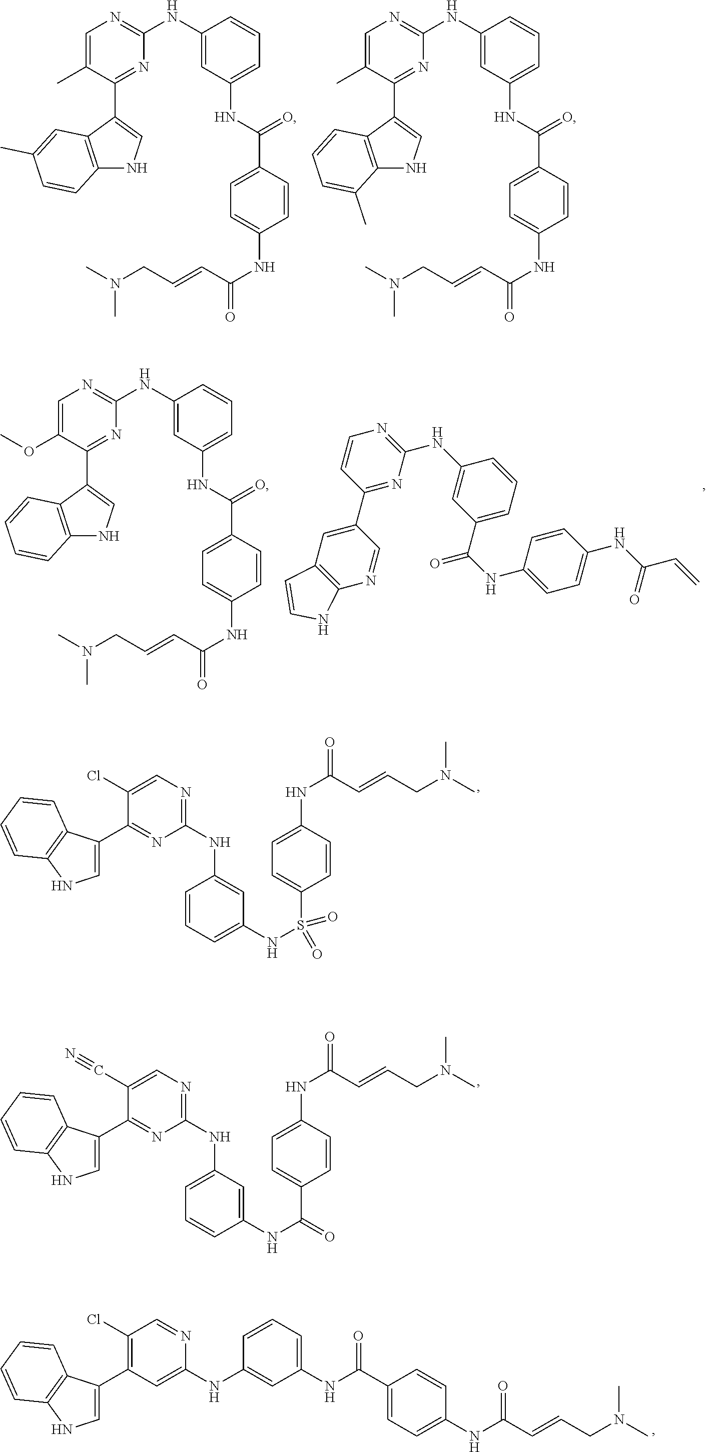

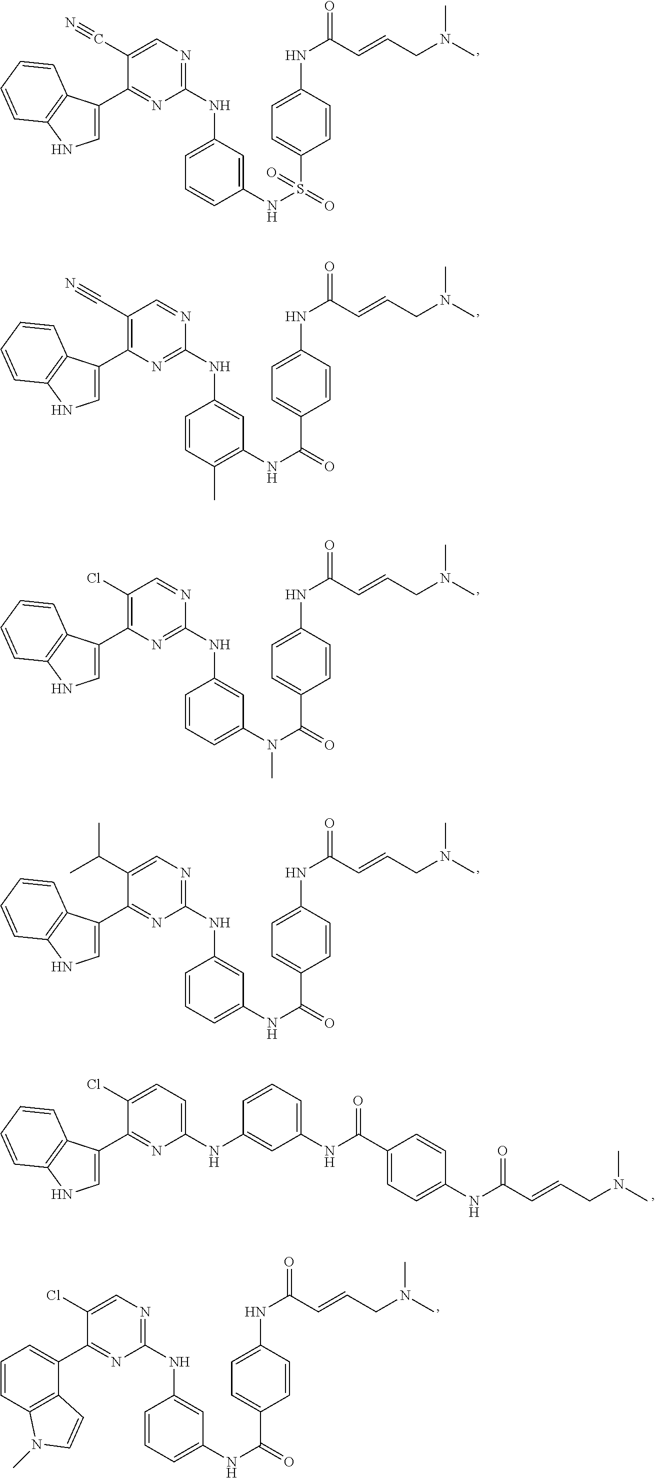 [SMILES: CN(C)C/C=C/C(=O)Nc1ccc(S(=O)(=O)Nc2cccc(Nc3ncc(C#N)c(-c4c[nH]c5ccccc45)n3)c2)cc1 ; Cc1ccc(Nc2ncc(C#N)c(-c3c[nH]c4ccccc34)n2)cc1NC(=O)c1ccc(NC(=O)/C=C/CN(C)C)cc1 ; CN(C)C/C=C/C(=O)Nc1ccc(C(=O)N(C)c2cccc(Nc3ncc(Cl)c(-c4c[nH]c5ccccc45)n3)c2)cc1 ; CC(C)c1cnc(Nc2cccc(NC(=O)c3ccc(NC(=O)/C=C/CN(C)C)cc3)c2)nc1-c1c[nH]c2ccccc12 ; CN(C)C/C=C/C(=O)Nc1ccc(C(=O)Nc2cccc(Nc3ccc(Cl)c(-c4c[nH]c5ccccc45)n3)c2)cc1 ; CN(C)C/C=C/C(=O)Nc1ccc(C(=O)Nc2cccc(Nc3ncc(Cl)c(-c4cccc5c4ccn5C)n3)c2)cc1]

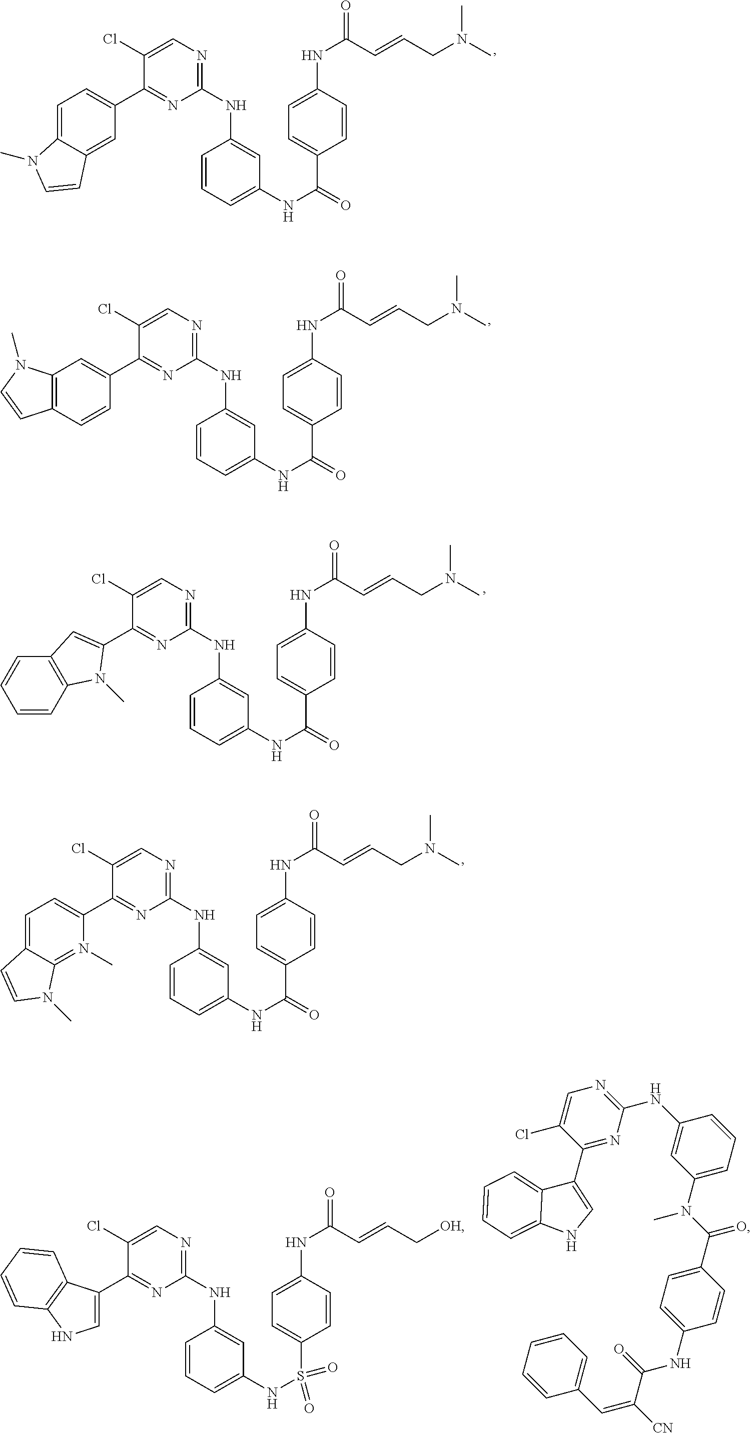

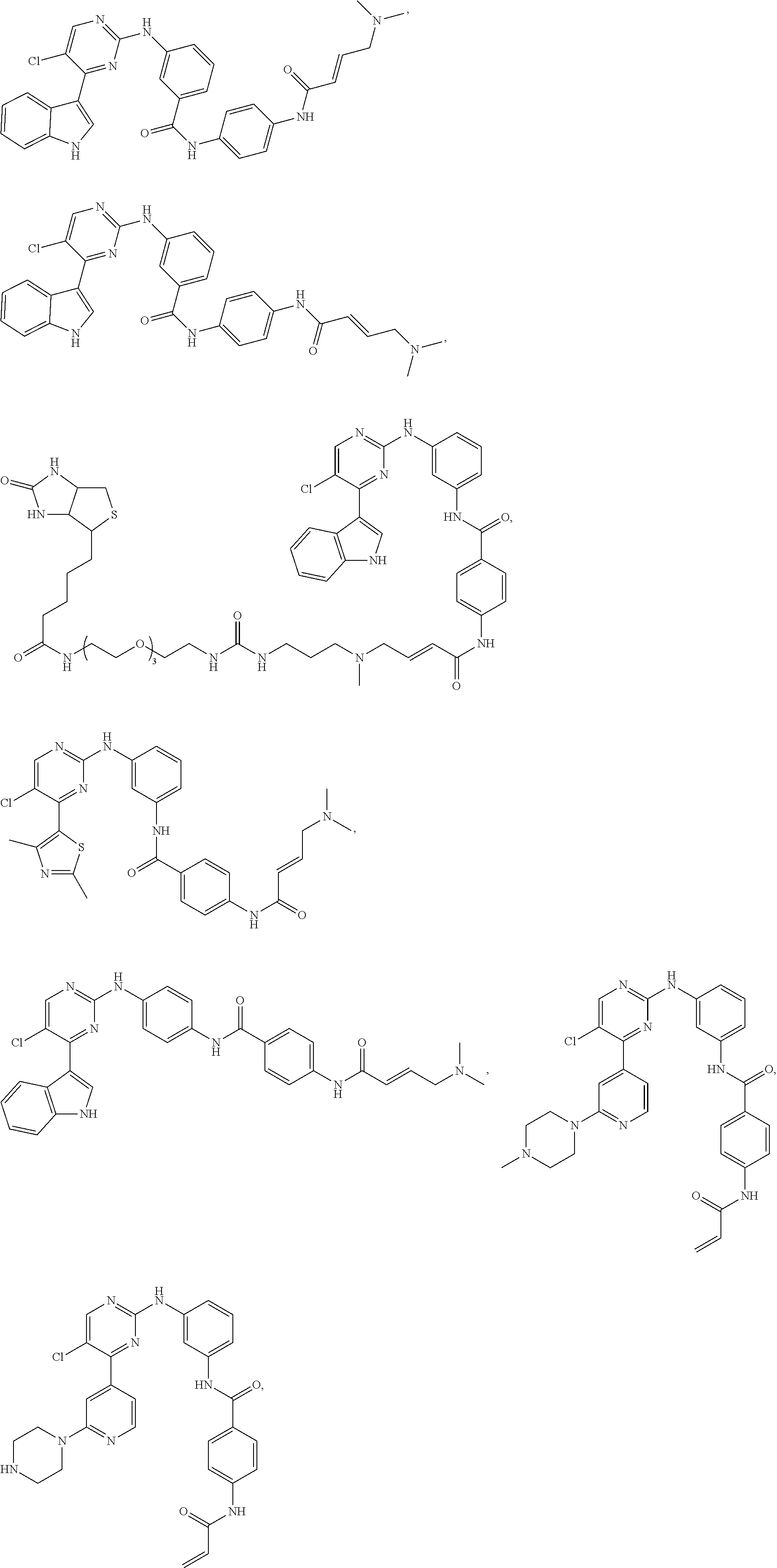

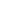

or a pharmaceutically acceptable salt, solvate, hydrate, polymorph, co-crystal, tautomer, stereoisomer, isotopically labeled derivative, or prodrug thereof.

In certain embodiments, the CDK7 inhibitor is a compound of Formula (XV):

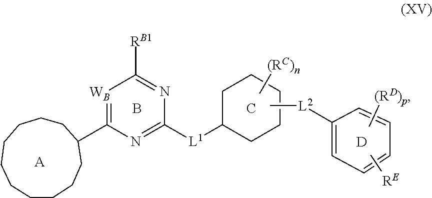

or a pharmaceutically acceptable salt, solvate, hydrate, polymorph, co-crystal, tautomer, stereoisomer, isotopically labeled derivative, or prodrug thereof, wherein:

Ring A is an optionally substituted heteroaryl ring of any one of the Formulae (i-1)-(i-6):

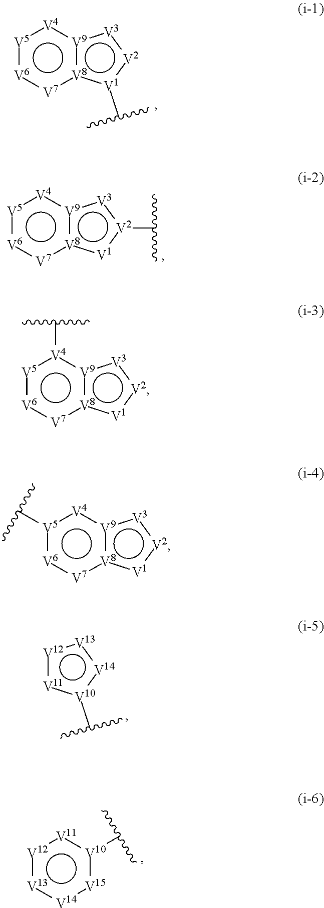

wherein:

each instance of V¹, V², V³, V⁴, V⁵, V⁶, V⁷, V⁸, V⁹, V¹⁰, V¹¹, V¹², V¹³, V¹⁴, and V¹⁵ is independently O, S, N, NR^{A1}, C, or CR^{A2};

each instance of R^{A1} is independently selected from the group consisting of hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, and a nitrogen protecting group;

each instance of R^{A2} is independently selected from the group consisting of hydrogen, halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{A2a}, —N(R^{A2a})₂, and —SR^{A2a}, wherein each occurrence of R^{A2a} is independently selected from the group consisting of hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, and a sulfur protecting group when attached to a sulfur atom, or two R^{A2a} groups are joined to form an optionally substituted heterocyclic ring; and
optionally any two of R^{A1}, R^{A2}, and R^{A2a} groups are joined to form an optionally substituted carbocyclic, optionally substituted heterocyclic, optionally substituted aryl, or optionally substituted heteroaryl ring;

R^{B1} is selected from the group consisting of hydrogen, halogen, optionally substituted acyl, optionally substituted alkyl, optionally substituted alkenyl, optionally substituted alkynyl, optionally substituted carbocyclyl, optionally substituted aryl, optionally substituted heteroaryl, —CN, —OR^{B1a}, —N(R^{B1a})₂, and —SR^{B1a}, wherein each occurrence of R^{B1a} is independently selected from the group consisting of hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, and a sulfur protecting group when attached to a sulfur atom, or R^{B1} and R^{B2} are joined to form an optionally substituted carbocyclic, optionally substituted heterocyclic, optionally substituted aryl, or optionally substituted heteroaryl ring;

W_{B} is N or CR^{B2}, wherein R^{B2} is selected from the group consisting of hydrogen, halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{B2a}, —N(R^{B2a})₂, and —SR^{B2a}, or R^{B2} and R^{B1} are joined to form an optionally substituted carbocyclic, optionally substituted heterocyclic, optionally substituted aryl, or optionally substituted heteroaryl ring, wherein each occurrence of R^{B2a} is independently selected from the group consisting of hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, and a sulfur protecting group when attached to a sulfur atom, or two R^{B2a} groups are joined to form an optionally substituted heterocyclic ring;

L¹ is a bond or an optionally substituted C₁₋₄ hydrocarbon chain, optionally wherein one or more carbon units of the optionally substituted C₁₋₄ hydrocarbon chain are independently replaced with —O—, —S—, —NR^{L1}—, —S(═O)—, or —S(═O)₂—, wherein R^{L1} is hydrogen, substituted or unsubstituted C₁₋₆ alkyl, or a nitrogen protecting group, and optionally wherein two substituents on the optionally substituted C₁₋₄ hydrocarbon chain are taken together to form an optionally substituted carbocyclic or optionally substituted heterocyclic ring;

L² is a bond or an optionally substituted C₁₋₄ hydrocarbon chain, optionally wherein one or more carbon units of the optionally substituted C₁₋₄ hydrocarbon chain are independently replaced with —O—, —S—, —NR^{L2}—, —S(═O)—, or —S(═O)₂—, wherein R^{L2} is hydrogen, substituted or unsubstituted C₁₋₆ alkyl, or a nitrogen protecting group, and optionally wherein two substituents on the optionally substituted C₁₋₄ hydrocarbon chain are taken together to form an optionally substituted carbocyclic or optionally substituted heterocyclic ring;

each instance of R^{C} is independently selected from the group consisting of hydrogen, halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, ═O, —CN, —OR^{C1}, —N(R^{C1})₂, and —SR^{C1}; or two R^{C} groups are taken together to form an optionally substituted, heterocyclic, carbocyclic, aryl, or heteroaryl ring, wherein two substituents on the substituted heterocyclic ring or substituted carbocyclic ring, or one substituent on the substituted heterocyclic ring or substituted carbocyclic ring and a third R^{C} group, are taken together to form another optionally substituted heterocyclic ring or optionally substituted carbocyclic ring; wherein each occurrence of R^{C1} is independently selected from the group consisting of hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, and a sulfur protecting group when attached to a sulfur atom, or two R^{C1} groups are joined to form an optionally substituted heterocyclic ring;

n is 0, 1, 2, 3, 4, 5, 6, 7, 8, 9, or 10;

each instance of R^{D} is independently selected from the group consisting of hydrogen, halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{D1}, —N(R^{D1})₂, and —SR^{D1}, wherein each occurrence of R^{D1} is independently selected from the group consisting of hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, and a sulfur protecting group when attached to a sulfur atom, or two R^{D1} groups are joined to form an optionally substituted heterocyclic ring;

p is 0, 1, 2, 3, or 4;

R^{E} is of any one of the Formulae (ii-1)-(ii-20):

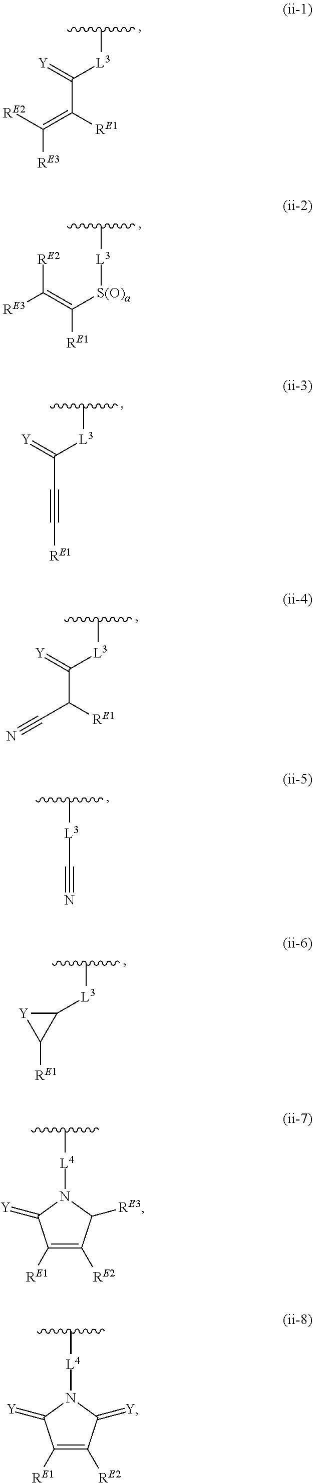

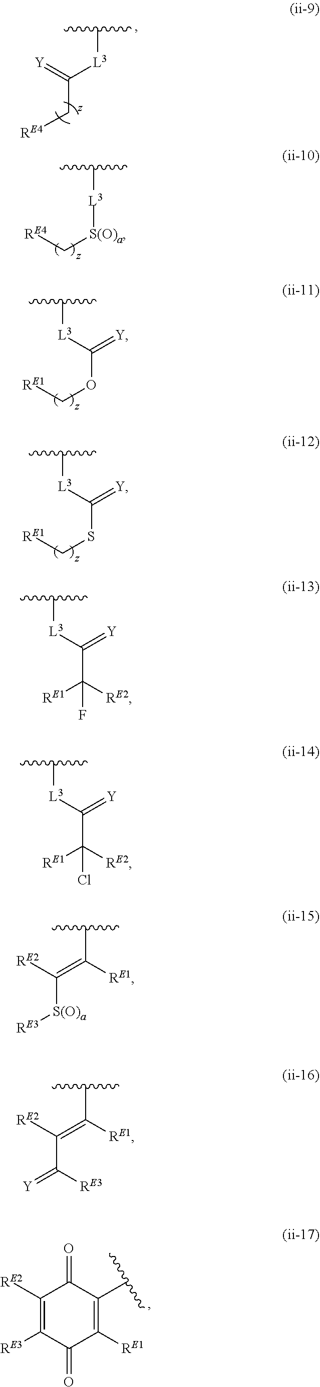

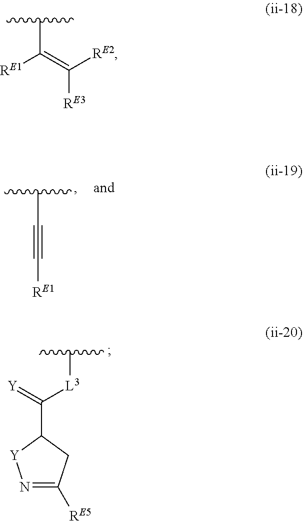

L³ is a bond, —O—, —S—, —NR^{L3a}—, or an optionally substituted C₁₋₄ hydrocarbon chain, optionally wherein one or more carbon units of the hydrocarbon chain are independently replaced with —O—, —S—, —NR^{L3a}—, —NR^{L3a}C(═O)—, —C(═O)NR^{L3a}—, —SC(═O)—, —C(═O)S—, —OC(═O)—, —C(═O)O—, —NR^{L3a}C(═S)—, —C(═S)NR^{L3a}—, trans-CR^{L3b}═CR^{L3b}—, cis-CR^{L3b}═CR^{L3b}—, —C≡C—, —S(═O)—, —S(═O)O—, —OS(═O)—, —S(═O)NR^{L3a}—, —NR^{L3a}S(═O)—, —S(═O)₂—, —S(═O)₂O—, —OS(═O)₂—, —S(═O)₂NR^{L3a}—, or —NR^{L3a}S(═O)₂—, wherein R^{L3a} is hydrogen, substituted or unsubstituted C₁₋₆ alkyl, or a nitrogen protecting group, and wherein each occurrence of R^{L3b} is independently selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{L3b} groups are joined to form an optionally substituted carbocyclic or optionally substituted heterocyclic ring;

L⁴ is a bond or an optionally substituted C₁₋₄ hydrocarbon chain;

R^{E1} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E1a}, —CH₂N(R^{E1a})₂, —CH₂SR^{E1a}, —OR^{E1a}, —N(R^{E1a})₂, —Si(R^{E1a})₃, and —SR^{E1a}, wherein each occurrence of R^{E1a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E1a} groups are joined to form an optionally substituted heterocyclic ring;

R^{E2} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E2a}, —CH₂N(R^{E2a})₂, —CH₂SR^{E2a}, —OR^{E2a}, —N(R^{E2a})₂, and —SR^{E2a}, wherein each occurrence of R^{E2a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E2a} groups are joined to form an optionally substituted heterocyclic ring;

R^{E3} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E3a}, —CH₂N(R^{E3a})₂, —CH₂SR^{E3a}, —OR^{E3a}, —N(R^{E3a})₂, and —SR^{E3a}, wherein each occurrence of R^{E3a} is independently selected from the group consisting of hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, and optionally substituted heteroaryl, or two R^{E3a} groups are joined to form an optionally substituted heterocyclic ring;

optionally R^{E1} and R^{E3}, or R^{E2} and R^{E3}, or R^{E1} and R^{E2} are joined to form an optionally substituted carbocyclic or optionally substituted heterocyclic ring;

R^{E4} is a leaving group;

R^{E5} is halogen;

Y is O, S, or NR^{E6}, wherein R^{E6} is hydrogen, substituted or unsubstituted C₁₋₆ alkyl, or a nitrogen protecting group;

a is 1 or 2; and

z is 0, 1, 2, 3, 4, 5, or 6.

In certain embodiments, the compound of Formula (XV) is of formula:

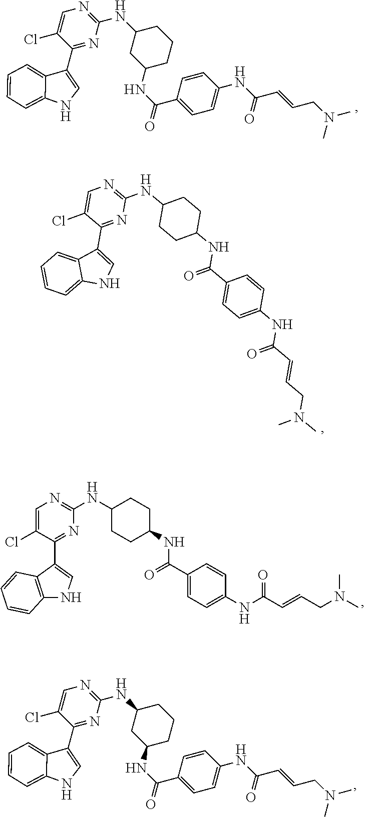

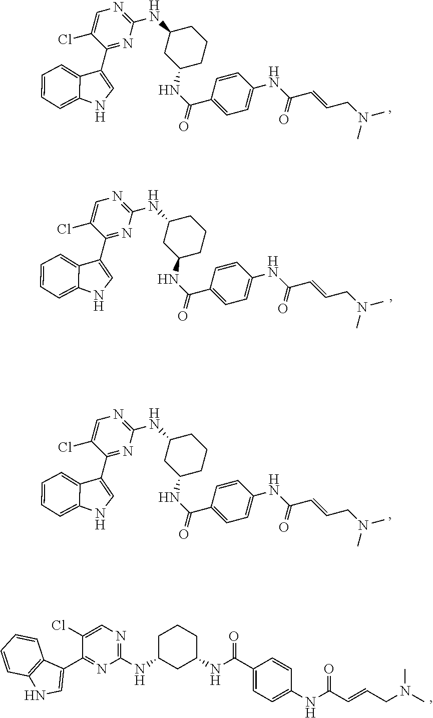 [SMILES: CN(C)C/C=C/C(=O)Nc1ccc(C(=O)N[C@H]2CCC[C@H](Nc3ncc(Cl)c(-c4c[nH]c5ccccc45)n3)C2)cc1 ; CN(C)C/C=C/C(=O)Nc1ccc(C(=O)N[C@@H]2CCC[C@@H](Nc3ncc(Cl)c(-c4c[nH]c5ccccc45)n3)C2)cc1 ; CN(C)C/C=C/C(=O)Nc1ccc(C(=O)N[C@H]2CCC[C@@H](Nc3ncc(Cl)c(-c4c[nH]c5ccccc45)n3)C2)cc1 ; CN(C)C/C=C/C(=O)Nc1ccc(C(=O)N[C@H]2CCC[C@@H](Nc3ncc(Cl)c(-c4c[nH]c5ccccc45)n3)C2)cc1]

or a pharmaceutically acceptable salt, solvate, hydrate, polymorph, co-crystal, tautomer, stereoisomer, isotopically labeled derivative, or prodrug thereof.

In certain embodiments, the CDK7 inhibitor is a compound of Formula (XVI):

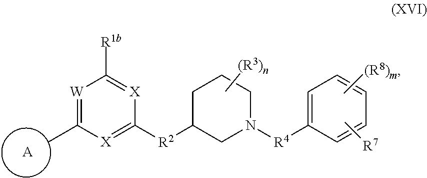

or a or a pharmaceutically acceptable salt, solvate, hydrate, polymorph, co-crystal, tautomer, stereoisomer, isotopically labeled derivative, or prodrug thereof, wherein:

ring A is an optionally substituted heteroaryl ring of any one of the Formulae (i-1)-(i-5):

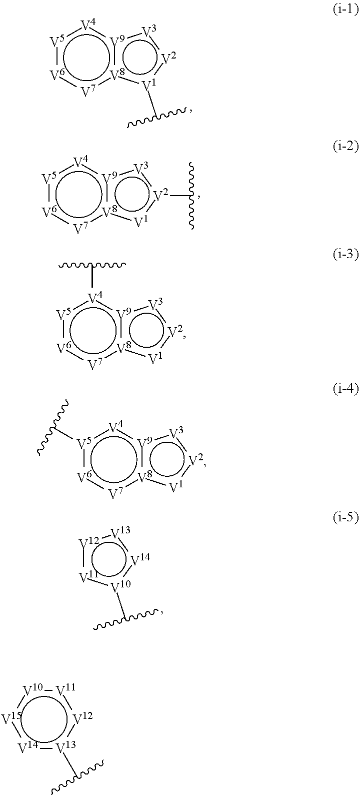

(i-6), wherein:

each instance of V¹, V², V³, V⁴, V⁵, V⁶, V⁷, V⁸, V⁹, V¹⁰, V¹¹, V¹², V¹³, V¹⁴ and V¹⁵ is independently O, S, N, N(R^{A1}), C, or C(R^{A2});

each instance of R^{A1} is independently selected from hydrogen, deuterium, optionally substituted acyl, optionally substituted alkyl, optionally substituted alkenyl, optionally substituted alkynyl, optionally substituted carbocyclyl, optionally substituted heterocyclyl, optionally substituted aryl, and optionally substituted heteroaryl;

each instance of R^{A2} is independently selected from hydrogen, deuterium, halogen, —CN, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{A2a}, —N(R^{A2a})₂, and —SR^{A2a}, wherein each occurrence of R^{A2a} is independently selected from hydrogen, optionally substituted acyl, optionally substituted alkyl, optionally substituted alkenyl, optionally substituted alkynyl, optionally substituted carbocyclyl, optionally substituted heterocyclyl, optionally substituted aryl, and optionally substituted heteroaryl, or

any two R^{A1}, any two R^{A2}, or one R^{A1} and one R^{A2} are joined to form an optionally substituted carbocyclic, optionally substituted heterocyclic, optionally substituted aryl, or optionally substituted heteroaryl ring;

each X is independently selected from N and CH, wherein at least one X is N;

W is selected from N and C(R^{1a});

each of R^{1a}, if present, and R^{1b} is independently selected from hydrogen, deuterium, halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{B1a}, —N(R^{B1a})₂, and —SR^{B1a}, wherein each occurrence of R^{B1a} is independently selected from hydrogen, optionally substituted acyl, optionally substituted alkyl, optionally substituted alkenyl, optionally substituted alkynyl, optionally substituted carbocyclyl, optionally substituted heterocyclyl, optionally substituted aryl, and optionally substituted heteroaryl, or

R^{1a} and R^{1b} are joined to form an optionally substituted carbocyclic, optionally substituted heterocyclic, optionally substituted aryl, or optionally substituted heteroaryl ring;

R² is an optionally substituted C₁-C₄ alkylene or an optionally substituted C₂-C₄ alkenylene or alkynylene, wherein one or more methylene units of the alkylene, alkenylene or alkynylene are optionally and independently replaced with —O—, —S—, or —N(R⁶)—;

each instance of R³, if present, is independently selected from deuterium, halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{C1}, —N(R^{C1})₂, and —SR^{C1}, wherein each occurrence of R^{C1} is independently selected from hydrogen, optionally substituted acyl, optionally substituted alkyl, optionally substituted alkenyl, optionally substituted alkynyl, optionally substituted carbocyclyl, optionally substituted heterocyclyl, optionally substituted aryl, and optionally substituted heteroaryl, or

two R³ groups bound to the same ring carbon atom are taken together to form ═O, or

two R³ groups bound to the same or different ring carbon atoms are joined to form an optionally substituted carbocyclyl, optionally substituted heterocyclyl, optionally substituted aryl, or optionally substituted heteroaryl ring;

R⁴ is selected from a bond, an optionally substituted C₁-C₄ alkylene, and an optionally substituted C₂-C₄ alkenylene or alkynylene, wherein:

one or more methylene units of the alkylene, alkenylene or alkynylene other than a methylene unit bound to a nitrogen atom is optionally and independently replaced with —O—, —S—, —N(R⁶)—, or —S(═O)₂—, and

two substituents on either the same or adjacent carbon atoms in the alkylene, alkenylene or alkynylene are taken together to form an optionally substituted carbocyclic or optionally substituted heterocyclic ring;

each R⁶ is independently selected from hydrogen, and —C₁-C₆ alkyl;

R⁷ is any one of the Formulae (ii-1)-(ii-20):

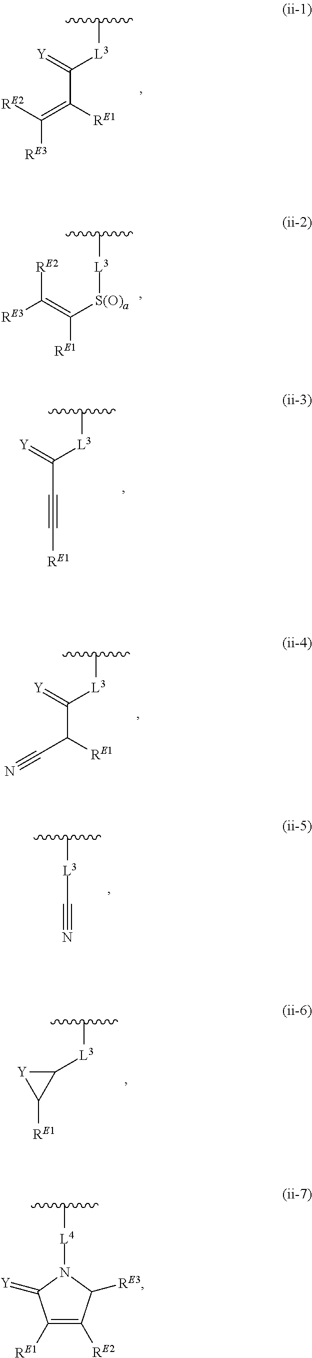

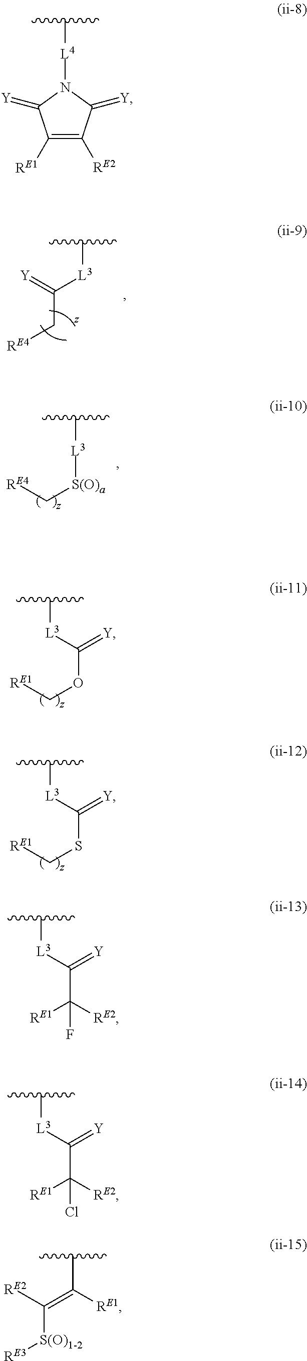

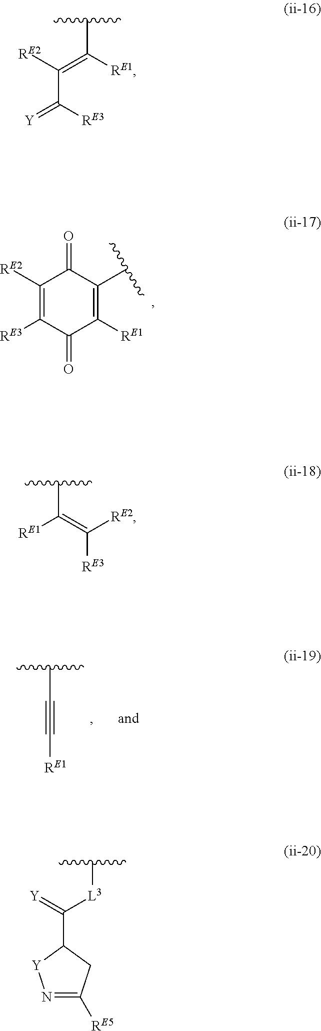

wherein:

L³ is a bond, an optionally substituted C₁-C₄ alkylene, or an optionally substituted C₂-C₄ alkenylene or alkynylene, wherein one or more methylene units of the alkylene, alkenylene or alkynylene are optionally and independently replaced with —O—, —S—, or —N(R⁶)—;

L⁴ is a bond, an optionally substituted C₁-C₄ alkylene, or an optionally substituted C₂-C₄ alkenylene or alkynylene;

R^{E1} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E1a}, —CH₂N(R^{E1a})₂, —CH₂SR^{E1a}, —OR^{E1a}, —N(R^{E1a})₂, —Si(R^{E1a})₃, and —SR^{E1a}, wherein each occurrence of R^{E1a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E1a} groups are joined to form an optionally substituted heterocyclic ring;

R^{E2} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E2a}, —CH₂N(R^{E2a})₂, —CH₂SR^{E2a}, —OR^{E2a}, —N(R^{E2a})₂, and —SR^{E2a}, wherein each occurrence of R^{E2a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E2a} groups are joined to form an optionally substituted heterocyclic ring;

R^{E3} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E3a}, —CH₂N(R^{E3a})₂, —CH₂SR^{E3a}, —OR^{E3a}, —N(R^{E3a})₂, and —SR^{E3a}, wherein each occurrence of R^{E3a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E3a} groups are joined to form an optionally substituted heterocyclic ring;

optionally R^{E1} and R^{E3}, or R^{E2} and R^{E3}, or R^{E1} and R^{E2} are joined to form an optionally substituted carbocyclic or optionally substituted heterocyclic ring;

R^{E4} is a leaving group;

R^{E5} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E5a}, —CH₂N(R^{E5a})₂, —CH₂SR^{E5a}, —OR^{E5a}, —N(R^{E5a})₂, and —SR^{a}, wherein each occurrence of R^{E5a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E5a} groups are joined to form an optionally substituted heterocyclic ring;

Y is O, S, or NR^{E6}, wherein R^{E6} is hydrogen, substituted or unsubstituted C₁₋₆ alkyl, or a nitrogen protecting group;

a is 1 or 2;

z is 0, 1, 2, 3, 4, 5, or 6.

each instance of R⁸, if present, is independently selected from deuterium, halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{D1}, —N(R^{D1})₂, and —SR^{D1}, wherein each occurrence of R^{D1} is independently selected from hydrogen, optionally substituted acyl, optionally substituted alkyl, optionally substituted alkenyl, optionally substituted alkynyl, optionally substituted carbocyclyl, optionally substituted heterocyclyl, and optionally substituted aryl, optionally substituted heteroaryl, or

two R⁸ groups are joined to form an optionally substituted carbocyclyl, optionally substituted heterocyclyl, optionally substituted aryl, or optionally substituted heteroaryl ring;

m is 0, 1, 2, 3 or 4; and

n is 0, 1, 2, 3, 4, 5 or 6.

In certain embodiments, the compound of Formula (XVI) is of formula:

In certain embodiments, the compound of Formula (I) is selected from

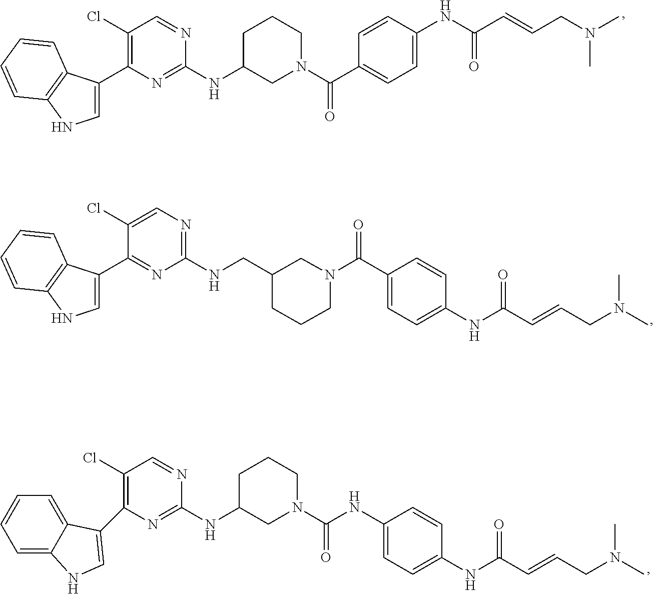 [SMILES: CN(C)C/C=C/C(=O)Nc1ccc(C(=O)N2CCCC(Nc3ncc(Cl)c(-c4c[nH]c5ccccc45)n3)C2)cc1 ; CN(C)C/C=C/C(=O)Nc1ccc(C(=O)N2CCCC(CNc3ncc(Cl)c(-c4c[nH]c5ccccc45)n3)C2)cc1 ; CN(C)C/C=C/C(=O)Nc1ccc(NC(=O)N2CCCC(Nc3ncc(Cl)c(-c4c[nH]c5ccccc45)n3)C2)cc1]

or a pharmaceutically acceptable salt, solvate, hydrate, polymorph, co-crystal, tautomer, stereoisomer, isotopically labeled derivative, or prodrug thereof.

In certain embodiments, the CDK7 inhibitor is a compound of Formula (XVII):

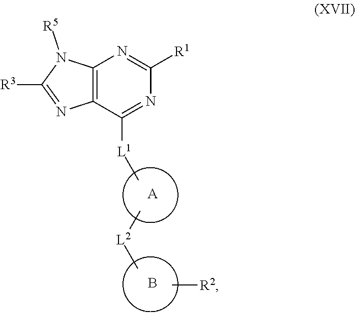

a pharmaceutically acceptable salt, solvate, hydrate, polymorph, co-crystal, tautomer, stereoisomer, isotopically labeled derivative, or prodrug thereof, wherein:

R¹ is optionally substituted alkyl, optionally substituted alkenyl, optionally substituted alkynyl, optionally substituted carbocyclyl, optionally substituted aryl, optionally substituted heterocyclyl, optionally substituted heteroaryl, —NR^{a}R^{b}, —OR^{b}, —SR^{b}, —C(═O)R^{b}, —C(═O)OR^{b}, or —C(═O)NR^{a}R^{b}, wherein each instance of R^{a} and R^{b} is independently hydrogen, optionally substituted alkyl, optionally substituted alkenyl, optionally substituted alkynyl, optionally substituted carbocyclyl, optionally substituted aryl, optionally substituted heterocyclyl, optionally substituted aryl, optionally substituted heteroaryl, or a nitrogen protecting group when attached to nitrogen, or an oxygen protecting group when attached to oxygen, or a sulfur protecting group when attached to sulfur; or R^{a} and R^{b} are joined to form an optionally substituted heterocyclic or optionally substituted heteroaryl ring;

R³ is hydrogen, halogen, or optionally substituted C₁-C₆ alkyl;

R⁵ is hydrogen, optionally substituted C₁-C₆ alkyl, or a nitrogen protecting group;

L¹ is a bond, —NR^{L1}—(CH₂)ₜ—, —O—, or —S—;

R^{L1} is hydrogen, optionally substituted C₁-C₆ alkyl, or a nitrogen protecting group;

t is 0 or an integer between 1 and 5, inclusive;

Ring A is optionally substituted carbocyclyl, optionally substituted heterocyclyl, optionally substituted aryl, or optionally substituted heteroaryl;

L² is a bond, optionally substituted C₁₋₄ alkylene, —C(═O)—, —NR^{L2}—, —C(═O)NR^{L2}—, —NR^{L2}C(═O)—, —O—, or —S—, wherein R^{L2} is hydrogen, optionally substituted C₁-C₆ alkyl, or a nitrogen protection group;

Ring B is absent, optionally substituted carbocyclyl, optionally substituted heterocyclyl, optionally substituted aryl, or optionally substituted heteroaryl; and

R² is any of Formulae (i-1)-(i-41):

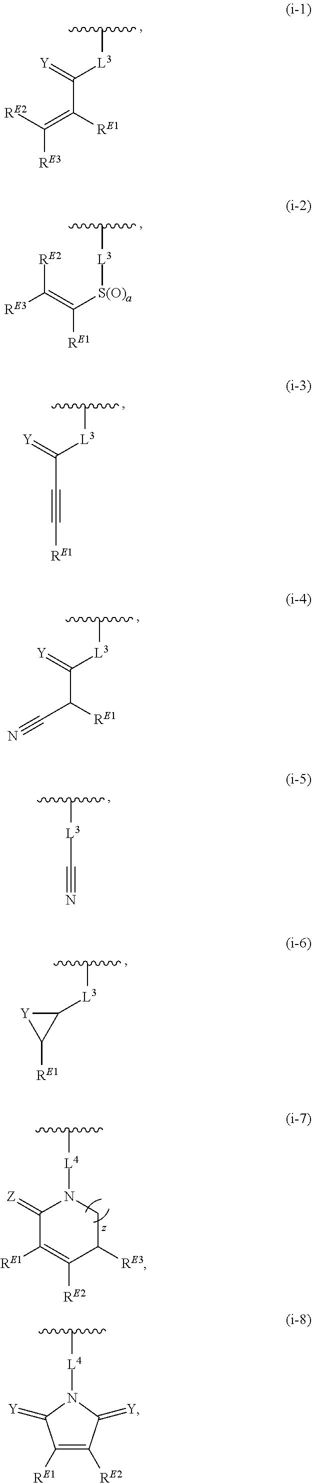

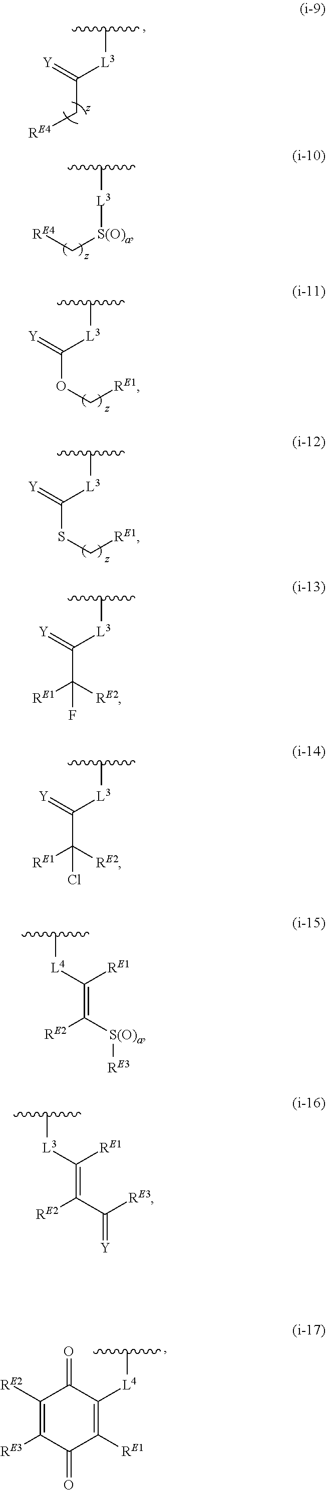

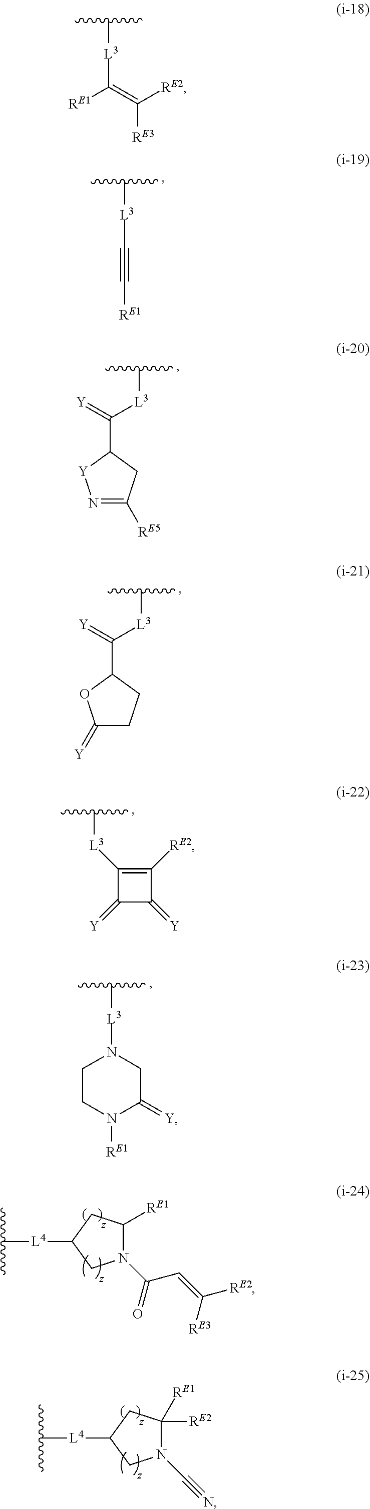

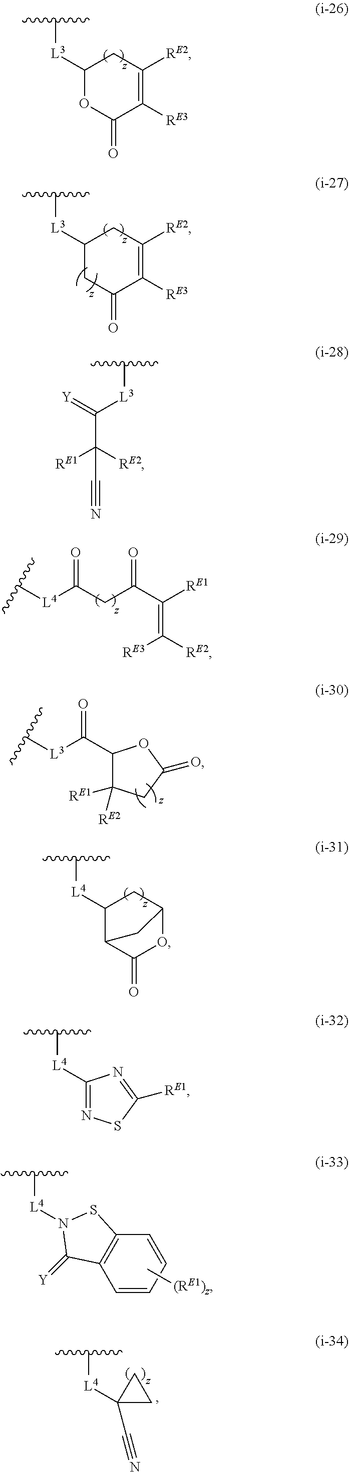

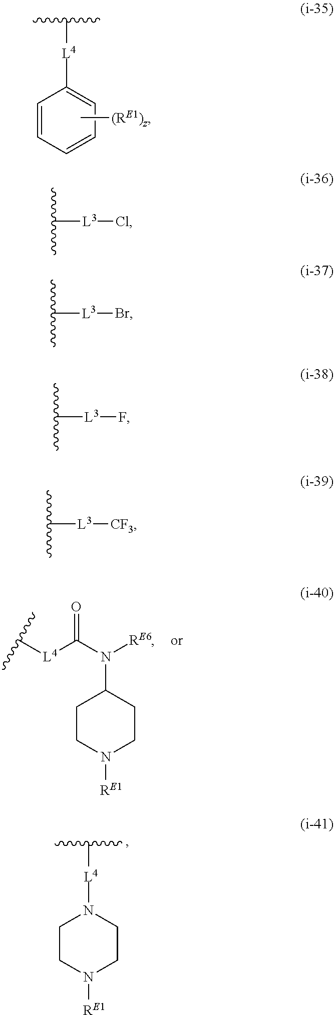

wherein:

L³ is a bond or an optionally substituted C₁₋₄ hydrocarbon chain, optionally wherein one or more carbon units of the hydrocarbon chain are independently replaced with —C═O—, —O—, —S—, —NR^{L3a}—, NR^{L3a}C(═O)—, —C(═O)NR^{L3a}—, —SC(═O)—, —C(═O)S—, —OC(═O)—, —C(═O)O—, —NR^{L3a}C(═S)—, —C(═S)NR^{L3a}—, trans-CR^{L3b}═CR^{L3b}—, cis-CR^{L3b}═CR^{L3b}—, —C≡C—, —S(═O)—, —S(═O)O—, —OS(═O)—, —S(═O)NR^{L3a}—, —NR^{L3a}S(═O)—, —S(═O)₂—, —S(═O)₂O—, —OS(═O)₂—, —S(═O)₂NR^{L3a}—, or —NR^{L3a}S(═O)₂—, wherein R^{L3a} is hydrogen, substituted or unsubstituted C₁₋₆ alkyl, or a nitrogen protecting group, and wherein each occurrence of R^{L3b} is independently hydrogen, halogen, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R^{L3b} groups are joined to form an optionally substituted carbocyclic or optionally substituted heterocyclic ring;

L⁴ is a bond or an optionally substituted, branched or unbranched C₁₋₆ hydrocarbon chain;

each of R^{E1}, R^{E2}, and R^{E3} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{EE}, —CH₂N(R^{EE})₂, —CH₂SR^{EE}, —OR^{EE}, —N(R^{EE})₂, —Si(R^{EE})₃, and —SR^{EE}, wherein each occurrence of R^{EE} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl, or two R^{EE} groups are joined to form an optionally substituted heterocyclic ring; or R^{E1} and R^{E3}, or R^{E2} and R^{E3}, or R^{E1} and R^{E2} are joined to form an optionally substituted carbocyclic or optionally substituted heterocyclic ring;

R^{E4} is a leaving group;

R^{E5} is halogen;

R^{E6} is hydrogen, substituted or unsubstituted C₁₋₆ alkyl, or a nitrogen protecting group;

each instance of Y is independently O, S, or NR^{E7}, wherein R^{E7} is hydrogen, substituted or unsubstituted C₁₋₆ alkyl, or a nitrogen protecting group;

a is 1 or 2; and

each instance of z is independently 0, 1, 2, 3, 4, 5, or 6, as valency permits.

In certain embodiments, the compound of Formula (XVII) is not

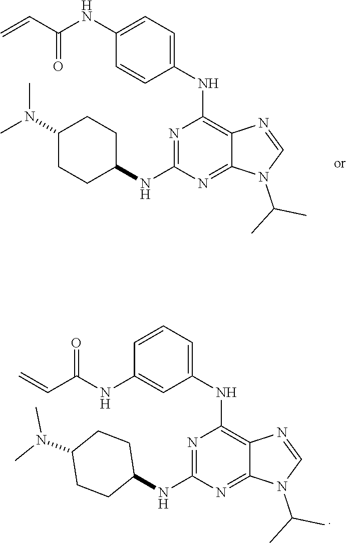

In certain embodiments, the compound of Formula (XVII) is of the formula:

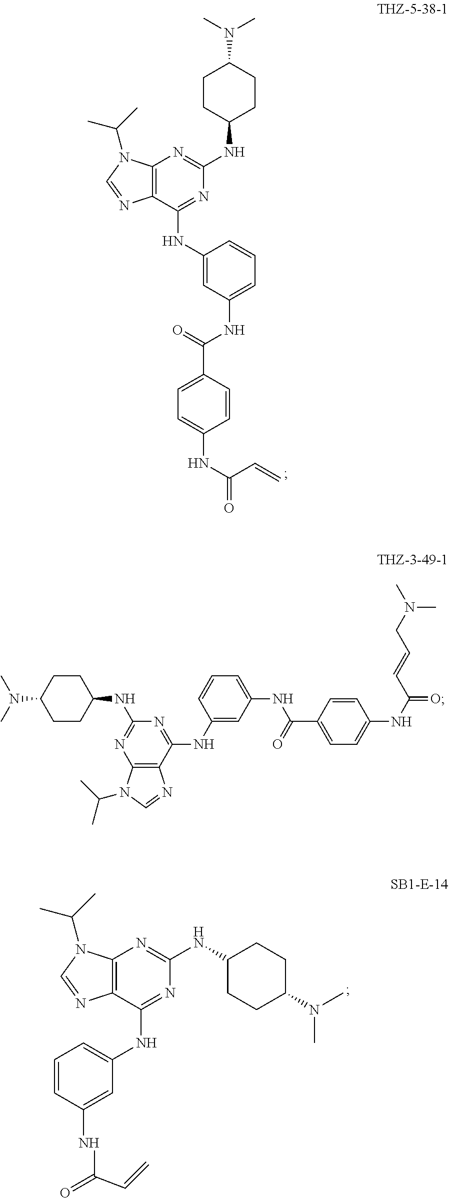

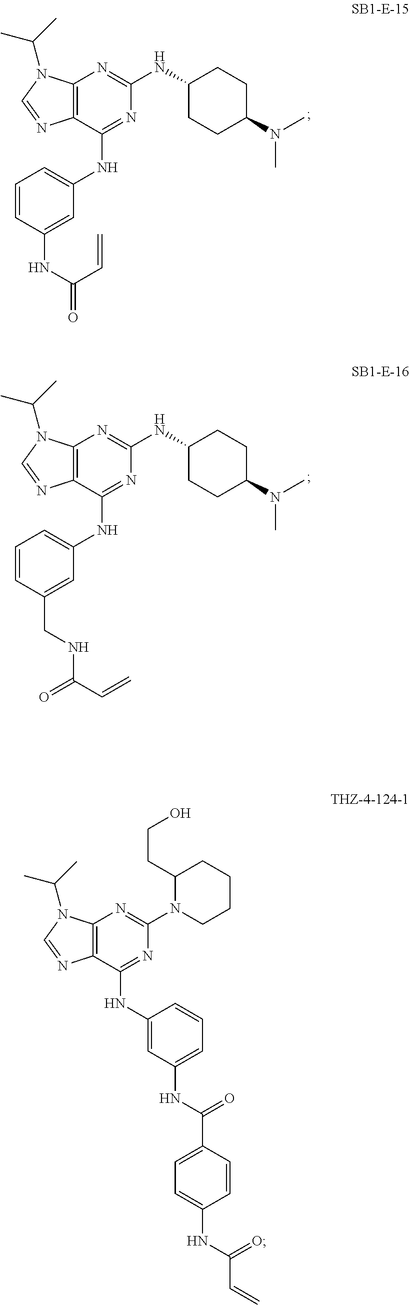

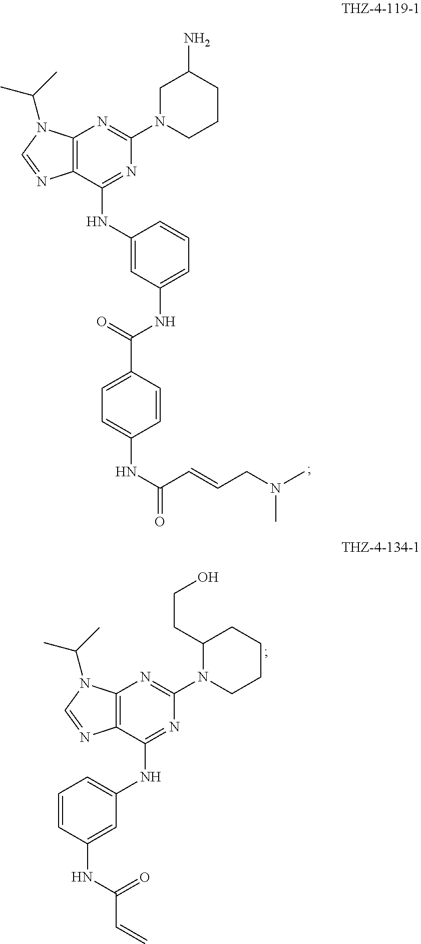

or a pharmaceutically acceptable salt, solvate, hydrate, polymorph, co-crystal, tautomer, stereoisomer, isotopically labeled derivative, or prodrug thereof.

In certain embodiments, the CDK7 inhibitor is a compound of Formula (XVIII):

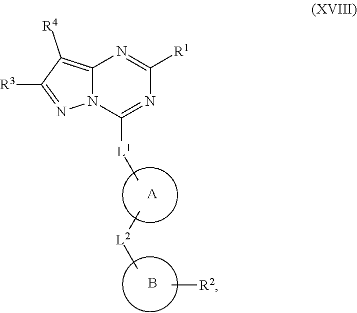

or a pharmaceutically acceptable salt, solvate, hydrate, polymorph, co-crystal, tautomer, stereoisomer, isotopically labeled derivative, or prodrug thereof, wherein:

R¹ is optionally substituted alkyl, optionally substituted alkenyl, optionally substituted alkynyl, optionally substituted carbocyclyl, optionally substituted aryl, optionally substituted heterocyclyl, optionally substituted heteroaryl, —NR^{a}R^{b}, —OR^{b}, —SR^{b}, —C(═O)R^{b}, —C(═O)OR^{b}, or —C(═O)NR^{a}R^{b}, wherein each instance of R^{a} and R^{b} is independently hydrogen, optionally substituted alkyl, optionally substituted alkenyl, optionally substituted alkynyl, optionally substituted carbocyclyl, optionally substituted aryl, optionally substituted heterocyclyl, optionally substituted aryl, optionally substituted heteroaryl, or a nitrogen protecting group when attached to nitrogen, or an oxygen protecting group when attached to oxygen, or a sulfur protecting group when attached to sulfur; or R^{a} and R^{b} are joined to form an optionally substituted heterocyclic or optionally substituted heteroaryl ring;

each of R³ and R⁴ is independently hydrogen, halogen, or optionally substituted C₁-C₆ alkyl;

L¹ is a bond, —NR^{L1}—(CH₂)ₜ—, —O—, or —S—;

R^{L1} is hydrogen, optionally substituted C₁-C₆ alkyl, or a nitrogen protecting group;

t is 0 or an integer between 1 and 5, inclusive;

Ring A is optionally substituted carbocyclyl, optionally substituted heterocyclyl, optionally substituted aryl, or optionally substituted heteroaryl;

L² is a bond, optionally substituted C₁₋₄ alkylene, —C(═O)—, —NR^{L2}—, —C(═O)NR^{L2}—, —NR^{L2}C(═O)—, —O—, or —S—, wherein R^{L2} is hydrogen, optionally substituted C₁-C₆ alkyl, or a nitrogen protection group;

Ring B is absent, optionally substituted carbocyclyl, optionally substituted heterocyclyl, optionally substituted aryl, or optionally substituted heteroaryl; and

R² is any of Formulae (i-1)-(i-41):

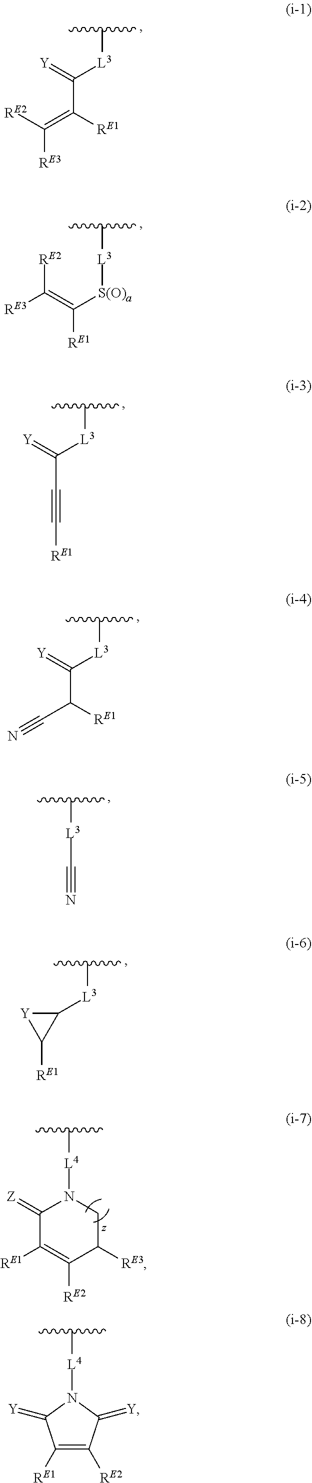

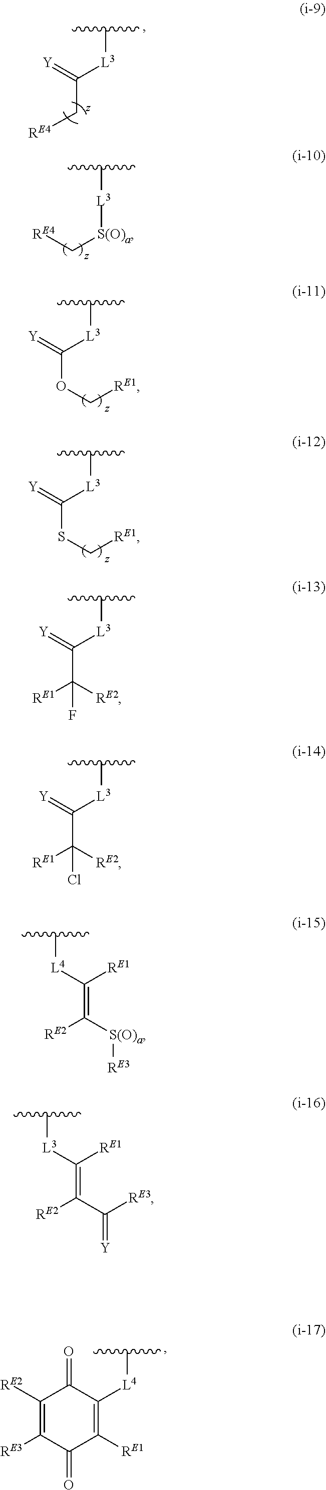

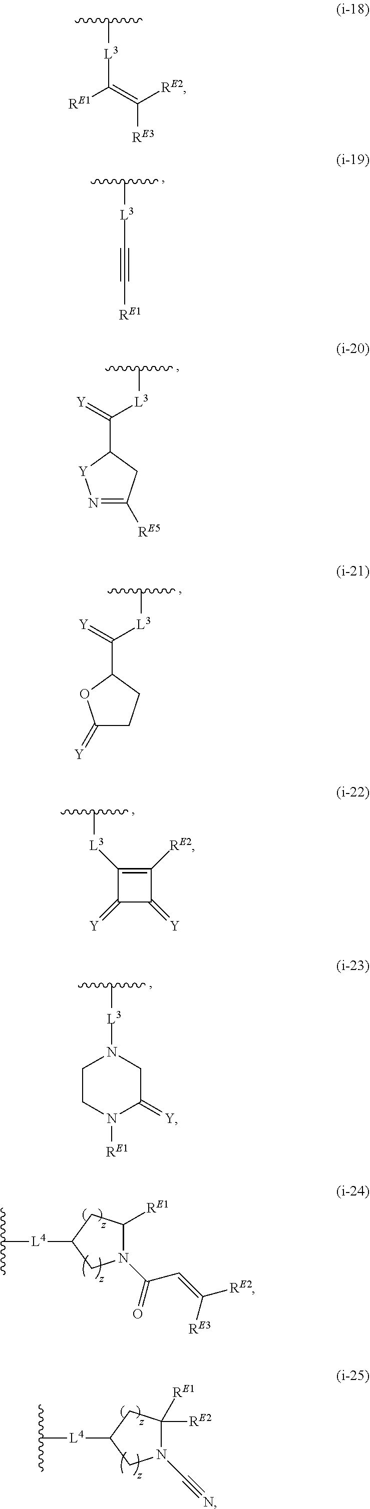

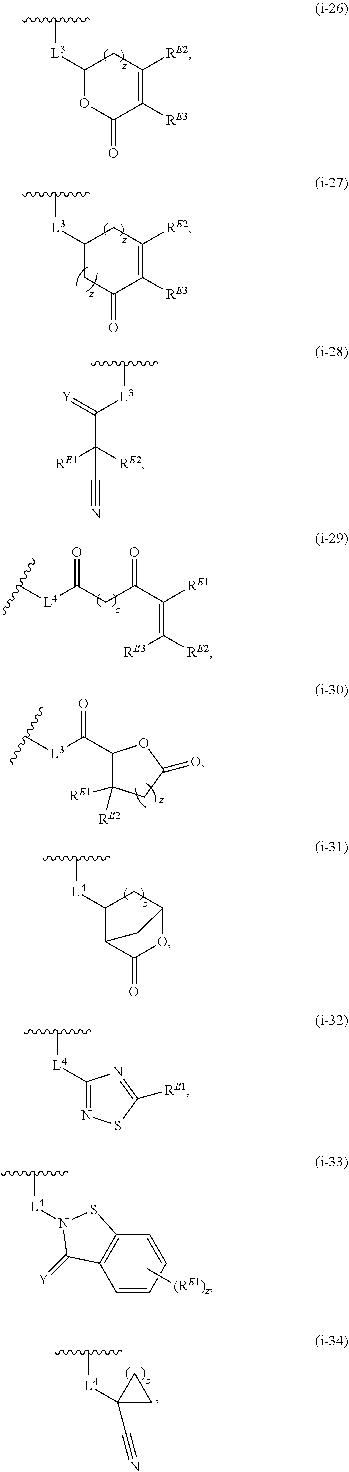

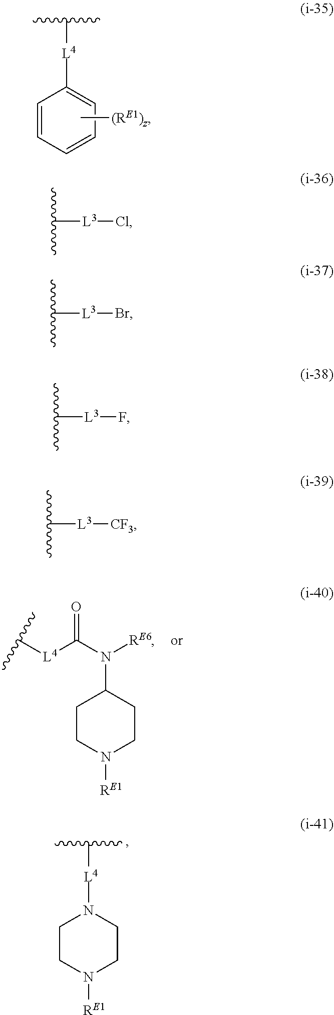

wherein:

L³ is a bond or an optionally substituted C₁₋₄ hydrocarbon chain, optionally wherein one or more carbon units of the hydrocarbon chain are independently replaced with —C═O—, —O—, —S—, —NR^{L3a}—, —NR^{L3a}C(═O)—, —C(═O)NR^{L3a}—, —SC(═O)—, —C(═O)S—, —OC(═O)—, —C(═O)O—, —NR^{L3a}C(═S)—, —C(═S)NR^{L3a}—, trans-CR^{L3b}═CR^{L3b}—, cis-CR^{L3b}═CR^{L3b}—, —C≡C—, —S(═O)—, —S(═O)O—, —OS(═O)—, —S(═O)NR^{L3a}, —NR^{L3a}S(═O)—, —S(═O)₂—, —S(═O)₂O—, —OS(═O)₂—, —S(═O)₂NR^{L3a}—, or —NR^{L3a}S(═O)₂—, wherein R^{L3a} is hydrogen, substituted or unsubstituted C₁₋₆ alkyl, or a nitrogen protecting group, and wherein each occurrence of R^{L3b} is independently hydrogen, halogen, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R^{L3b} groups are joined to form an optionally substituted carbocyclic or optionally substituted heterocyclic ring;

L⁴ is a bond or an optionally substituted, branched or unbranched C₁₋₆ hydrocarbon chain;

each of R^{E1}, R^{E2}, and R^{E3} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{EE}, —CH₂N(R^{E}E)₂, —CH₂SR^{EE}, —OR^{EE}, —N(R^{EE})₂, —Si(R^{EE})₃, and —SR^{EE}, wherein each occurrence of R^{EE} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl, or two R^{EE} groups are joined to form an optionally substituted heterocyclic ring;

or R^{E1} and R^{E3}, or R^{E2} and R^{E3}, or R^{E1} and R^{E2} are joined to form an optionally substituted carbocyclic or optionally substituted heterocyclic ring;

R^{E4} is a leaving group;

R^{E5} is halogen;

R^{E6} is hydrogen, substituted or unsubstituted C₁₋₆ alkyl, or a nitrogen protecting group;

each instance of Y is independently O, S, or NR^{E7}, wherein R^{E7} is hydrogen, substituted or unsubstituted C₁₋₆ alkyl, or a nitrogen protecting group;

a is 1 or 2; and

each instance of z is independently 0, 1, 2, 3, 4, 5, or 6, as valency permits.

In certain embodiments, the compound of Formula (XVIII) is of the formula:

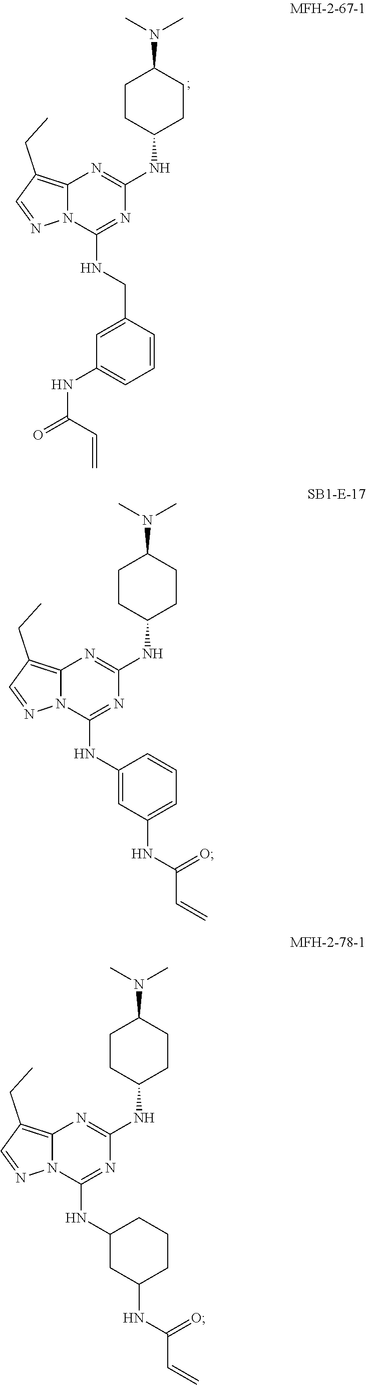

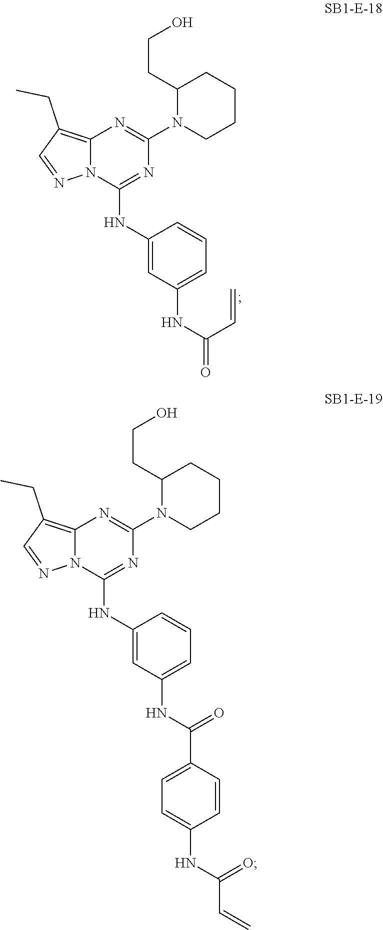 [SMILES: C=CC(=O)Nc1cccc(Nc2nc(N3CCCCC3CCO)nc3c(CC)cnn23)c1 ; C=CC(=O)Nc1ccc(C(=O)Nc2cccc(Nc3nc(N4CCCCC4CCO)nc4c(CC)cnn34)c2)cc1]

or a pharmaceutically acceptable salt, solvate, hydrate, polymorph, co-crystal, tautomer, stereoisomer, isotopically labeled derivative, or prodrug thereof.

In certain embodiments, the CDK7 inhibitor is a compound of Formula (XIX):

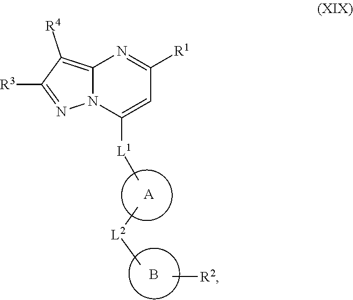

or a pharmaceutically acceptable salt, solvate, hydrate, polymorph, co-crystal, tautomer, stereoisomer, isotopically labeled derivative, or prodrug thereof, wherein:

R¹ is optionally substituted alkyl, optionally substituted alkenyl, optionally substituted alkynyl, optionally substituted carbocyclyl, optionally substituted aryl, optionally substituted heterocyclyl, optionally substituted heteroaryl, —NR^{a}R^{b}, —OR^{b}, —SR^{b}, —C(═O)R^{b}, —C(═O)OR^{b}, or —C(═O)NR^{a}R^{b}, wherein each instance of R^{a} and R^{b} is independently hydrogen, optionally substituted alkyl, optionally substituted alkenyl, optionally substituted alkynyl, optionally substituted carbocyclyl, optionally substituted aryl, optionally substituted heterocyclyl, optionally substituted aryl, optionally substituted heteroaryl, or a nitrogen protecting group when attached to nitrogen, or an oxygen protecting group when attached to oxygen, or a sulfur protecting group when attached to sulfur; or R^{a} and R^{b} are joined to form an optionally substituted heterocyclic or optionally substituted heteroaryl ring;

each of R³ and R⁴ is independently hydrogen, halogen, or optionally substituted C₁-C₆ alkyl;

L¹ is a bond, —NR^{L1}—(CH₂)ₜ—, —O—, or —S—;

R^{L1} is hydrogen, optionally substituted C₁-C₆ alkyl, or a nitrogen protecting group;

t is 0 or an integer between 1 and 5, inclusive;

Ring A is optionally substituted carbocyclyl, optionally substituted heterocyclyl, optionally substituted aryl, or optionally substituted heteroaryl;

L² is a bond, optionally substituted C₁₋₄ alkylene, —C(═O)—, —NR^{L2}—, —C(═O)NR^{L2}—, —NR^{L2}C(═O)—, —O—, or —S—, wherein R^{L2} is hydrogen, optionally substituted C₁-C₆ alkyl, or a nitrogen protection group;

Ring B is absent, optionally substituted carbocyclyl, optionally substituted heterocyclyl, optionally substituted aryl, or optionally substituted heteroaryl; and

R² is any of Formulae (i-1)-(i-41):

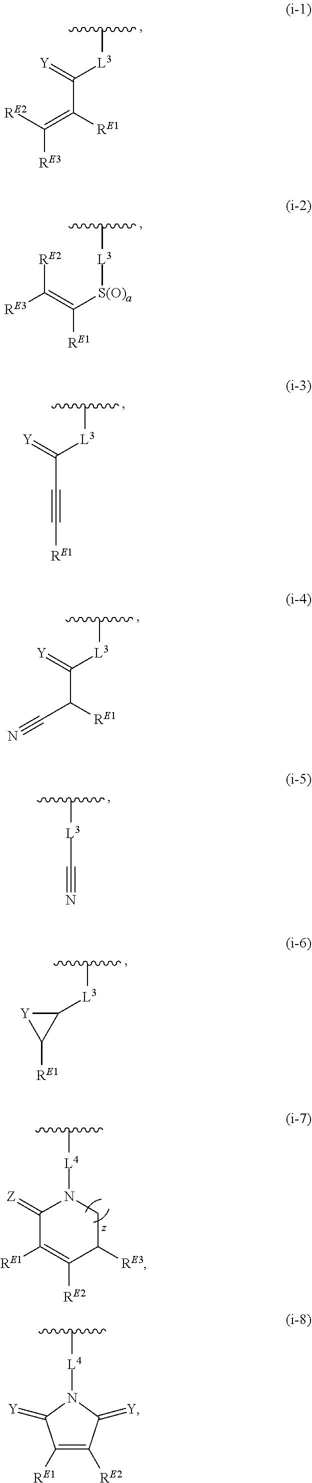

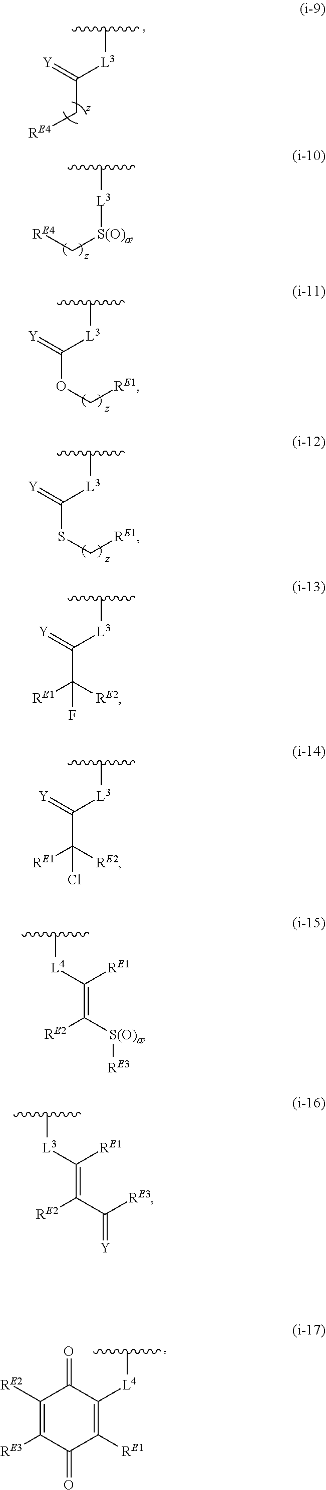

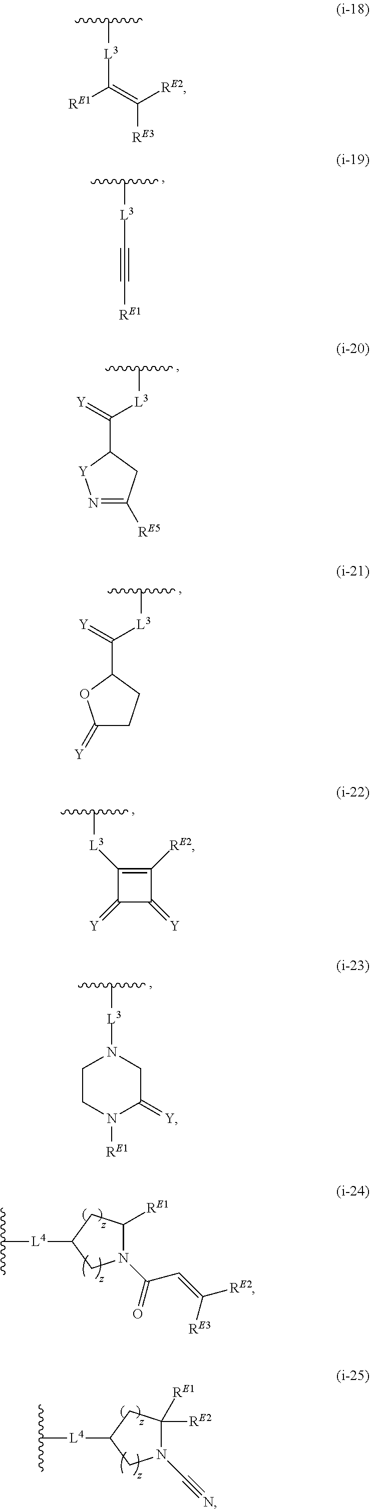

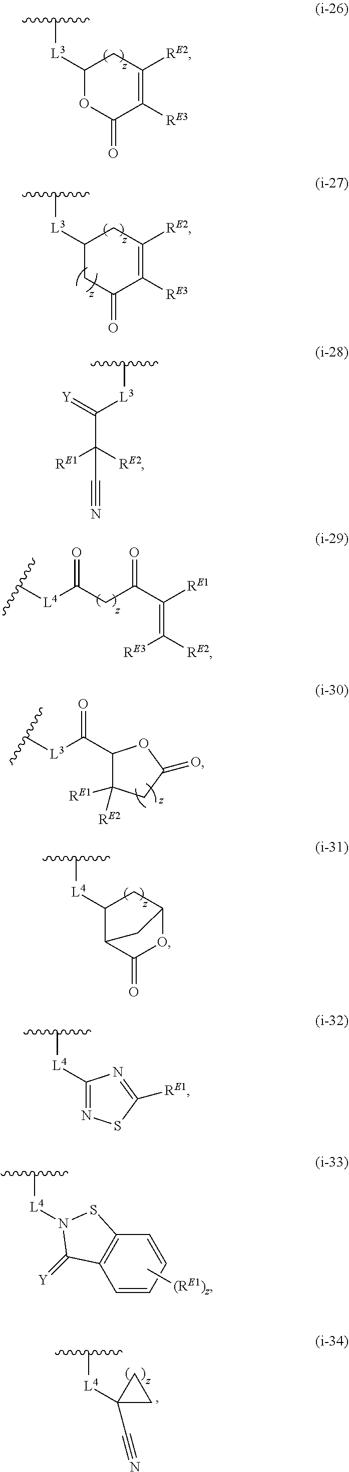

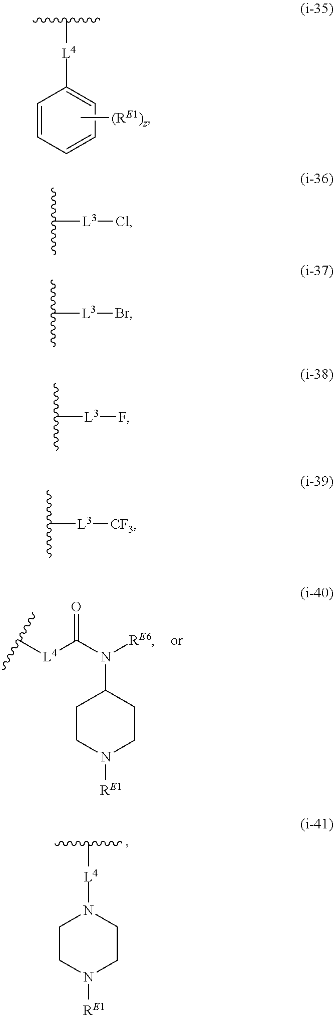

wherein:

L³ is a bond or an optionally substituted C₁₋₄ hydrocarbon chain, optionally wherein one or more carbon units of the hydrocarbon chain are independently replaced with —C═O—, —O—, —S—, —NR^{L3a}—, —NR^{L3a}C(═O)—, —C(═O)NR^{L3a}—, —SC(═O)—, —C(═O)S—, —OC(═O)—, —C(═O)O—, —NR^{L3a}C(═S)—, —C(═S)NR^{L3a}—, trans-CR^{L3b}═CR^{L3b}—, cis-CR^{L3b}═CR^{L3b}—, —C≡C—, —S(═O)—, —S(═O)O—, —OS(═O)—, —S(═O)NR^{L3a}, —NR^{L3a}S(═O)—, —S(═O)₂—, —S(═O)₂O—, —OS(═O)₂—, —S(═O)₂NR^{L3a}—, or —NR^{L3a}S(═O)₂—, wherein R^{L3a} is hydrogen, substituted or unsubstituted C₁₋₆ alkyl, or a nitrogen protecting group, and wherein each occurrence of R^{L3b} is independently hydrogen, halogen, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R^{L3b} groups are joined to form an optionally substituted carbocyclic or optionally substituted heterocyclic ring;

L⁴ is a bond or an optionally substituted, branched or unbranched C₁₋₆ hydrocarbon chain;

each of R^{E1}, R^{E2}, and R^{E3} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{EE}, —CH₂N(R^{E}E)₂, —CH₂SR^{EE}, —OR^{EE}, —N(R^{EE})₂, —Si(R^{EE})₃, and —SR^{EE}, wherein each occurrence of R^{EE} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl, or two R^{EE} groups are joined to form an optionally substituted heterocyclic ring;

or R^{E1} and R^{E3}, or R^{E2} and R^{E3}, or R^{E1} and R^{E2} are joined to form an optionally substituted carbocyclic or optionally substituted heterocyclic ring;

R^{E4} is a leaving group;

R^{E5} is halogen;

R^{E6} is hydrogen, substituted or unsubstituted C₁₋₆ alkyl, or a nitrogen protecting group;

each instance of Y is independently O, S, or NR^{E7}, wherein R^{E7} is hydrogen, substituted or unsubstituted C₁₋₆ alkyl, or a nitrogen protecting group;

a is 1 or 2; and

each instance of z is independently 0, 1, 2, 3, 4, 5, or 6, as valency permits.

In certain embodiments, the compound of Formula (XIX) is of formula:

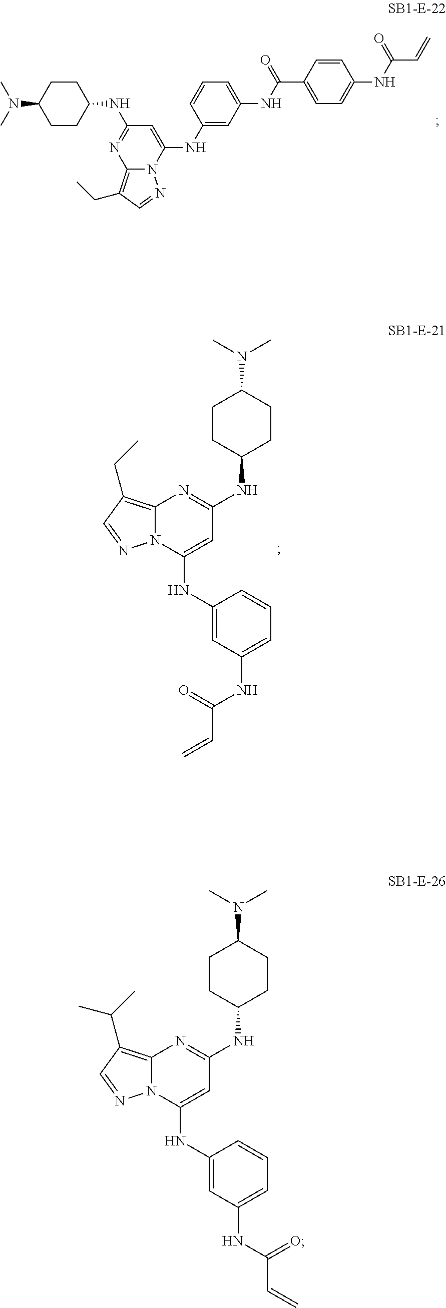

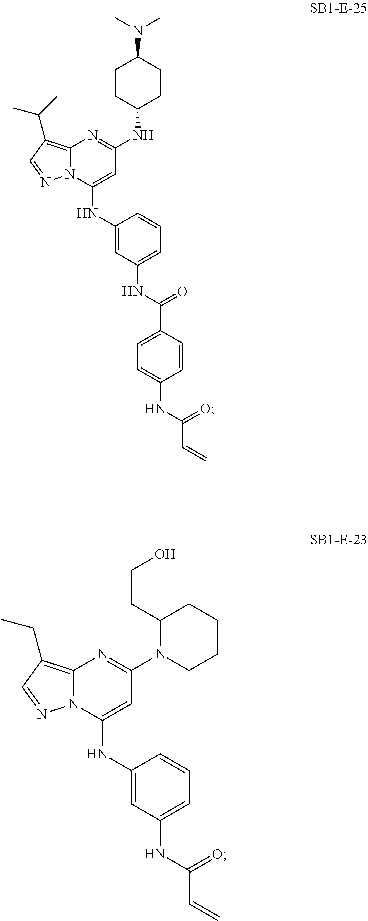

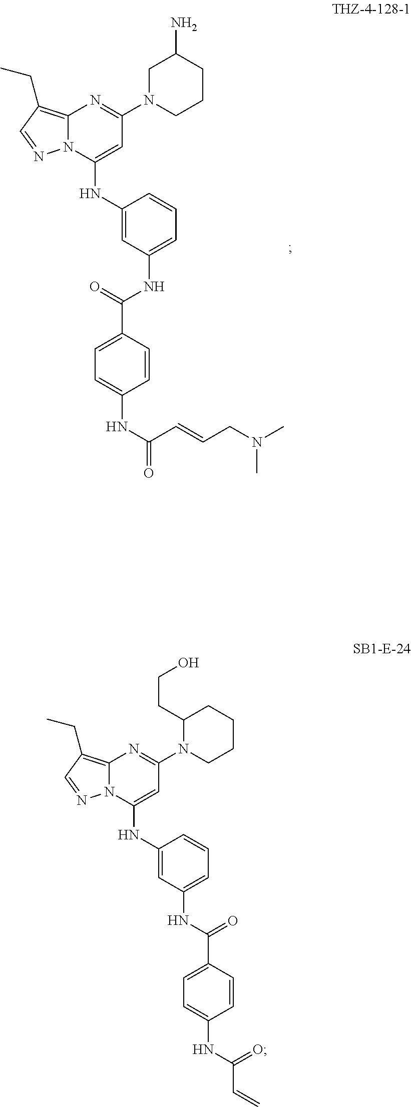

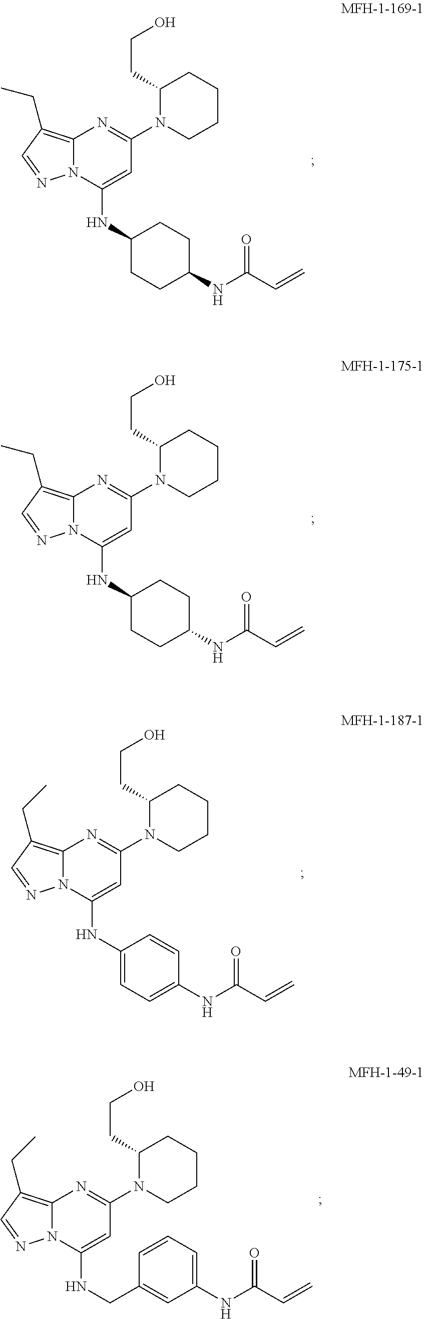

or a pharmaceutically acceptable salt, solvate, hydrate, polymorph, co-crystal, tautomer, stereoisomer, isotopically labeled derivative, or prodrug thereof.

In certain embodiments, the CDK7 inhibitor is a compound of Formula (XX):

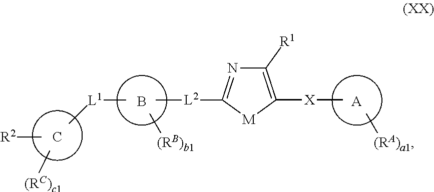

or a pharmaceutically acceptable salt, solvate, hydrate, polymorph, co-crystal, tautomer, stereoisomer, isotopically labeled derivative, or prodrug thereof, wherein:

R¹ is hydrogen, halogen, or optionally substituted alkyl;

M is O, S, or NR^{M};

R^{M} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, or a nitrogen protecting group;

Ring A is optionally substituted monocyclic carbocyclyl, optionally substituted monocyclic heterocyclyl, optionally substituted phenyl, or optionally substituted monocyclic heteroaryl;

Ring B is optionally substituted monocyclic carbocyclyl, optionally substituted monocyclic heterocyclyl, optionally substituted phenyl, or optionally substituted monocyclic heteroaryl;

Ring C is optionally substituted monocyclic carbocyclyl, optionally substituted monocyclic heterocyclyl, optionally substituted monocyclic or bicyclic aryl, or optionally substituted monocyclic or bicyclic heteroaryl;

each instance of R^{A}, R^{B}, and R^{C} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{a}, —N(R^{a})₂, —SR^{a}, —CN, —SCN, —NO₂, —N₃, or optionally substituted acyl;

each instance of R^{a} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, a nitrogen protecting group when attached to a nitrogen atom, an oxygen protecting group when attached to an oxygen atom, or a sulfur protecting group when attached to a sulfur atom, or two instances of R^{a} are joined to form a substituted or unsubstituted, heterocyclic ring, or substituted or unsubstituted, heteroaryl ring;

each of a1, b1, and c1 is independently 0, 1, 2, 3, 4, 5, or 6, as valency permits;

L¹ is —CH₂—, ^{lc}—S(═O)₂—^{lb}, —O—, —S—, —NR^{L1}—, —C(═O)—, ^{lc}—NR^{L1}C(═O)—^{lb}, ^{lc}—C(═O)NR^{L1}—^{lb}, ^{lc}—OC(═O)—^{lb}, or ^{lc}—C(═O)O—^{lb}; wherein ^{lb} indicates the point of attachment is to Ring B; and ^{lc} indicates the point of attachment is to Ring C;

L² is —O—, —S—, —NR^{L2}—, ^{lb}—NR^{L2}C(═O)—^{lm}, ^{lb}—C(═O)NR^{L2}—^{lm}; wherein ^{lb} indicates the point of attachment is to Ring B; and ^{lm} indicates the point of attachment is to the heteroaryl ring with M;

X is ^{xm}—CH₂CH₂—^{xa}, ^{xm}—CH═CH—^{xa}, ^{xm}—CH₂—NR^{LX xa}, ^{xm}—CH₂—O—CH₂—^{xa}, ^{xm}—CH₂ NR^{LX}—CH₂—^{xa}, —O—, —S—, —NR^{LX}—, ^{xm}—O—CH₂—^{xa}, ^{xm}—S—CH₂—^{xa}, ^{xm}—S—C(═O)CH₂—^{xa}, or ^{xm}—NR^{LX}—CH₂—^{xa}; wherein ^{xa} indicates the point of attachment is to Ring A; and ^{xm} indicates the point of attachment is to the heteroaryl ring with M;

each of R^{L1}, R^{L2}, and R^{LX} is independently hydrogen, optionally substituted C₁₋₆ alkyl, or a nitrogen protecting group;

R² is any of Formulae (i-1)-(i-41):

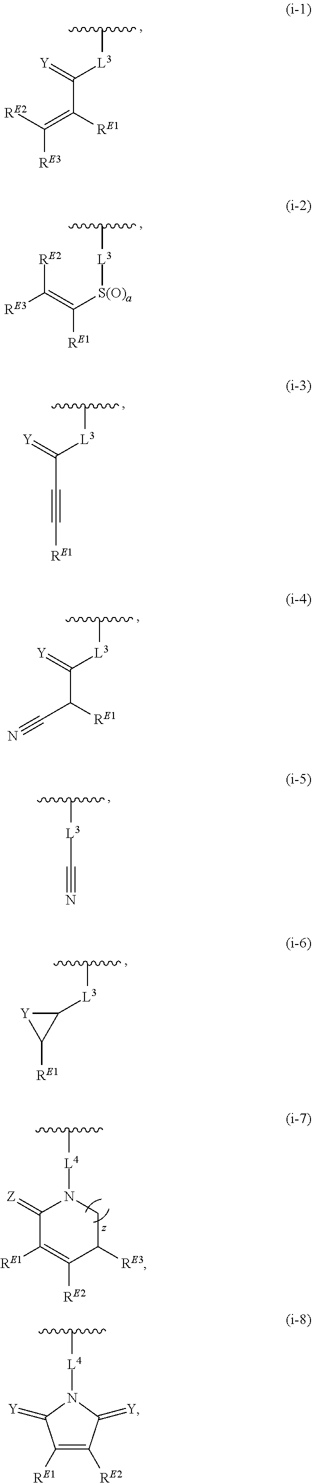

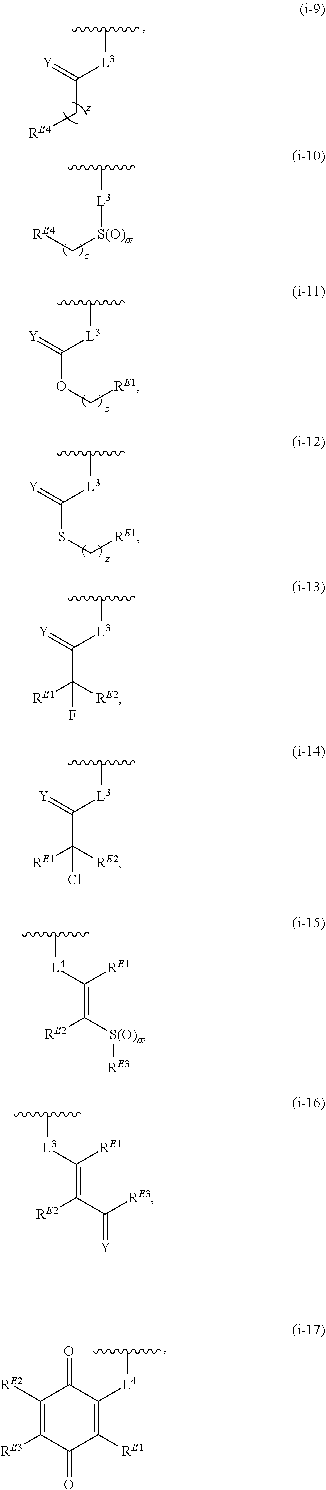

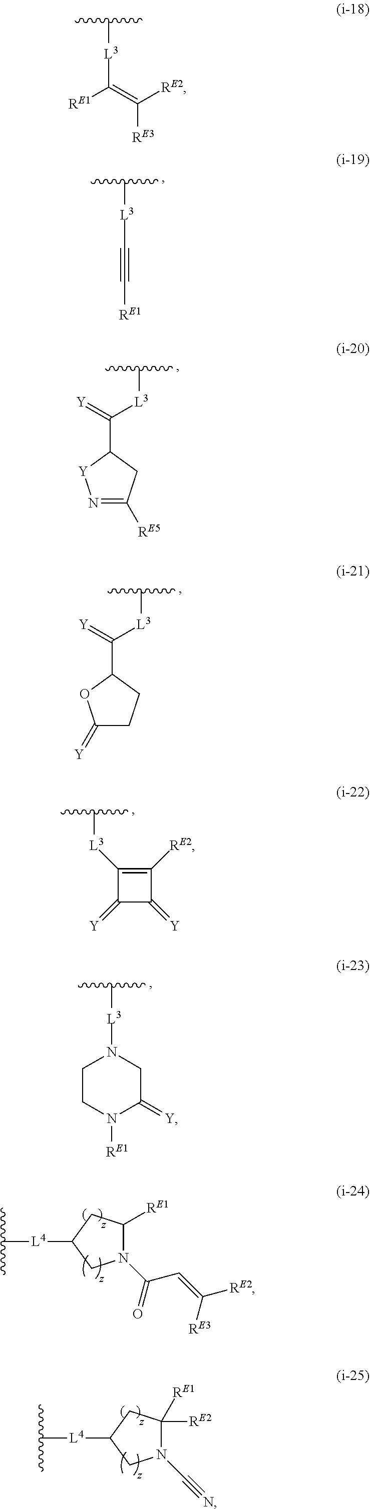

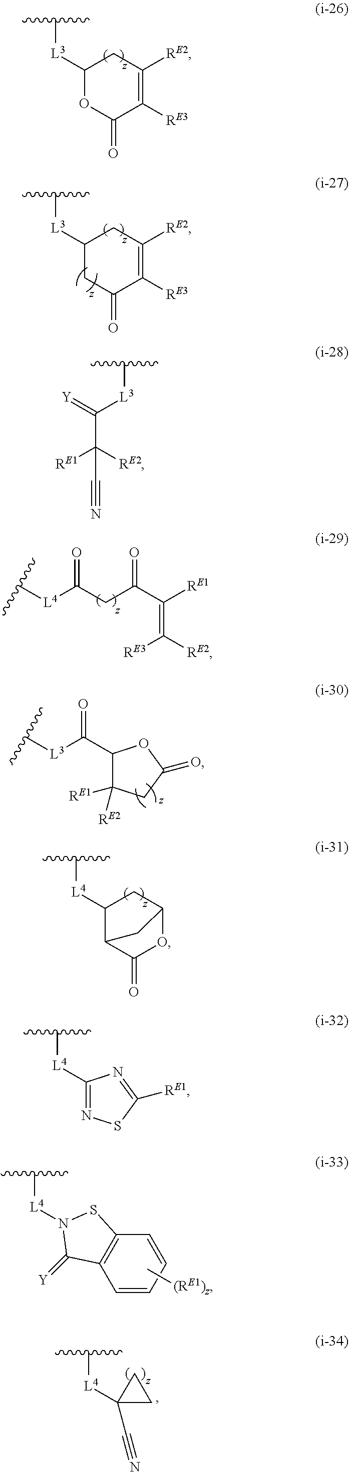

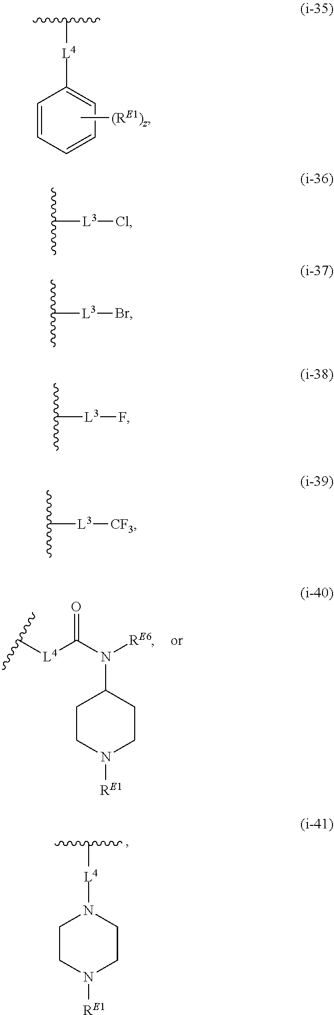

wherein:
L³ is a bond or an optionally substituted C₁₋₄ hydrocarbon chain, optionally wherein one or more carbon units of the hydrocarbon chain are independently replaced with —C═O—, —O—, —S—, —NR^{L3a}—, —NR^{L3a}C(═O)—, —C(═O)NR^{L3a}—, —SC(═O)—, —C(═O)S—, —OC(═O)—, —C(═O)O—, —NR^{L3a}C(═S)—, —C(═S)NR^{L3a}—, trans-CR^{L3b}═CR^{L3b}—, cis-CR^{L3b}=CR^{L3b}—, —C≡C—, —S(═O)—, —S(═O)O—, —OS(═O)—, —S(═O)NR^{L3a}—, —NR^{L3a}, —S(═O)₂—, —S(═O)₂O—, —OS(═O)₂—, —S(═O)₂NR^{L3a}—, or —NR^{L3a}S(═O)₂—, wherein R^{L3a} is hydrogen, optionally substituted C₁₋₆ alkyl, or a nitrogen protecting group, and wherein each occurrence of R^{L3b} is independently hydrogen, halogen, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R^{L3b} groups are joined to form an optionally substituted carbocyclic or optionally substituted heterocyclic ring;
L⁴ is a bond or an optionally substituted, branched or unbranched C₁₋₆ hydrocarbon chain;
each of R^{E1}, R^{E2}, and R^{E3} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{EE}, —CH₂N(R^{EE})₂, —CH₂SR^{EE}, —OR^{EE}, —N(R^{EE})₂, —Si(R^{EE})₃, and —SR^{EE}, wherein each occurrence of R^{EE} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl, or two R^{EE} groups are joined to form an optionally substituted heterocyclic ring;
or R^{E1} and R^{E3}, or R^{E2} and R^{E3}, or R^{E1} and R^{E2} are joined to form an optionally substituted carbocyclic or optionally substituted heterocyclic ring;
R^{E4} is a leaving group;
R^{E5} is halogen;
R^{E6} is hydrogen, optionally substituted C₁₋₆ alkyl, or a nitrogen protecting group;
each instance of Y is independently O, S, or NR^{E7}, wherein R^{E7} is hydrogen, optionally substituted C₁₋₆ alkyl, or a nitrogen protecting group;
a is 1 or 2; and
each instance of z is independently 0, 1, 2, 3, 4, 5, or 6, as valency permits.

In certain embodiments, the compound of Formula (XX) is of formula:

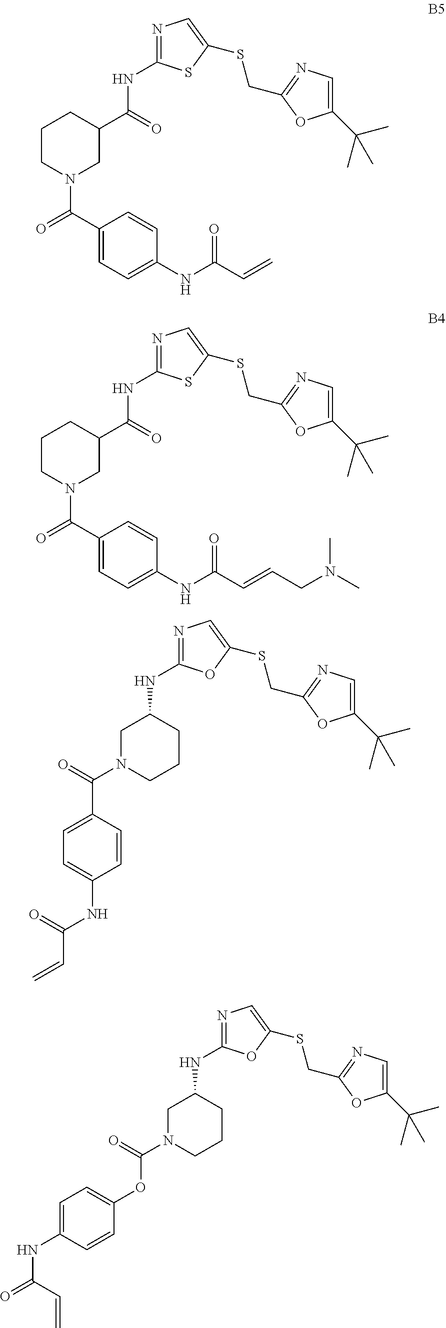

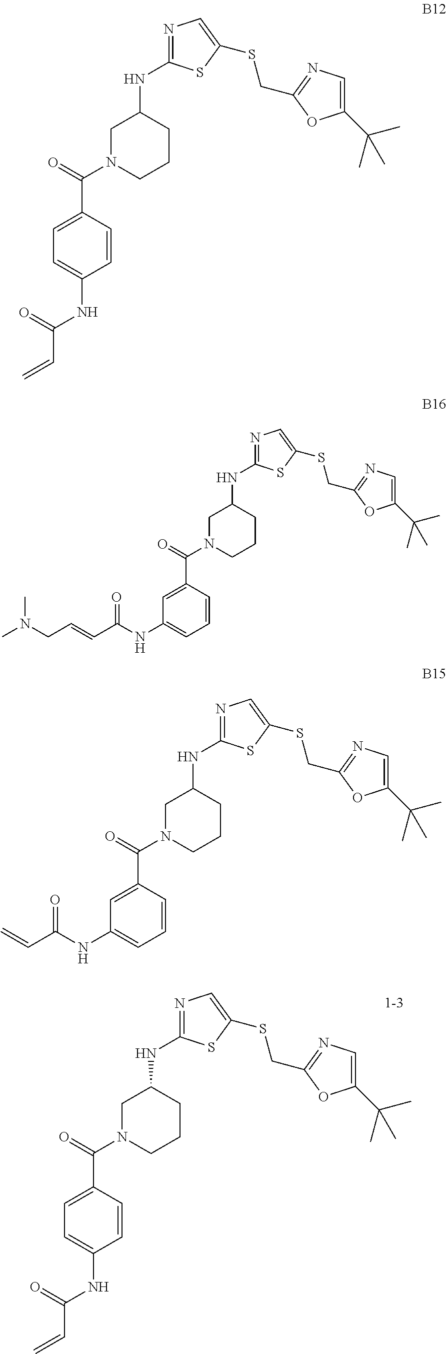

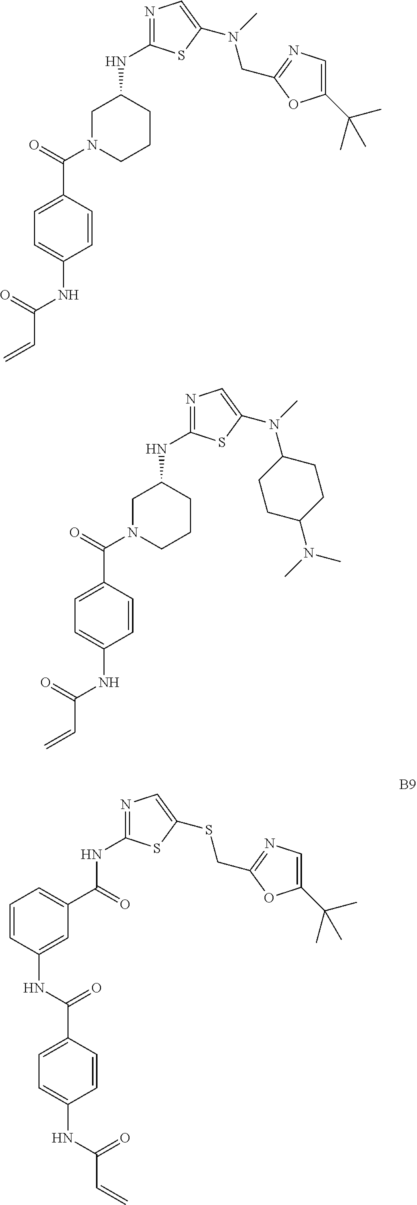

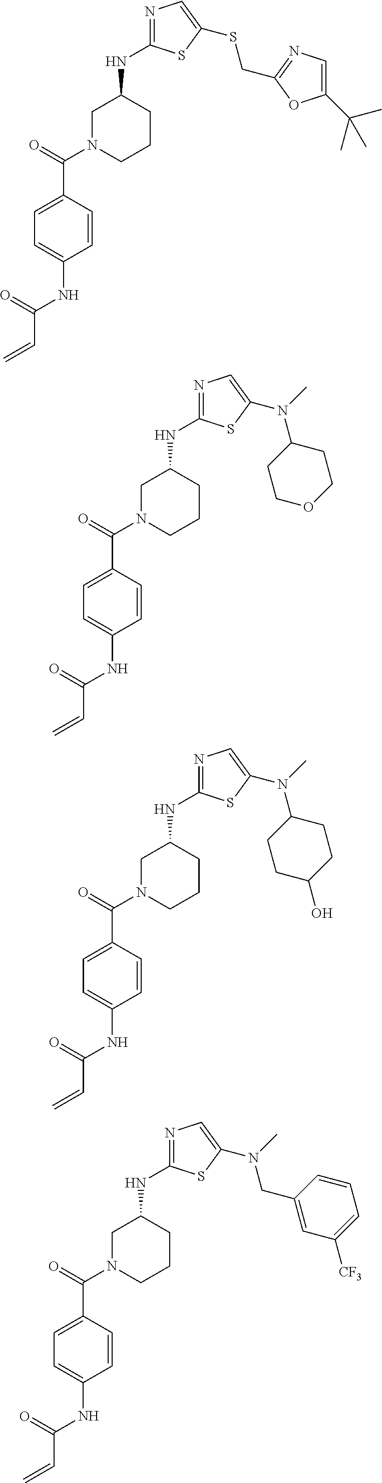

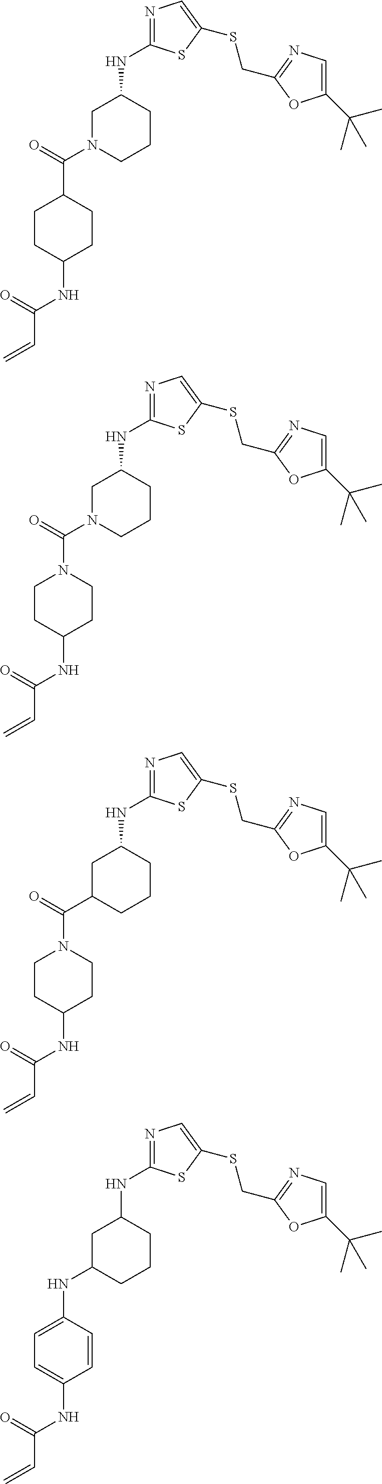

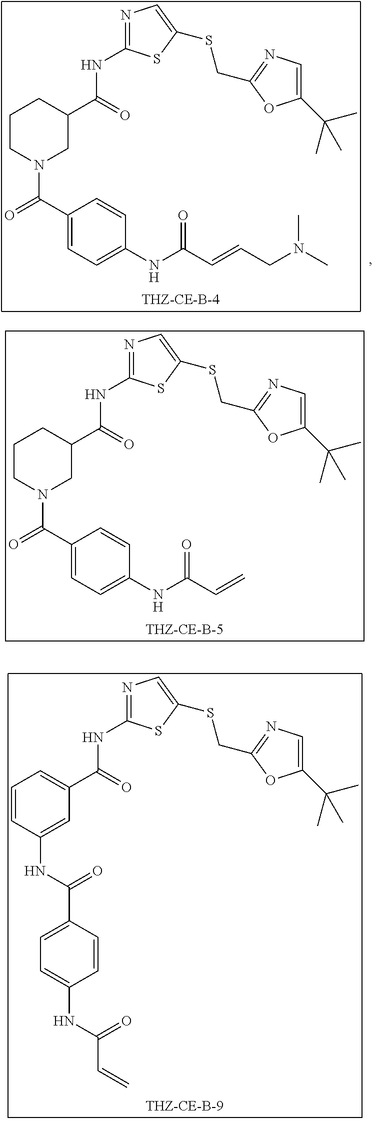 [SMILES: CN(C)C/C=C/C(=O)Nc1ccc(C(=O)N2CCCC(C(=O)Nc3ncc(SCc4ncc(C(C)(C)C)o4)s3)C2)cc1 ; C=CC(=O)Nc1ccc(C(=O)N2CCCC(C(=O)Nc3ncc(SCc4ncc(C(C)(C)C)o4)s3)C2)cc1 ; C=CC(=O)Nc1ccc(C(=O)Nc2cccc(C(=O)Nc3ncc(SCc4ncc(C(C)(C)C)o4)s3)c2)cc1]

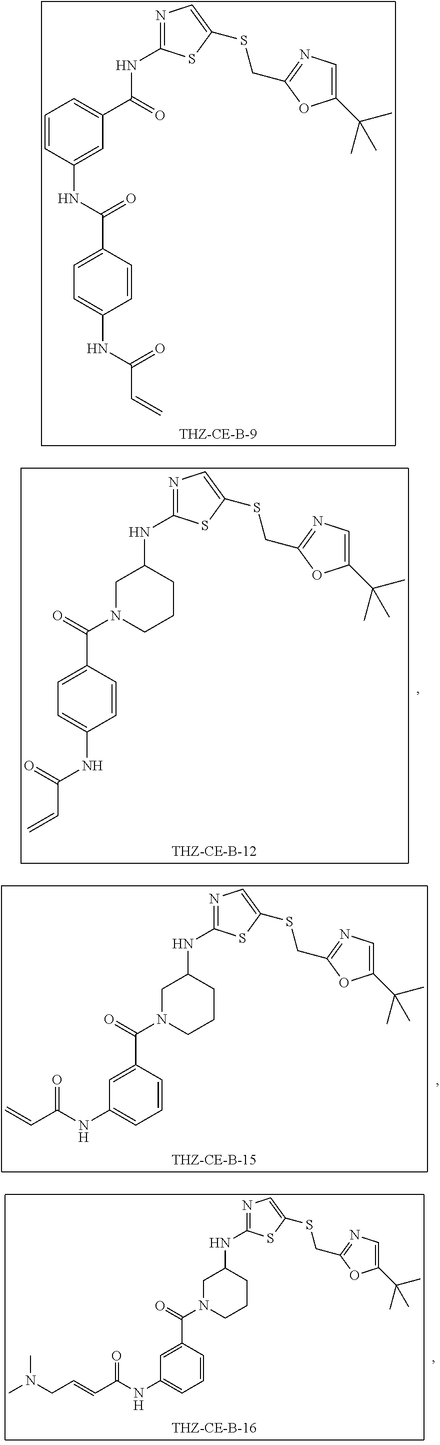

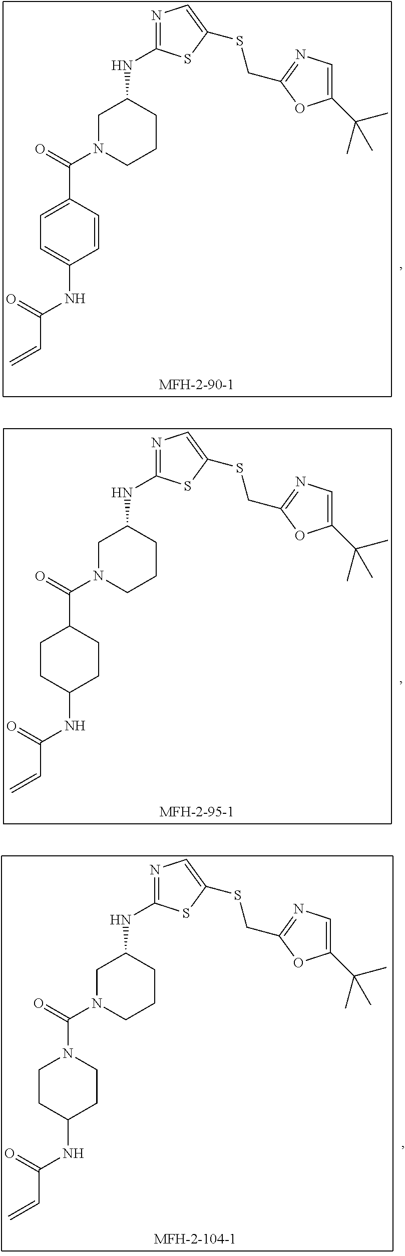 [SMILES: C=CC(=O)Nc1ccc(C(=O)N2CCC[C@@H](Nc3ncc(SCc4ncc(C(C)(C)C)o4)s3)C2)cc1 ; C=CC(=O)NC1CCC(C(=O)N2CCC[C@@H](Nc3ncc(SCc4ncc(C(C)(C)C)o4)s3)C2)CC1 ; C=CC(=O)NC1CCN(C(=O)N2CCC[C@@H](Nc3ncc(SCc4ncc(C(C)(C)C)o4)s3)C2)CC1]

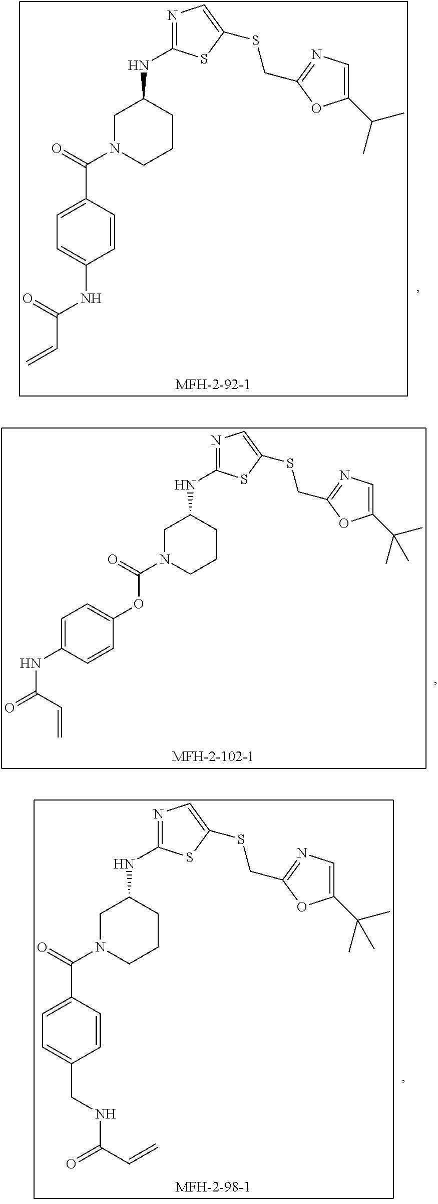 [SMILES: C=CC(=O)Nc1ccc(C(=O)N2CCC[C@H](Nc3ncc(SCc4ncc(C(C)C)o4)s3)C2)cc1 ; C=CC(=O)Nc1ccc(OC(=O)N2CCC[C@@H](Nc3ncc(SCc4ncc(C(C)(C)C)o4)s3)C2)cc1 ; C=CC(=O)NCc1ccc(C(=O)N2CCC[C@@H](Nc3ncc(SCc4ncc(C(C)(C)C)o4)s3)C2)cc1]

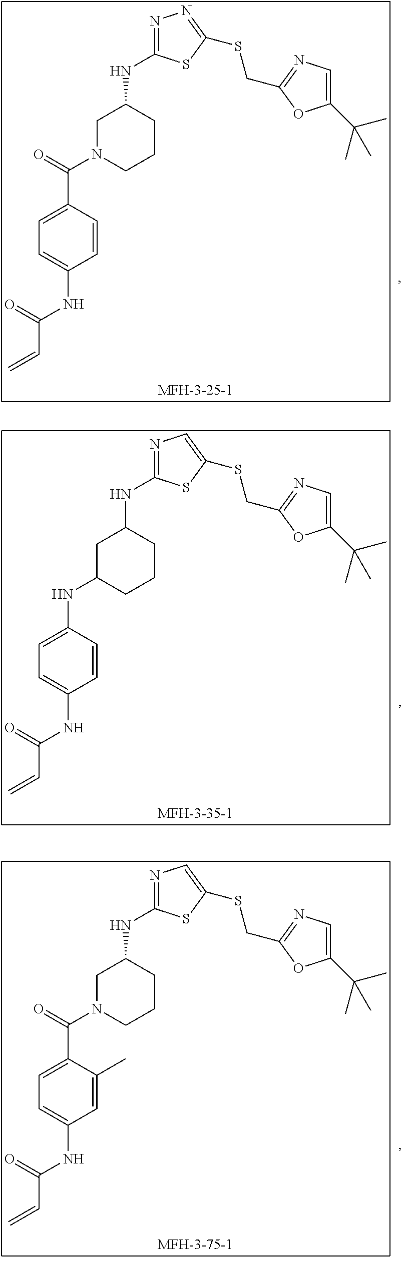 [SMILES: C=CC(=O)Nc1ccc(C(=O)N2CCC[C@@H](Nc3nnc(SCc4ncc(C(C)(C)C)o4)s3)C2)cc1 ; C=CC(=O)Nc1ccc(NC2CCCC(Nc3ncc(SCc4ncc(C(C)(C)C)o4)s3)C2)cc1 ; C=CC(=O)Nc1ccc(C(=O)N2CCC[C@@H](Nc3ncc(SCc4ncc(C(C)(C)C)o4)s3)C2)c(C)c1]

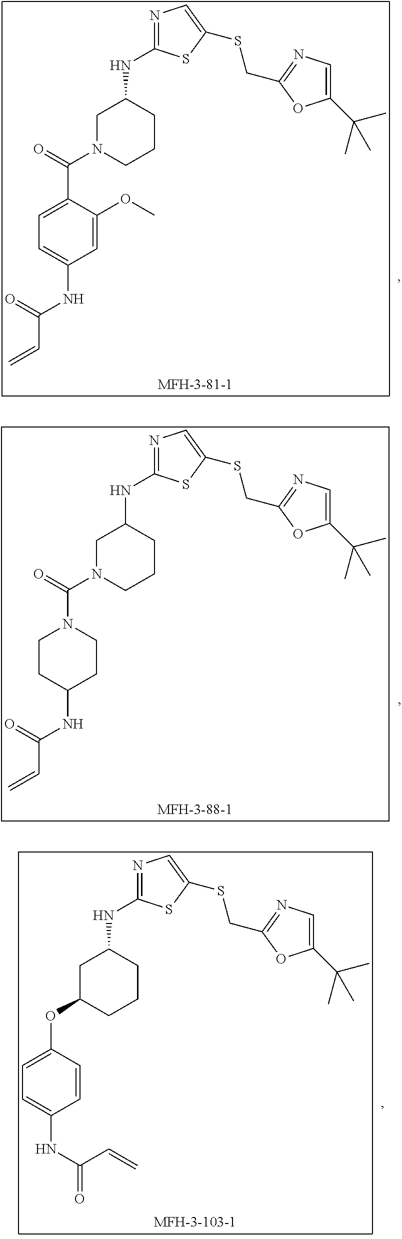 [SMILES: C=CC(=O)Nc1ccc(C(=O)N2CCC[C@@H](Nc3ncc(SCc4ncc(C(C)(C)C)o4)s3)C2)c(OC)c1 ; C=CC(=O)NC1CCN(C(=O)N2CCCC(Nc3ncc(SCc4ncc(C(C)(C)C)o4)s3)C2)CC1 ; C=CC(=O)Nc1ccc(O[C@@H]2CCC[C@@H](Nc3ncc(SCc4ncc(C(C)(C)C)o4)s3)C2)cc1]

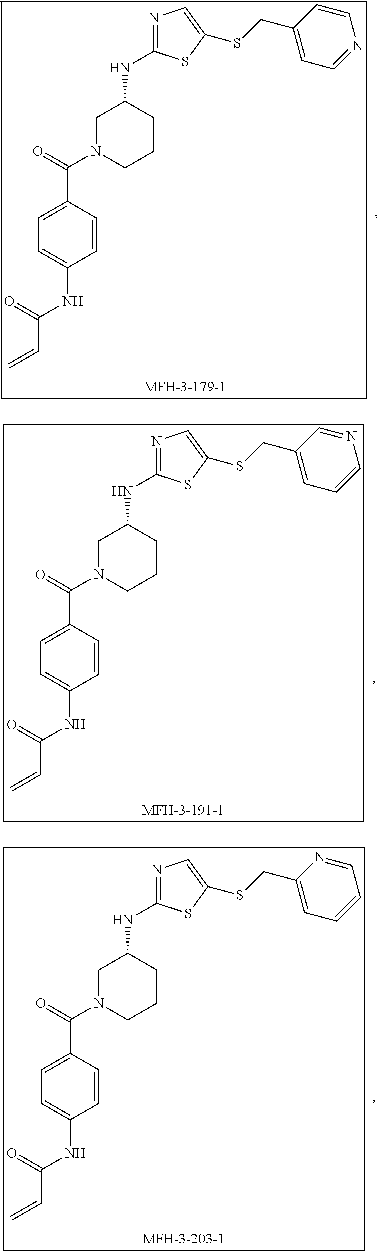 [SMILES: C=CC(=O)Nc1ccc(C(=O)N2CCC[C@@H](Nc3ncc(SCc4ccncc4)s3)C2)cc1 ; C=CC(=O)Nc1ccc(C(=O)N2CCC[C@@H](Nc3ncc(SCc4cccnc4)s3)C2)cc1 ; C=CC(=O)Nc1ccc(C(=O)N2CCC[C@@H](Nc3ncc(SCc4ccccn4)s3)C2)cc1]

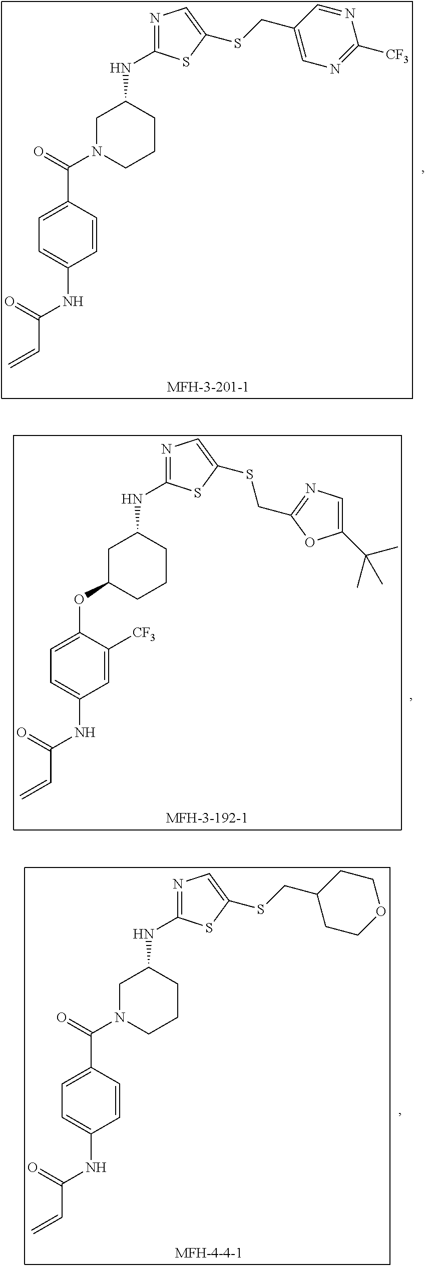 [SMILES: C=CC(=O)Nc1ccc(C(=O)N2CCC[C@@H](Nc3ncc(SCc4cnc(C(F)(F)F)nc4)s3)C2)cc1 ; C=CC(=O)Nc1ccc(O[C@@H]2CCC[C@@H](Nc3ncc(SCc4ncc(C(C)(C)C)o4)s3)C2)c(C(F)(F)F)c1 ; C=CC(=O)Nc1ccc(C(=O)N2CCC[C@@H](Nc3ncc(SCC4CCOCC4)s3)C2)cc1]

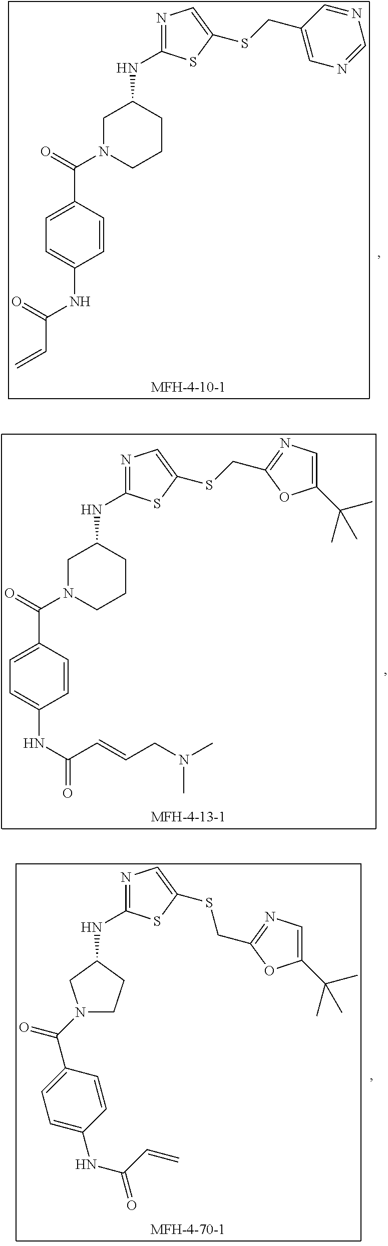 [SMILES: C=CC(=O)Nc1ccc(C(=O)N2CCC[C@@H](Nc3ncc(SCc4cncnc4)s3)C2)cc1 ; CN(C)C/C=C/C(=O)Nc1ccc(C(=O)N2CCC[C@@H](Nc3ncc(SCc4ncc(C(C)(C)C)o4)s3)C2)cc1 ; C=CC(=O)Nc1ccc(C(=O)N2CC[C@@H](Nc3ncc(SCc4ncc(C(C)(C)C)o4)s3)C2)cc1]

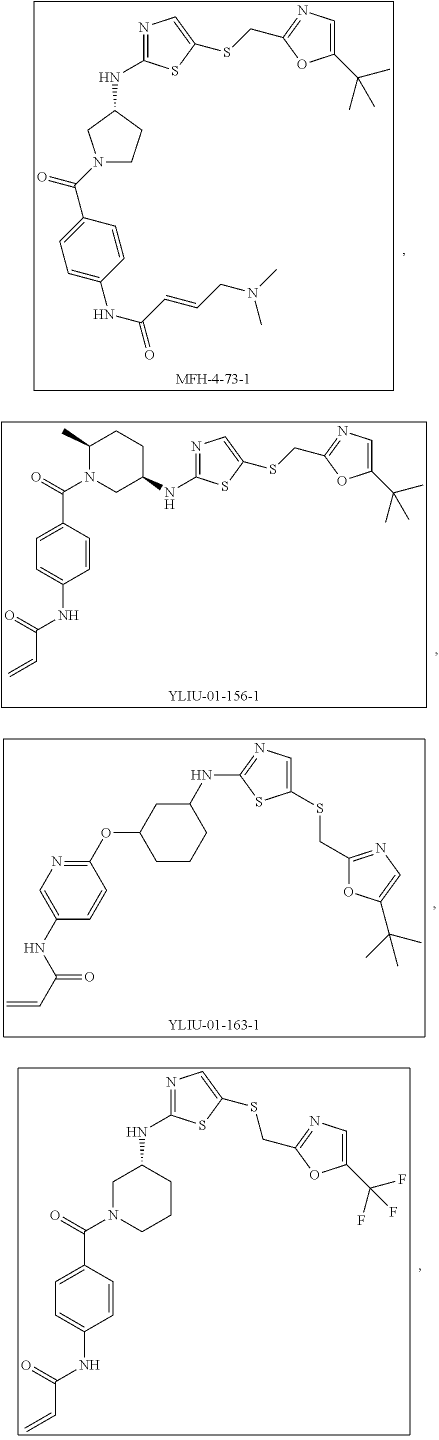

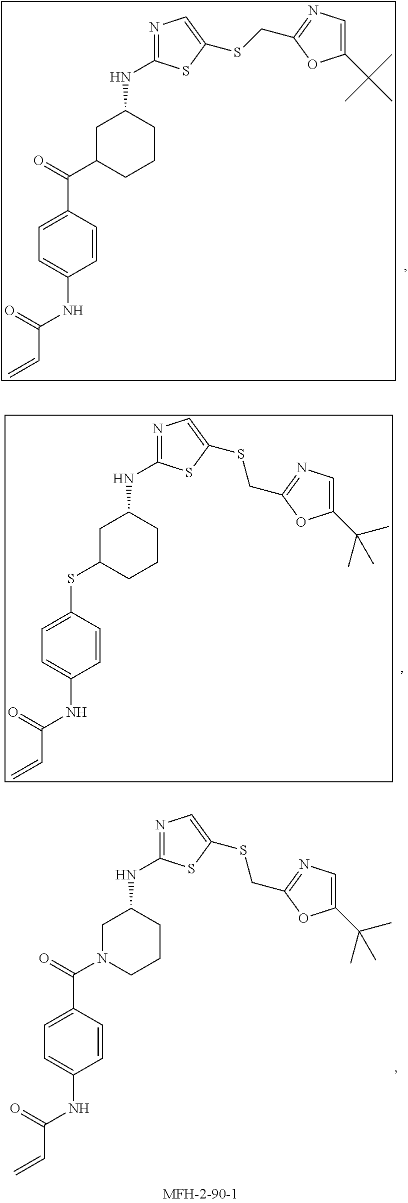 [SMILES: C=CC(=O)Nc1ccc(C(=O)C2CCC[C@@H](Nc3ncc(SCc4ncc(C(C)(C)C)o4)s3)C2)cc1 ; C=CC(=O)Nc1ccc(SC2CCC[C@@H](Nc3ncc(SCc4ncc(C(C)(C)C)o4)s3)C2)cc1 ; C=CC(=O)Nc1ccc(C(=O)N2CCC[C@@H](Nc3ncc(SCc4ncc(C(C)(C)C)o4)s3)C2)cc1]

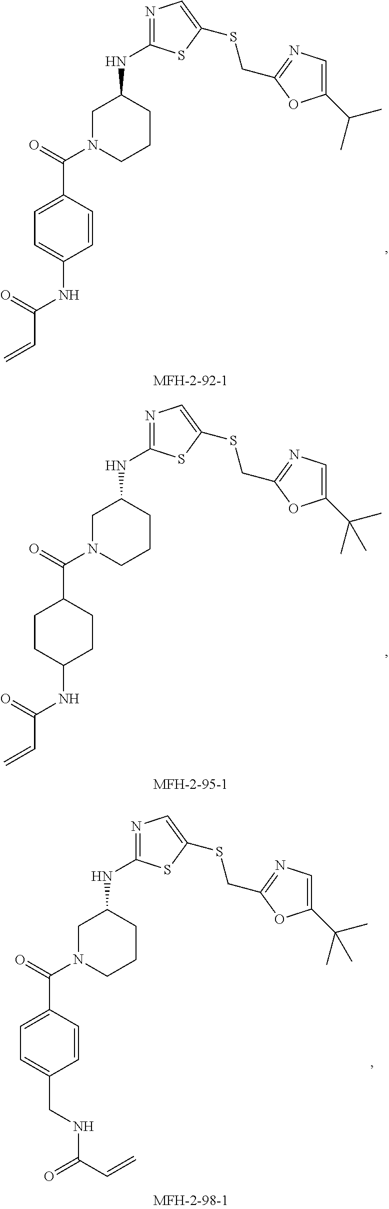 [SMILES: C=CC(=O)Nc1ccc(C(=O)N2CCC[C@H](Nc3ncc(SCc4ncc(C(C)C)o4)s3)C2)cc1 ; C=CC(=O)NC1CCC(C(=O)N2CCC[C@@H](Nc3ncc(SCc4ncc(C(C)(C)C)o4)s3)C2)CC1 ; C=CC(=O)NCc1ccc(C(=O)N2CCC[C@@H](Nc3ncc(SCc4ncc(C(C)(C)C)o4)s3)C2)cc1]

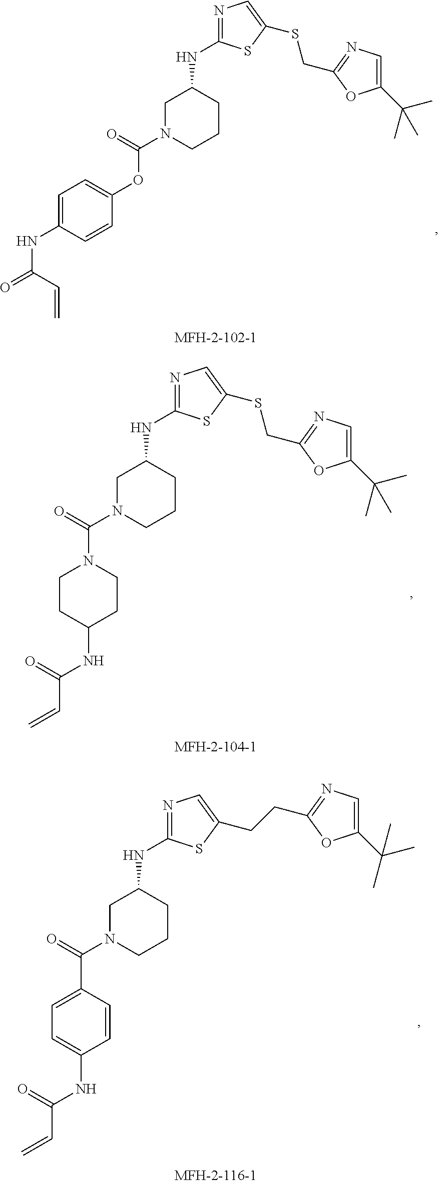 [SMILES: C=CC(=O)Nc1ccc(OC(=O)N2CCC[C@@H](Nc3ncc(SCc4ncc(C(C)(C)C)o4)s3)C2)cc1 ; C=CC(=O)NC1CCN(C(=O)N2CCC[C@@H](Nc3ncc(SCc4ncc(C(C)(C)C)o4)s3)C2)CC1 ; C=CC(=O)Nc1ccc(C(=O)N2CCC[C@@H](Nc3ncc(CCc4ncc(C(C)(C)C)o4)s3)C2)cc1]

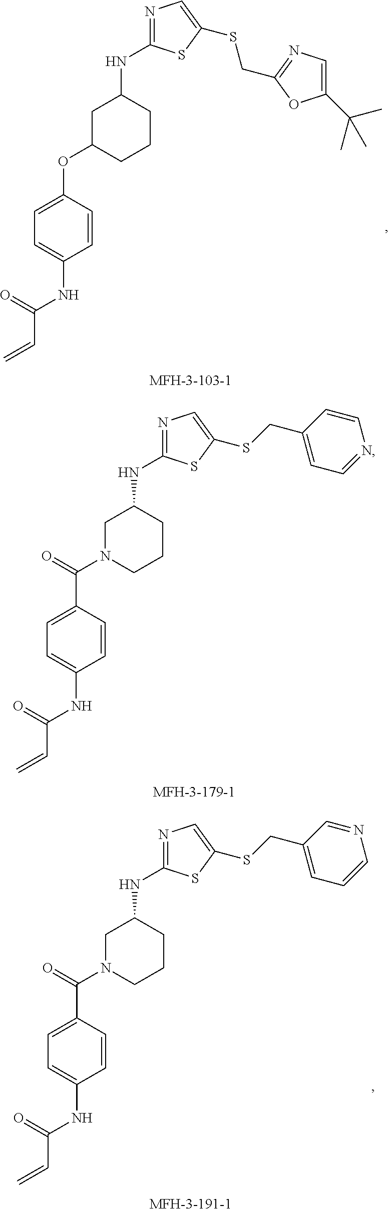 [SMILES: C=CC(=O)Nc1ccc(OC2CCCC(Nc3ncc(SCc4ncc(C(C)(C)C)o4)s3)C2)cc1 ; C=CC(=O)Nc1ccc(C(=O)N2CCC[C@@H](Nc3ncc(SCc4ccncc4)s3)C2)cc1 ; C=CC(=O)Nc1ccc(C(=O)N2CCC[C@@H](Nc3ncc(SCc4cccnc4)s3)C2)cc1]

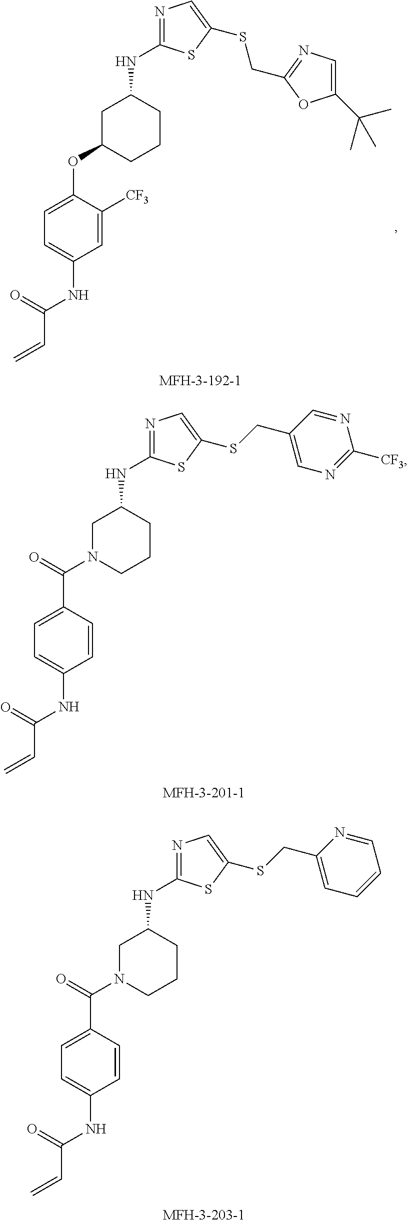 [SMILES: C=CC(=O)Nc1ccc(O[C@@H]2CCC[C@@H](Nc3ncc(SCc4ncc(C(C)(C)C)o4)s3)C2)c(C(F)(F)F)c1 ; C=CC(=O)Nc1ccc(C(=O)N2CCC[C@@H](Nc3ncc(SCc4cnc(C(F)(F)F)nc4)s3)C2)cc1 ; C=CC(=O)Nc1ccc(C(=O)N2CCC[C@@H](Nc3ncc(SCc4ccccn4)s3)C2)cc1]

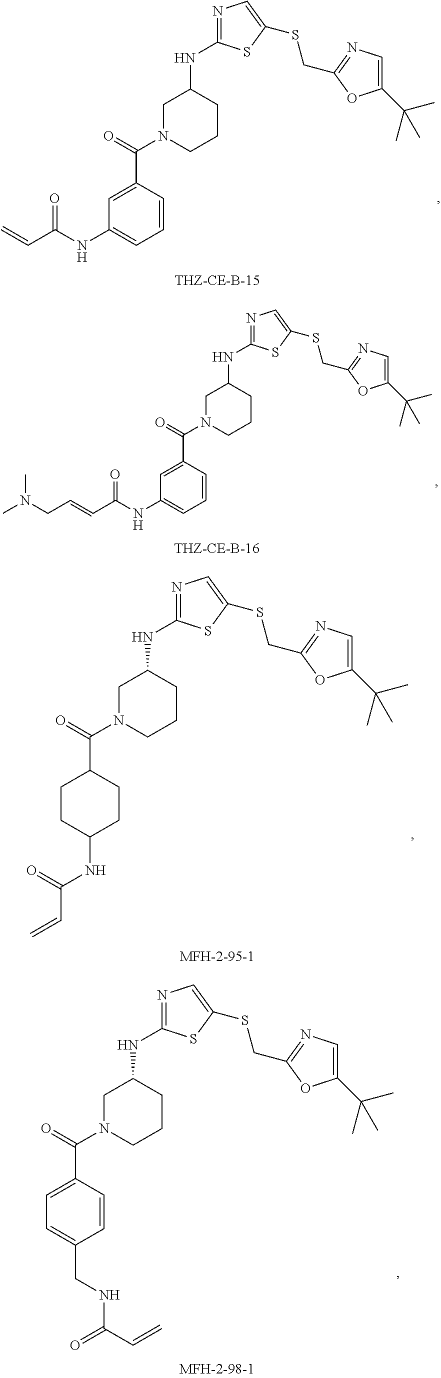

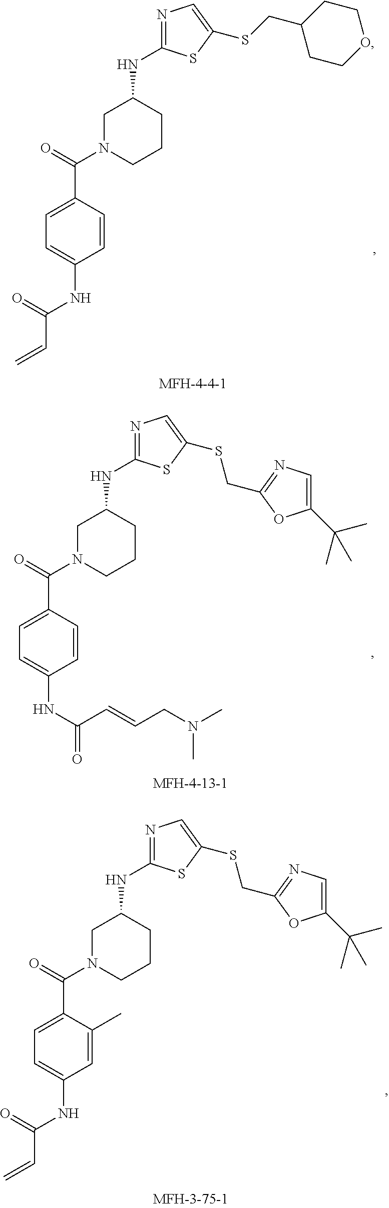 [SMILES: C=CC(=O)Nc1ccc(C(=O)N2CCC[C@@H](Nc3ncc(SCC4CCOCC4)s3)C2)cc1 ; CN(C)C/C=C/C(=O)Nc1ccc(C(=O)N2CCC[C@@H](Nc3ncc(SCc4ncc(C(C)(C)C)o4)s3)C2)cc1 ; C=CC(=O)Nc1ccc(C(=O)N2CCC[C@@H](Nc3ncc(SCc4ncc(C(C)(C)C)o4)s3)C2)c(C)c1]

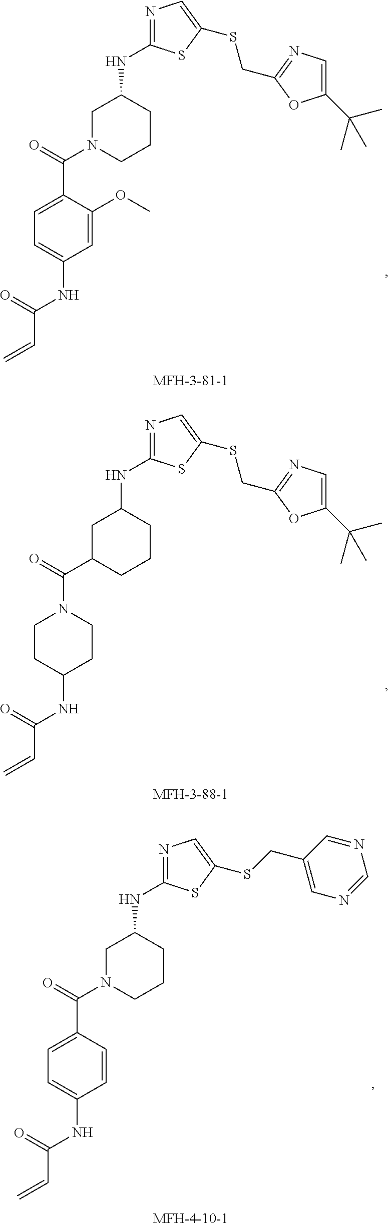 [SMILES: C=CC(=O)Nc1ccc(C(=O)N2CCC[C@@H](Nc3ncc(SCc4ncc(C(C)(C)C)o4)s3)C2)c(OC)c1 ; C=CC(=O)NC1CCN(C(=O)C2CCCC(Nc3ncc(SCc4ncc(C(C)(C)C)o4)s3)C2)CC1 ; C=CC(=O)Nc1ccc(C(=O)N2CCC[C@@H](Nc3ncc(SCc4cncnc4)s3)C2)cc1]

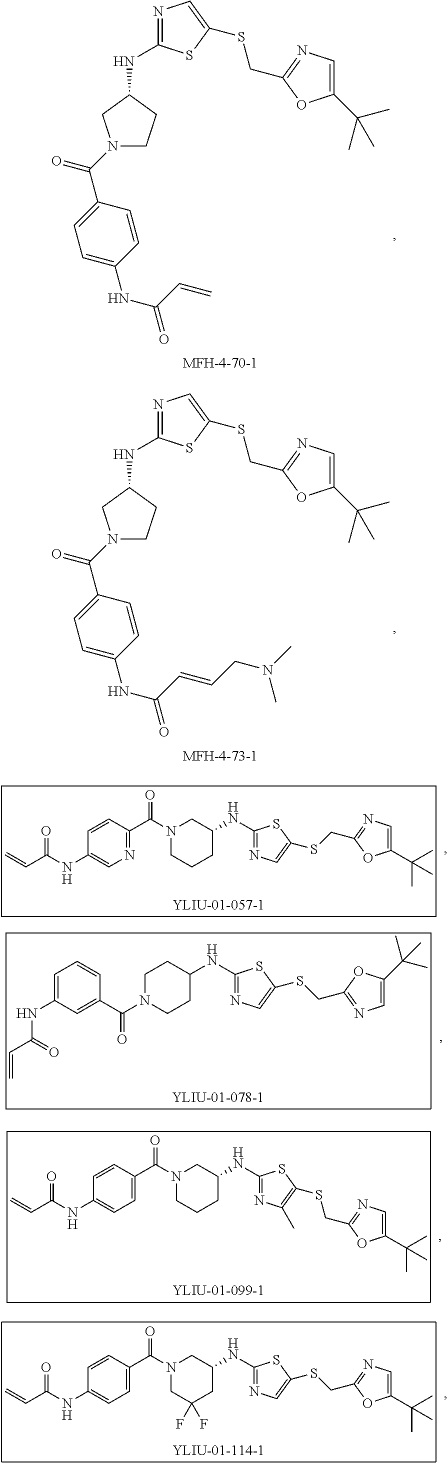

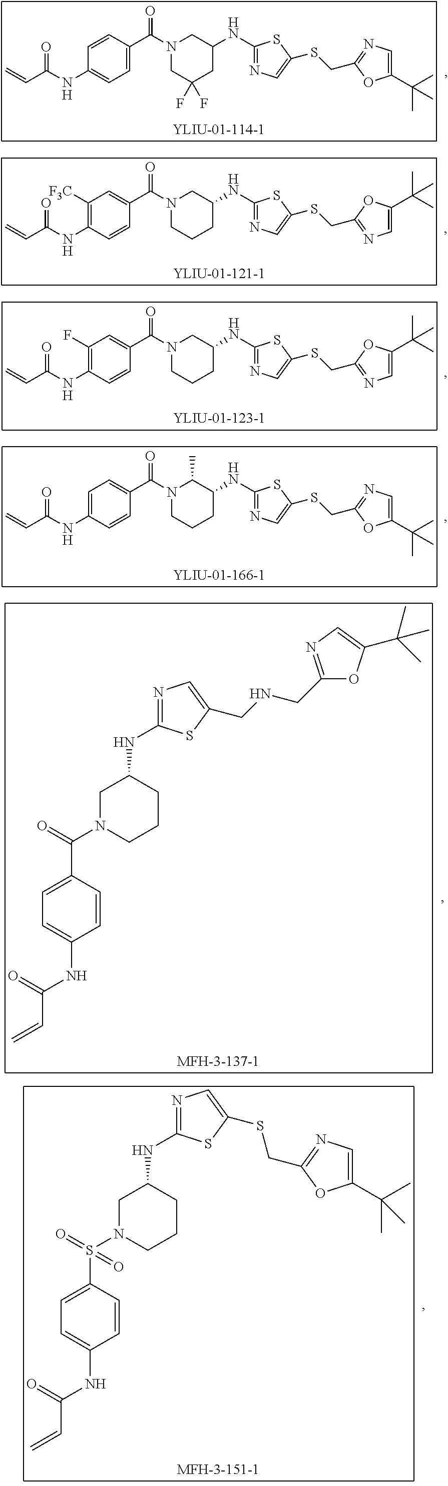

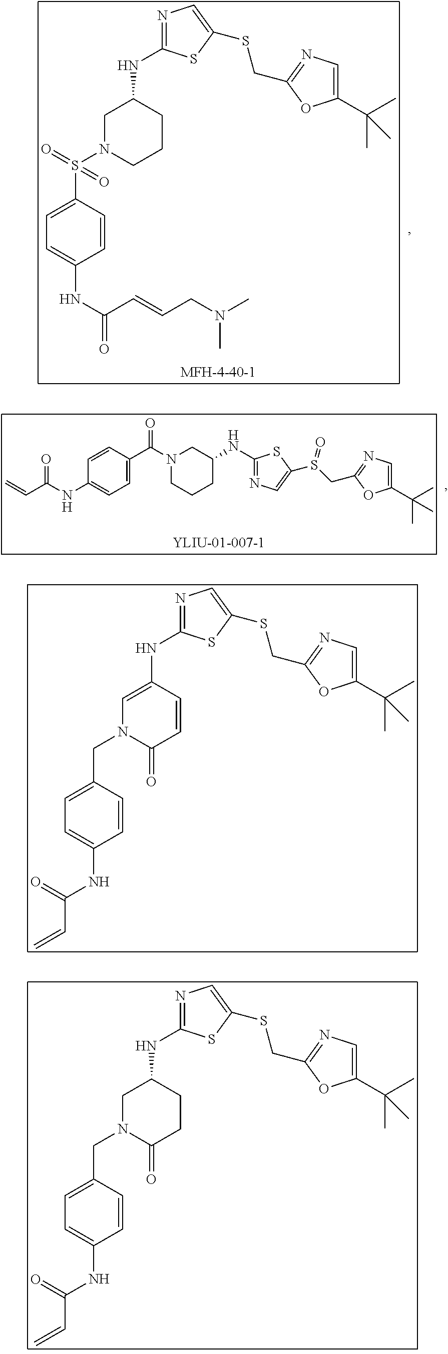

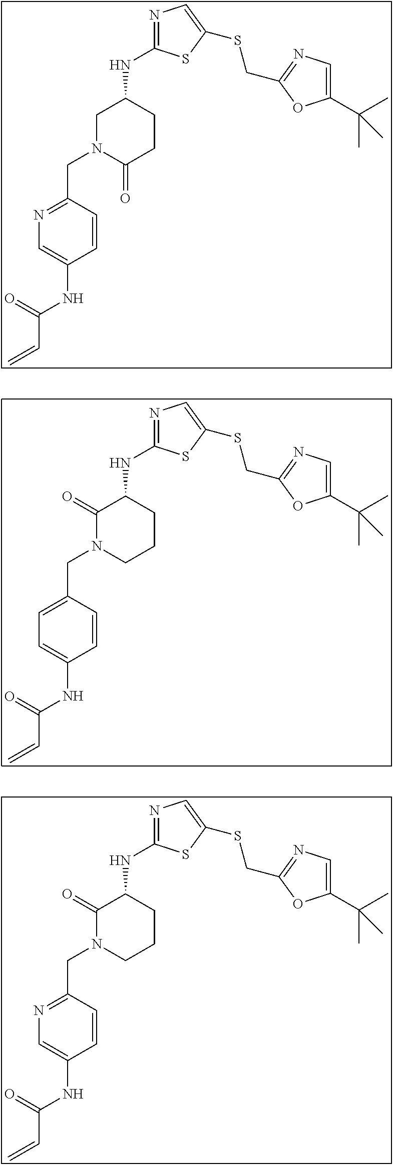 [SMILES: C=CC(=O)Nc1ccc(CN2C[C@H](Nc3ncc(SCc4ncc(C(C)(C)C)o4)s3)CCC2=O)nc1 ; C=CC(=O)Nc1ccc(CN2CCC[C@@H](Nc3ncc(SCc4ncc(C(C)(C)C)o4)s3)C2=O)cc1 ; C=CC(=O)Nc1ccc(CN2CCC[C@@H](Nc3ncc(SCc4ncc(C(C)(C)C)o4)s3)C2=O)nc1]

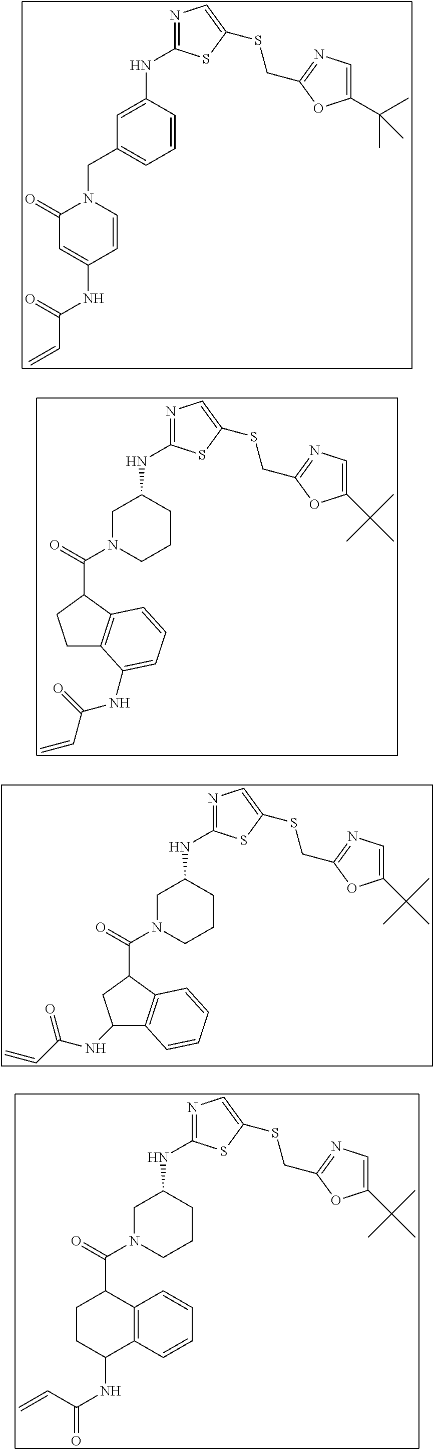

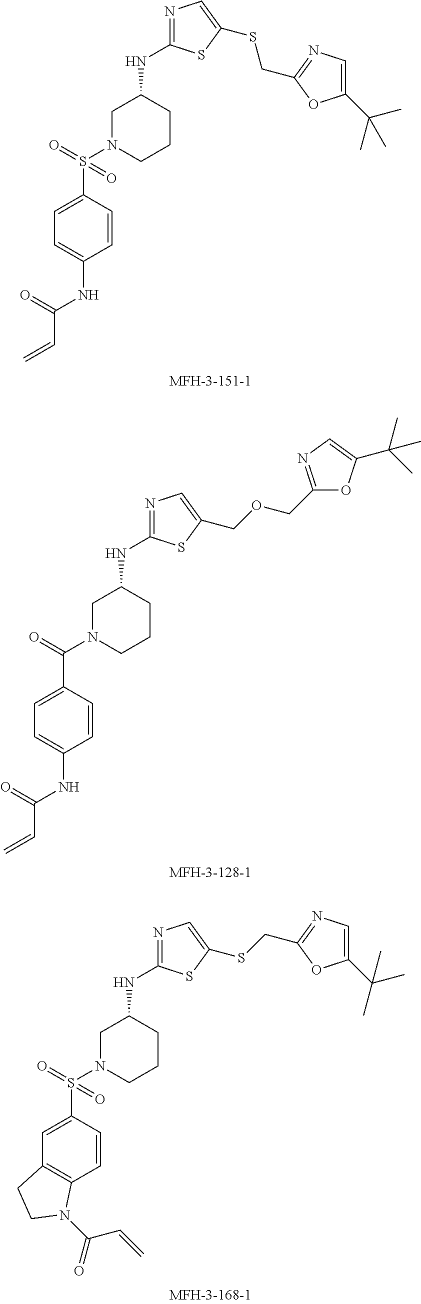 [SMILES: C=CC(=O)Nc1ccc(S(=O)(=O)N2CCC[C@@H](Nc3ncc(SCc4ncc(C(C)(C)C)o4)s3)C2)cc1 ; C=CC(=O)Nc1ccc(C(=O)N2CCC[C@@H](Nc3ncc(COCc4ncc(C(C)(C)C)o4)s3)C2)cc1 ; C=CC(=O)N1CCc2cc(S(=O)(=O)N3CCC[C@@H](Nc4ncc(SCc5ncc(C(C)(C)C)o5)s4)C3)ccc21]

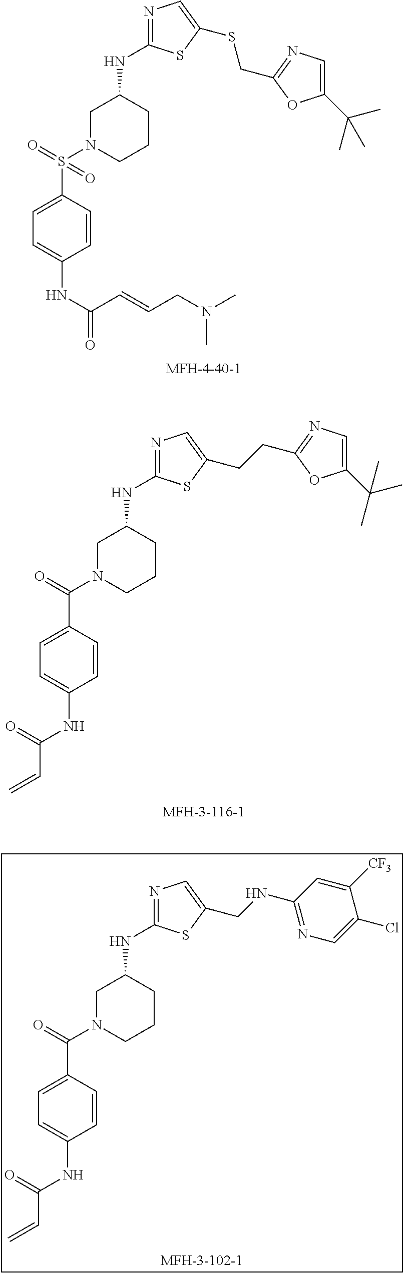 [SMILES: CN(C)C/C=C/C(=O)Nc1ccc(S(=O)(=O)N2CCC[C@@H](Nc3ncc(SCc4ncc(C(C)(C)C)o4)s3)C2)cc1 ; C=CC(=O)Nc1ccc(C(=O)N2CCC[C@@H](Nc3ncc(CCc4ncc(C(C)(C)C)o4)s3)C2)cc1 ; C=CC(=O)Nc1ccc(C(=O)N2CCC[C@@H](Nc3ncc(CNc4cc(C(F)(F)F)c(Cl)cn4)s3)C2)cc1]

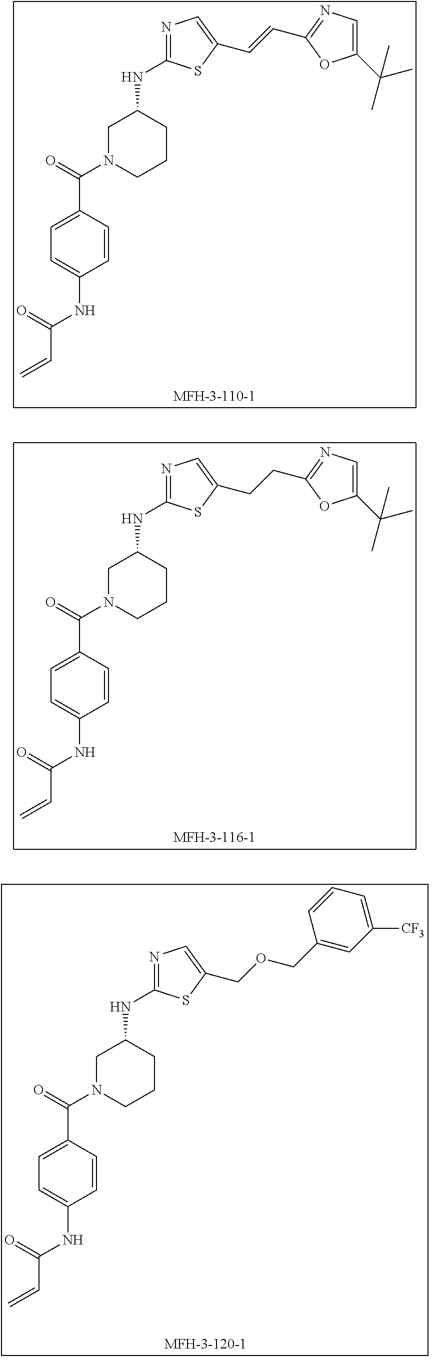 [SMILES: C=CC(=O)Nc1ccc(C(=O)N2CCC[C@@H](Nc3ncc(/C=C/c4ncc(C(C)(C)C)o4)s3)C2)cc1 ; C=CC(=O)Nc1ccc(C(=O)N2CCC[C@@H](Nc3ncc(CCc4ncc(C(C)(C)C)o4)s3)C2)cc1 ; C=CC(=O)Nc1ccc(C(=O)N2CCC[C@@H](Nc3ncc(COCc4cccc(C(F)(F)F)c4)s3)C2)cc1]

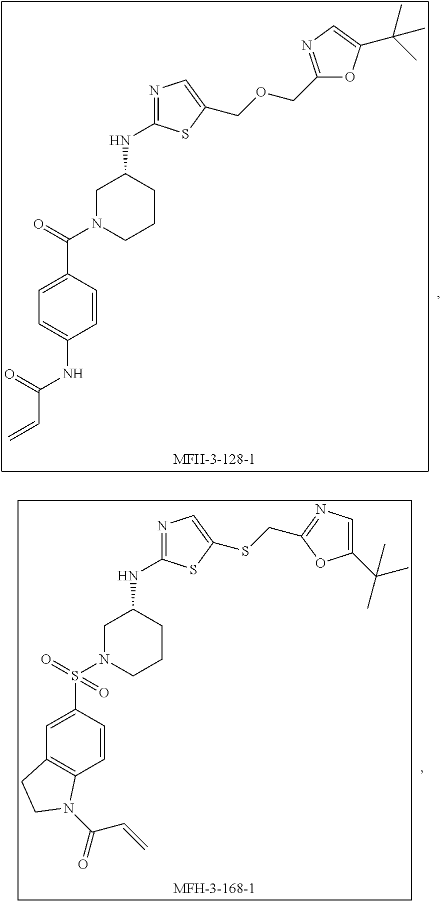 [SMILES: C=CC(=O)Nc1ccc(C(=O)N2CCC[C@@H](Nc3ncc(COCc4ncc(C(C)(C)C)o4)s3)C2)cc1 ; C=CC(=O)N1CCc2cc(S(=O)(=O)N3CCC[C@@H](Nc4ncc(SCc5ncc(C(C)(C)C)o5)s4)C3)ccc21]

or a pharmaceutically acceptable salt, solvate, hydrate, polymorph, co-crystal, tautomer, stereoisomer, isotopically labeled derivative, or prodrug thereof.

In certain embodiments, the CDK7 inhibitor is a compound of Formula (XXI):

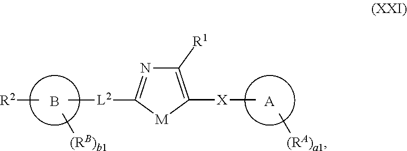

or a pharmaceutically acceptable salt, solvate, hydrate, polymorph, co-crystal, tautomer, stereoisomer, isotopically labeled derivative, or prodrug thereof, wherein:

R¹ is hydrogen, halogen, or optionally substituted alkyl;

M is O, S, or NR^{M};

R^{M} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, or a nitrogen protecting group;

Ring A is optionally substituted monocyclic carbocyclyl, optionally substituted monocyclic heterocyclyl, optionally substituted phenyl, or optionally substituted monocyclic heteroaryl;

Ring B is optionally substituted monocyclic carbocyclyl, optionally substituted monocyclic heterocyclyl, optionally substituted phenyl, or optionally substituted monocyclic heteroaryl;

each instance of R^{A} and R^{B} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;

each instance of R^{a} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{a} are joined to form a substituted or unsubstituted, heterocyclic ring, or substituted or unsubstituted, heteroaryl ring;

each of a1 and b1 is independently 0, 1, 2, 3, 4, 5, or 6, as valency permits;

L² is —O—, —S—, —NR^{L2}—, ^{lb}—NR^{L2}C(═O)—^{lm}, ^{lb}—C(═O)NR^{L2}—^{lm}; wherein ^{lb} indicates the point of attachment is to Ring B; and ^{lm} indicates the point of attachment is to the heteroaryl ring with M;

X is a bond, —O—, —S—, —NR^{LX}—, ^{xm}—O—CH₂—^{xa}, ^{xm}—S—CH₂—^{xa}, or ^{xm}—NR^{LX}—CH₂—^{xa}; wherein ^{xa} indicates the point of attachment is to Ring A; and ^{xm} indicates the point of attachment is to the heteroaryl ring with M;

each of R^{L2} and R^{LX} is independently hydrogen, optionally substituted C₁₋₆ alkyl, or a nitrogen protecting group;

R² is any of Formulae (i-1)-(i-41):

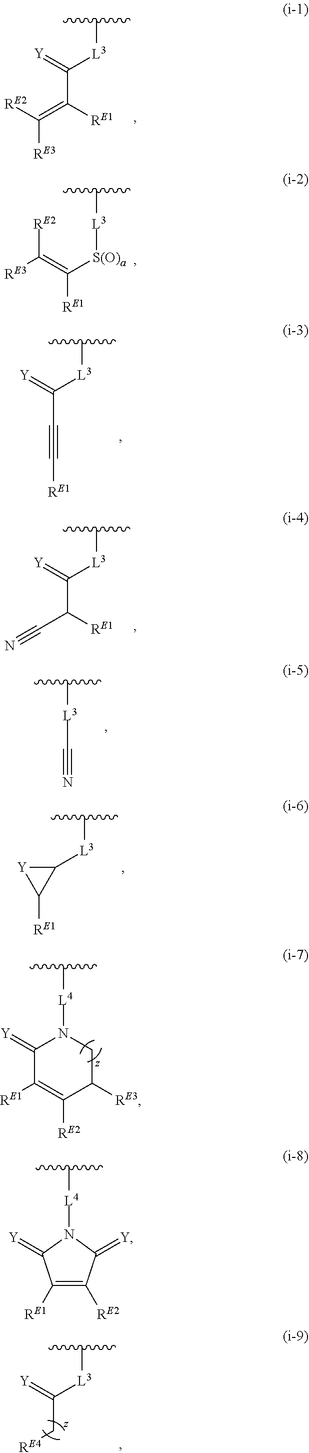

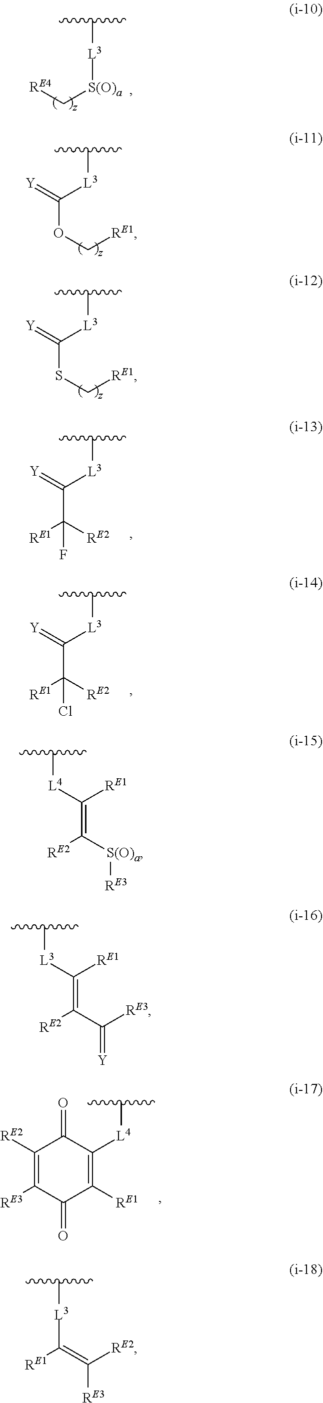

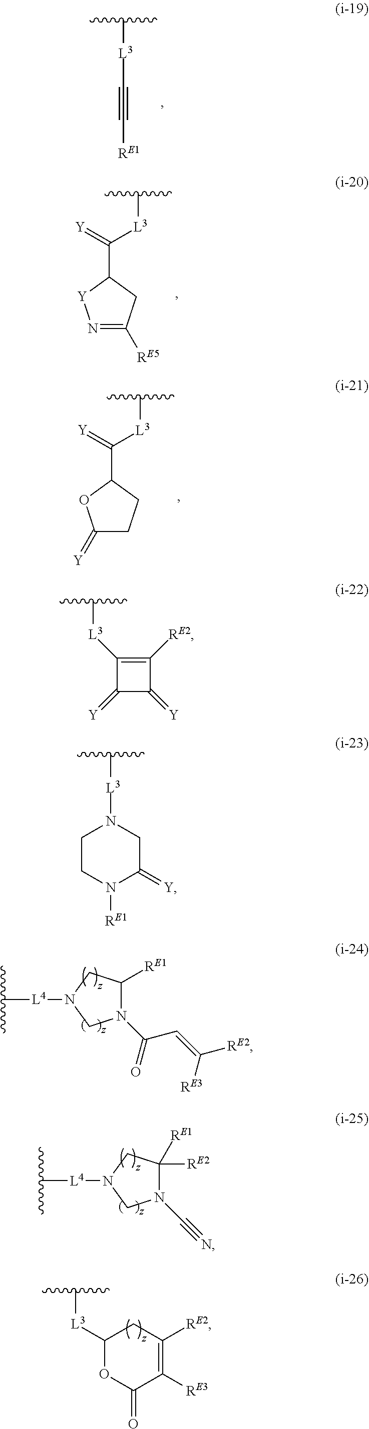

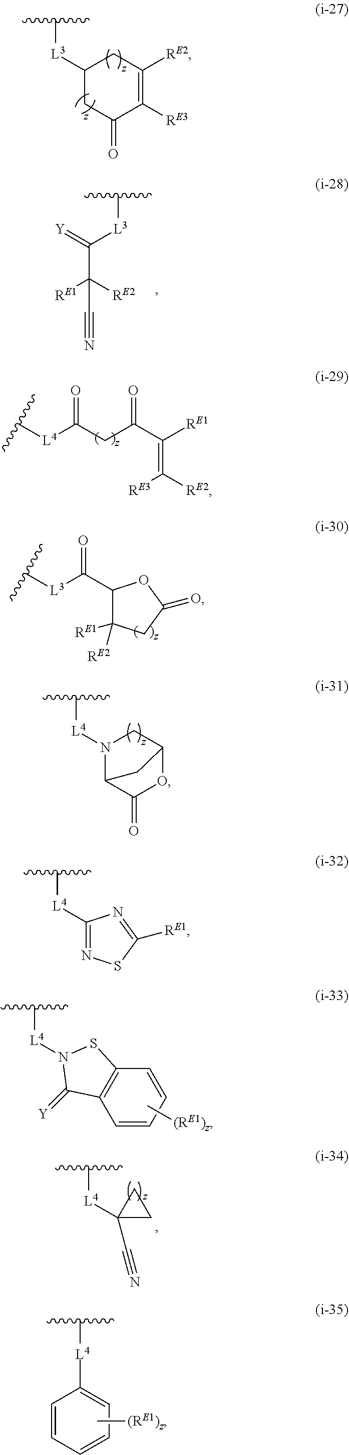

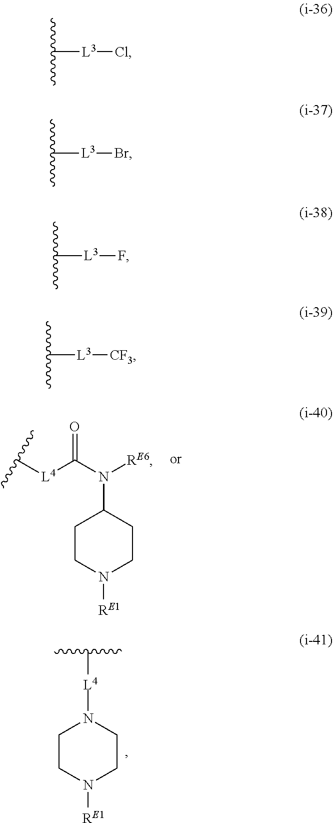

wherein:
L³ is a bond or an optionally substituted C₁₋₄ hydrocarbon chain, optionally wherein one or more carbon units of the hydrocarbon chain are independently replaced with —C═O—, —O—, —S—, —NR^{L3a}—, —NR^{L3a}C(═O)—, —C(═O)NR^{L3a}—, —SC(═O)—, —C(═O)S—, —OC(═O)—, —C(═O)O—, —NR^{L3a}C(═S)—, —C(═S)NR^{L3a}—, trans-CR^{L3b}═CR^{L3b}—, cis-CR^{L3b}=CR^{L3b}—, —C≡C—, —S(═O)—, —S(═O)O—, —OS(═O)—, —S(═O)NR^{L3a}—, —NR^{L3a}, —S(═O)₂—, —S(═O)₂O—, —OS(═O)₂—, —S(═O)₂NR^{L3a}—, or —NR^{L3a}S(═O)₂—, wherein R^{L3a} is hydrogen, optionally substituted C₁₋₆ alkyl, or a nitrogen protecting group, and wherein each occurrence of R^{L3b} is independently hydrogen, halogen, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R^{L3b} groups are joined to form an optionally substituted carbocyclic or optionally substituted heterocyclic ring;
L⁴ is a bond or an optionally substituted, branched or unbranched C₁₋₆ hydrocarbon chain;
each of R^{E1}, R^{E2}, and R^{E3} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{EE}, —CH₂N(R^{EE})₂, —CH₂SR^{EE}, —OR^{EE}, —N(R^{EE})₂, —Si(R^{EE})₃, and —SR^{EE}, wherein each occurrence of R^{EE} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl, or two R^{EE} groups are joined to form an optionally substituted heterocyclic ring;
or R^{E1} and R^{E3}, or R^{E2} and R^{E3}, or R^{E1} and R^{E2} are joined to form an optionally substituted carbocyclic or optionally substituted heterocyclic ring;
R^{E4} is a leaving group;
R^{E5} is halogen;
R^{E6} is hydrogen, optionally substituted C₁₋₆ alkyl, or a nitrogen protecting group;
each instance of Y is independently O, S, or NR^{E7}, wherein R^{E7} is hydrogen, optionally substituted C₁₋₆ alkyl, or a nitrogen protecting group;
a is 1 or 2; and
each instance of z is independently 0, 1, 2, 3, 4, 5, or 6, as valency permits.

In certain embodiments, the compound of Formula (XXI) is of formula:

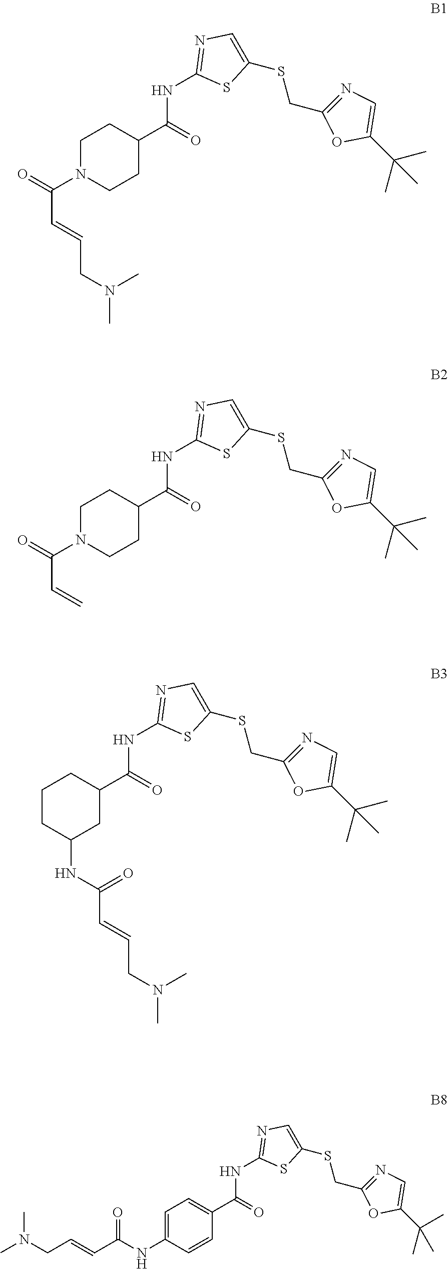

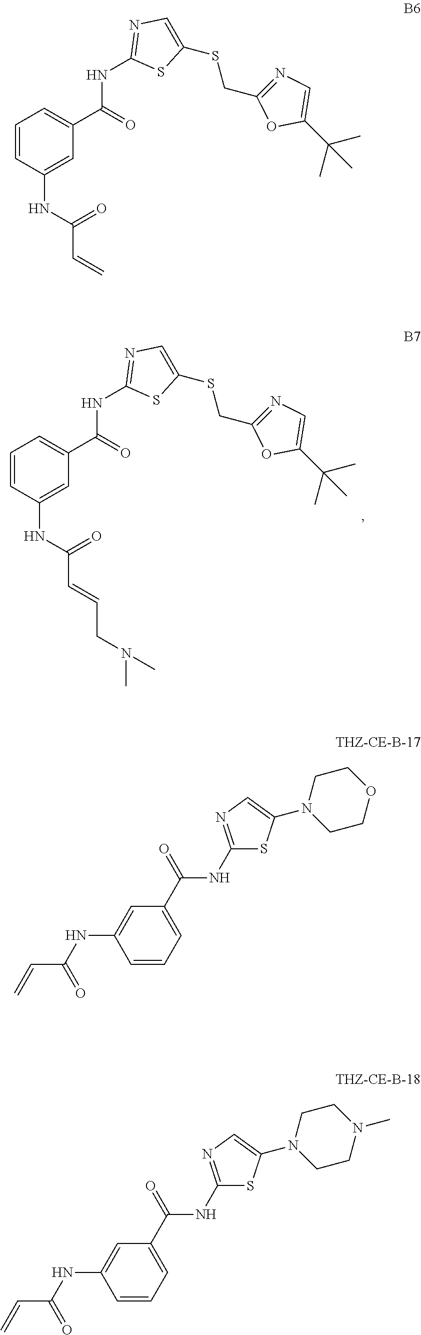

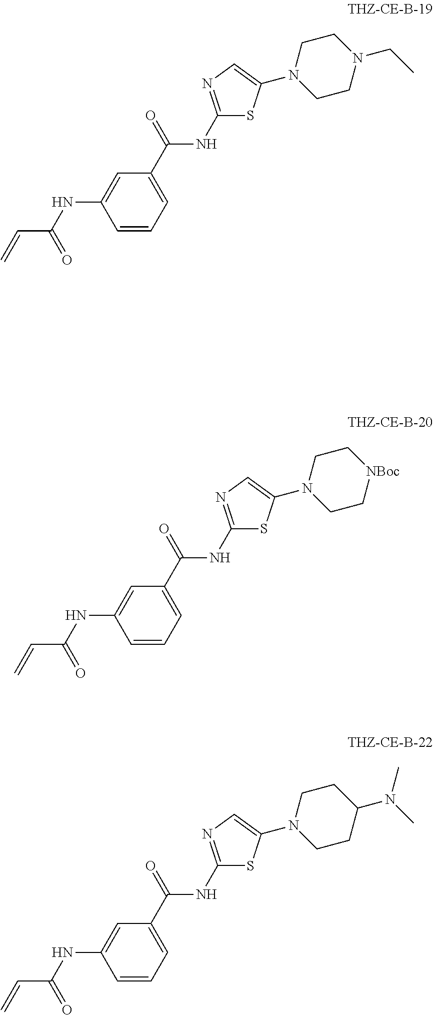

or a pharmaceutically acceptable salt, solvate, hydrate, polymorph, co-crystal, tautomer, stereoisomer, isotopically labeled derivative, or prodrug thereof.

In certain embodiments, the CDK7 inhibitor is a compound of Formula (XXII):

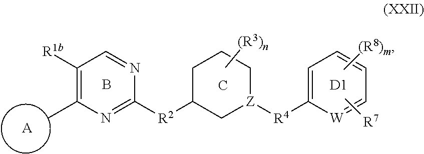

or a pharmaceutically acceptable salt, solvate, hydrate, polymorph, co-crystal, tautomer, stereoisomer, isotopically labeled derivative, or prodrug thereof, wherein:

Ring A is an optionally substituted heteroaryl ring of any one of the Formulae (ii-1)-(ii-5):

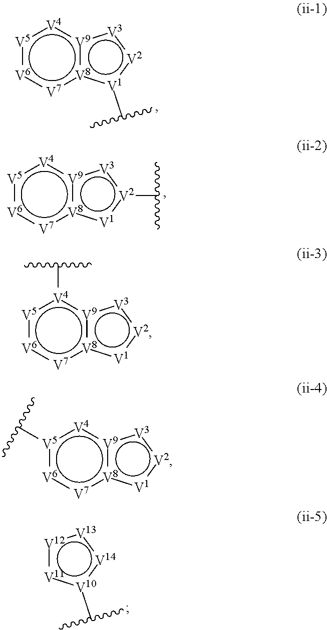

or an optionally substituted 6-membered aryl or heteroaryl ring;

each instance of V¹, V², V³, V⁴, V⁵, V⁶, V⁷, V⁸, V⁹, V¹⁰, V¹¹, V¹², V¹³, and V¹⁴ is independently O, S, N, N(R^{A1}), C, or C(R^{A2});

Z is —CH— or —N—;

each instance of R^{A1} is independently selected from hydrogen, optionally substituted acyl, optionally substituted alkyl, optionally substituted alkenyl, optionally substituted alkynyl, optionally substituted carbocyclyl, optionally substituted heterocyclyl, optionally substituted aryl, and optionally substituted heteroaryl;

each instance of R^{A2} is independently selected from hydrogen, halogen, —CN, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{A2a}, N(R^{A2b})₂, and —SR^{A2a}, wherein R^{A2a} is independently selected from hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, and a sulfur protecting group when attached to a sulfur atom;

wherein each occurrence of R^{A2b} is independently selected from hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, and a nitrogen protecting group, or optionally two instances of R^{A2b} are taken together with their intervening atoms to form a substituted or unsubstituted heterocyclic or substituted or unsubstituted heteroaryl ring; or

any two R^{A1}, any two R^{A2}, or one R^{A1} and one R^{A2} are joined to form an optionally substituted carbocyclic, optionally substituted heterocyclic, optionally substituted aryl, or optionally substituted heteroaryl ring;

each of R^{1b} is independently selected from hydrogen, halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{B1a}, —N(R^{B1b})₂, and —SR^{B1a}, wherein each occurrence of R^{B1a} is independently selected from hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, and a sulfur protecting group when attached to a sulfur atom,

wherein each occurrence of R^{B1b} is independently selected from hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, and a nitrogen protecting group, or optionally two instances of R^{B}1b are taken together with their intervening atoms to form a substituted or unsubstituted heterocyclic or substituted or unsubstituted heteroaryl ring;

R² is —O—, —S—, —N(R⁶)—, or an optionally substituted C₁-C₄ alkylene, wherein one or more methylene units of the alkylene are optionally and independently replaced with —O—, —S—, or —N(R⁶)—;

each instance of R³, if present, is independently selected from halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{C1}, —N(R^{C1a})₂, and —SR^{C1}, wherein each occurrence of R^{C1} is independently selected from hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, and a sulfur protecting group when attached to a sulfur atom;

wherein each occurrence of R^{C1a} is independently selected from hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, and a nitrogen protecting group, or optionally two instances of R^{C1a} are taken together with their intervening atoms to form a substituted or unsubstituted heterocyclic or substituted or unsubstituted heteroaryl ring; or

two R³ groups bound to the same ring carbon atom are taken together to form ═O, or

two R³ groups bound to the same or different ring carbon atoms are joined to form an optionally substituted carbocyclyl, optionally substituted heterocyclyl, optionally substituted aryl, or optionally substituted heteroaryl ring;

R⁴ is selected from a bond, —C(═O)—, —O—, —S—, —N(R⁶)—, —S(═O)₂—, and optionally substituted C₁-C₄ alkylene, wherein:

one or more methylene units of the alkylene other than a methylene unit bound to a nitrogen atom is optionally and independently replaced with

—C(═O), —O—, —S—, —N(R⁶)—, or —S(═O)₂—;

each R⁶ is independently selected from hydrogen and —C₁-C₆ alkyl;

R⁷ is a warhead of formula:

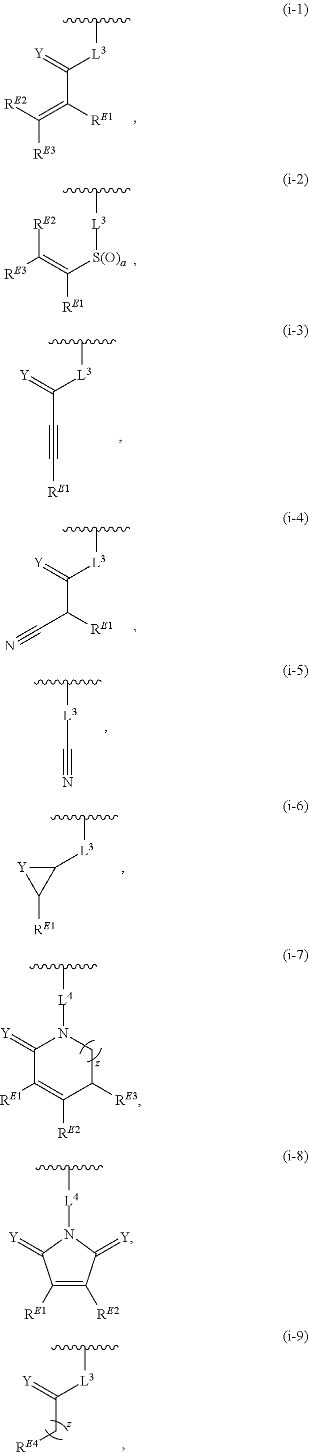

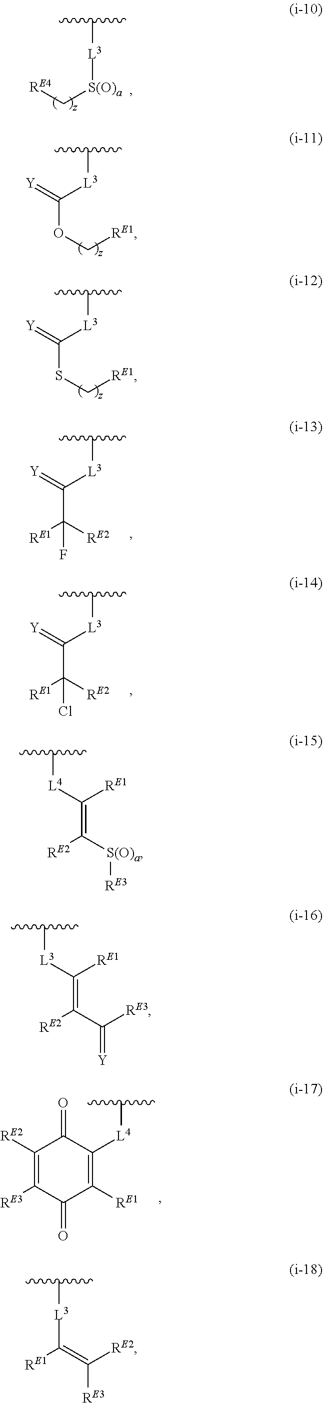

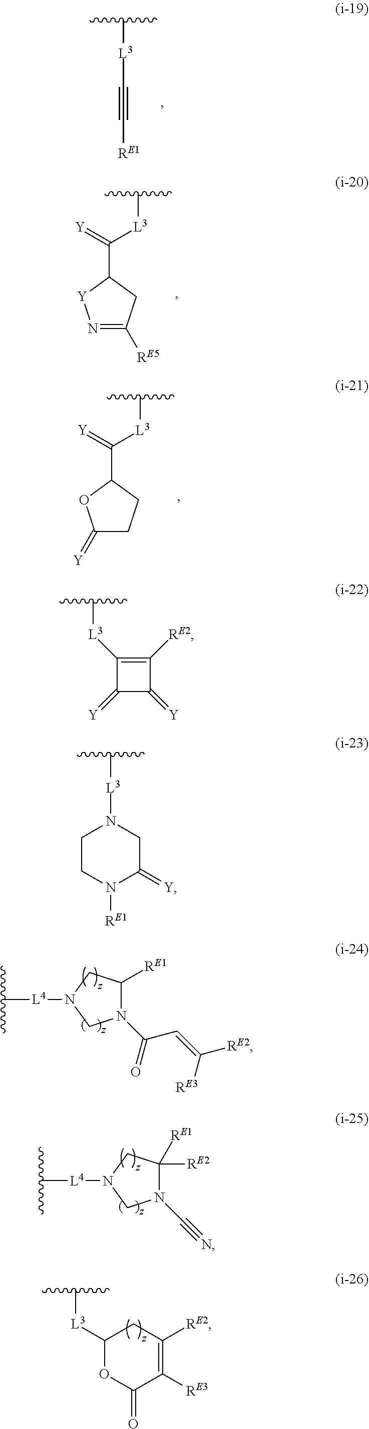

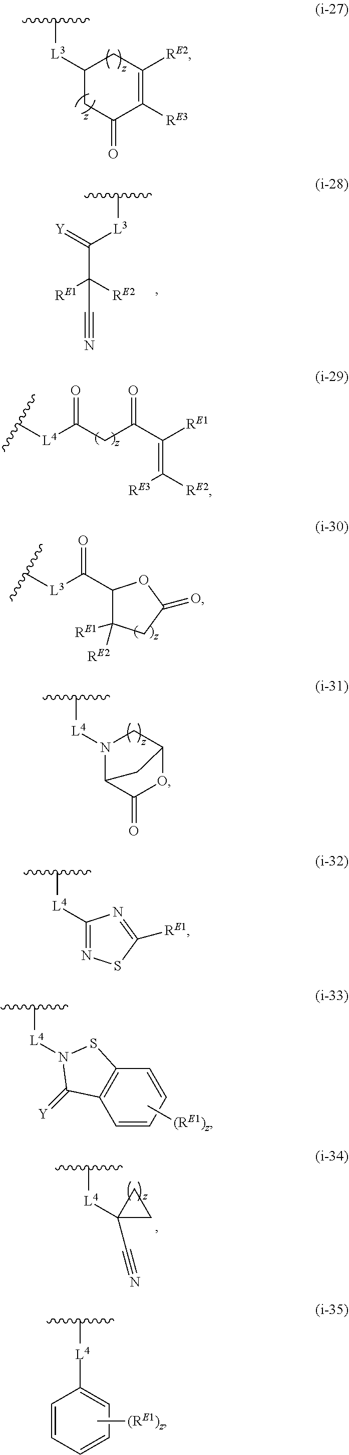

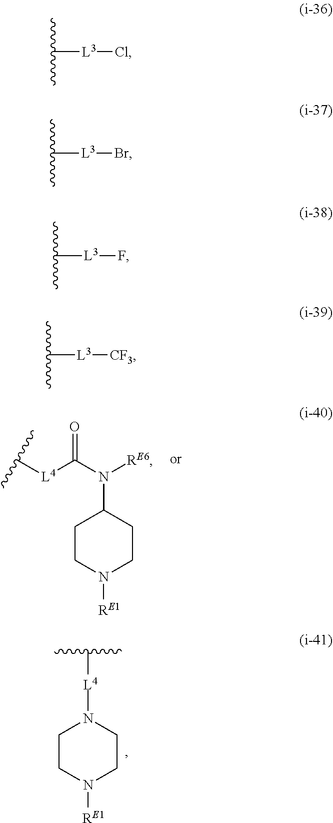

wherein:
L³ is a bond or an optionally substituted C₁₋₄ hydrocarbon chain, optionally wherein one or more carbon units of the hydrocarbon chain are independently replaced with —C═O—, —O—, —S—, —NR^{L3a}—, —NR^{L3a}C(═O)—, —C(═O)NR^{L3a}—, —SC(═O)—, —C(═O)S—, —OC(═O)—, —C(═O)O—, —NR^{L3a}C(═S)—, —C(═S)NR^{L3a}—, trans-CR^{L3b}═CR^{L3b}—, cis-CR^{L3b}=CR^{L3b}—, —C═C—, —S(═O)—, —S(═O)O—, —OS(═O)—, —S(═O)NR^{L3a}—, —NR^{L3a}S(═O)—, —S(═O)₂—, —S(═O)₂O—, —OS(═O)₂—, —S(═O)₂NR^{L3a}—, or —NR^{L3a}S(═O)₂—, wherein R^{L3a} is hydrogen, substituted or unsubstituted C₁₋₆ alkyl, or a nitrogen protecting group, and wherein each occurrence of R^{L3b} is independently hydrogen, halogen, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R^{L3b} groups are joined to form an optionally substituted carbocyclic or optionally substituted heterocyclic ring;
L⁴ is a bond or an optionally substituted, branched or unbranched C₁₋₆ hydrocarbon chain;
each of R^{E1}, R^{E2}, and R^{E3} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{EE}, —CH₂N(R^{EE})₂, —CH₂SR^{EE}, —OR^{EE}, —N(R^{EE})₂, —Si(R^{EE})₃, or —SR^{EE}, wherein each instance of R^{EE} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl, or two R^{EE} groups are joined to form an optionally substituted heterocyclic ring; or R^{E1} and R^{E3}, or R^{E2} and R^{E3}, or R^{E1} and R^{E2} are joined to form an optionally substituted carbocyclic or optionally substituted heterocyclic ring;
R^{E4} is a leaving group;
R^{E5} is halogen;
R^{E6} is hydrogen, substituted or unsubstituted C₁₋₆ alkyl, or a nitrogen protecting group;
each instance of Y is independently O, S, or NR^{E7}, wherein R^{E7} is hydrogen, substituted or unsubstituted C₁₋₆ alkyl, or a nitrogen protecting group;
a is 1 or 2;
each instance of z is independently 0, 1, 2, 3, 4, 5, or 6, as valency permits;
and

W is —CR^{D1}— or —N═;

each instance of R⁸, if present, is independently selected from hydrogen, halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{D1}, —N(R^{D1a})₂, and —SR^{D1}, wherein each occurrence of R^{D1} is independently selected from hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, and a sulfur protecting group when attached to a sulfur atom,

wherein each occurrence of R^{D1a} is independently selected from hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, and a nitrogen protecting group, or optionally two instances of R^{D1a} are taken together with their intervening atoms to form a substituted or unsubstituted heterocyclic or substituted or unsubstituted heteroaryl ring; or

two R⁸ groups are joined to form an optionally substituted carbocyclyl, optionally substituted heterocyclyl, optionally substituted aryl, or optionally substituted heteroaryl ring;

m is 0, 1, 2, 3 or 4; and

n is 0, 1, 2, 3, 4, 5 or 6.

In certain embodiments, the compound of Formula (XXII) is of formula:

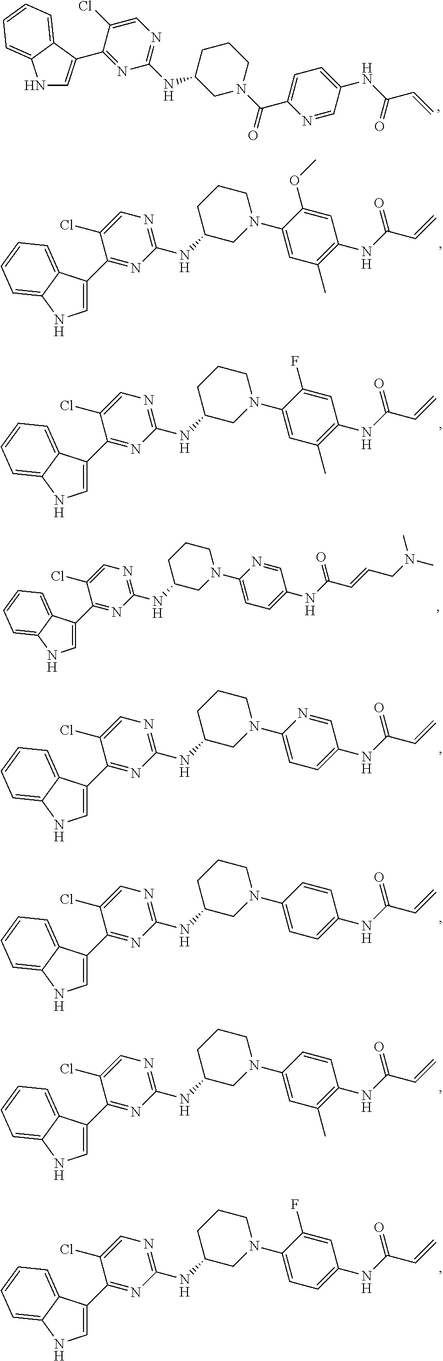

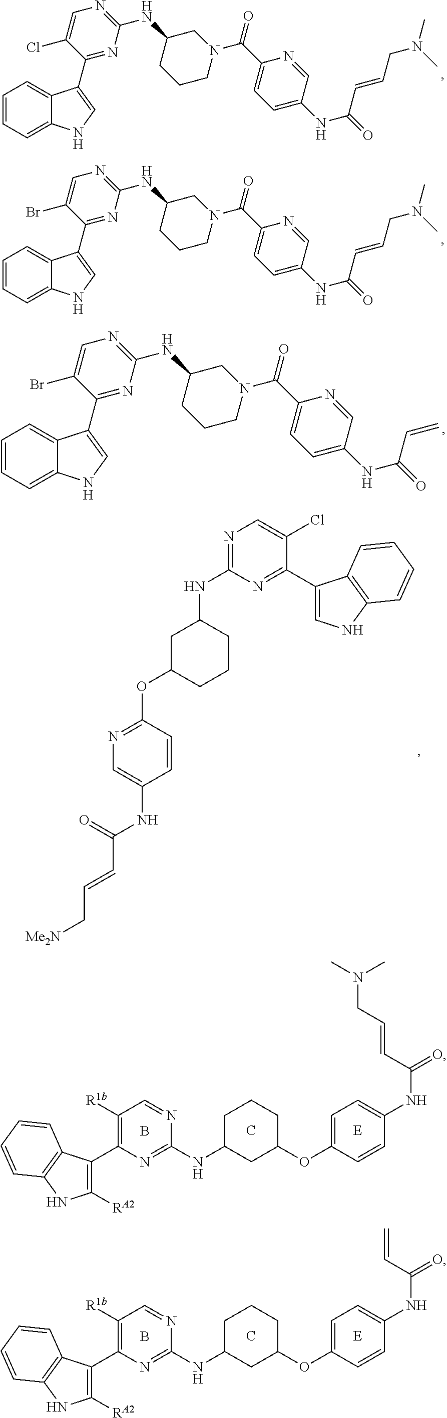

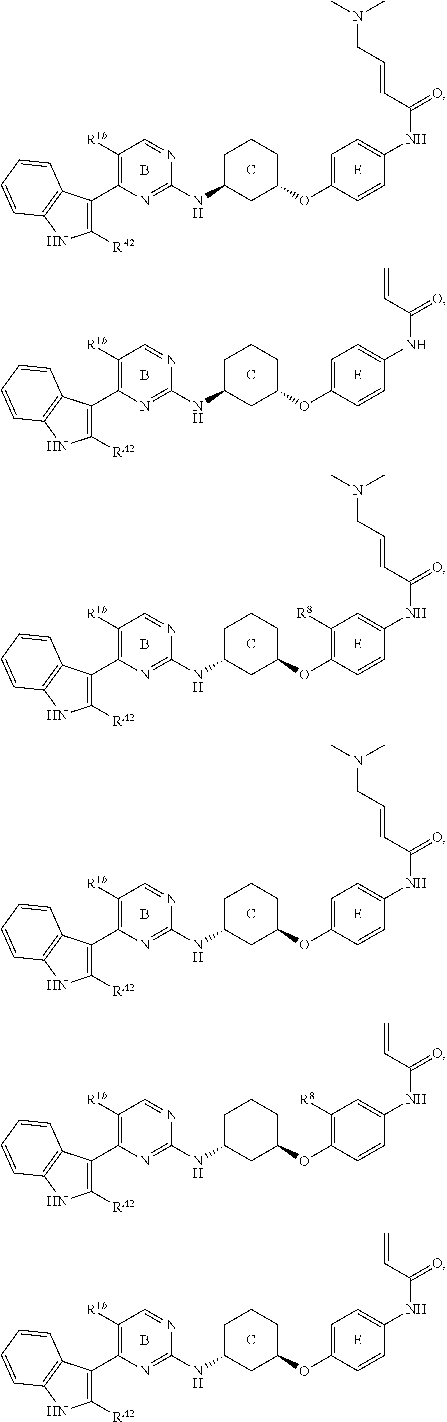

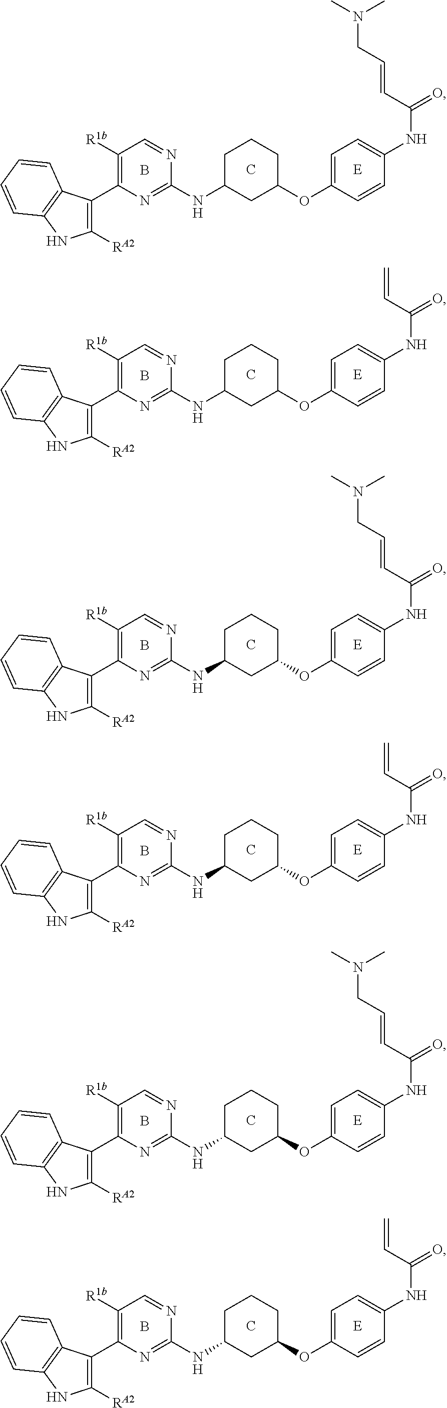

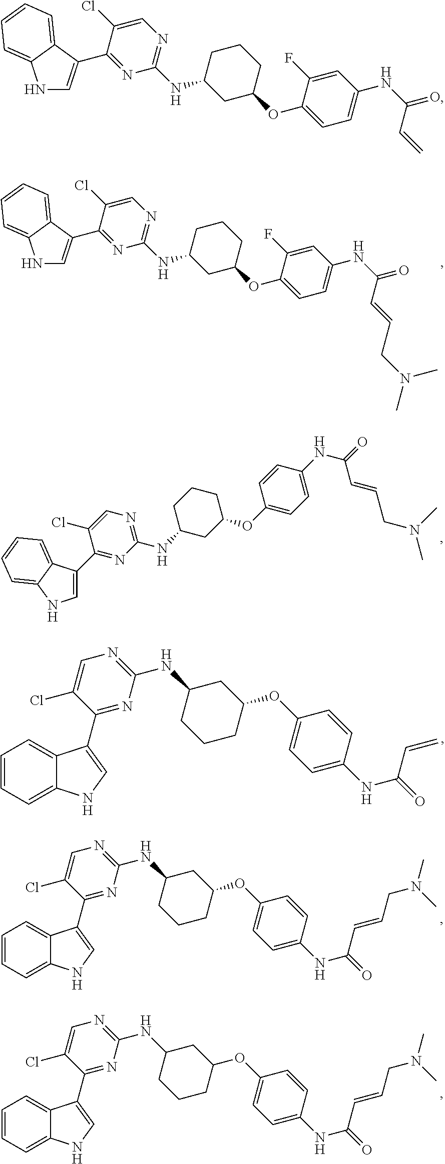 [SMILES: C=CC(=O)Nc1ccc(O[C@@H]2CCC[C@@H](Nc3ncc(Cl)c(-c4c[nH]c5ccccc45)n3)C2)c(F)c1 ; CN(C)C/C=C/C(=O)Nc1ccc(O[C@@H]2CCC[C@@H](Nc3ncc(Cl)c(-c4c[nH]c5ccccc45)n3)C2)c(F)c1 ; CN(C)C/C=C/C(=O)Nc1ccc(O[C@H]2CCC[C@@H](Nc3ncc(Cl)c(-c4c[nH]c5ccccc45)n3)C2)cc1 ; C=CC(=O)Nc1ccc(O[C@@H]2CCC[C@@H](Nc3ncc(Cl)c(-c4c[nH]c5ccccc45)n3)C2)cc1 ; CN(C)C/C=C/C(=O)Nc1ccc(O[C@@H]2CCC[C@@H](Nc3ncc(Cl)c(-c4c[nH]c5ccccc45)n3)C2)cc1 ; CN(C)C/C=C/C(=O)Nc1ccc(OC2CCCC(Nc3ncc(Cl)c(-c4c[nH]c5ccccc45)n3)C2)cc1]

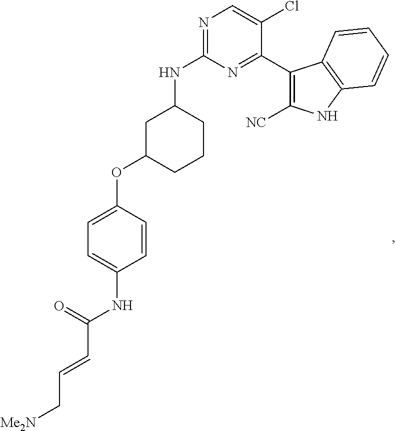 [SMILES: CN(C)C/C=C/C(=O)Nc1ccc(OC2CCCC(Nc3ncc(Cl)c(-c4c(C#N)[nH]c5ccccc45)n3)C2)cc1]

or a pharmaceutically acceptable salt, solvate, hydrate, polymorph, co-crystal, tautomer, stereoisomer, isotopically labeled derivative, or prodrug thereof.

In certain embodiments, the CDK7 inhibitor is a compound of Formula (XXIII):

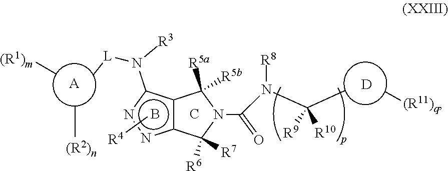

or a pharmaceutically acceptable salt, solvate, hydrate, polymorph, co-crystal, tautomer, stereoisomer, isotopically labeled derivative, or prodrug thereof, wherein:

Ring A is carbocyclyl, heterocyclyl, aryl, or heteroaryl;

R¹ is of the formula:

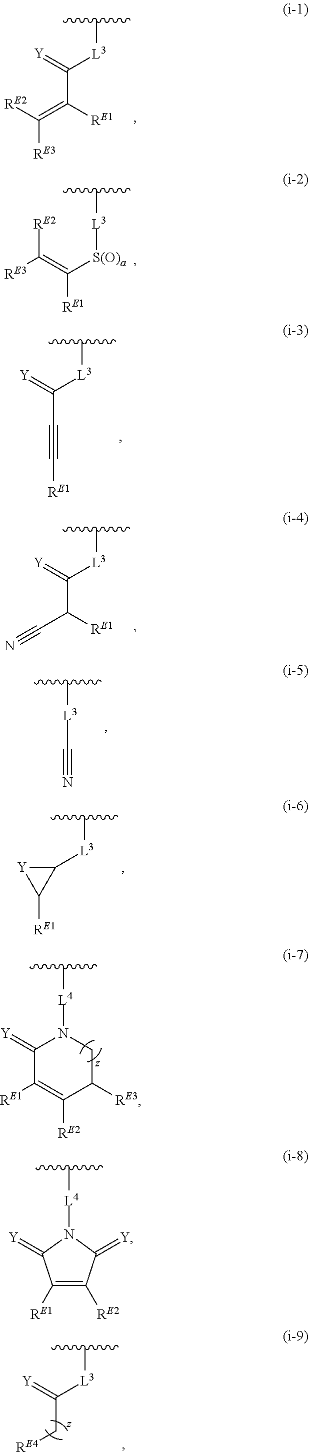

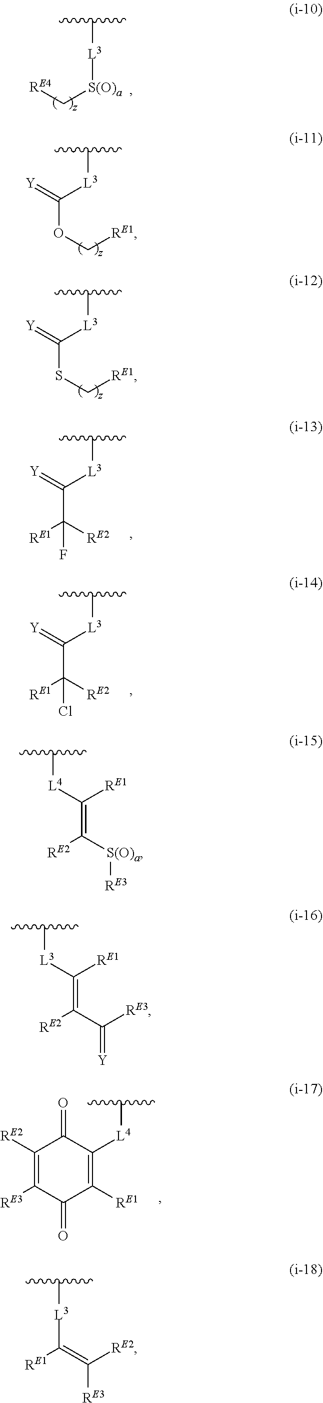

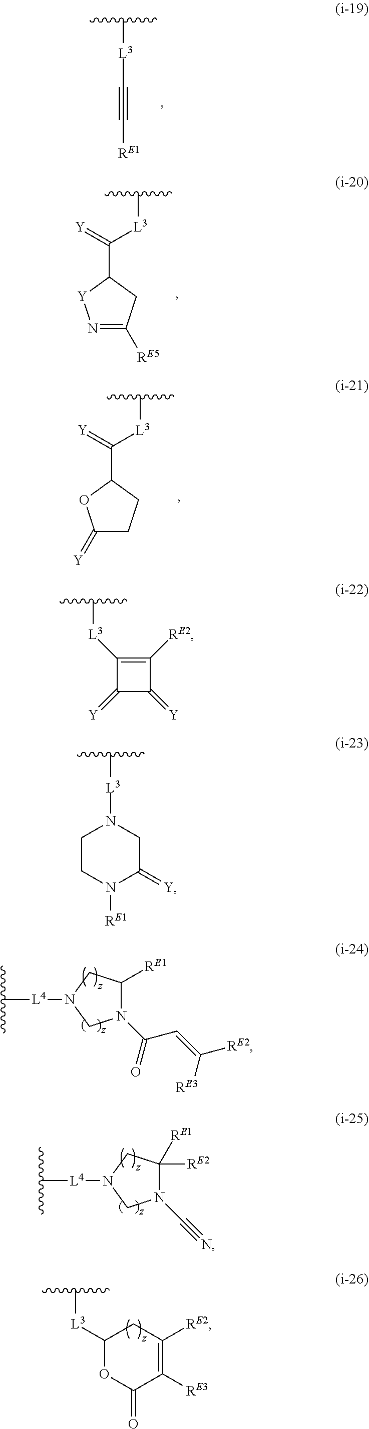

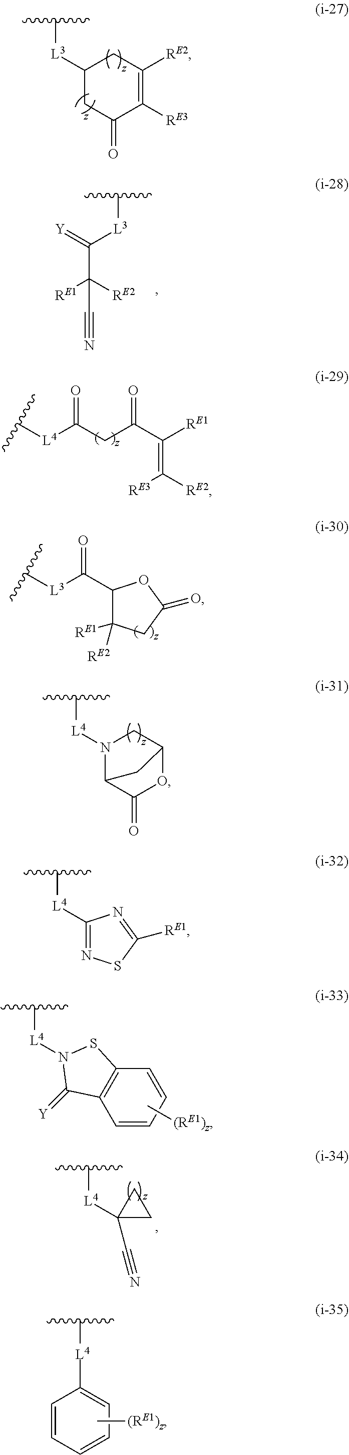

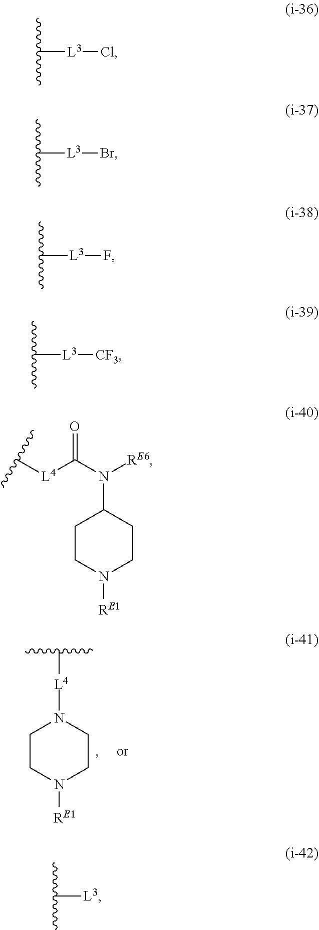

wherein:

L³ is a bond or a substituted or unsubstituted C₁₋₄ hydrocarbon chain, optionally wherein one or more carbon units of the hydrocarbon chain are independently replaced with —C═O—, —O—, —S—, —NR^{L3a}—, —NR^{L3a}C(═O)—, —C(═O)NR^{L3a}—, —SC(═O)—, —C(═O)S—, —OC(═O)—, —C(═O)O—, —NR^{L3a}C(═S)—, —C(═S)NR^{L3a}—, trans-CR^{L3b}═CR^{L3b}—, cis-CR^{L3b}═CR^{L3b}—, —C≡C—, —S(═O)—, —S(═O)O—, —OS(═O)—, —S(═O)NR^{L3a}—, —NR^{L3a}S(═O)—, —S(═O)₂—, —S(═O)₂O—, —OS(═O)₂—, —S(═O)₂NR^{L3a}—, or —NR^{L3a}S(═O)₂—, wherein R^{L3a} is hydrogen, substituted or unsubstituted acyl, substituted or unsubstituted, C₁₋₆ alkyl, or a nitrogen protecting group, and wherein each instance of R^{L3b} is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, or two instances of R^{L3b} are joined to form substituted or unsubstituted carbocyclyl or substituted or unsubstituted heterocyclyl;

L⁴ is a bond or a substituted or unsubstituted, branched or unbranched, C₁₋₆ hydrocarbon chain;

each of R^{E1}, R^{E2}, and R^{E3} is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —CN, —CH₂OR^{EE}, —CH₂N(R^{EE})₂, —CH₂SR^{L}E, —OR^{EE}, —N(R^{EE})₂, —Si(R^{EE})₃, or —SR^{EE}, wherein each instance of R^{EE} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, or two instances of R^{EE} are joined to form substituted or unsubstituted heterocyclyl;

or R^{E1} and R^{E3}, or R^{E2} and R^{E3}, or R^{E1} and R^{E2} are joined to form substituted or unsubstituted carbocyclyl or substituted or unsubstituted heterocyclyl;

R^{E4} is a leaving group;

R^{E5} is halogen;

R^{E6} is hydrogen, substituted or unsubstituted, C₁₋₆ alkyl, or a nitrogen protecting group;

each instance of Y is independently O, S, or NR^{E7}, wherein R^{E7} is hydrogen, substituted or unsubstituted, C₁₋₆ alkyl, or a nitrogen protecting group;

a is 1 or 2; and

each instance of z is independently 0, 1, 2, 3, 4, 5, or 6, as valency permits;

m is 0 or 1;

each instance of R² is independently halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;

n is 0, 1, 2, 3, 4, 5, 6, 7, 8, 9, 10, or 11, as valency permits;

L is a single bond or —C(═O)—;

R³ is hydrogen, substituted or unsubstituted acyl, substituted or unsubstituted alkyl, or a nitrogen protecting group;

R⁴ is hydrogen, substituted or unsubstituted acyl, substituted or unsubstituted alkyl, or a nitrogen protecting group;

each of R^{5a} and R^{5b} is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂, or R^{5a} and R^{5b} are joined to form substituted or unsubstituted carbocyclyl or substituted or unsubstituted heterocyclyl;

each instance of R⁶ and R⁷ is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂, or R⁶ and R⁷ are joined to form substituted or unsubstituted carbocyclyl or substituted or unsubstituted heterocyclyl;

R⁸ is hydrogen, substituted or unsubstituted acyl, substituted or unsubstituted alkyl, or a nitrogen protecting group;

each instance of R⁹ and R¹⁰ is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂, or R⁹ and R¹⁰ are joined to form substituted or unsubstituted carbocyclyl or substituted or unsubstituted heterocyclyl;

p is 0, 1, 2, 3, or 4;

Ring D is carbocyclyl or heterocyclyl;

each instance of R¹¹ is independently halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted aryl, substituted or unsubstituted heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, or a nitrogen protecting group when attached to a nitrogen atom;

q is 0, 1, 2, 3, 4, 5, 6, 7, 8, 9, 10, or 11, as valency permits; and

each instance of R^{a} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{a} are joined to form substituted or unsubstituted heterocyclyl or substituted or unsubstituted heteroaryl.

In certain embodiments, the compound of Formula (XXIII) is not of the formula:

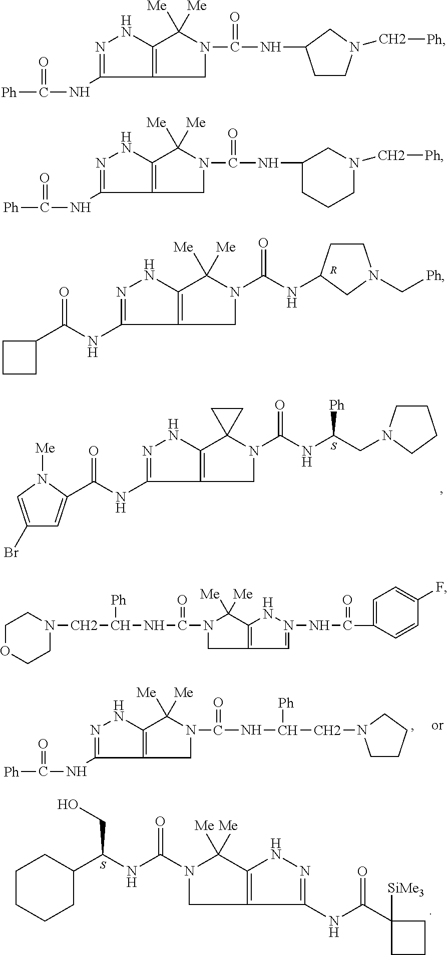

In certain embodiments, the compound of Formula (XXIII) is of formula:

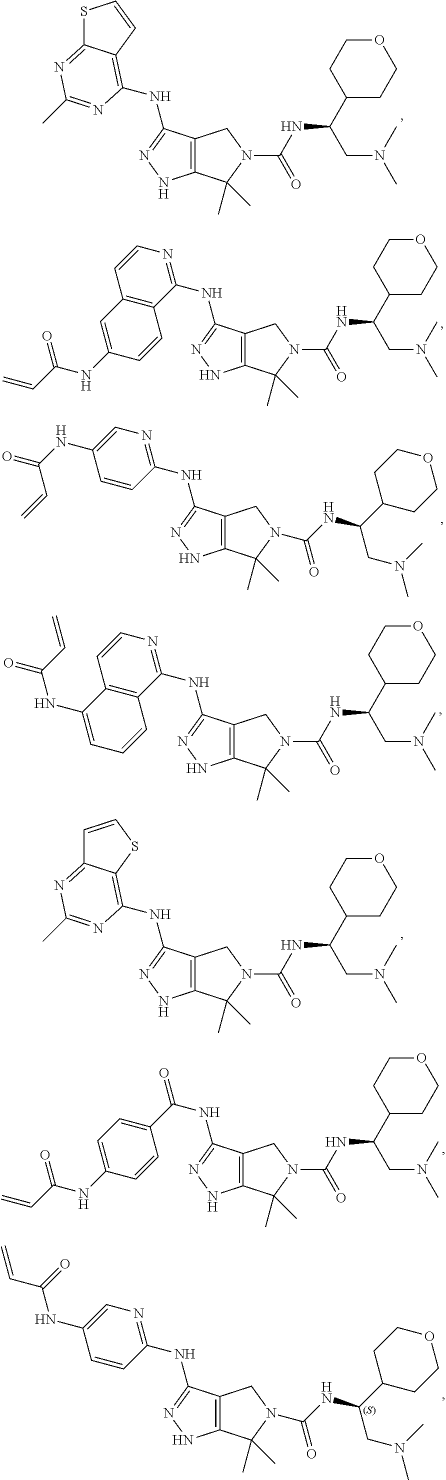

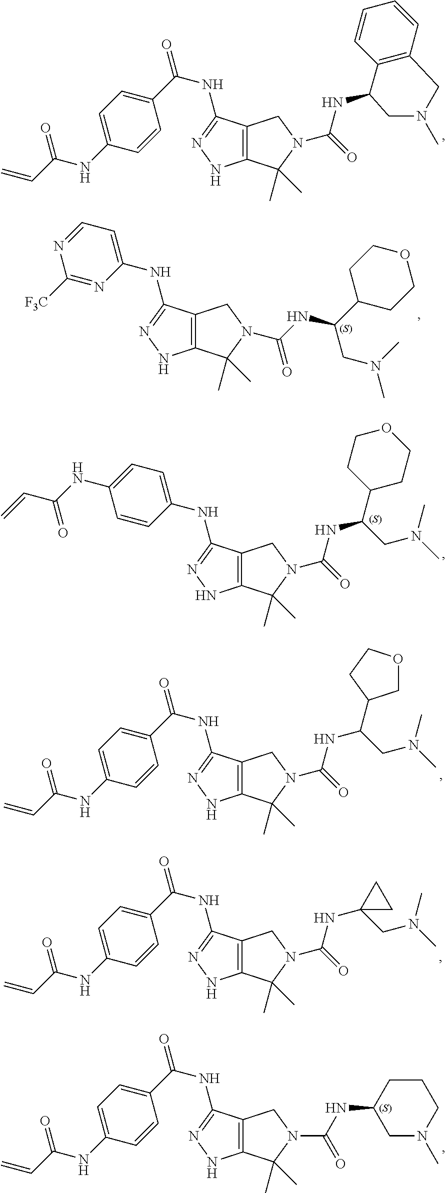

or a pharmaceutically acceptable salt, solvate, hydrate, polymorph, co-crystal, tautomer, stereoisomer, isotopically labeled derivative, or prodrug thereof.

In certain embodiments, the CDK7 inhibitor inhibits the activity of CDK7 at an IC₅₀ less than or equal to 30 μM, less than or equal to 10 μM, less than or equal to 3 μM, less than or equal to 1 μM, less than or equal to 0.3 μM, or less than or equal to 0.1 μM.

In certain embodiments, the CDK7 inhibitor selectively inhibits the activity of CDK7 compared to the activity of a different protein. In certain embodiments, the CDK7 inhibitor selectively inhibits the activity of CDK7 compared to the activity of a different CDK isoform. In certain embodiments, the CDK7 inhibitor selectively inhibits the activity of CDK7 compared to the activity of CDK12. In certain embodiments, the CDK7 inhibitor selectively inhibits the activity of CDK7 compared to the activity of CDK13. In certain embodiments, the CDK7 inhibitor selectively inhibits the activity of CDK7 compared to the activity of CDK12 and the activity of CDK13.

The selectivity of the CDK7 inhibitor over a different protein (e.g., a different CDK isoform) may be measured by the quotient of the IC₅₀ value of the CDK7 inhibitor in inhibiting the activity of the different protein over the IC₅₀ value of the CDK7 inhibitor in inhibiting the activity of CDK7. The selectivity of the CDK7 inhibitor over a different protein may also be measured by the quotient of the K_{d} value of an adduct of the CDK7 inhibitor and the different protein over the K_{d} value of an adduct of the CDK7 inhibitor and CDK7. In certain embodiments, the selectivity is at least 2-fold, at least 3-fold, at least 5-fold, at least 10-fold, at least 30-fold, at least 100-fold, at least 300-fold, at least 1,000-fold, at least 3,000-fold, at least 10,000-fold, at least 30,000-fold, or at least 100,000-fold. In certain embodiments, the selectivity is not more than 100,000-fold, not more than 10,000-fold, not more than 1,000-fold, not more than 100-fold, not more than 10-fold, or not more than 2-fold. Combinations of the above-referenced ranges (e.g., at least 2-fold and not more than 10,000-fold) are also within the scope of the disclosure.

### Immunotherapy

In certain embodiments, the immunotherapy is an immunotherapeutic agent. In certain embodiments, the immunotherapy is an activator of adaptive immune response. In certain embodiments, the immunotherapeutic agent is an activator of adaptive immune response.

The adaptive immune response system, also known as the acquired immune system, is a subsystem of the overall immune system that includes highly specialized systemic cells and processes that eliminate or prevent pathogen growth. The adaptive immune system is one of the two main immunity strategies found in vertebrates (the other being the innate immune system). Adaptive immunity creates immunological memory after an initial response to a specific pathogen and leads to an enhanced response to subsequent encounters with that pathogen. This process of acquired immunity is the basis of vaccination. Like the innate system, the adaptive system includes both humoral immunity components and cell-mediated immunity components. Unlike the innate immune system, the adaptive immune system is highly specific to a particular pathogen.

The adaptive immune response system is triggered in vertebrates when a pathogen evades the innate immune response system, generates a threshold level of antigen, and generates “stranger” or “danger” signals activating dendritic cells. The major functions of the acquired immune system include recognition of specific “non-self” antigens in the presence of “self” during the process of antigen presentation; generation of responses that are tailored to eliminate specific pathogens or pathogen-infected cells; and development of immunological memory, in which pathogens are “remembered” through memory B cells and memory T cells.

Useful approaches to activating the adaptive immune response system (e.g., activating therapeutic antitumor immunity) include the blockade of immune checkpoints. Immune checkpoints refer to a plethora of inhibitory pathways hardwired into the immune system that are crucial for maintaining self-tolerance and modulating the duration and amplitude of physiological immune responses in peripheral tissues in order to minimize collateral tissue damage. Tumors co-opt certain immune-checkpoint pathways as a major mechanism of immune resistance, particularly against T cells that are specific for tumor antigens. Because many of the immune checkpoints are initiated by ligand-receptor interactions, they can be readily blocked by antibodies or modulated by recombinant forms of ligands or receptors. Cytotoxic T-lymphocyte-associated antigen 4 (CTLA-4) antibodies were the first of this class of immunotherapeutic agents to receive FDA approval (ipilimumab). Clinical findings with inhibitors of additional immune-checkpoint proteins, such as programmed cell death protein 1 (PD-1), also provide broad and diverse opportunities to enhance antitumor immunity by producing durable clinical responses.

PD-1, functioning as an immune checkpoint, plays an important role in down-regulating the immune system by preventing the activation of T cells, which in turn reduces autoimmunity and promotes self-tolerance. The inhibitory effect of PD-1 is accomplished through a dual mechanism of promoting apoptosis (programmed cell death) in antigen-specific T cells in lymph nodes while simultaneously reducing apoptosis in regulatory T cells (suppressor T cells). A new class of therapeutics that block PD-1, the PD-1 inhibitors (e.g., anti-PD-1 antibodies), activate the immune system to attack tumors and are therefore used to treat some types of cancer. In addition, antibodies of Programmed death-ligand 1 (PD-L1) provide a similar impact on activating the adaptive immune response as antibodies targeting PD-1. Accordingly, methods and compositions comprising administration of anti-PD-L1 antibodies are expected to provide a similar therapeutic effect as those comprising anti-PD-1 antibodies.

In certain embodiments, the immunotherapy is an immune checkpoint inhibitor. In certain embodiments, the immunotherapeutic agent is an immune checkpoint inhibitor. In certain embodiments, the activator of adaptive immune response is an immune checkpoint inhibitor.

In certain embodiments, the immunotherapeutic agent is a small molecule. In certain embodiments, the immunotherapeutic agent is a biologic. In certain embodiments, the biologic is a protein. In certain embodiments, the biologic is an antibody or fragment thereof. In certain embodiments, the biologic is a nucleic acid that encodes a protein.

In certain embodiments, the immunotherapeutic agent is an anti-PD-1 antibody, an anti-PD-L1 antibody, an anti-CTLA-4 antibody, an anti-TIM3 antibody, an anti-OX40 antibody, an anti-GITR antibody, an anti-LAG-3 antibody, an anti-CD137 antibody, an anti-CD3 antibody, an anti-CD27 antibody, an anti-CD28 antibody, an anti-CD28H antibody, an anti-CD30 antibody, an anti-CD39 antibody, an anti-CD40 antibody, an anti-CD43 antibody, an anti-CD47 antibody, an anti-CD48 antibody, an anti-CD70 antibody, an anti-CD73 antibody, an anti-CD96 antibody, an anti-CD123 antibody, an anti-CD155 antibody, an anti-CD160 antibody, an anti-CD200 antibody, an anti-CD244 antibody, an anti-ICOS antibody, an anti-TNFRSF25 antibody, an anti-TMIGD2 antibody, an anti-DNAM1 antibody, an anti-BTLA antibody, an anti-LIGHT antibody, an anti-TIGIT antibody, an anti-VISTA antibody, an anti-HVEM antibody, an anti-Siglec antibody, an anti-GAL1 antibody, an anti-GAL3 antibody, an anti-GAL9 antibody, an anti-BTNL2 (butrophylins) antibody, an anti-B7-H3 antibody, an anti-B7-H4 antibody, an anti-B7-H5 antibody, an anti-B7-H6 antibody, an anti-KIR antibody, an anti-LIR antibody, an anti-ILT antibody, an anti-CEACAMi antibody, an anti-CEACAM5 antibody, an anti-CEACAM6 antibody, an anti-MICA antibody, an anti-MICB antibody, an anti-NKG2D antibody, an anti-NKG2A antibody, an anti-A2AR antibody, an anti-C5aR antibody, an anti-TGFβ antibody, an anti-TGFβR antibody, an anti-CXCR4 antibody, an anti-CXCL12 antibody, an anti-CCL2 antibody, an anti-IL-10 antibody, an anti-IL-13 antibody, an anti-IL-23 antibody, an anti-phosphatidylserine antibody, an anti-neuropilin antibody, an anti-GalCer antibody, an anti-HER2 antibody, an anti-VEGFA antibody, an anti-VEGFR antibody, an anti-EGFR antibody, an anti-Tie2 antibody, an anti-CCR4 antibody, or an anti-TRAIL-DR5 antibody.

In certain embodiments, the immunotherapy is an inhibitor of PD-1, PD-L1, or CTLA-4. In certain embodiments, the immunotherapeutic agent is an inhibitor of PD-1, PD-L1, or CTLA-4. In certain embodiments, the activator of adaptive immune response is an inhibitor of PD-1, PD-L1, or CTLA-4. In certain embodiments, the immune checkpoint inhibitor is an inhibitor of PD-1, PD-L1, or CTLA-4.

In certain embodiments, the immunotherapy is an inhibitor of PD-1. In certain embodiments, the immunotherapeutic agent is an inhibitor of PD-1. In certain embodiments, the activator of adaptive immune response is an inhibitor of PD-1. In certain embodiments, the immune checkpoint inhibitor is an inhibitor of PD-1.

In certain embodiments, the immunotherapy is an anti-PD-1 antibody, an anti-PD-L1 antibody, or an anti-CTLA-4-antibody. In certain embodiments, the immunotherapeutic agent is an anti-PD-1 antibody, an anti-PD-L1 antibody, or an anti-CTLA-4-antibody. In certain embodiments, the activator of adaptive immune response is an anti-PD-1 antibody, an anti-PD-L1 antibody, or an anti-CTLA-4-antibody. In certain embodiments, the immune checkpoint inhibitor is an anti-PD-1 antibody, an anti-PD-L1 antibody, or an anti-CTLA-4-antibody.

In certain embodiments, the immunotherapy is an anti-PD-1 antibody. In certain embodiments, the immunotherapeutic agent is an anti-PD-1 antibody. In certain embodiments, the activator of adaptive immune response is an anti-PD-1 antibody. In certain embodiments, the immune checkpoint inhibitor is an anti-PD-1 antibody.

In certain embodiments, the immunotherapeutic agent is pembrolizumab, nivolumab, spartalizumab, pidilizumab, ipilimumab, tremelimumab, tislelizumab, durvalumab, atezolizumab, avelumab, cemiplimab, PF-06801591, utomilumab, PDR001, PBF-509, MGB453, LAG525, AMP-224, INCSHR1210, INCAGN1876, INCAGN1949, samalizumab, PF-05082566, urelumab, lirilumab, lulizumab, BMS-936559, BMS-936561, BMS-986004, BMS-986012, BMS-986016, BMS-986178, IMP321, IPH2101, IPH2201, IPH5401, IPH4102, IPH4301, IPH52, IPH53, varlilumab, ulocuplumab, monalizumab, MEDI0562, MEDIO680, MEDI1873, MEDI6383, MEDI6469, MEDI9447, AMG228, AMG820, CC-90002, CDX-1127, CGEN15001T, CGEN15022, CGEN15029, CGEN15049, CGEN15027, CGEN15052, CGEN15092, CX-072, CX-2009, CP-870893, lucatumumab, dacetuzumab, Chi Lob 7/4, RG6058, RG7686, RG7876, RG7888, TRX518, MK-4166, IMC-CS4, emactuzumab, trastuzumab, pertuzumab, obinutuzumab, cabiralizumab, margetuximab, enoblituzumab, mogamulizumab, panitumumab, carlumab, ramucirumab, bevacizumab, rituximab, cetuximab, fresolimumab, denosumab, MGA012, AGEN1884, AGEN2034, LY3300054, JTX-4014, teplizumab, FPA150, PF-04136309, PF-06747143, AZD5069, GSK3359609, FAZ053, TSR022, MBG453, REGN2810, REGN3767, MOXR0916, PF-04518600, RO7009789, BMS986156, GWN323, JTX-2011, NKTR-214, GSK3174998, DS-8273a, NIS793, or BGB-A317.

In certain embodiments, the immunotherapy is ipilimumab, nivolumab, pembrolizumab, spartalizumab, atezolizumab, tislelizumab, tremelimumab, durvalumab, avelumab, or cemiplimab. In certain embodiments, the immunotherapeutic agent is ipilimumab, nivolumab, pembrolizumab, spartalizumab, atezolizumab, tislelizumab, tremelimumab, durvalumab, avelumab, or cemiplimab. In certain embodiments, the activator of adaptive immune response is ipilimumab, nivolumab, pembrolizumab, spartalizumab, atezolizumab, tislelizumab, tremelimumab, durvalumab, avelumab, or cemiplimab. In certain embodiments, the immune checkpoint inhibitor is ipilimumab, nivolumab, pembrolizumab, spartalizumab, atezolizumab, tislelizumab, tremelimumab, durvalumab, avelumab, or cemiplimab.

In certain embodiments, the immunotherapy is nivolumab, pembrolizumab, spartalizumab, tislelizumab, or cemiplimab. In certain embodiments, the immunotherapeutic agent is nivolumab, pembrolizumab, spartalizumab, tislelizumab, or cemiplimab. In certain embodiments, the activator of adaptive immune response is nivolumab, pembrolizumab, spartalizumab, tislelizumab, or cemiplimab. In certain embodiments, the immune checkpoint inhibitor is nivolumab, pembrolizumab, spartalizumab, tislelizumab, or cemiplimab.

In certain embodiments, the immunotherapeutic agent is a fragment of any of the antibodies listed above. In certain embodiments, the immunotherapeutic agent response is a humanized form of any of the antibodies listed above. In certain embodiments, the immunotherapeutic agent is a single chain of any of the antibodies listed above. In certain embodiments, the immunotherapeutic agent is a multimeric form of any of the antibodies listed above (e.g., dimeric IgA molecules, pentavalent IgM molecules).

In certain embodiments, the immunotherapeutic agent is an antibody mimetic or antibody fusion. In certain embodiments, the immunotherapeutic agent is a bispecific antibody. In certain embodiments, the bispecific antibody is RG7802 (antibody targeting carcinoembryonic antigen (CEA) and the CD3 receptor), RG7828 (a bispecific monoclonal antibody that targets CD20 on B cells and CD3 on T cells), RG7221 (a bispecific monoclonal antibody that targets VEGF and angiopoietin 2), RG7386 (a bispecific monoclonal antibody that targets FAP and DR5), ERY974 (a bispecific monoclonal antibody that targets CD3 and glypican-3), MGD012 (a bispecific monoclonal antibody that targets PD-1 and LAG-3), AMG211 (a bispecific T cell engager that targets CD3 and CEA), MEDI573 (a bispecific monoclonal antibody that targets IGF1 and IGF2), MEDI565 (a bispecific monoclonal antibody that targets CD3 and CEA), FS17 (undisclosed targets), FS18 (a bispecific monoclonal antibody that targets LAG3 and an undisclosed target), FS20 (undisclosed targets), FS22 (undisclosed targets), FS101 (a bispecific monoclonal antibody that targets EGFR and HGF), FS117 (undisclosed targets), FS118 (a bispecific monoclonal antibody that targets LAG3 and PD-L1), RO6958688 (a bispecific monoclonal antibody that targets CD3 and CEA), MCLA-128 (a bispecific monoclonal antibody that targets HER2 and HER3), M7824 (bi-functional fusion-protein targeting PD-L1 and TGFβ), MGD009 (a humanized antibody that recognizes both B7-H3 and CD3), or MGD013 (a bispecific PD-1 and LAG-3 antibody).

In certain embodiments, the immunotherapeutic agent is an antibody-drug conjugate. In certain embodiments, the antibody-drug conjugate is trastuzumab emtansine, inotuzumab ozogamicin, PF-06647020, PF-06647263, PF-06650808, RG7596, RG7841, RG7882, RG7986, DS-8201, ABBV-399, glembatumumab vedotin, inotuzumab ozogamicin, MEDI4276, or pharmaceutically acceptable salts thereof.

### Chemotherapy

In certain embodiments, the methods disclosed herein further comprise administering chemotherapy. In certain embodiments, chemotherapy comprises one or more chemotherapeutic agent. In certain embodiments the chemotherapeutic agent includes, but is not limited to, anti-estrogens (e.g., tamoxifen, raloxifene, and megestrol), LHRH agonists (e.g., goscrclin and leuprolide), anti-androgens (e.g., flutamide and bicalutamide), photodynamic therapies (e.g., vertoporfin (BPD-MA), phthalocyanine, photosensitizer Pc4, and demethoxy-hypocrellin A (2BA-2-DMHA)), nitrogen mustards (e.g., cyclophosphamide, ifosfamide, trofosfamide, chlorambucil, estramustine, and melphalan), nitrosoureas (e.g., carmustine (BCNU) and lomustine (CCNU)), alkylsulphonates (e.g., busulfan and treosulfan), triazenes (e.g., dacarbazine and temozolomide), platinum-containing compounds (e.g., cisplatin, carboplatin, and oxaliplatin), vinca alkaloids (e.g., vincristine, vinblastine, vindesine, and vinorelbine), taxoids (e.g., paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g., etoposide, etoposide phosphate, teniposide, topotecan, 9-aminocamptothecin, camptoirinotecan, irinotecan, crisnatol, and mytomycin C), anti-metabolites, DHFR inhibitors (e.g., methotrexate, dichloromethotrexate, trimetrexate, and edatrexate), IMP dehydrogenase inhibitors (e.g., mycophenolic acid, tiazofurin, ribavirin, and EICAR), ribonuclotide reductase inhibitors (e.g., hydroxyurea and deferoxamine), uracil analogs (e.g., 5-fluorouracil (5-FU), floxuridine, doxifluridine, ratitrexed, tegafur-uracil, and capecitabine), cytosine analogs (e.g., cytarabine (ara C), cytosine arabinoside, and fludarabine), purine analogs (e.g., mercaptopurine and thioguanine), Vitamin D3 analogs (e.g., EB 1089, CB 1093, and KH 1060), isoprenylation inhibitors (e.g., lovastatin), dopaminergic neurotoxins (e.g., 1-methyl-4-phenylpyridinium ion), cell cycle inhibitors (e.g., staurosporine), actinomycin (e.g., actinomycin D, dactinomycin), bleomycin (e.g., bleomycin A2, bleomycin B2, and peplomycin), anthracycline (e.g., daunorubicin, doxorubicin, pegylated liposomal doxorubicin, idarubicin, epirubicin, pirarubicin, zorubicin, and mitoxantrone), MDR inhibitors (e.g., verapamil), Ca²⁺ ATPase inhibitors (e.g., thapsigargin), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, hexamethyl melamine, and pharmaceutically acceptable salts thereof. In certain embodiments the chemotherapeutic agent is etoposide. In certain embodiments the chemotherapeutic agent is cisplatin.

### Targeted Therapy

In certain embodiments, the methods disclosed herein further comprise administering a targeted therapy. In certain embodiments, targeted therapy comprises one or more targeted agent.

In certain embodiments, the targeted agent includes, but is not limited to, an IDO inhibitor, a TGFβR inhibitor, an arginase inhibitor, an iNOS inhibitor, a HIF1α inhibitor, a STAT3 inhibitor, a CSF1R inhibitor, a PGE2 inhibitor, a PDE5 inhibitor, a RON inhibitor, an mTOR inhibitor, a JAK2 inhibitor, an HSP90 inhibitor, a PI3K-AKT inhibitor, a β-catenin inhibitor, a GSK3β inhibitor, an IAP inhibitor, an HDAC inhibitor, a DNMT inhibitor, a BET inhibitor, an A2AR inhibitor, a BRAF+MEK inhibitor, a pan-RAF inhibitor, a PI3Kγ inhibitor, a PI3Kδ inhibitor, an EGFR inhibitor, a VEGF inhibitor, a PARP inhibitor, a glutaminase inhibitor, a BTK inhibitor, an ITK inhibitor, a WNT inhibitor, a FAK inhibitor, an ALK inhibitor, a CDK4/6 inhibitor, a or an FGFR3 inhibitor.

In certain embodiments, the targeted agent includes, but is not limited to, imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), epacadostat, indoximid, GDC0919, BMS986205, AZD4635, CPI-444, PBF509, LCL161, CB-839, CB-1158, FPA008, BLZ945, IPI-549, pexidartinib, galunisertib, birinapant, trametinib, dabrafenib, vemurafenib, cobimetinib, binimetinib, ensartib, pazopanib, nintedanib, SYM004, veliparib, olaparib, BGB-290, LXH254, azacitidine, decitabine, guadecitabine, RRX001, CC486, romidepsin, entinostat, vorinostat, panobinostat, tamoxifen, ibrutinib, idelalisib, capmatinib, selumetinib, abemaciclib, palbociclib, glasdegib, enzalutamide, AZD9150, PF-06840003, SRF231, Hu5F9-G4, CC-900002, TTI-621, WNT974, BGJ398, LY2874455, an anti-Tie2 antibody, or pharmaceutically acceptable salts thereof.

### CERTAIN EMBODIMENTS

In certain embodiments, the method comprises administering a compound of Formula (I) and an immunotherapy. In certain embodiments, the method comprises administering a compound of Formula (I) and an immunotherapeutic agent. In certain embodiments, the method comprises administering a compound of Formula (I) and an activator of adaptive immune response. In certain embodiments, the method comprises administering a compound of Formula (I) and an immune checkpoint inhibitor. In certain embodiments, the method comprises administering a compound of Formula (I) and an inhibitor of PD-1, PD-L1, or CTLA-4. In certain embodiments, the method comprises administering a compound of Formula (I) and an inhibitor of PD-1. In certain embodiments, the method comprises administering a compound of Formula (I) and an anti-PD-1 antibody. In certain embodiments, the method comprises administering a compound of Formula (I) and ipilimumab, nivolumab, pembrolizumab, spartalizumab, atezolizumab, tislelizumab, tremelimumab, durvalumab, avelumab, or cemiplimab. In certain embodiments, the method comprises administering a compound of Formula (I) and nivolumab, pembrolizumab, spartalizumab, tislelizumab, or cemiplimab.

In certain embodiments, the method comprises administering

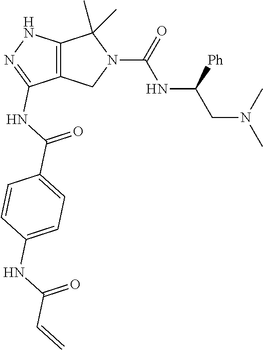 [SMILES: C=CC(=O)Nc1ccc(C(=O)Nc2n[nH]c3c2CN(C(=O)N[C@H](CN(C)C)c2ccccc2)C3(C)C)cc1]

or a pharmaceutically acceptable salt, solvate, hydrate, polymorph, co-crystal, tautomer, stereoisomer, isotopically labeled derivative, or prodrug thereof, and an immunotherapy. In certain embodiments, the method comprises administering

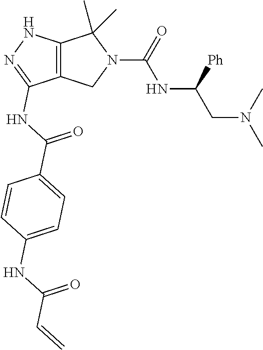 [SMILES: C=CC(=O)Nc1ccc(C(=O)Nc2n[nH]c3c2CN(C(=O)N[C@H](CN(C)C)c2ccccc2)C3(C)C)cc1]

or a pharmaceutically acceptable salt, solvate, hydrate, polymorph, co-crystal, tautomer, stereoisomer, isotopically labeled derivative, or prodrug thereof, and an immunotherapeutic agent. In certain embodiments, the method comprises administering

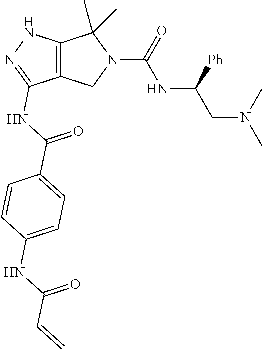 [SMILES: C=CC(=O)Nc1ccc(C(=O)Nc2n[nH]c3c2CN(C(=O)N[C@H](CN(C)C)c2ccccc2)C3(C)C)cc1]

or a pharmaceutically acceptable salt, solvate, hydrate, polymorph, co-crystal, tautomer, stereoisomer, isotopically labeled derivative, or prodrug thereof, and an activator of adaptive immune response. In certain embodiments, the method comprises administering

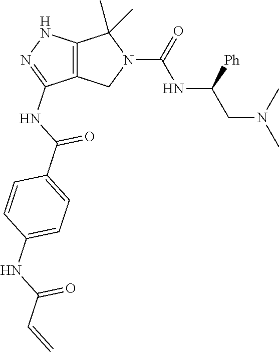 [SMILES: C=CC(=O)Nc1ccc(C(=O)Nc2n[nH]c3c2CN(C(=O)N[C@H](CN(C)C)c2ccccc2)C3(C)C)cc1]

or a pharmaceutically acceptable salt, solvate, hydrate, polymorph, co-crystal, tautomer, stereoisomer, isotopically labeled derivative, or prodrug thereof, and an immune checkpoint inhibitor. In certain embodiments, the method comprises administering

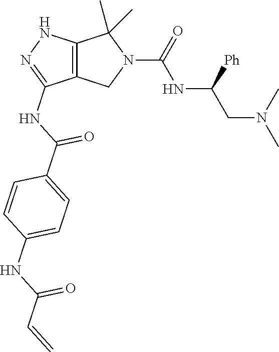 [SMILES: C=CC(=O)Nc1ccc(C(=O)Nc2n[nH]c3c2CN(C(=O)N[C@H](CN(C)C)c2ccccc2)C3(C)C)cc1]

or a pharmaceutically acceptable salt, solvate, hydrate, polymorph, co-crystal, tautomer, stereoisomer, isotopically labeled derivative, or prodrug thereof, and an inhibitor of PD-1, PD-L1, or CTLA-4. In certain embodiments, the method comprises administering

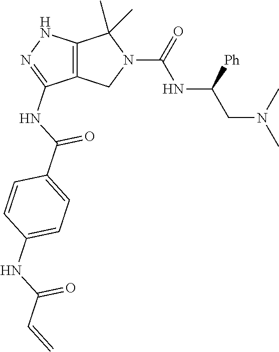 [SMILES: C=CC(=O)Nc1ccc(C(=O)Nc2n[nH]c3c2CN(C(=O)N[C@H](CN(C)C)c2ccccc2)C3(C)C)cc1]

or a pharmaceutically acceptable salt, solvate, hydrate, polymorph, co-crystal, tautomer, stereoisomer, isotopically labeled derivative, or prodrug thereof, and an inhibitor of PD-1. In certain embodiments, the method comprises administering

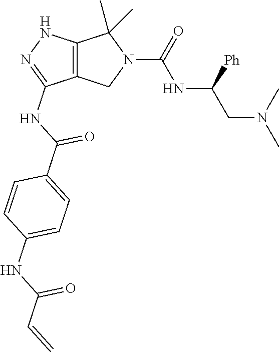 [SMILES: C=CC(=O)Nc1ccc(C(=O)Nc2n[nH]c3c2CN(C(=O)N[C@H](CN(C)C)c2ccccc2)C3(C)C)cc1]

or a pharmaceutically acceptable salt, solvate, hydrate, polymorph, co-crystal, tautomer, stereoisomer, isotopically labeled derivative, or prodrug thereof, and an anti-PD-1 antibody. In certain embodiments, the method comprises administering

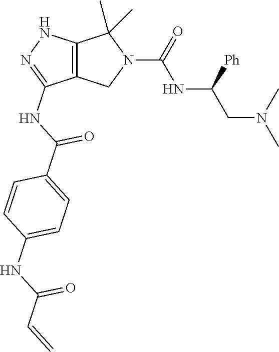 [SMILES: C=CC(=O)Nc1ccc(C(=O)Nc2n[nH]c3c2CN(C(=O)N[C@H](CN(C)C)c2ccccc2)C3(C)C)cc1]

or a pharmaceutically acceptable salt, solvate, hydrate, polymorph, co-crystal, tautomer, stereoisomer, isotopically labeled derivative, or prodrug thereof, and ipilimumab, nivolumab, pembrolizumab, spartalizumab, atezolizumab, tislelizumab, tremelimumab, durvalumab, avelumab, or cemiplimab. In certain embodiments, the method comprises administering

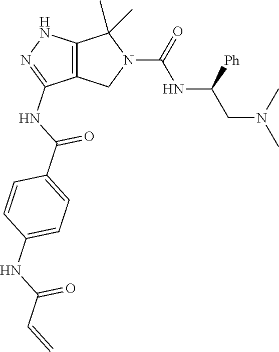 [SMILES: C=CC(=O)Nc1ccc(C(=O)Nc2n[nH]c3c2CN(C(=O)N[C@H](CN(C)C)c2ccccc2)C3(C)C)cc1]

or a pharmaceutically acceptable salt, solvate, hydrate, polymorph, co-crystal, tautomer, stereoisomer, isotopically labeled derivative, or prodrug thereof, and nivolumab, pembrolizumab, spartalizumab, tislelizumab, or cemiplimab.

In certain embodiments, the method comprises administering a compound of Formula (I), an immunotherapy, and one or more chemotherapeutic agent. In certain embodiments, the method comprises administering a compound of Formula (I), an immunotherapeutic agent, and one or more chemotherapeutic agent. In certain embodiments, the method comprises administering a compound of Formula (I), an activator of adaptive immune response, and one or more chemotherapeutic agent. In certain embodiments, the method comprises administering a compound of Formula (I), an immune checkpoint inhibitor, and one or more chemotherapeutic agent. In certain embodiments, the method comprises administering a compound of Formula (I), an inhibitor of PD-1, PD-L1, or CTLA-4, and one or more chemotherapeutic agent. In certain embodiments, the method comprises administering a compound of Formula (I), an inhibitor of PD-1, and one or more chemotherapeutic agent. In certain embodiments, the method comprises administering a compound of Formula (I), an anti-PD-1 antibody, and one or more chemotherapeutic agent. In certain embodiments, the method comprises administering a compound of Formula (I), ipilimumab, nivolumab, pembrolizumab, spartalizumab, atezolizumab, tislelizumab, tremelimumab, durvalumab, avelumab, or cemiplimab, and one or more chemotherapeutic agent. In certain embodiments, the method comprises administering a compound of Formula (I), nivolumab, pembrolizumab, spartalizumab, tislelizumab, or cemiplimab, and one or more chemotherapeutic agent.

In certain embodiments, the method comprises administering

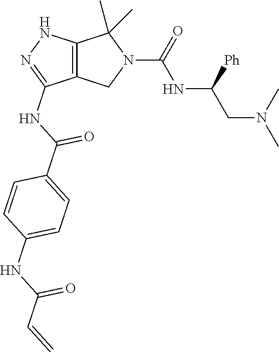 [SMILES: C=CC(=O)Nc1ccc(C(=O)Nc2n[nH]c3c2CN(C(=O)N[C@H](CN(C)C)c2ccccc2)C3(C)C)cc1]

or a pharmaceutically acceptable salt, solvate, hydrate, polymorph, co-crystal, tautomer, stereoisomer, isotopically labeled derivative, or prodrug thereof, an immunotherapy, and at least one of cisplatin and etoposide. In certain embodiments, the method comprises administering

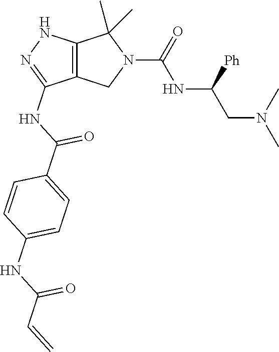 [SMILES: C=CC(=O)Nc1ccc(C(=O)Nc2n[nH]c3c2CN(C(=O)N[C@H](CN(C)C)c2ccccc2)C3(C)C)cc1]

or a pharmaceutically acceptable salt, solvate, hydrate, polymorph, co-crystal, tautomer, stereoisomer, isotopically labeled derivative, or prodrug thereof, an immunotherapeutic agent, and at least one of cisplatin and etoposide. In certain embodiments, the method comprises administering

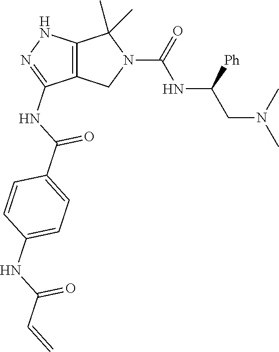 [SMILES: C=CC(=O)Nc1ccc(C(=O)Nc2n[nH]c3c2CN(C(=O)N[C@H](CN(C)C)c2ccccc2)C3(C)C)cc1]

or a pharmaceutically acceptable salt, solvate, hydrate, polymorph, co-crystal, tautomer, stereoisomer, isotopically labeled derivative, or prodrug thereof, an activator of adaptive immune response, and at least one of cisplatin and etoposide. In certain embodiments, the method comprises administering

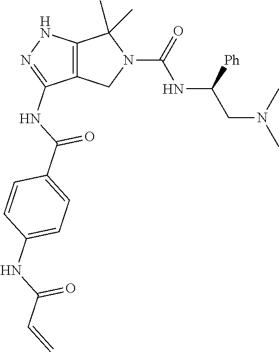 [SMILES: C=CC(=O)Nc1ccc(C(=O)Nc2n[nH]c3c2CN(C(=O)N[C@H](CN(C)C)c2ccccc2)C3(C)C)cc1]

or a pharmaceutically acceptable salt, solvate, hydrate, polymorph, co-crystal, tautomer, stereoisomer, isotopically labeled derivative, or prodrug thereof, an immune checkpoint inhibitor, and at least one of cisplatin and etoposide. In certain embodiments, the method comprises administering

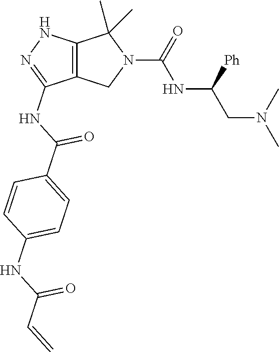 [SMILES: C=CC(=O)Nc1ccc(C(=O)Nc2n[nH]c3c2CN(C(=O)N[C@H](CN(C)C)c2ccccc2)C3(C)C)cc1]

or a pharmaceutically acceptable salt, solvate, hydrate, polymorph, co-crystal, tautomer, stereoisomer, isotopically labeled derivative, or prodrug thereof, an inhibitor of PD-1, PD-L1, or CTLA-4, and at least one of cisplatin and etoposide. In certain embodiments, the method comprises administering

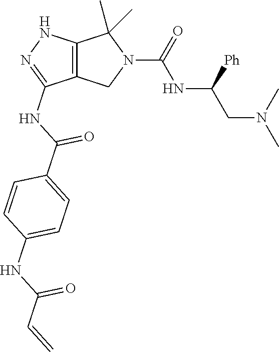 [SMILES: C=CC(=O)Nc1ccc(C(=O)Nc2n[nH]c3c2CN(C(=O)N[C@H](CN(C)C)c2ccccc2)C3(C)C)cc1]

or a pharmaceutically acceptable salt, solvate, hydrate, polymorph, co-crystal, tautomer, stereoisomer, isotopically labeled derivative, or prodrug thereof, an inhibitor of PD-1, and at least one of cisplatin and etoposide. In certain embodiments, the method comprises administering

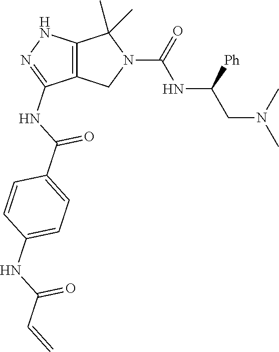 [SMILES: C=CC(=O)Nc1ccc(C(=O)Nc2n[nH]c3c2CN(C(=O)N[C@H](CN(C)C)c2ccccc2)C3(C)C)cc1]

or a pharmaceutically acceptable salt, solvate, hydrate, polymorph, co-crystal, tautomer, stereoisomer, isotopically labeled derivative, or prodrug thereof, an anti-PD-1 antibody, at least one of cisplatin and etoposide. In certain embodiments, the method comprises administering

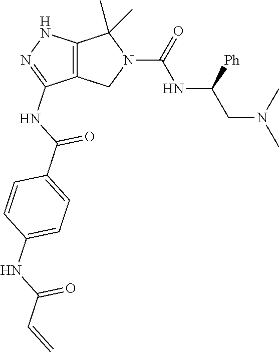 [SMILES: C=CC(=O)Nc1ccc(C(=O)Nc2n[nH]c3c2CN(C(=O)N[C@H](CN(C)C)c2ccccc2)C3(C)C)cc1]

or a pharmaceutically acceptable salt, solvate, hydrate, polymorph, co-crystal, tautomer, stereoisomer, isotopically labeled derivative, or prodrug thereof, ipilimumab, nivolumab, pembrolizumab, spartalizumab, atezolizumab, tislelizumab, tremelimumab, durvalumab, avelumab, or cemiplimab, and at least one of cisplatin and etoposide. In certain embodiments, the method comprises administering

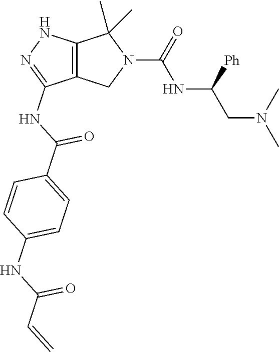 [SMILES: C=CC(=O)Nc1ccc(C(=O)Nc2n[nH]c3c2CN(C(=O)N[C@H](CN(C)C)c2ccccc2)C3(C)C)cc1]

or a pharmaceutically acceptable salt, solvate, hydrate, polymorph, co-crystal, tautomer, stereoisomer, isotopically labeled derivative, or prodrug thereof, nivolumab, pembrolizumab, spartalizumab, tislelizumab, or cemiplimab, and at least one of cisplatin and etoposide.

In certain embodiments, the method comprises administering a compound of any of Formulae (XIV)-(XXIII) and an immunotherapy. In certain embodiments, the method comprises administering a compound of any of Formulae (XIV)-(XXIII) and an immunotherapeutic agent. In certain embodiments, the method comprises administering a compound of any of Formulae (XIV)-(XXIII) and an activator of adaptive immune response. In certain embodiments, the method comprises administering a compound of any of Formulae (XIV)-(XXIII) and an immune checkpoint inhibitor. In certain embodiments, the method comprises administering a compound of any of Formulae (XIV)-(XXIII) and an inhibitor of PD-1, PD-L1, or CTLA-4. In certain embodiments, the method comprises administering a compound of any of Formulae (XIV)-(XXIII) and an inhibitor of PD-1. In certain embodiments, the method comprises administering a compound of any of Formulae (XIV)-(XXIII) and an anti-PD-1 antibody. In certain embodiments, the method comprises administering a compound of any of Formulae (XIV)-(XXIII) and ipilimumab, nivolumab, pembrolizumab, spartalizumab, atezolizumab, tislelizumab, tremelimumab, durvalumab, avelumab, or cemiplimab. In certain embodiments, the method comprises administering a compound of any of Formulae (XIV)-(XXIII) and nivolumab, pembrolizumab, spartalizumab, tislelizumab, or cemiplimab.

In certain embodiments, the method comprises administering a compound of any of Formulae (XIV)-(XXIII), an immunotherapy, and one or more chemotherapeutic agent. In certain embodiments, the method comprises administering a compound of any of Formulae (XIV)-(XXIII), an immunotherapeutic agent, and one or more chemotherapeutic agent. In certain embodiments, the method comprises administering a compound of any of Formulae (XIV)-(XXIII), an activator of adaptive immune response, and one or more chemotherapeutic agent. In certain embodiments, the method comprises administering a compound of any of Formulae (XIV)-(XXIII), an immune checkpoint inhibitor, and one or more chemotherapeutic agent. In certain embodiments, the method comprises administering a compound of any of Formulae (XIV)-(XXIII), an inhibitor of PD-1, PD-L1, or CTLA-4, and one or more chemotherapeutic agent. In certain embodiments, the method comprises administering a compound of any of Formulae (XIV)-(XXIII), an inhibitor of PD-1, and one or more chemotherapeutic agent. In certain embodiments, the method comprises administering a compound of any of Formulae (XIV)-(XXIII), an anti-PD-1 antibody, and one or more chemotherapeutic agent. In certain embodiments, the method comprises administering a compound of any of Formulae (XIV)-(XXIII), ipilimumab, nivolumab, pembrolizumab, spartalizumab, atezolizumab, tislelizumab, tremelimumab, durvalumab, avelumab, or cemiplimab, and one or more chemotherapeutic agent. In certain embodiments, the method comprises administering a compound of any of Formulae (XIV)-(XXIII), nivolumab, pembrolizumab, spartalizumab, tislelizumab, or cemiplimab, and one or more chemotherapeutic agent.

### Pharmaceutical Compositions, Kits, and Administration

One aspect of the present disclosure relates to pharmaceutical compositions that comprise a CDK7 inhibitor and an immunotherapeutic agent, and optionally a pharmaceutically acceptable excipient. The pharmaceutical compositions described herein may be useful in treating and/or preventing cancer in a subject in need thereof, such as cancers that are resistant to or are at risk of becoming resistant to a CDK7 inhibitor and/or an immunotherapeutic agent. The pharmaceutical compositions described herein may also be useful in reducing, delaying, and/or preventing in a subject in need thereof, the resistance of a cancer to treatment with a CDK7 inhibitor and/or an immunotherapeutic agent. The pharmaceutical compositions described herein may further be useful in inhibiting the proliferation of a cell, and/or reducing, delaying, and/or preventing the resistance of a cell to a CDK7 inhibitor and/or an immunotherapeutic agent. The pharmaceutical compositions described herein are expected to be synergistic in treating and/or preventing cancer in the subject; in reducing, delaying, and/or preventing the resistance of cancer in the subject to a CDK7 inhibitor and/or an immunotherapeutic agent; in inhibiting the proliferation of the cell, and/or reducing, delaying, and/or preventing the resistance of the cell to a CDK7 inhibitor and/or an immunotherapeutic agent, compared to the CDK7 inhibitor and/or the immunotherapeutic agent alone.

A pharmaceutical composition described herein comprises a CDK7 inhibitor. In certain embodiments, the CDK7 inhibitor is any CDK7 inhibitor as described herein. In certain embodiments, the CDK inhibitor is compound of Formula (I). In certain embodiments, the CDK inhibitor is compound of Formula (I-a). In certain embodiments, the CDK inhibitor is compound of Formula (I-b). In certain embodiments, the CDK inhibitor is compound of Formula (III). In certain embodiments, the CDK inhibitor is compound of Formula (IV-b). In certain embodiments, the CDK inhibitor is compound of Formula (V-d). In certain embodiments, the CDK inhibitor is compound of Formula (VI-c). In certain embodiments, the CDK inhibitor is compound of Formula (VII-c). In certain embodiments, the CDK inhibitor is compound of Formula (VIII-c). In certain embodiments, the CDK inhibitor is compound of Formula (IX-c). In certain embodiments, the CDK inhibitor is compound of Formula (X-c). In certain embodiments, the CDK inhibitor is compound of Formula (XI-c). In certain embodiments, the CDK inhibitor is compound of Formula (XII-c). In certain embodiments, the CDK inhibitor is compound of Formula (XIII-c). In certain embodiments, the CDK inhibitor is compound of Formula (XIV). In certain embodiments, the CDK inhibitor is compound of Formula (XV). In certain embodiments, the CDK inhibitor is compound of Formula (XVI). In certain embodiments, the CDK inhibitor is compound of Formula (XVII). In certain embodiments, the CDK inhibitor is compound of Formula (XVIII). In certain embodiments, the CDK inhibitor is compound of Formula (XIX). In certain embodiments, the CDK inhibitor is compound of Formula (XX). In certain embodiments, the CDK inhibitor is compound of Formula (XXI). In certain embodiments, the CDK inhibitor is compound of Formula (XXII). In certain embodiments, the CDK inhibitor is compound of Formula (XXIII).

A pharmaceutical composition described herein further comprises an immunotherapeutic agent. In certain embodiments, the immunotherapeutic agent is any immunotherapeutic agent as described herein. In certain embodiments, the immunotherapeutic agent is an activator of adaptive immune response. In certain embodiments, the immunotherapeutic agent is an immune checkpoint inhibitor. In certain embodiments, the immunotherapeutic agent is an inhibitor of PD-1, PD-L1, or CTLA-4. In certain embodiments, the immunotherapeutic agent is an inhibitor of PD-1. In certain embodiments, the immunotherapeutic agent is an anti-PD-1 antibody. In certain embodiments, the immunotherapeutic agent is ipilimumab, nivolumab, pembrolizumab, spartalizumab, atezolizumab, tislelizumab, tremelimumab, durvalumab, avelumab, or cemiplimab. In certain embodiments, the immunotherapeutic agent is nivolumab, pembrolizumab, spartalizumab, tislelizumab, or cemiplimab.

A pharmaceutical composition described herein may further comprise one or more chemotherapeutic agents. In certain embodiments, the chemotherapeutic agent is any chemotherapeutic agent as described herein. In certain embodiments, the chemotherapeutic agent is a platinum-containing compound. In certain embodiments, the chemotherapeutic agent is cisplatin. In certain embodiments, the chemotherapeutic agent is an epipodophyllin. In certain embodiments, the chemotherapeutic agent is etoposide. In certain embodiments, the pharmaceutical composition comprises two chemotherapeutic agents. In certain embodiments, the pharmaceutical composition comprises a platinum-containing compound and an epipodophyllin. In certain embodiments, the pharmaceutical composition comprises cisplatin and etoposide.

A pharmaceutical composition described herein may further comprise one or more targeted agents. In certain embodiments, the targeted agent is any targeted agent as described herein.

In certain embodiments, the CDK7 inhibitor and the immunotherapeutic agent are provided in an effective amount in the pharmaceutical composition. In certain embodiments, the effective amount is a therapeutically effective amount. In certain embodiments, a therapeutically effective amount is an amount effective for treating a cancer in a subject in need thereof. In certain embodiments, therapeutically effective amount is an amount effective for reducing, delaying, and/or preventing in a subject in need thereof the resistance of a cancer to a CDK7 inhibitor and/or immunotherapeutic agent. In certain embodiments, the effective amount is a prophylactically effective amount (e.g., amount effective for preventing a cancer in a subject in need thereof).

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile. In certain embodiments, the subject is with a cancer. In certain embodiments, the subject is with a cancer and has failed therapy of the cancer with a CDK7 inhibitor alone. In certain embodiments, the subject is with a cancer and has failed therapy of the cancer with an immunotherapeutic agent alone.

In certain embodiments, the cell is in vitro. In certain embodiments, the cell is in vivo. In certain embodiments, the cell is a cell of a tissue or biological sample. In certain embodiments, the cell is a cancer cell.

Pharmaceutical compositions described herein can be prepared by any method known in the art of pharmacology. In general, such preparatory methods include bringing the CDK7 inhibitors and/or immunotherapeutic agents described herein (i.e., the “active ingredients”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj© 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a CDK7 inhibitor and/or immunotherapeutic agent described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the CDK7 inhibitor and/or immunotherapeutic agent in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

The CDK7 inhibitors and/or immunotherapeutic agents provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The CDK7 inhibitors, immunotherapeutic agents, and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the CDK7 inhibitors, immunotherapeutic agents, and pharmaceutical compositions described herein are suitable for topical administration to the eye of a subject.

The exact amount (e.g., combined amount) of a CDK7 inhibitor and an immunotherapeutic agent required to achieve an effective amount will vary from subject to subject, depending, for example, on species, age, and general condition of a subject, severity of the side effects or disorder, identity of the particular CDK7 inhibitor, identity of the particular immunotherapeutic agent, mode of administration, and the like. An effective amount may be included in a single dose (e.g., single oral dose) or multiple doses (e.g., multiple oral doses). Each dose is a combination of the CDK7 inhibitor and the immunotherapeutic agent. For each dose, the CDK7 inhibitor and the immunotherapeutic agent may be independently administered at the same time or administered separately at different times in any order. In certain embodiments, the duration between an administration of the CDK7 inhibitor and an administration of the immunotherapeutic agent is about one hour, about two hours, about six hours, about twelve hours, about one day, about two days, about four days, or about one week, wherein the administration of the CDK7 inhibitor and the administration of the immunotherapeutic agent are consecutive administrations. The CDK7 inhibitor in each dose may be independently administered at the same time or administered separately at different times. The immunotherapeutic agent in each dose may also be independently administered at the same time or administered separately at different times. For example, in the following administrations: the immunotherapeutic agent in amount A, followed by the CDK7 inhibitor in amount B1, and followed by the CDK7 inhibitor in amount B2, the dose is the immunotherapeutic agent in amount A plus the CDK7 inhibitor in amount (B1+B2). In certain embodiments, when multiple doses (e.g., multiple combinations of the CDK7 inhibitor and the immunotherapeutic agent) are administered to a subject or applied to a biological sample, tissue, or cell, any about two doses of the multiple doses include different or substantially the same amounts of a CDK7 inhibitor and/or immunotherapeutic agent described herein. In certain embodiments, when multiple doses are administered to a subject or applied to a biological sample, tissue, or cell, the frequency of administering the multiple doses to the subject or applying the multiple doses to the biological sample, tissue, or cell is about three doses a day, about two doses a day, about one dose a day, about one dose every other day, about one dose every third day, about one dose every week, about one dose every about two weeks, about one dose every about three weeks, or about one dose every about four weeks. In certain embodiments, the frequency of administering the multiple doses to the subject or applying the multiple doses to the biological sample, tissue, or cell is about one dose per day. In certain embodiments, the frequency of administering the multiple doses to the subject or applying the multiple doses to the biological sample, tissue, or cell is about two doses per day. In certain embodiments, the frequency of administering the multiple doses to the subject or applying the multiple doses to the biological sample, tissue, or cell is about three doses per day. In certain embodiments, when multiple doses are administered to a subject or applied to a biological sample, tissue, or cell, the duration between the first dose and last dose of the multiple doses is about one day, about two days, about four days, about one week, about two weeks, about three weeks, about one month, about two months, about three months, about four months, about six months, about nine months, about one year, about two years, about three years, about four years, about five years, about seven years, about ten years, fifteen years, twenty years, or the lifetime of the subject, tissue, or cell. In certain embodiments, the duration between the first dose and last dose of the multiple doses is about three months, about six months, or about one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, as the combined weight of a CDK7 inhibitor and an immunotherapeutic agent described herein. In certain embodiments, a dose described herein includes independently between 1 mg and 3 mg, inclusive, as the combined weight of a CDK7 inhibitor and an immunotherapeutic agent described herein. In certain embodiments, a dose described herein includes independently between 3 mg and 10 mg, inclusive, as the combined weight of a CDK7 inhibitor and an immunotherapeutic agent described herein. In certain embodiments, a dose described herein includes independently between 10 mg and 30 mg, inclusive, as the combined weight of a CDK7 inhibitor and an immunotherapeutic agent described herein. In certain embodiments, a dose described herein includes independently between 30 mg and 100 mg, inclusive, as the combined weight of a CDK7 inhibitor and an immunotherapeutic agent described herein.

Doses and dose ranges described herein provide guidance for the administration of provided pharmaceutical compositions to an adult (e.g., an adult whose body weight is 70 kg). The amount to be administered to, for example, a child or an adolescent can be determined by a medical practitioner or person skilled in the art and can be lower or the same as that administered to an adult.

The combinations of the CDK7 inhibitor and the immunotherapeutic agent are expected to be synergistic in treating and/or preventing in the subject the cancers, in reducing, delaying, and/or preventing in the subject the resistance of cancers to a CDK7 inhibitor and/or immunotherapeutic agent, in inhibiting the proliferation of the cell, and/or reducing, delaying, and/or preventing the resistance of the cell to a CDK7 inhibitor and/or immunotherapeutic agent, compared to the CDK7 inhibitor alone or the immunotherapeutic agent alone. To result in the same effect in treating and/or preventing in the subject the cancers, in reducing, delaying, and/or preventing in the subject the resistance of cancers to a CDK7 inhibitor and/or immunotherapeutic agent, in inhibiting the proliferation of the cell, and/or reducing, delaying, and/or preventing the resistance of the cell to a CDK7 inhibitor and/or immunotherapeutic agent, a dose of a combination of the CDK7 inhibitor and the immunotherapeutic agent may be lower than (e.g., lower than 0.1%, lower than 1%, lower than 10%, or lower than 30%) a dose of the CDK7 inhibitor alone and lower than a dose of the immunotherapeutic agent alone. To result in the same effect in treating and/or preventing in the subject the cancers, in reducing, delaying, and/or preventing in the subject the resistance of cancers to a CDK7 inhibitor and/or immunotherapeutic agent, in inhibiting the proliferation of the cell, and/or reducing, delaying, and/or preventing the resistance of the cell to a CDK7 inhibitor and/or immunotherapeutic agent, the frequency of multiple doses of a combination of the CDK7 inhibitor and the immunotherapeutic agent may be lower than (e.g., lower than 0.1%, lower than 1%, lower than 10%, or lower than 30%) the frequency of multiple doses of the CDK7 inhibitor alone and lower than a dose of the immunotherapeutic agent alone. To result in the same effect in treating and/or preventing in the subject the cancers, in reducing, delaying, and/or preventing in the subject the resistance of cancers to a CDK7 inhibitor and/or immunotherapeutic agent, in inhibiting the proliferation of the cell, and/or reducing, delaying, and/or preventing the resistance of the cell to a CDK7 inhibitor and/or immunotherapeutic agent, the total amount of multiple doses of a combination of the CDK7 inhibitor and the immunotherapeutic agent may be lower than (e.g., lower than 0.1%, lower than 1%, lower than 10%, or lower than 30%) the total amount of multiple doses of the CDK7 inhibitor alone and lower than a dose of the immunotherapeutic agent alone.

A CDK7 inhibitor, immunotherapeutic agent, or composition, as described herein, can be administered in combination with one or more additional pharmaceutical agents (e.g., therapeutically and/or prophylactically active agents). The CDK7 inhibitor, immunotherapeutic agent, or composition can be administered in combination with additional pharmaceutical agents that improve their activity (e.g., activity (e.g., potency and/or efficacy) in treating a cancer in a subject in need thereof, in preventing a cancer in a subject in need thereof, in reducing, delaying, and/or preventing in a subject in need thereof the resistance of cancers to a CDK7 inhibitor and/or immunotherapeutic agent, in inhibiting the proliferation of a cell, in reducing, delaying, and/or preventing the resistance of a cell to a CDK7 inhibitor and/or immunotherapeutic agent), improve bioavailability, improve safety, reduce drug resistance, reduce and/or modify metabolism, inhibit excretion, and/or modify distribution in a subject, biological sample, tissue, or cell. It will also be appreciated that the therapy employed may achieve a desired effect for the same disorder, and/or it may achieve different effects. In certain embodiments, a pharmaceutical composition described herein including (1) a CDK7 inhibitor and an immunotherapeutic agent described herein, and (2) an additional pharmaceutical agent shows a synergistic effect, compared with a pharmaceutical composition including one of (1) and (2), but not both (1) and (2).

The CDK7 inhibitor, immunotherapeutic agent, or composition can be independently administered concurrently with, prior to, or subsequent to one or more additional pharmaceutical agents. In certain embodiments, the additional pharmaceutical agents and the CDK7 inhibitor are not the same, and the additional pharmaceutical agents and the immunotherapeutic agent are not the same. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, inflammatory disease, autoimmune disease, genetic disease, hematological disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the CDK7 inhibitor, immunotherapeutic agent, or composition described herein at the same time or administered separately at different times. The particular combination to employ in a regimen will take into account compatibility of the CDK7 inhibitor and/or immunotherapeutic agent described herein with the additional pharmaceutical agent(s), and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, anti-inflammatory agents, immunosuppressants, pain-relieving agents, and a combination thereof. In certain embodiments, the additional pharmaceutical agent is an anti-proliferative agent (e.g., anti-cancer agent, cytotoxic agent). In certain embodiments, the additional pharmaceutical agent is abiraterone acetate (e.g., ZYTIGA), ABVD, ABVE, ABVE-PC, AC, AC-T, ADE, ado-trastuzumab emtansine (e.g., KADCYLA), afatinib dimaleate (e.g., GILOTRIF), aldesleukin (e.g., PROLEUKIN), alemtuzumab (e.g., CAMPATH), anastrozole (e.g., ARIMIDEX), arsenic trioxide (e.g., TRISENOX), asparaginase Erwinia chrysanthemi (e.g., ERWINAZE), axitinib (e.g., INLYTA), azacitidine (e.g., MYLOSAR, VIDAZA), BEACOPP, belinostat (e.g., BELEODAQ), bendamustine hydrochloride (e.g., TREANDA), BEP, bevacizumab (e.g., AVASTIN), bicalutamide (e.g., CASODEX), bleomycin (e.g., BLENOXANE), blinatumomab (e.g., BLINCYTO), bortezomib (e.g., VELCADE), bosutinib (e.g., BOSULIF), brentuximab vedotin (e.g., ADCETRIS), busulfan (e.g., BUSULFEX, MYLERAN), cabazitaxel (e.g., JEVTANA), cabozantinib-s-malate (e.g., COMETRIQ), CAF, capecitabine (e.g., XELODA), CAPOX, carboplatin (e.g., PARAPLAT, PARAPLATIN), carboplatin-taxol, carfilzomib (e.g., KYPROLIS), carmustine (e.g., BECENUM, BICNU, CARMUBRIS), carmustine implant (e.g., GLIADEL WAFER, GLIADEL), ceritinib (e.g., ZYKADIA), cetuximab (e.g., ERBITUX), chlorambucil (e.g., AMBOCHLORIN, AMBOCLORIN, LEUKERAN, LINFOLIZIN), chlorambucil-prednisone, CHOP, cisplatin (e.g., PLATINOL, PLATINOL-AQ), clofarabine (e.g., CLOFAREX, CLOLAR), CMF, COPP, COPP-ABV, crizotinib (e.g., XALKORI), CVP, cyclophosphamide (e.g., CLAFEN, CYTOXAN, NEOSAR), cytarabine (e.g., CYTOSAR-U, TARABINE PFS), dabrafenib (e.g., TAFINLAR), dacarbazine (e.g., DTIC-DOME), dactinomycin (e.g., COSMEGEN), dasatinib (e.g., SPRYCEL), daunorubicin hydrochloride (e.g., CERUBIDINE), decitabine (e.g., DACOGEN), degarelix, denileukin diftitox (e.g., ONTAK), denosumab (e.g., PROLIA, XGEVA), Dinutuximab (e.g., UNITUXIN), docetaxel (e.g., TAXOTERE), doxorubicin hydrochloride (e.g., ADRIAMYCIN PFS, ADRIAMYCIN RDF), doxorubicin hydrochloride liposome (e.g., DOXIL, DOX-SL, EVACET, LIPODOX), enzalutamide (e.g., XTANDI), epirubicin hydrochloride (e.g., ELLENCE), EPOCH, erlotinib hydrochloride (e.g., TARCEVA), etoposide (e.g., TOPOSAR, VEPESID), etoposide phosphate (e.g., ETOPOPHOS), everolimus (e.g., AFINITOR DISPERZ, AFINITOR), exemestane (e.g., AROMASIN), FEC, fludarabine phosphate (e.g., FLUDARA), fluorouracil (e.g., ADRUCIL, EFUDEX, FLUOROPLEX), FOLFIRI, FOLFIRI-BEVACIZUMAB, FOLFIRI-CETUXIMAB, FOLFIRINOX, FOLFOX, FU-LV, fulvestrant (e.g., FASLODEX), gefitinib (e.g., IRESSA), gemcitabine hydrochloride (e.g., GEMZAR), gemcitabine-cisplatin, gemcitabine-oxaliplatin, goserelin acetate (e.g., ZOLADEX), Hyper-CVAD, ibritumomab tiuxetan (e.g., ZEVALIN), ibrutinib (e.g., IMBRUVICA), ICE, idelalisib (e.g., ZYDELIG), ifosfamide (e.g., CYFOS, IFEX, IFOSFAMIDUM), imatinib mesylate (e.g., GLEEVEC), imiquimod (e.g., ALDARA), ipilimumab (e.g., YERVOY), irinotecan hydrochloride (e.g., CAMPTOSAR), ixabepilone (e.g., IXEMPRA), lanreotide acetate (e.g., SOMATULINE DEPOT), lapatinib ditosylate (e.g., TYKERB), lenalidomide (e.g., REVLIMID), lenvatinib (e.g., LENVIMA), letrozole (e.g., FEMARA), leucovorin calcium (e.g., WELLCOVORIN), leuprolide acetate (e.g., LUPRON DEPOT, LUPRON DEPOT-3 MONTH, LUPRON DEPOT-4 MONTH, LUPRON DEPOT-PED, LUPRON, VIADUR), liposomal cytarabine (e.g., DEPOCYT), lomustine (e.g., CEENU), mechlorethamine hydrochloride (e.g., MUSTARGEN), megestrol acetate (e.g., MEGACE), mercaptopurine (e.g., PURINETHOL, PURIXAN), methotrexate (e.g., ABITREXATE, FOLEX PFS, FOLEX, METHOTREXATE LPF, MEXATE, MEXATE-AQ), mitomycin c (e.g., MITOZYTREX, MUTAMYCIN), mitoxantrone hydrochloride, MOPP, nelarabine (e.g., ARRANON), nilotinib (e.g., TASIGNA), nivolumab (e.g., OPDIVO), obinutuzumab (e.g., GAZYVA), OEPA, ofatumumab (e.g., ARZERRA), OFF, olaparib (e.g., LYNPARZA), omacetaxine mepesuccinate (e.g., SYNRIBO), OPPA, oxaliplatin (e.g., ELOXATIN), paclitaxel (e.g., TAXOL), paclitaxel albumin-stabilized nanoparticle formulation (e.g., ABRAXANE), PAD, palbociclib (e.g., IBRANCE), pamidronate disodium (e.g., AREDIA), panitumumab (e.g., VECTIBIX), panobinostat (e.g., FARYDAK), pazopanib hydrochloride (e.g., VOTRIENT), pegaspargase (e.g., ONCASPAR), peginterferon alfa-2b (e.g., PEG-INTRON), peginterferon alfa-2b (e.g., SYLATRON), pembrolizumab (e.g., KEYTRUDA), pemetrexed disodium (e.g., ALIMTA), pertuzumab (e.g., PERJETA), plerixafor (e.g., MOZOBIL), pomalidomide (e.g., POMALYST), ponatinib hydrochloride (e.g., ICLUSIG), pralatrexate (e.g., FOLOTYN), prednisone, procarbazine hydrochloride (e.g., MATULANE), radium 223 dichloride (e.g., XOFIGO), raloxifene hydrochloride (e.g., EVISTA, KEOXIFENE), ramucirumab (e.g., CYRAMZA), R-CHOP, recombinant HPV bivalent vaccine (e.g., CERVARIX), recombinant human papillomavirus (e.g., HPV) nonavalent vaccine (e.g., GARDASIL 9), recombinant human papillomavirus (e.g., HPV) quadrivalent vaccine (e.g., GARDASIL), recombinant interferon alfa-2b (e.g., INTRON A), regorafenib (e.g., STIVARGA), rituximab (e.g., RITUXAN), romidepsin (e.g., ISTODAX), ruxolitinib phosphate (e.g., JAKAFI), siltuximab (e.g., SYLVANT), sipuleucel-t (e.g., PROVENGE), sorafenib tosylate (e.g., NEXAVAR), STANFORD V, sunitinib malate (e.g., SUTENT), TAC, tamoxifen citrate (e.g., NOLVADEX, NOVALDEX), temozolomide (e.g., METHAZOLASTONE, TEMODAR), temsirolimus (e.g., TORISEL), thalidomide (e.g., SYNOVIR, THALOMID), thiotepa, topotecan hydrochloride (e.g., HYCAMTIN), toremifene (e.g., FARESTON), tositumomab and iodine 1131 tositumomab (e.g., BEXXAR), TPF, trametinib (e.g., MEKINIST), trastuzumab (e.g., HERCEPTIN), VAMP, vandetanib (e.g., CAPRELSA), VEIP, vemurafenib (e.g., ZELBORAF), vinblastine sulfate (e.g., VELBAN, VELSAR), vincristine sulfate (e.g., VINCASAR PFS), vincristine sulfate liposome (e.g., MARQIBO), vinorelbine tartrate (e.g., NAVELBINE), vismodegib (e.g., ERIVEDGE), vorinostat (e.g., ZOLINZA), XELIRI, XELOX, ziv-aflibercept (e.g., ZALTRAP), zoledronic acid (e.g., ZOMETA), or a combination thereof. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors, modulators of protein stability (e.g., proteasome inhibitors), Hsp90 inhibitors, glucocorticoids, all-trans retinoic acids, and other agents that promote differentiation.

Also encompassed by the disclosure are kits (e.g., pharmaceutical packs). The kits provided may comprise a CDK7 inhibitor and an immunotherapeutic agent described herein, or a pharmaceutical composition described herein. The kits may comprise a CDK7 inhibitor and an immunotherapeutic agent in a first container. The kits may comprise a CDK7 inhibitor in a first container and an immunotherapeutic agent in a second container. The kits may comprise a pharmaceutical composition in a first container. In some embodiments, the kits further include a third container comprising a pharmaceutical excipient for dilution or suspension of the CDK7 inhibitor, immunotherapeutic agent, and/or pharmaceutical composition. In some embodiments, the CDK7 inhibitor, immunotherapeutic agent, or pharmaceutical composition provided in the first container, optionally the second container, and optionally the third container are combined to form one unit dosage form. Each of the first container, second container, and third container may independently be a vial, ampule, bottle, syringe, and/or dispenser package, or other suitable container. In certain embodiments, the kits are useful for treating a cancer (e.g., cancer that is resistant to a CDK7 inhibitor and/or immunotherapeutic agent) in a subject in need thereof. In certain embodiments, the kits are useful for preventing a cancer (e.g., cancer that is resistant to a CDK7 inhibitor and/or immunotherapeutic agent) in a subject in need thereof. In certain embodiments, the kits are useful for reducing, delaying, and/or preventing in a subject in need thereof the resistance of a cancer to a CDK7 inhibitor and/or immunotherapeutic agent. In certain embodiments, the kits are useful in inhibiting the proliferation of a cell. In certain embodiments, the kits are useful in reducing, delaying, and/or preventing the resistance of a cell to a CDK7 inhibitor and/or immunotherapeutic agent. In certain embodiments, a kit described herein further includes instructions for using the CDK7 inhibitor and immunotherapeutic agent included in the kit, or for using the pharmaceutical composition included in the kit. A kit described herein may also include information as required by a regulatory agency such as the U.S. Food and Drug Administration (FDA). In certain embodiments, the information included in the kits is prescribing information. In certain embodiments, the kits and instructions provide for treating a cancer (e.g., cancer that is resistant to a CDK7 inhibitor and/or immunotherapeutic agent) in a subject in need thereof. In certain embodiments, the kits and instructions provide for preventing a cancer (e.g., cancer that is resistant to a CDK7 inhibitor and/or immunotherapeutic agent) in a subject in need thereof. In certain embodiments, the kits and instructions provide for reducing, delaying, and/or preventing in a subject in need thereof the resistance of a cancer to a CDK7 inhibitor and/or immunotherapeutic agent. In certain embodiments, the kits and instructions provide for inhibiting the proliferation of a cell. In certain embodiments, the kits and instructions provide for reducing, delaying, and/or preventing the resistance of a cell to a CDK7 inhibitor and/or immunotherapeutic agent. A kit described herein may include one or more additional pharmaceutical agents described herein as a separate composition.

### EXAMPLES

In order that the present disclosure may be more fully understood, the following examples are set forth. The examples described in this application are offered to illustrate the methods, CDK7 inhibitors, immunotherapeutic agents, chemotherapeutic agents, and pharmaceutical compositions provided herein and are not to be construed in any way as limiting their scope.

### YKL-5-124 Specifically Targets CDK7 and Disrupts Cell Cycle Progression Through Inhibition of CDK7 CAK Activity

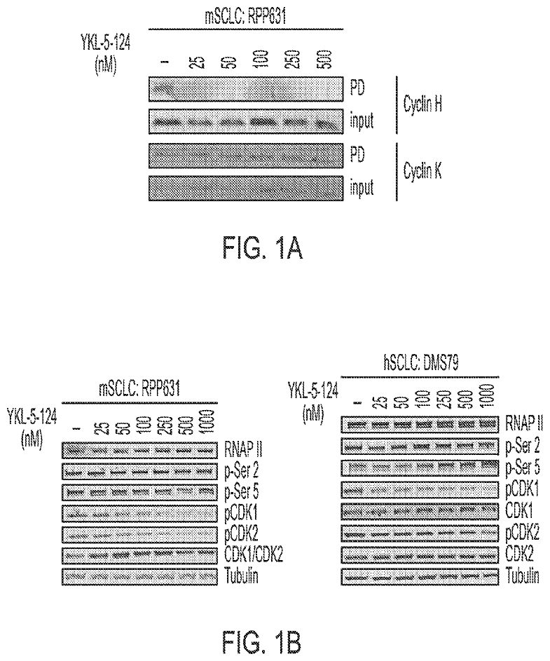

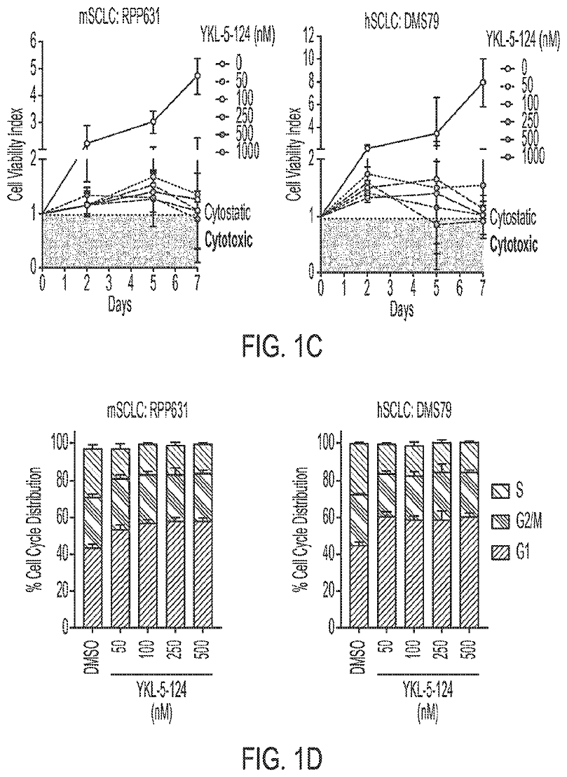

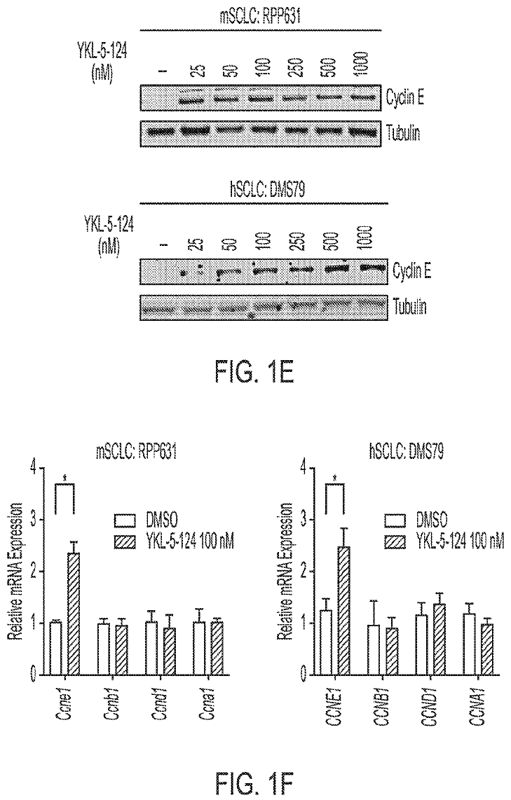

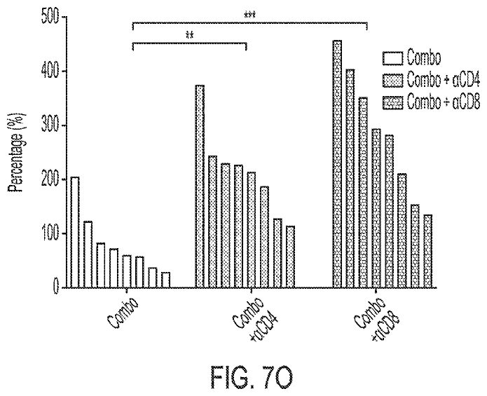

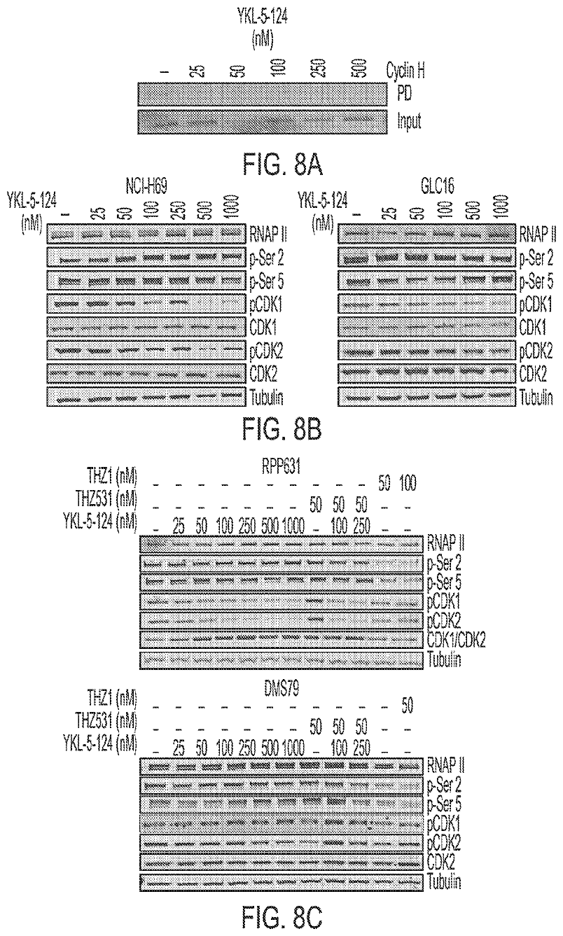

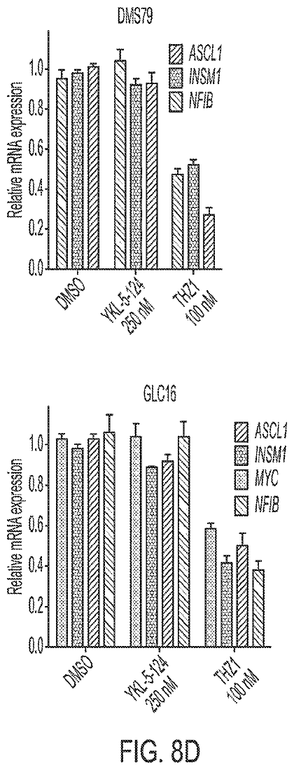

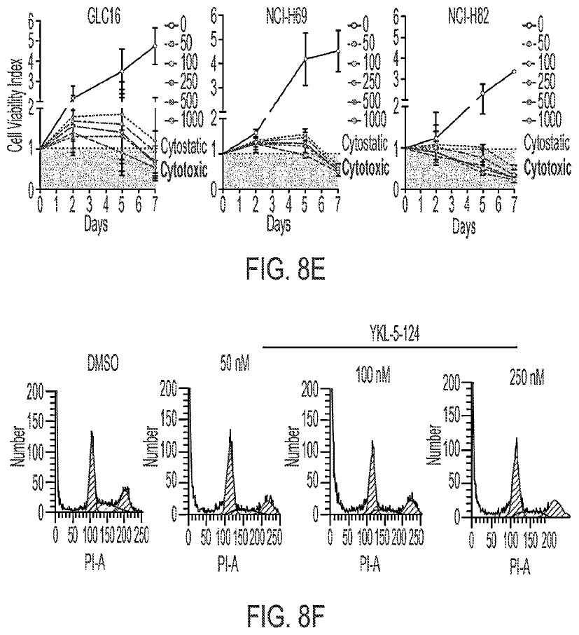

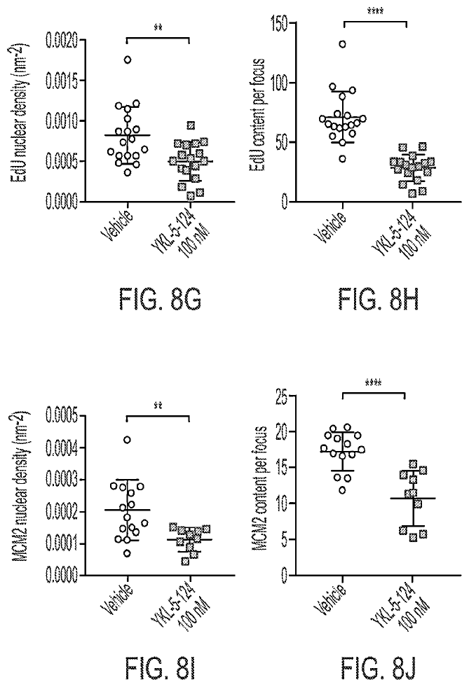

To confirm that YKL-5-124 confers selective engagement of CDK7 over CDK12/13 in SCLC cells, a competitive pulldown assay was performed examining the ability of YKL-5-124 to block the pulldown of CDK7-Cyclin H complexes or Cyclin K, the obligate binding partner of CDK12/13. YKL-5-124 efficiently prevented Cyclin H pulldown, but failed to block pulldown of Cyclin K (FIGS. 1A and 8A). These results confirm selective targeting of CDK7 by YKL-5-124, consistent with our recent findings in other cellular models.

Direct targets of CDK7, CDK1 and CDK2 were next examined. YKL-5-124 robustly inhibited CDK1 and CDK2 T-loop phosphorylation in a representative panel of SCLC lines at concentrations as low as 50 nM (FIGS. 1B and 8B). In contrast, YKL-5-124 treatment had no effect on C-terminal domain (CTD) phosphorylation of RNA Pol II (FIG. 1B), indicating that selective CDK7 inhibition does not inhibit global transcription. Dual inhibition of CDK7 and CDK12/13 is required for inhibition of RNA Pol II CTD phosphorylation by co-treating cells with YKL-5-124 and the CDK12/13 inhibitor THZ531. Concurrent inhibition of CDK7 and CDK12/13 reduced levels of phosphorylated Pol II Ser2 and Ser5 to levels similar to treatment with the CDK7/12/13 inhibitor THZ1 (FIG. 8C). In summary, selective CDK7 inhibition with YKL-5-124 is sufficient to reduce the phosphorylation levels of CDK1 and CDK2 but not that of RNA Pol II CTD. Furthermore, YKL-5-124 treatment had no effect on expression of super enhancer (SE)-associated genes including INSM1, ASCL1, NFIB and MYC, while THZ1 treatment caused a significant reduction in their expression (FIG. 8D).

Whether YKL-5-124 treatment affected cell viability and cell cycle progression was next investigated. Cell viability was measured at different time points (up to 7 days) upon treatment with increasing concentrations of YKL-5-124. A significant reduction in growth rate was observed at all tested concentrations compared to control cells (FIGS. 1C and 8E). YKL-5-124 resulted in reduced growth and a cytostatic effect with a tendency of cytotoxic effects at longer time points (day 7) (FIGS. 1C and 8E). Cell cycle analysis further showed that YKL-5-124 induced a significant accumulation of cells in G1 phase with a corresponding loss of cells in S phase (FIGS. 1D and 8F). The percentage of cells in G2/M phase was not significantly changed at concentrations up to 500 nM. In parallel, an increase of Cyclin E protein and mRNA (CCNE1) levels was detected, supporting the observed accumulation of cells at the G1-S phase checkpoint, whereas expression of Cyclins A (CCNA1), B (CCNB1), and D (CCND1) remained unchanged (FIGS. 1E and 1F).

The data in SCLC supports YKL-5-124 as a selective CDK7 inhibitor that suppresses CDK1 and CDK2 activity and impairs cell growth and cell cycle progression.

### CDK7 Inhibition Impairs DNA Replication and Causes DNA Damage and Micronuclei Formation

The inhibitory effect of YKL-5-124 on CDK1 and CDK2 activity and the consequent G1-S progression defect prompted an address to determine whether CDK7 inhibition affects DNA replication and hexameric minichromosome maintenance 2-7 (MCMs) complex at replication sites, subsequently causing DNA damage and genome instability.

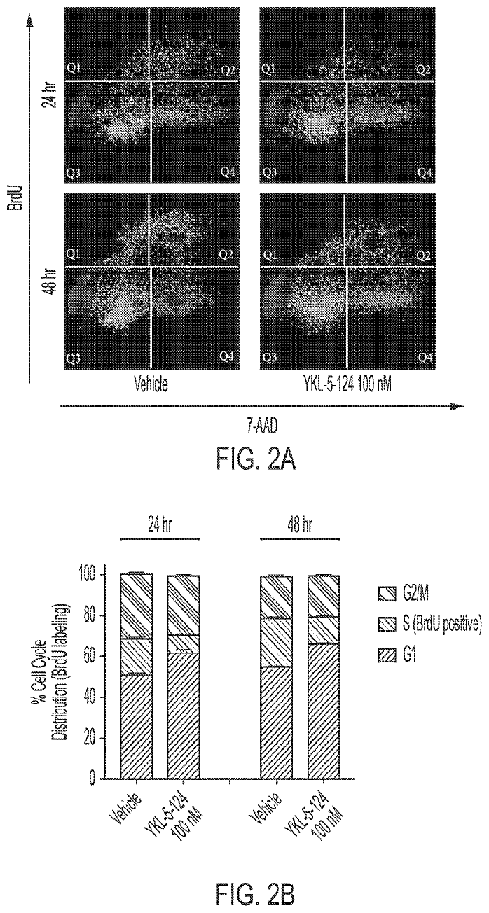

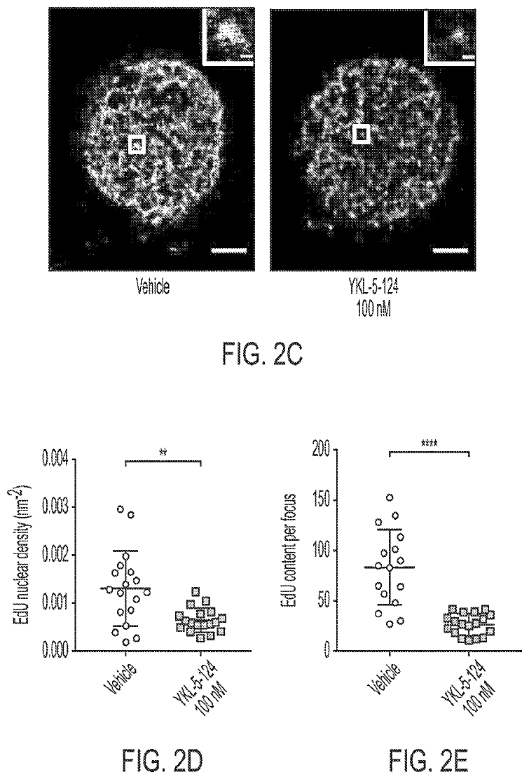

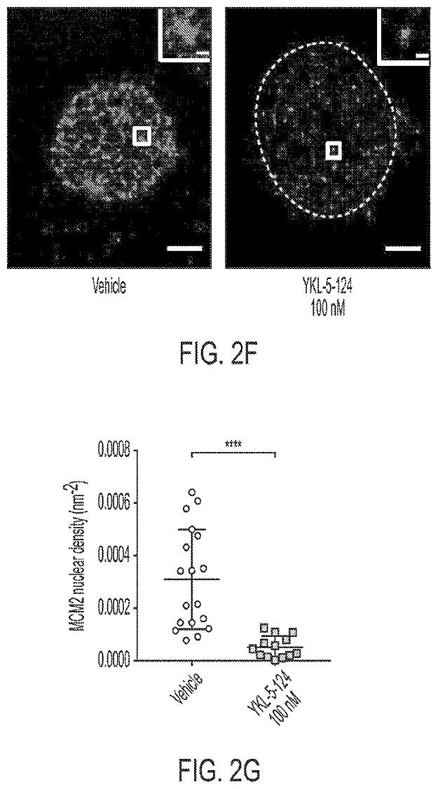

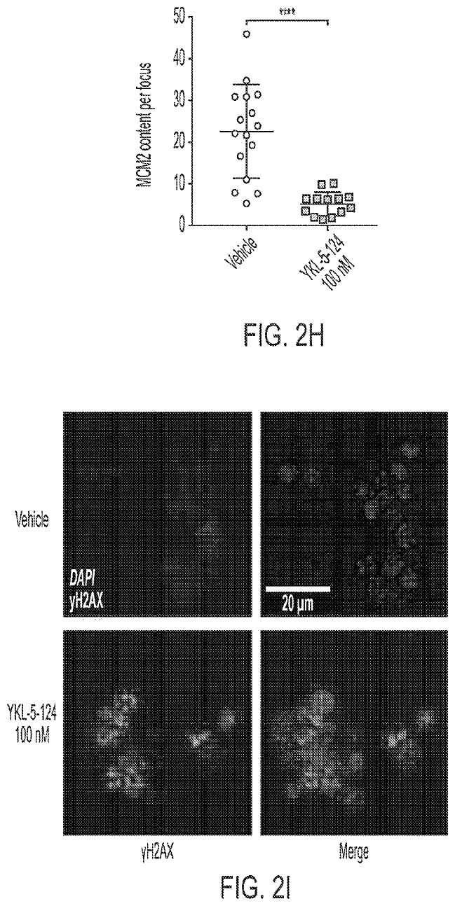

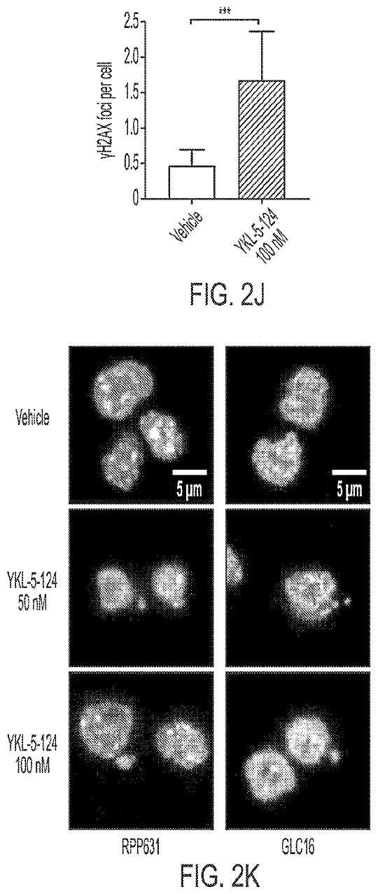

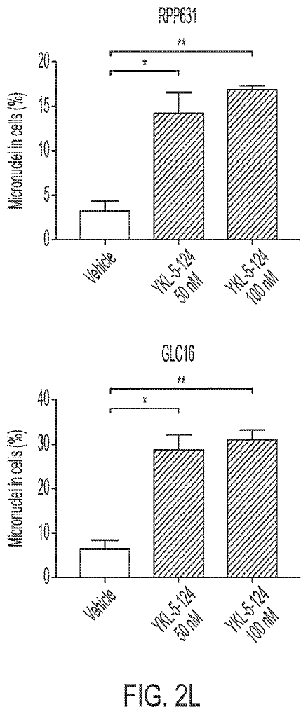

The effect of YKL-5-124 on active DNA replication by measuring BrdU incorporation was first examined. A significant decrease in BrdU-incorporated S-phase cells was revealed (FIGS. 2A and 2B), indicating impaired DNA replication. Experiments were carried out to further characterize whether YKL-5-124 stalls DNA synthesis indicated by EdU incorporation in the nucleus, as well as impairs the loading of MCM2 at individual replication foci. Single-molecule stochastic optical reconstruction microscopy (STORM) of fluorescently labeled EdU and MCM2 were employed. A dramatic decrease in the EdU content in each nucleus as well as in each focus upon YKL-5-124 exposure after 48 and 72 hr was observed (FIGS. 2C-2E, 8G and 8H), implying reduced origin firing events. Similarly, a significant decline of MCM2 content in each nucleus and replication focus was detected (FIGS. 2F-2H, 8I and 8J). Taken together, these results support an inhibitory impact of YKL-5-124 on DNA replication and MCM2 initiation complex assembly.

Reduction of MCM2 at the DNA replication initiation complex has been shown to cause replication deficiency and stress, leading to DNA damage and genomic instability, manifested by elevated levels of γH2AX and increased micronuclei formation. A significant increase of γH2AX foci in YKL-5-124-treated cells after 48 hr (FIGS. 2I and 2J) was observed, suggesting activation of the DNA damage response. Whether CDK7 inhibition can increase micronuclei was then examined. Immunofluorescence images of DAPI-stained nuclei were recorded and quantified (FIG. 2K). Percentages of cells containing micronuclei were considerably increased after YKL-5-124 treatment (FIG. 2L).

### YKL-5-124 Triggers Immune Response Signaling and Induces Pro-Inflammatory Cytokines/Chemokines Production

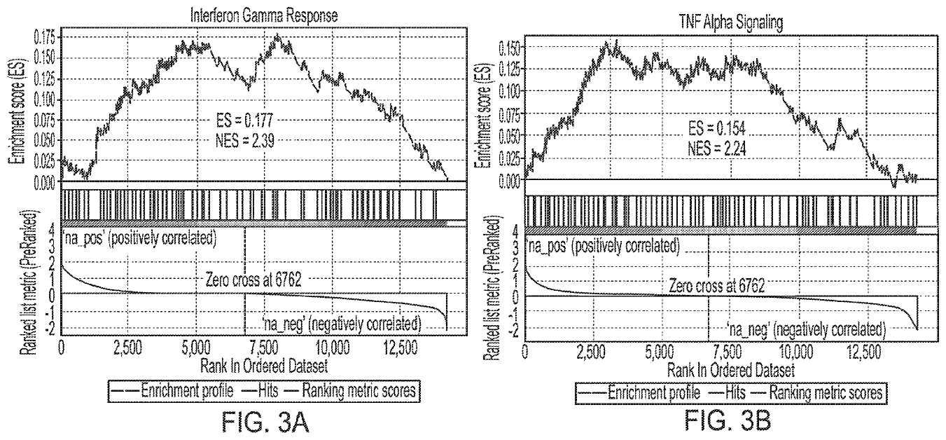

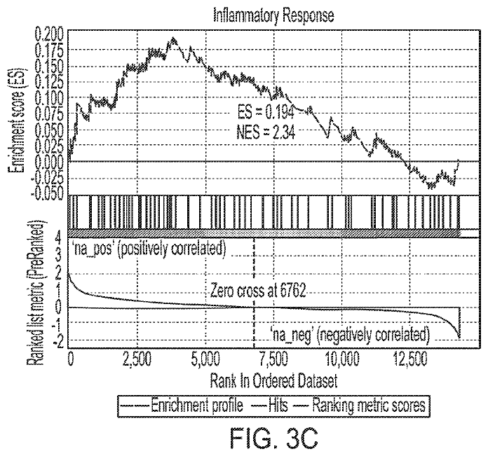

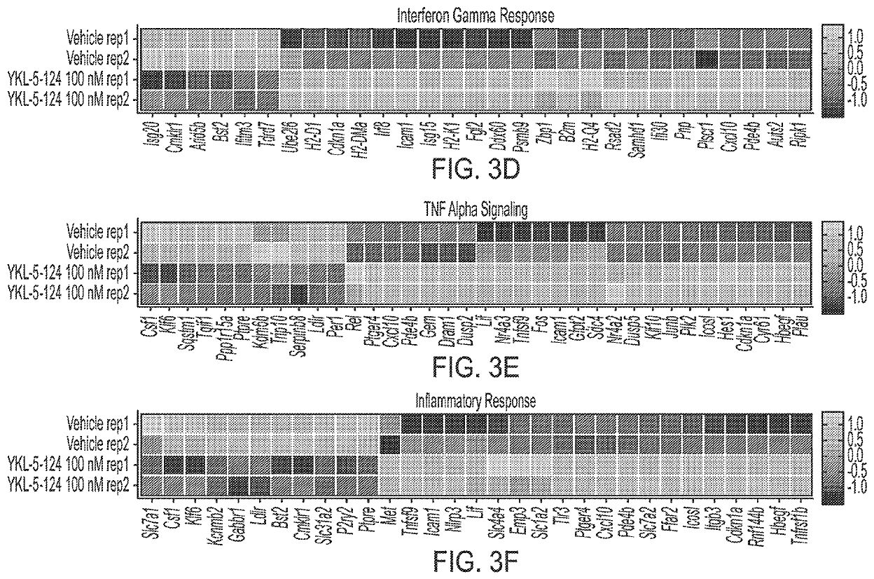

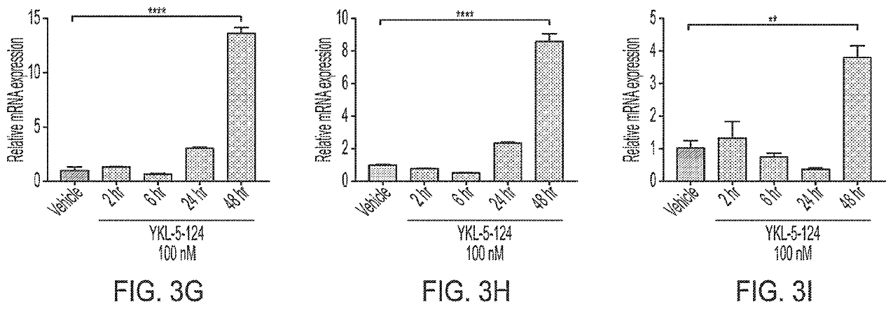

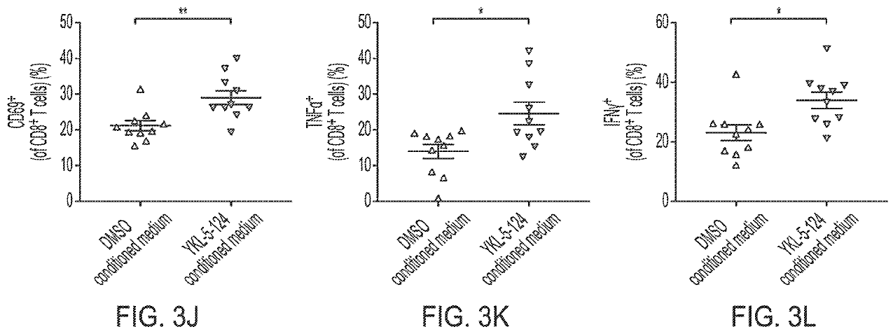

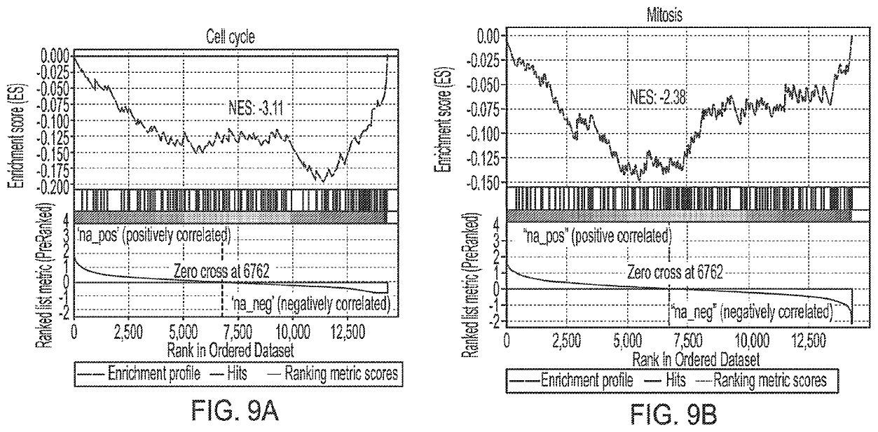

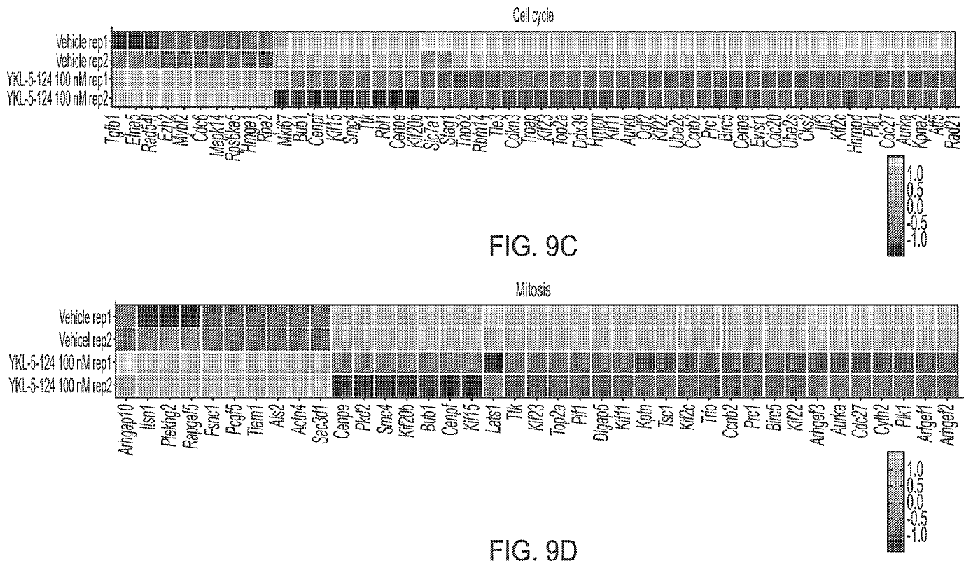

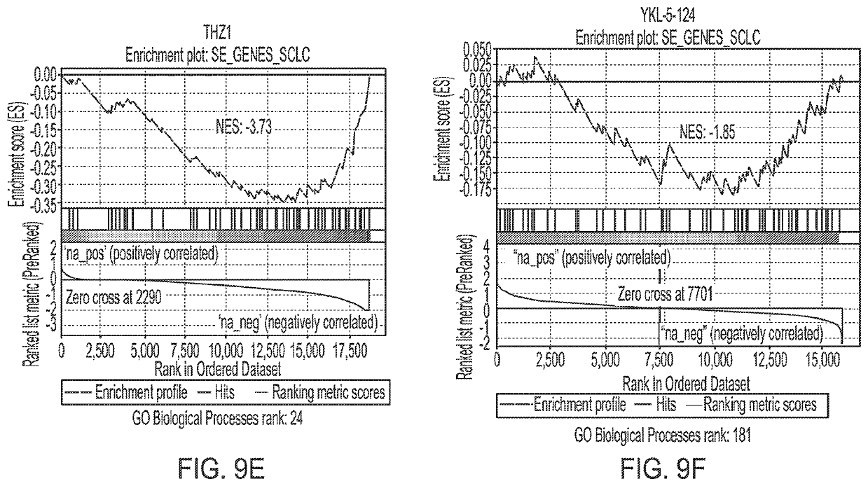

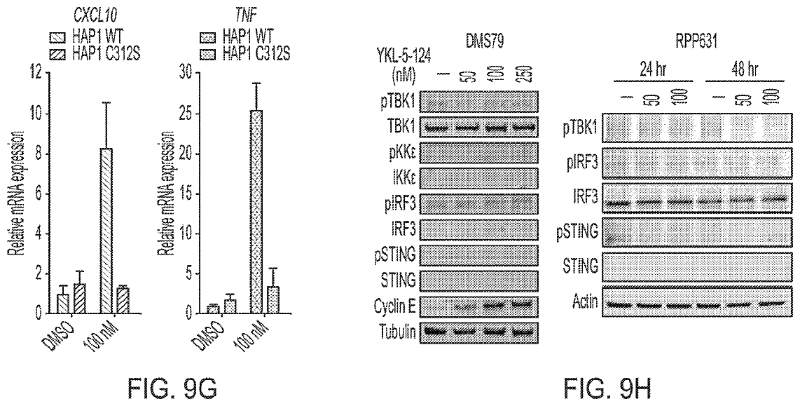

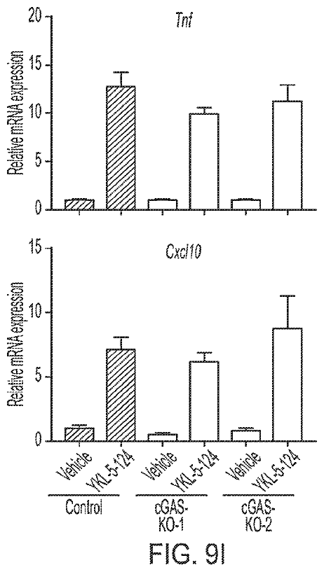

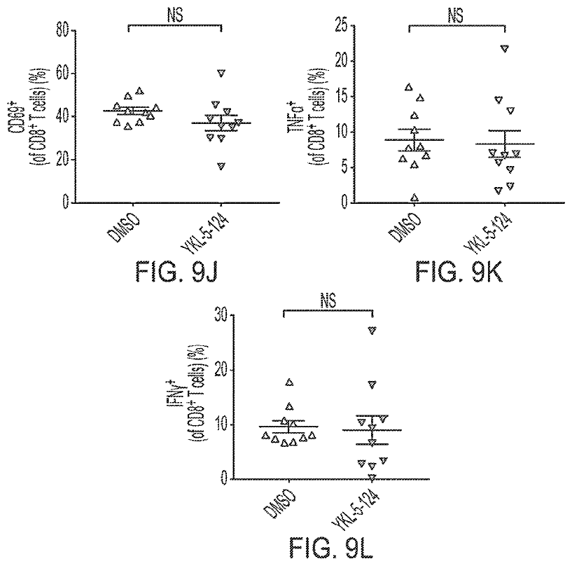

A transcriptomic analysis to comprehensively explore whether CDK7 inhibition affects immune response signaling in vitro was next performed. RPP631 cells were treated with YKL-5-124 for 48 hr and were then harvested for RNA-sequencing. As expected, expression analysis across significantly modulated genes revealed that YKL-5-124 downregulated genes within gene set enrichment analysis (GSEA) terms associated with cell cycle, mitosis and E2F targets (FIGS. 9A-9D). Further analysis confirmed that YKL-5-124 treatment had little effect on expression of SCLC SE-associated genes, in comparison to dual CDK7 and CDK12/13 inhibition upon THZ1 treatment (FIGS. 9E and 9F). Intriguingly, GSEA analysis of the differentially expressed genes revealed three of the top five most positively regulated ‘Hallmarks’ signatures were ‘Interferon gamma response’ (FIG. 3A), ‘Tumor necrosis factor alpha (TNFα) signaling’ (FIG. 3B) and ‘Inflammatory response’ (FIG. 3C).

Heatmaps for the most differentially regulated genes in the top GSEA signatures induced by YKL-5-124 showed an increased expression of numerous central pro-inflammatory cytokines and chemokines, including TNFα pathway components and C—X—C motif chemokine ligand 10 (Cxcl10) (FIGS. 3D-3F). TNFα signaling plays an important role in dendritic cells recruitment, maturation and activation, while CXCL10/CXCL9 are involved in regulating T cell recruitment and activity. These factors secreted in the TME can potentially contribute to an optimal anti-tumor T cell response. The expression levels of Tnf, Cxcl10 and Cxcl9 were then measured upon YKL-5-124 exposure by quantitative reverse transcription polymerase chain reaction (RT-qPCR). YKL-5-124 significantly stimulated tumor cell expression of Tnf (FIG. 3G), Cxcl10 (FIG. 3H) and Cxcl9 (FIG. 3I) after 48 hr. To further confirm the cytokine/chemokine production by YKL-5-124 is mediated through specific inhibition of CDK7, an isogenic HAP1 cell system expressing CDK7 wild-type (WT) or a mutated form of CDK7 (CDK7-Cys312Ser) in which YKL-5-124 cannot bind was used. Consistently, a significant increase in TNF and CXCL10 expression was detected after YKL-5-124 treatment in the WT cells, but not in the C312S mutant cells (FIG. 9G). In addition, the cGAS-STING pathway is one of the most investigated cytosolic DNA sensing mechanism that activates immunity. However, interestingly, the immune stimulatory effect by YKL-5-124 appears to be independent of this pathway (FIGS. 9H and 9I).

Whether this above tumor cell-intrinsic effect by YKL-5-124 could affect T cell activation ex vivo was examined. To address this, the OT-I mouse model in which the OT-I CD8⁺ T cells can recognize ovalbumin peptide residues 257-264 (OVA₂₅₇₋₂₆₄) and become activated was utilized. Mouse SCLC cells were treated with either DMSO or YKL-5-124 for 48 hr. DMSO-conditioned or YKL-5-124-conditioned medium was added to OT-I T cells culture in the presence of OVA₂₅₇₋₂₆₄ peptide. Intriguingly, a significant increase in the percentage of CD69⁺, TNFα⁺ and IFNγ⁺ CD8⁺ T cells was detected in the YKL-5-124-conditioned medium, in comparison to DMSO-conditioned medium group (FIGS. 3J-3L), indicating elevated T cell activity. No significant effect on CD8⁺ T cell activity was observed when OT-I T cells were directly treated with YKL-5-124 (FIGS. 9J-9L). These findings indicate that the activation of CD8⁺ T cells is a tumor intrinsic effect of CDK7 inhibition rather that the drug's direct action on CD8⁺ T cells.

Collectively, these findings demonstrate that CDK7 inhibition activates immune response signaling in SCLC cells leading to secretion of essential pro-inflammatory cytokines/chemokines, which in turn can activate CD8⁺ T cells.

### YKL-5-124 is a Well-Tolerated CDK7 Inhibitor In Vivo and Inhibits SCLC Tumor Growth

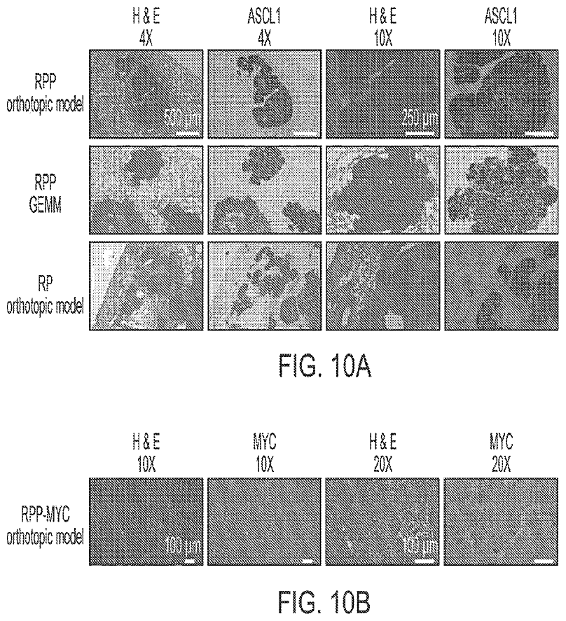

Next, it was determined whether YKL-5-124 inhibits tumor growth and prolong survival in four immunocompetent murine SCLC models, including Rb1^{L/L}p53^{L/L}p130^{L/L} (RPP) genetically engineered mouse models (GEMMs), RPP, Rb1^{-/-}p53^{-/-} (RP) and RPP-MYC orthotopic models (FIGS. 10A and 10B).

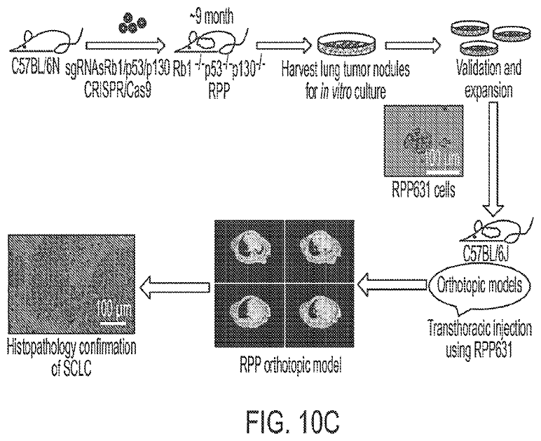

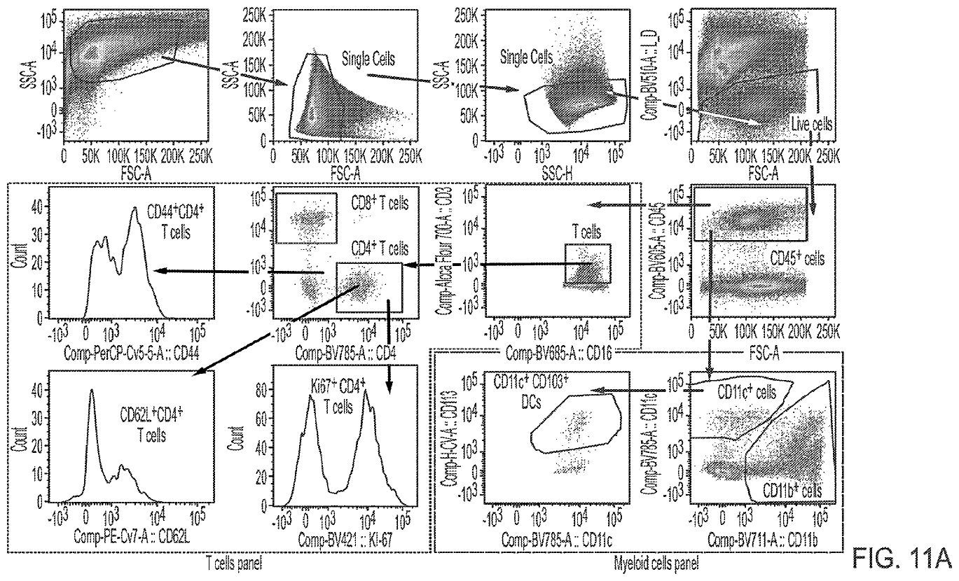

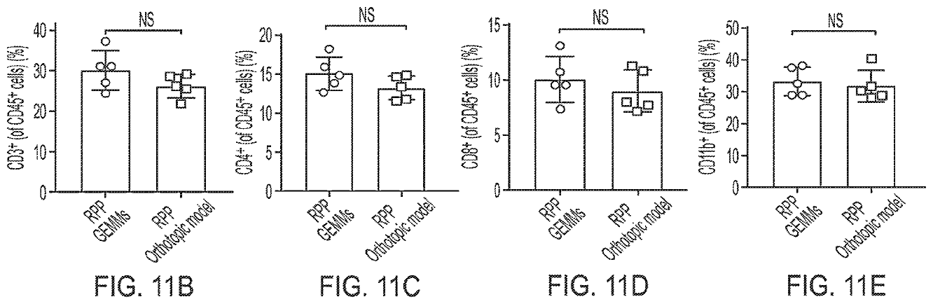

To overcome the long and variable latency (6 to 9 months) of conventional RPP GEMMs, an orthotopic syngeneic SCLC model was established and characterized. This was conducted via transthoracic injection using tumor cells from a CRISPR/Cas9-RPP model of C57BL/6 (B6) background (FIG. 9C). These tumor-bearing mice have a much shorter and consistent latency of 7 to 8 weeks. Furthermore, a highly similar frequency of different immune infiltrating populations (CD3⁺, CD4⁺, CD8⁺ and CD11b⁺) in the TME between the orthotopic model and GEMMs was observed (FIGS. 11A-11E). To this end, using the same strategy, three syngeneic murine models (RPP, RP and RPP-MYC) were successfully generated, which are fast and reliable for oncoimmunology studies for SCLC (FIG. 10).

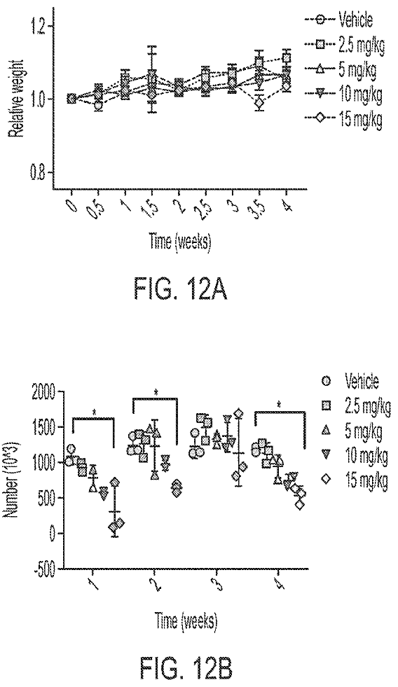

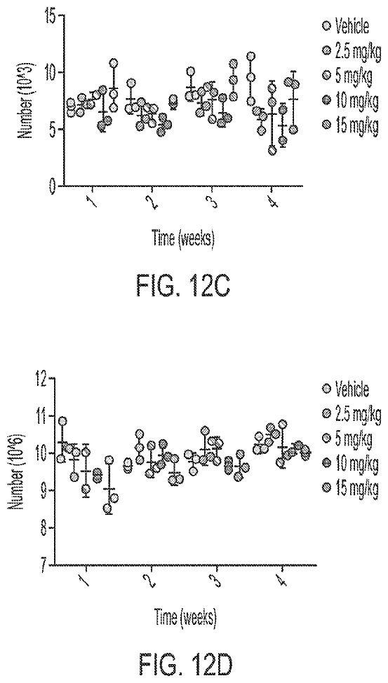

To evaluate the optimal YKL-5-124 dosage with minimum toxicity, a dose-escalating study in B6 mice in which body weight and blood cell counts were monitored and measured was performed (FIGS. 12A-12D). YKL-5-124 was well tolerated at a dose up to 10 mg/kg and caused no significant change in body weight and blood counts. Target engagement of YKL-5-124 in tumor-bearing mice at 10 mg/kg was further confirmed (FIG. 12E).

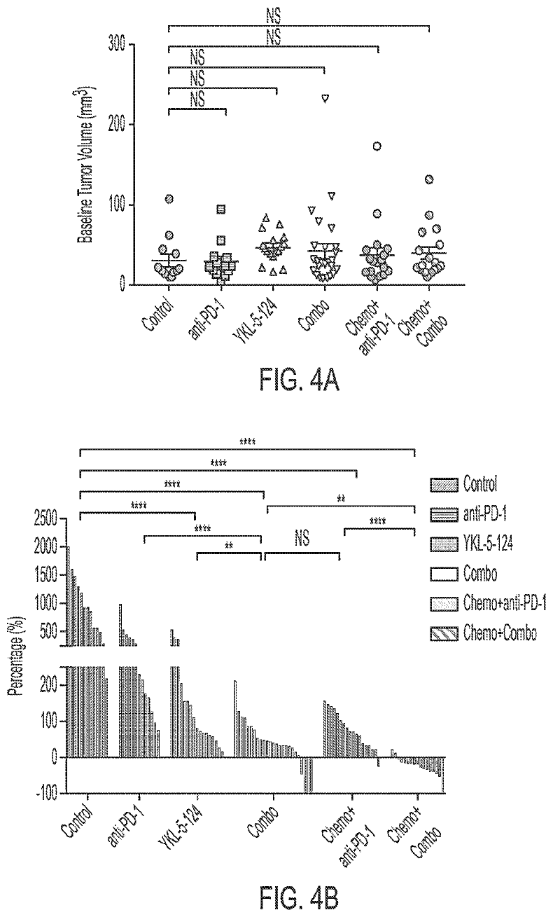

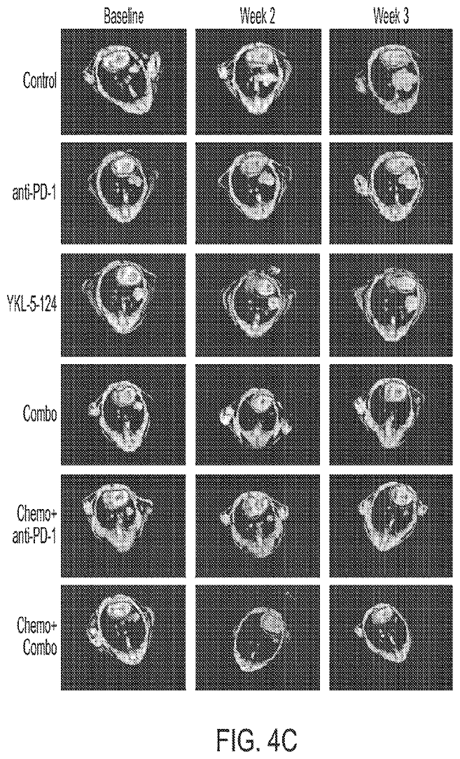

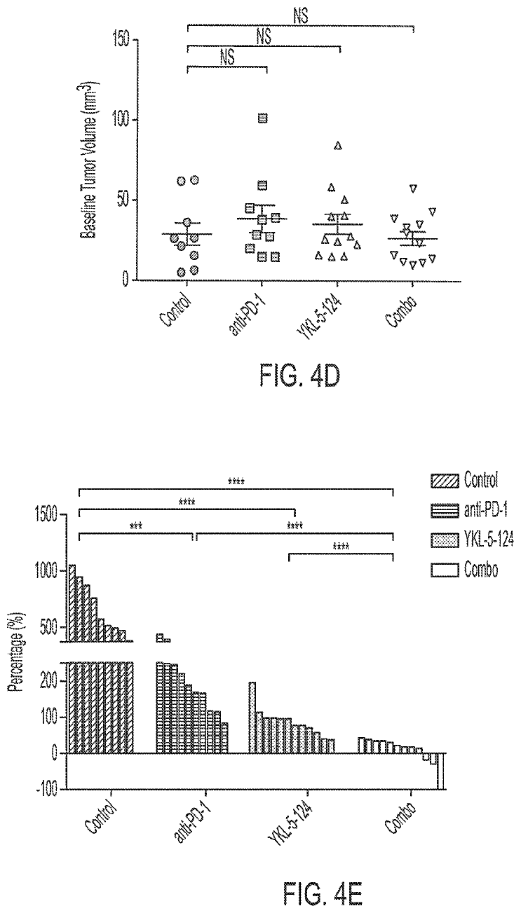

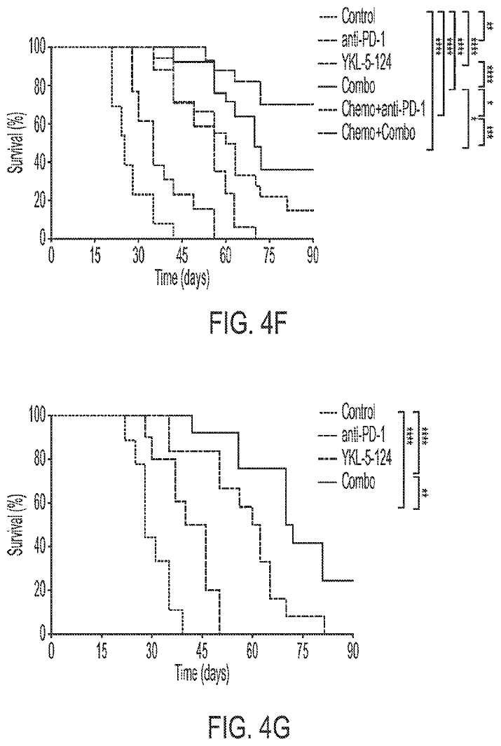

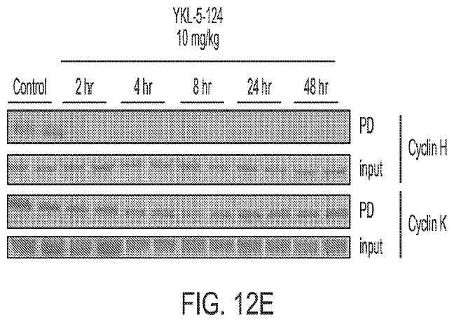

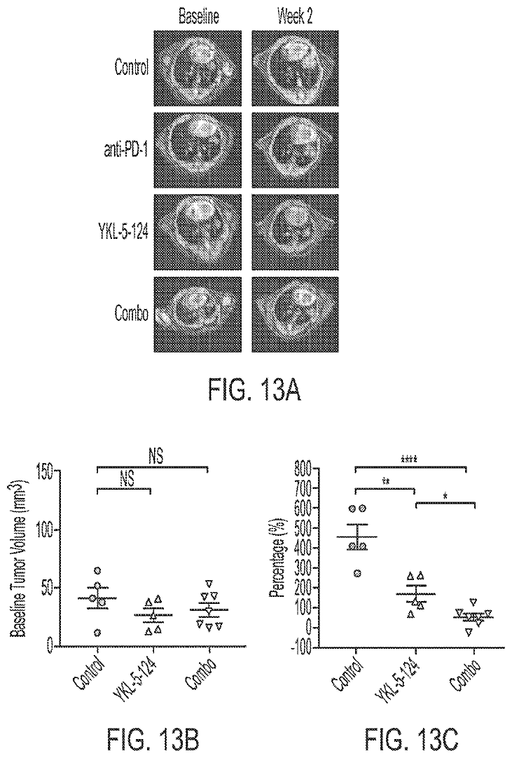

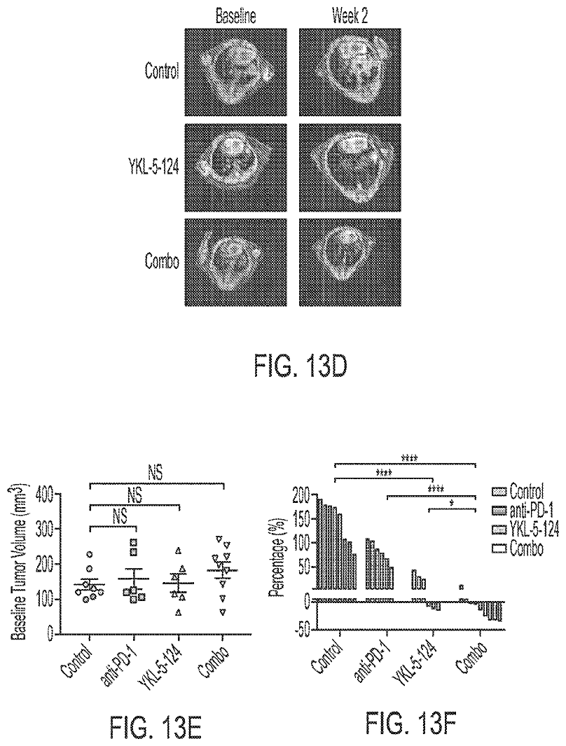

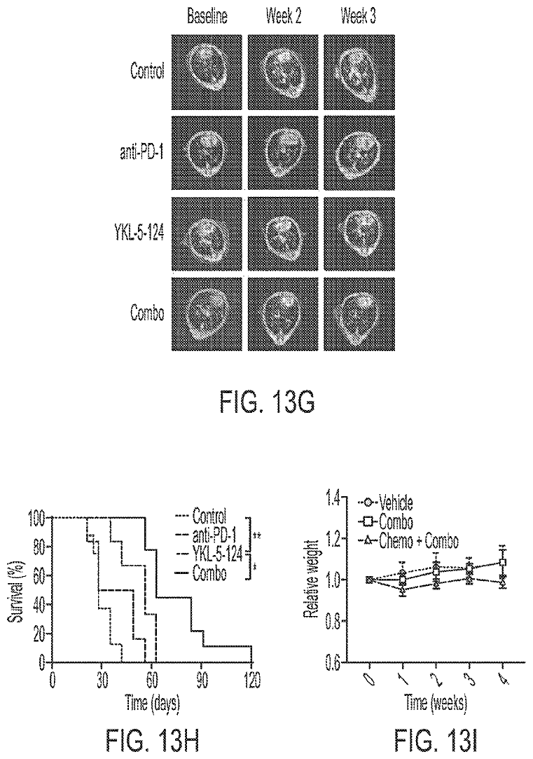

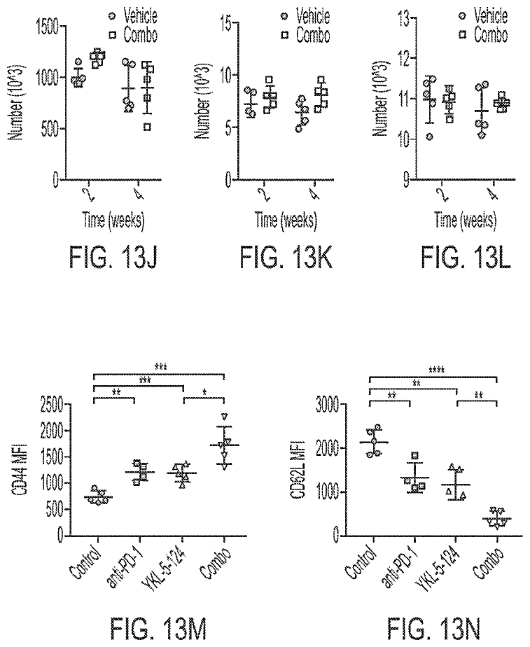

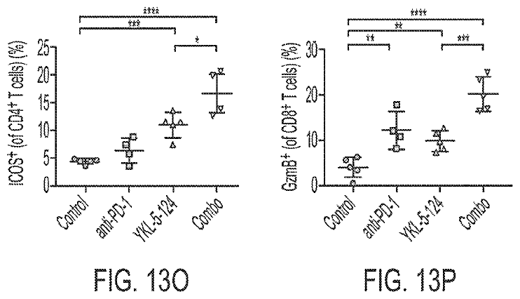

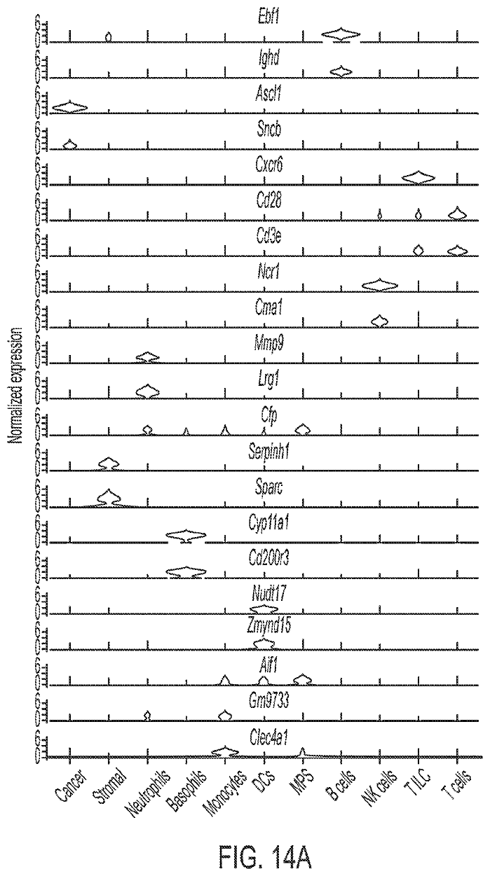

YKL-5-124 also affects the tumor growth in the RPP orthotopic model. Disease development was followed by MRI. Upon confirmation of tumor burden by MRI, mice were randomized to control and YKL-5-124 treatment, respectively (FIG. 4A). All mice in the control group displayed aggressive disease with tumor volumes doubling after a 3-week period (FIGS. 4B and 4C). YKL-5-124-treated mice had significant tumor response at the 2-week and 3-week time points (FIGS. 4B and 4C). The efficacy of YKL-5-124 in the RP and RPP-MYC orthotopic models (FIGS. 4D and 13A-13D). Consistently, YKL-5-124 demonstrated notably delayed tumor growth in the RP and RPP-MYC (FIGS. 4E and 13A-13D). Remarkably, YKL-5-124 significantly prolonged survival, with an added median survival benefit of approximately 30 days in both RPP and RP models (FIGS. 4F and 4G). In contrast, most vehicle-treated mice in the RPP model succumbed to their tumor burden before the 6-week time point, highlighting the aggressive disease course (FIG. 4F).

To further confirm the above observed tumor response, the efficacy of YKL-5-124 in autochthonous RPP GEMMs was evaluated (FIGS. 13E-13H). Consistently, the majority of mice in control group (seven of eight) had doubled their tumor volumes at the 3-week time point, while none of the mice treated with YKL-5-124 had more than 50% increase of tumor burden (FIGS. 13F and 13G). Similarly, YKL-5-124 greatly improved median overall survival from 28 days to 56 days (FIG. 13H).

### YKL-5-124 Enhances Tumor Response to Anti-PD-1 Immunotherapy

The above in vitro findings show a role of CDK7 inhibition in enhancing immune response, thus prompting investigation into whether CDK7 inhibition augments immunotherapy in vivo. As a result, YKL-5-124 was combined with anti-PD-1 to determine if this combination would result in more durable tumor inhibition than each single agent alone. Of note, no toxicity was detected in the body weight and blood cell counts in the combination treatment group (FIGS. 13I-13L).

Compared to the control group, anti-PD-1 alone significantly reduced tumor growth after 3-week treatment in the RPP and RP models (FIGS. 4B and 4E), although to a lesser extent when compared to YKL-5-124, as the added median overall survival benefit by anti-PD-1 was only 10 days in the RPP and 15 days in the RP model (FIGS. 4F and 4G). Strikingly, mice in the combined anti-PD-1+YKL-5-124 treatment regimen (Combo) had the best response across all models tested (FIGS. 4B, 4E, 13C and 13F). 7 out of 25 mice exhibited stable disease (volume increase <30%) including three mice with complete response (CR) in the RPP model (FIG. 4B) and more than half of the mice had stable disease with one CR in the RP model (FIG. 4E). Combining YKL-5-124 and anti-PD-1 dramatically increased the overall survival of tumor-bearing mice in comparison to either YKL-5-124 or anti-PD-1 alone (FIG. 4F). In comparison to control mice, combination treatment led to more than two times increase in survival with an added median survival benefit of 45 days and 43 days in the RPP and RP models, respectively (FIGS. 4F and 4G). These results were also confirmed in a separate cohort of RPP GEMMs. Whereas mice treated with anti-PD-1 alone had slower tumor growth compared to vehicle-treated mice after 3 weeks, the majority of YKL-5-124 plus anti-PD-1 treated mice (seven of nine) had substantially reduced tumor burdens (FIGS. 13F and 13G). Combination treatment was superior to either YKL-5-124 or anti-PD-1 alone in tumor response (FIGS. 13F and 13G) and led to the longest survival (FIG. 13H).

The efficacy of the Combo with standard chemotherapy (cisplatin+etoposide) was evaluated and compared with survival benefit of recently approved chemotherapy+anti-PD-1. Strikingly, the four-drug combination group (Chemo+Combo) had the best efficacy (FIG. 4B) with no toxicity in the body weight (FIG. 13I). All mice had stable disease and roughly half of the 17 mice had >20% reduction in tumor volume after 3-week, whereas there was no significant difference in efficacy between YKL-5-124+anti-PD-1 (Combo, FIG. 4B) and cisplatin+etoposide (Chemo+anti-PD-1, FIG. 4B). The impressive efficacy of Chemo+Combo was directly translated into the longest improved survival, as 12 out of 17 mice in this group survived more than 90 days (FIG. 4F). Of note, mice under Combo treatment appear to live significantly longer than those in the Chemo+anti-PD-1 group (FIG. 4F), demonstrating a better survival benefit in the Combo group.

In summary, YKL-5-124 enhances the response to anti-PD-1 immunotherapy, and combining YKL-5-124 with anti-PD-1 offers significant survival benefit in multiple models. Adding YKL-5-124 and anti-PD-1 to standard chemotherapy further improves tumor response and leads to the longest survival.

YKL-5-124 Provokes a Robust Anti-Tumor Immune Program In Vivo, which is Further Enhanced by Anti-PD-1 Immunotherapy

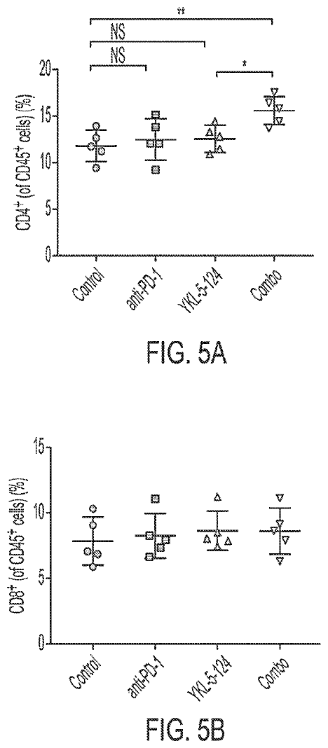

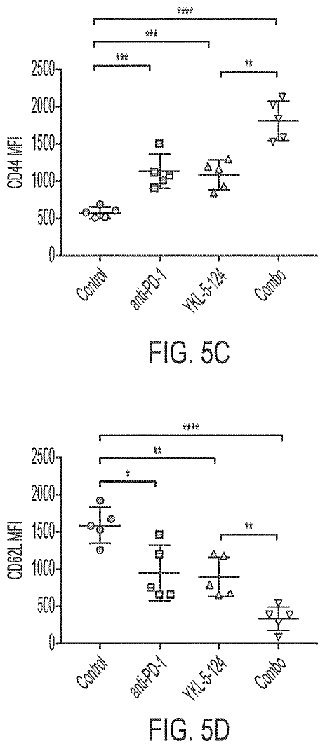

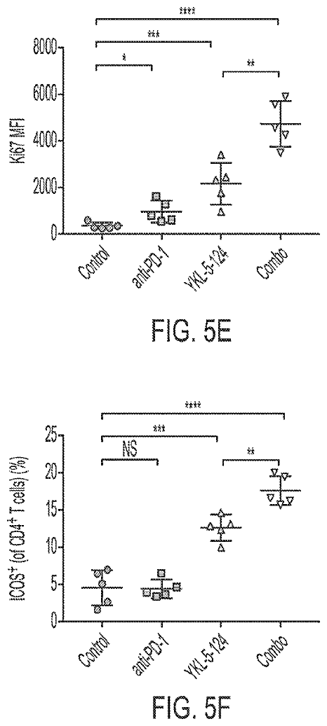

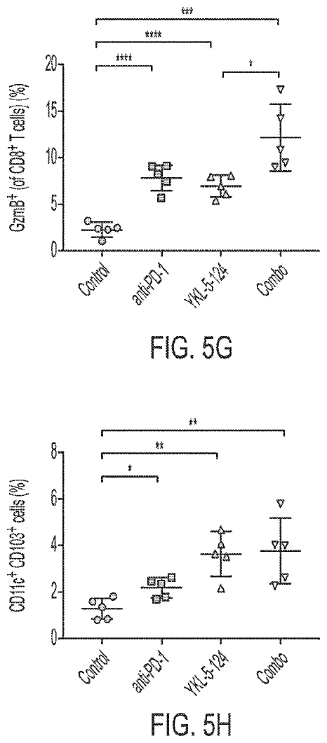

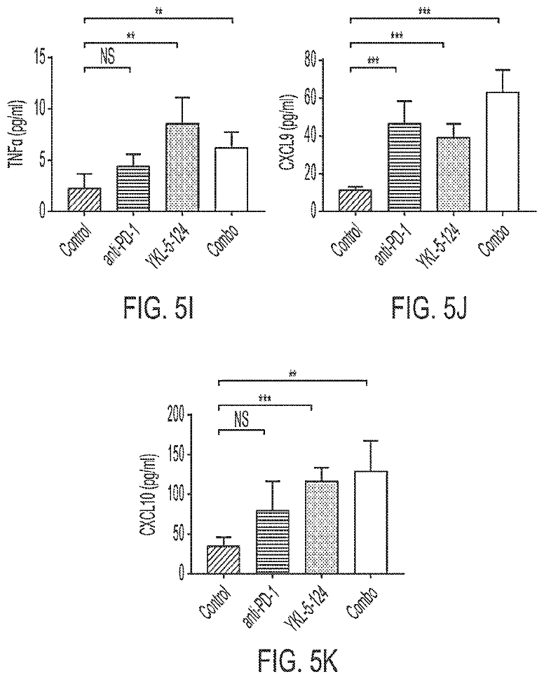

The survival benefit from combining YKL-5-124 and anti-PD1 treatment showed that combined CDK7 and PD-1 inhibition might further alter tumor immune milieu to achieve optimal immune response. To investigate the phenotypical and functional alterations of T cells, mouse tumor-bearing lung was harvested for immune profiling after 7-day treatment as shown in FIG. 11A. There was no significant change of the frequencies of total CD4⁺ and CD8⁺ T cell infiltrates after either anti-PD-1 or YKL-5-124 compared to control tumors (FIGS. 5A and 5B). However, a notable increase of total CD4⁺ T cell infiltrates was observed in the combination treated mice in comparison to control or YKL-5-124 alone treated tumors, and this trend was not seen in total CD8⁺ T cells (FIGS. 5A and 5B). Profiling of CD4⁺ T cells showed a substantial increase of CD44^{high}CD62L⁺ effector CD4⁺ T cells after either YKL-5-124 or anti-PD-1 treatment (FIGS. 5C and 5D), while combination treatment induced the highest increase of CD44^{high}CD62L^{low} CD4⁺ T cells (FIGS. 5C and 5D), which was further confirmed in a separate cohort of RPP GEMMs (FIGS. 13M and 13N).

To further assess the functional activity of CD4⁺ T cells, the expression of a proliferation marker, Ki67, and an activation/costimulatory marker ICOS were analyzed. YKL-5-124 led to a modest increase of tumor-infiltrating CD4⁺ T cells expressing Ki67, and significantly higher frequencies of ICOS⁺ CD4⁺ T cells (FIGS. 5E and 5F). Anti-PD-1 also caused a moderate elevation of Ki67⁺ CD4⁺ T cells, but had no effect on the levels of ICOS⁺ CD4⁺ T cells (FIGS. 5E and 5F), demonstrating that YKL-5-124 may be more effective in promoting activation of CD4⁺ T cells than anti-PD-1. The most marked increase of Ki67⁺ CD4⁺ (FIG. 5E) and ICOS⁺ CD4⁺ T cells (FIGS. 5F and 13O) was detected in mice treated with the YKL-5-124 and anti-PD-1 combination. The activity of CTLs were next assessed by staining for Granzyme B (GzmB), a cytotoxic granule protein secreted by CD8⁺T cells. A considerable but equivalent increase of GzmB⁺ CD8⁺ T cells percentage was observed in YKL-5-124 or anti-PD-1-treated mice (FIG. 5G), suggesting an enhanced cytotoxic T-cell-mediated clearance of tumor cells. Of note, combination treatment resulted in the highest percentage of GzmB⁺ CD8⁺ cytotoxic T cells (FIGS. 5G and 13P), supporting the superior efficacy and improved survival induced by YKL-5-124 and anti-PD-1.

A useful and efficient anti-tumor immune program requires cooperation between DCs and T cells. CD4⁺ T cells provide the key input signals for the DCs to relay the help signal to elicit CD8⁺ CTLs responses. In particular, recent work highlighted the critical role of CD11c⁺ CD103⁺ DCs in the priming and effector phase of the anti-tumor T cell response. Whether YKL-5-124 and anti-PD-1 alone or in combination treatment would have an impact on DCs population (MHCII⁺ CD11c⁺ CD103⁺) was characterized. As compared to control mice, a significant increase in percentages of tumor resident DCs was observed in mice after treatment with YKL-5-124 or combination, but to a lesser extent in mice treated with anti-PD-1 (FIG. 5H). In addition, to directly examine the in vivo secretion of TNFα, CXCL9 and CXCL10 in the TME, bronchoalveolar lavage fluid (BALF) was collected from mouse lung after 7-day treatment. Consistent with the in vitro data, a pronounced increase of TNFα, CXCL9 and CXCL10 was detected upon YKL-5-124 and combination treatment (FIGS. 5I-5K).

These findings demonstrate YKL-5-124 provokes a robust anti-tumor immune program elicited by cooperation between DCs, effector CD4⁺ T cells and cytotoxic CD8⁺ T cells. This effect is further enhanced by the addition of PD-1 blockade.

### Single-Cell Transcriptomic Analysis Identifies Intratumoral Cell Populations

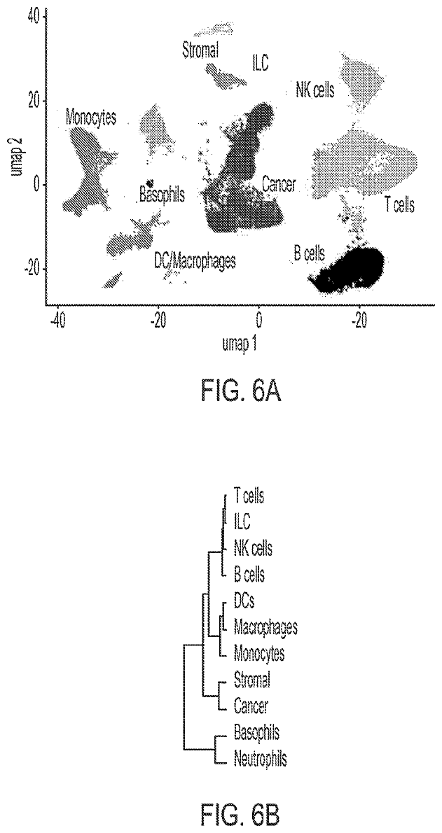

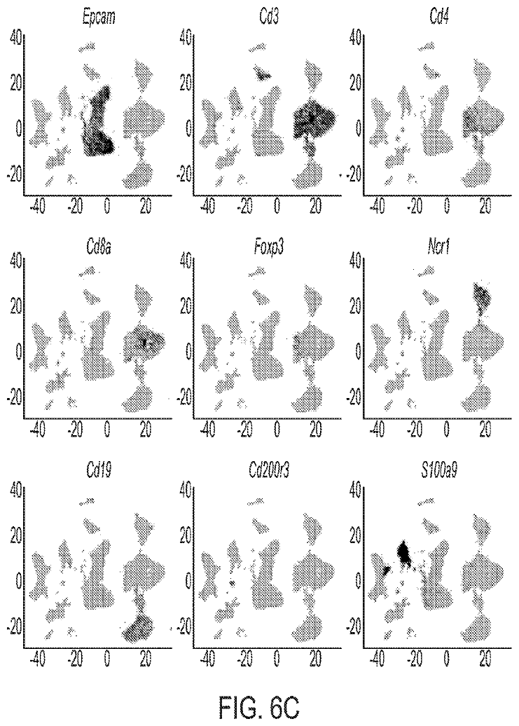

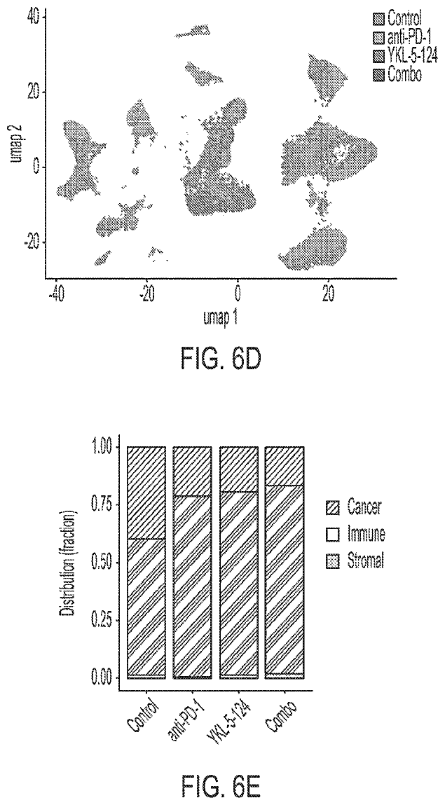

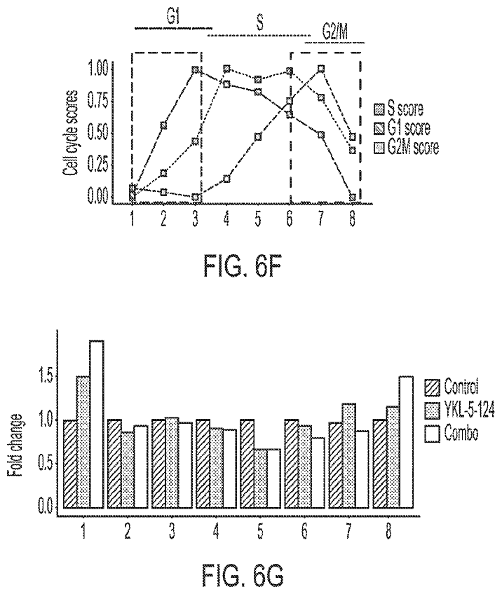

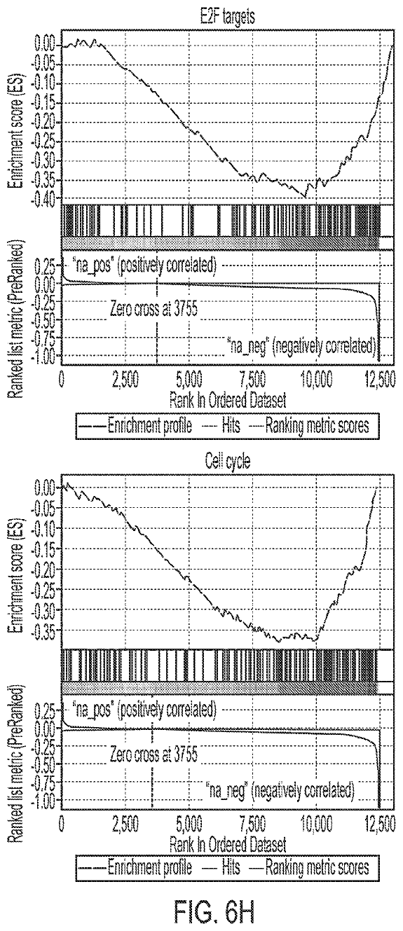

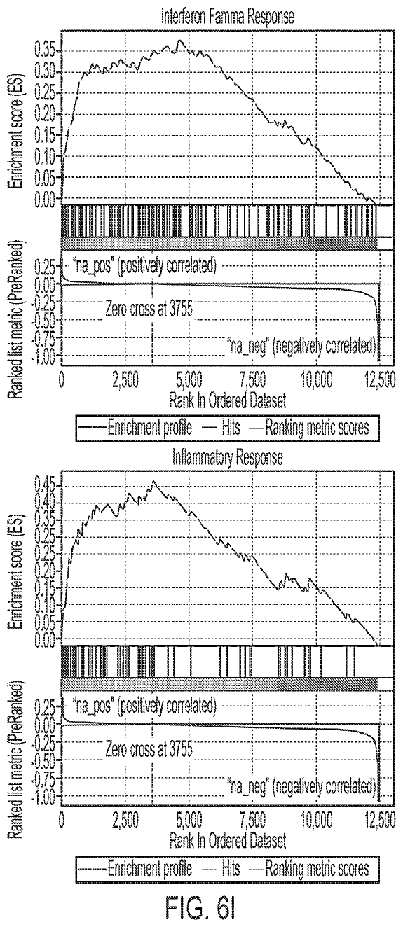

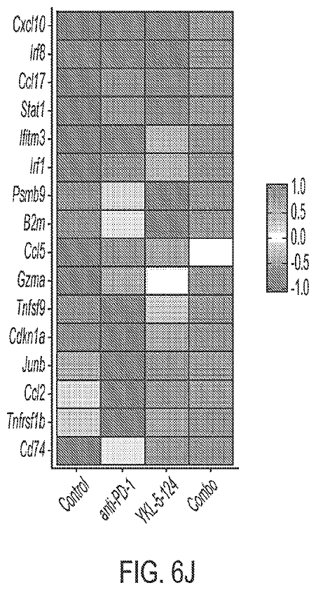

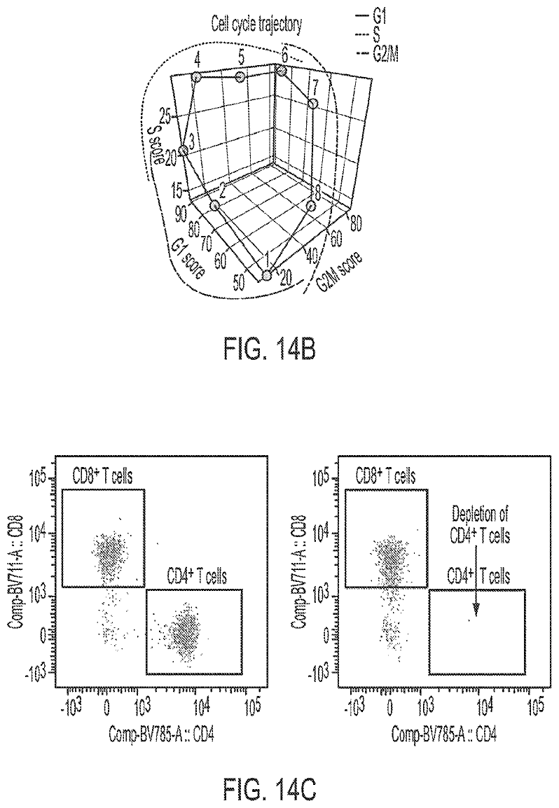

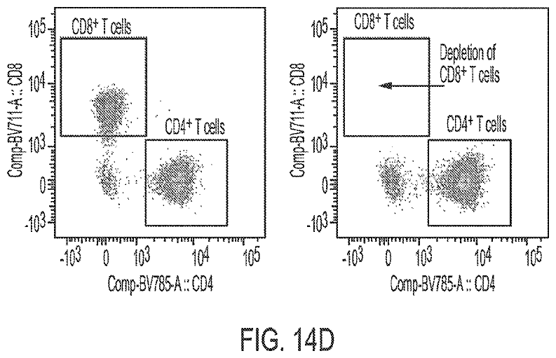

To provide a more comprehensive and unbiased assessment of immunotherapeutic responses, single-cell RNA sequencing (scRNAseq) analysis was performed on the whole TME including tumor cells and immune counterparts. Single-cell transcriptomes were obtained for 9,307 cells in the control group, 10,018 in YKL-5-124, 5,944 in anti-PD-1, and 5,911 in Combo. To define the intratumoral cell populations, data from the four treatment groups using canonical correlation analysis was computationally combined, representing a total of 31,180 cells. Graph-based clustering and dimensionality reduction with UMAP were then used to respectively identify and visualize transcriptionally homogeneous clusters of cells (FIG. 6A). Clusters were further annotated by directly comparing their transcriptional state with that of known sorted populations using SingleR package and assessment of known cell-type specific markers. Cancer cells expressing the epithelial cell adhesion molecule (Epcam), lymphoid populations represented by T cells and innate lymphoid cells (ILC) expressing Cd3d, NK cells expressing natural cytotoxicity triggering receptor 1 (Ncr1), B cells expressing Cd19, and myeloid populations such as monocytes, DCs and macrophages, neutrophils expressing S100a9, basophils expressing Cd200r3 and stromal cells expressing Sparc were all identified (FIGS. 6A-6C and 14A). Notably, compared with distribution of cell populations in the control group, YKL-5-124, anti-PD-1 and Combo treatment resulted in an increase in the frequencies of total immune cell components (FIGS. 6D and 6E).

### Single-Cell Transcriptomic Analysis Confirms Connection of CDK7 Inhibition in Tumor Intrinsic Signaling to Immunity

As shown above, CDK7 inhibition impedes cell cycle progression and DNA replication, leading to genome instability and elevated immune response. To further delineate the temporal events that occur upon in vivo treatment, a continuous cell cycle trajectory from the scRNAseq data was inferred. First, scores for genes specific to the G1, G2/M or S-phase for each cell were calculated and applied to cluster cells in eight groups that represent a cell-cycle state. The center of each group was then connected with its two nearest neighbors in the computed 3D cell-cycle score space (FIG. 14B). The 3D visualization was projected in 2D by retaining the order of the eight cell-cycle states, which provides a portrait of the dynamic phases of cell-cycle progression (FIG. 6F). Analysis of cell distribution illustrated that cells mainly arrested at G1 phase (cluster 1) and to a lesser extent at G2/M (cluster 8) upon YKL-5-124 and combination treatment, whereas a significant decrease of percentage of S phase cells (cluster 4, 5 and 6) was observed (FIG. 6G). Furthermore, GSEA analysis showed that YKL-5-124 significantly downregulated genes within the ‘cell-cycle’ and ‘E2F target’ signatures (FIG. 6H). This data further supports that cell cycle progression is substantially disrupted in vivo by CDK7 inhibition. Finally, YKL-5-124 and combination treatment triggered robust immune response signaling, particularly gene signatures related to ‘Interferon gamma response’ and ‘Inflammatory response’ (FIGS. 6I and 6J).

### Combinatorial Therapy Reinvigorates Anti-Tumor Immunity

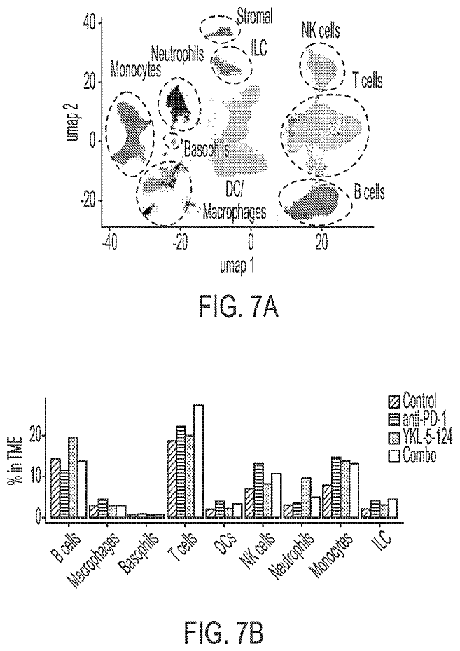

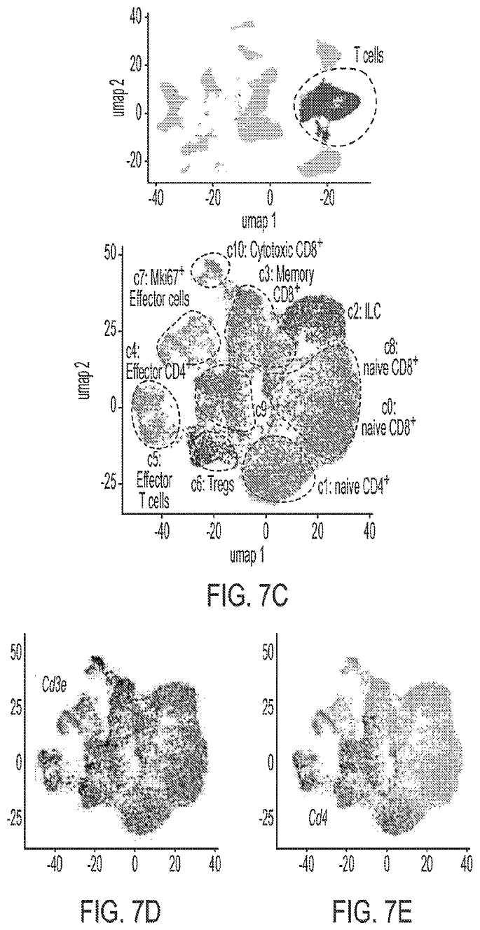

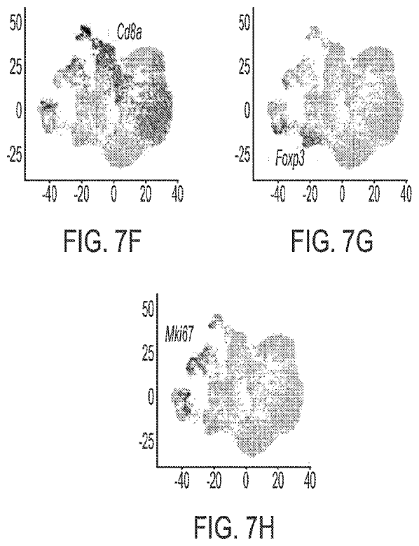

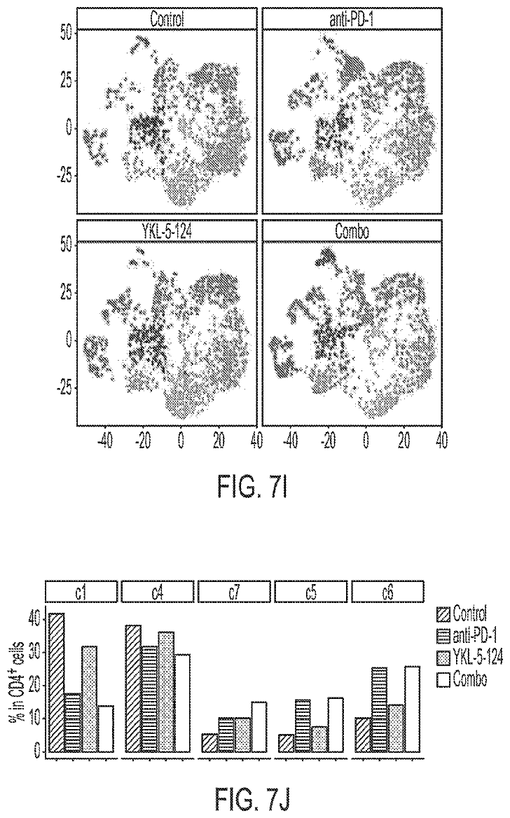

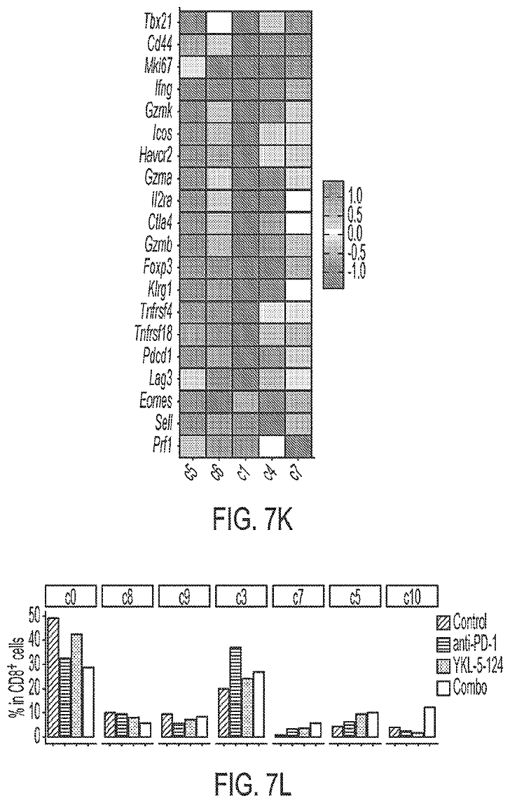

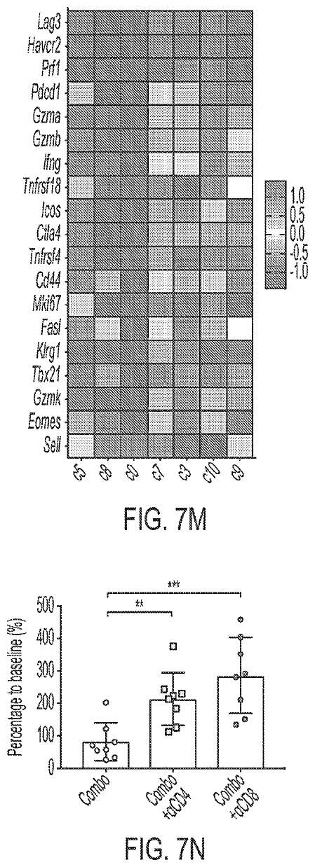

To delineate an overall immune landscape remodeling associated with treatment, the alterations of the subpopulations of the tumor-infiltrating immune cells were characterized (FIGS. 7A and 7B). In comparison to control, a significant increase in percentage of total T cells, NK cells and ILC cells was observed upon YKL-5-124, anti-PD-1 and combination (FIG. 7B). In the myeloid compartments, anti-PD-1 induced an increase of monocytes and macrophages, while YKL-5-124 treatment induced higher numbers of monocytes and neutrophils (FIG. 7B). Of note, although DCs accounted for a small fraction of the overall population, YKL-5-124, anti-PD-1 and combination all induced an increase of DCs (FIG. 7B).

The in vivo immune profiling demonstrates that YKL-5-124 and combination therapy provoke a robust anti-tumor immune program centered on effector CD4⁺ T cells and CD8⁺ T cells. Consistently, scRNAseq analysis showed a particularly prominent impact on T cells (FIG. 7B). To more accurately dissect the T cell subpopulations, T cells (6,698) were separated and the data analyzed at higher granularity (FIG. 7C). This approach yielded 11 distinct T cell subpopulations (c0 to c10) broadly defined by the distribution of classical marker genes, representing high plasticity and complexity (FIGS. 7C-7H). It was evident that YKL-5-124, anti-PD-1, and their combination prompted changes in subpopulation proportions and their transcriptional profiles (FIGS. 7I-7M).

### CD4⁺ T Cells

Cells in c1 expressed high levels of Cd4 and the naive T cell marker Sell (CD62L), but lacked the expression of effector/memory marker Cd44 and T cell activation genes (detailed in method section), including Ifng and Icos (FIG. 7K). c1 appeared to be naive T cells, whose percentage was prominently reduced following YKL-5-124, anti-PD-1 and combination (FIGS. 71 and 7J). c5 showed the highest levels of activated markers and intermediate levels of Sell and Cd44 (FIG. 7K), representing an activated/effector T cell signature. c4 expressed much lower levels of these activation markers, and lower Sell and higher Cd44 (FIG. 7K). YKL-5-124, anti-PD-1, and combination treatment led to an increase in frequency of c5 and a slight reduction of c4 percentage (FIGS. 71 and 7J).

c6 cells expressed high levels of Cd4 and Foxp3, which are markers for regulatory T cells (Tregs). Upon YKL-5-124, anti-PD-1 and Combo treatment, the percentage of Tregs increased significantly in comparison to mice treated with control (FIGS. 7I-7K). This increased frequency of Tregs indicate an acute positive feedback to an activated tumor immune environment by short-term treatment.

c7 displayed highest levels of Cd44 and low of Sell, intermediate levels of T cell activation markers, indicating an effector/activation T cell signature. Of note, this cluster of cells expressed uniquely Mki67, implying proliferative capacity of these cells. In response to YKL-5-124, anti-PD-1 and combination treatment, the numbers of Mki67⁺ CD4⁺ T cells significantly increased (FIG. 7J).

### CD8⁺ T Cells

Both c0 and c8 expressed high levels of Cd8a and Sell, but low Cd44 and T cell activation genes. c9 expressed intermediate level of Sell and relatively low of the T cell activation genes. Thus, c0, c8 and c9 appeared to be naive-like cells and in an inactivated state. Notably, YKL-5-124, anti-PD-1 and Combo all caused a reduction of these three clusters of CD8⁺ T cells (FIGS. 7I, 7L and 7M).

c3 expressed high levels of memory T cell marker Klrg1, intermediate levels of cytotoxic T cells markers (Gzma and Gzmb), and Tbx21 (T-bet) and Eomes. This cluster represents “memory” population with cytotoxicity and low proliferation capability, which has the potential to differentiate into CTLs. c10 showed the highest levels of T cell activation genes and Gzma and Gzmb, suggesting these cells are fully differentiated CTLs. c5 expressed low levels of Sell but high Cd44 and activation markers, representing an “effector” T cell signature (FIGS. 7I, 7L and 7M). In comparison to control, proportions of cells in both c3 and c5 increased in all other groups, whereas the number of cells in c10 increased the most upon combination treatment (FIGS. 7I, 7L and 7M). These findings demonstrate that combining YKL-5-124 and anti-PD-1 remodels the intratumoral CD8⁺ T cell population from cells that are more naive-like to those with more effector-like, activated and cytotoxic properties.

The clustering herein also revealed that numerous clusters were categorized more based on their functional markers rather than classic cellular subtype. c7 cells with high levels of Mki67 are comprised of a mixture of CD4⁺ T cells and CD8⁺ T cells, highlighting their cell proliferation capability (FIGS. 7E, 7F, 7H, 7K and 7M). c5 is another functionally defined cluster of effector/activated T cells, which contain both CD4⁺ and CD8⁺ T cells (FIGS. 7E, 7F, 7H, 7K and 7M). In response to YKL-5-124, anti-PD-1 and combination, the number of cells in c7 and c5 increased substantially (FIGS. 7J and 7L), demonstrating a major impact on proliferation and activation of CD4⁺ and CD8⁺ T cells.

Collectively, the data highlight anti-tumor immunity alterations occurring in the intratumoral T cells compartment following YKL-5-124, anti-PD-1 and particularly combinational therapy, including (1) a significant reduction in CD4⁺ and CD8⁺ naive T cells frequency; (2) a dramatic expansion of CD4⁺ and CD8⁺ effector/memory T cells and CD8⁺ CTLs, with an increase in the number of Mki67⁺ proliferating T cells; and (3) upregulation of an anti-tumor gene signatures of T cell activation/function.

Anti-Tumor Immunity by Combining YKL-5-124 and PD-1 is Partly Dependent on CD4⁺ and CD8⁺ T Cells

The in vivo data demonstrate that combining YKL-5-124 and anti-PD-1 resulted in the best tumor response and optimal immune surveillance centered on T cells. To determine whether CD4⁺ or CD8⁺ T cells directly contributes to combination therapy response, the impact of perturbing immune cell function by in vivo neutralization antibodies against CD4 (αCD4) or CD8 (αCD8) was assessed (FIGS. 14C and 14D). Tumor-bearing mice were randomized to either combination treatment, or combination treatment plus αCD4 or αCD8. Indeed, compared to non-depletion mice in the combination group, CD4⁺ T cell-depleted mice had significantly higher tumor burdens (FIGS. 7N and 7O). Similarly, a dramatic increase of tumor volumes was observed in CD8⁺ T cell-ablated mice (FIGS. 7N and 7O). Depleting either CD4⁺ or CD8⁺ T cells mitigated the anti-tumor effect of combining YKL-5-124 and anti-PD-1 (FIGS. 7N and 7O). These observations further support findings that T cells are required for anti-tumor immunity induced by combination treatment.

### DISCUSSION

These results demonstrate that targeting CDK7 with an inhibitor (e.g., YKL-5-124) disrupts cell cycle progression and causes DNA replicative stress and genome instability in tumor cells, leading to cellular responses including release of multiple pro-inflammatory cytokines/chemokines. These tumor cell-intrinsic events provoke a robust immune surveillance, which leads to T-cell-mediated tumor control in mouse SCLC models. Combining YKL-5-124 with PD-1 blockade promotes strong anti-tumor immunity and confers remarkable survival benefit in this highly aggressive cancer. The findings herein provide a rationale for combining CDK7 inhibition and immunotherapy for SCLC patients.

Selectively targeting CDK7 by YKL-5-124 did not result in changes in CTD phosphorylation of RNA Pol II and SE-associated genes expression in SCLC. These findings reveal potential redundancies in CDK control of gene transcription. YKL-5-124 treatment leads to cells predominantly arrested at the G1 to S phase transition and unable to advance to S phase. Conventionally, at the GUS transition, CDKs activate and facilitate the conversion of MCMs in the pre-initiation complex to an active DNA helicase, leading to initiation of DNA synthesis. When the regulatory mechanisms fail, replicative stress and DNA damage ensue. CDK7 inhibition dramatically impairs the levels of MCM2 at the replication forks, leading to decreased replication origins, elevated replicative stress and DNA damage. Genome instability such as micronuclei formation has recently emerged as a crucial cue to activate immune response. These micronuclei represent an important source of immunostimulatory DNA and precede activation of inflammatory signaling. Indeed, the series of events provoked by CDK7 inhibition in cancer cells trigger robust immune response signaling and secretion of pro-inflammatory factors, which potentiates T cell activity, resulting in tumor control.

The therapeutic potential of combining CDK7 inhibitor with ICB in murine models with lung TME to closely mimic human disease was explored. A key strength of this current study lies in the use of four histopathology validated SCLC models. Of note, combining YKL-5-124 with anti-PD-1 significantly improved efficacy and extended overall survival in all tested SCLC models, supporting the potential of CDK7 inhibition in enhancing immunity as backbone for combinational immunotherapy. Strikingly, adding YKL-5-125 and anti-PD-1 to standard chemotherapy resulted in the best tumor response and the longest survival among all treatment groups with no observed toxicities. This data provides strong evidence for combining CDK7 inhibitors in first line treatment with chemotherapy and ICB in clinical trials and highlights the translational significance of this work.

This represents the first unbiased scRNAseq analysis of TME in mouse SCLC, which was performed without prior marker selection. In the cancer cells, scRNAseq analysis confirms a unique signature of disrupted cell cycle progression and connects the tumor intrinsic effect by YKL-5-124 to immune response signaling. This work uncovered several key observations and mechanisms of action on immune compartments: (1) complexity of tumor-immune ecosystem dynamics; YKL-5-124, anti-PD-1 and combination therapy (2) trigger the expansion/reduction of certain subtypes of tumor-infiltrating lymphoid and myeloid cells, (3) cause a dramatic shift of naive CD4⁺ and CD8⁺ T cells to effector/activated T cells and (4) enhance proliferative capacity of Mki67⁺ effector T cells; combination therapy (5) results in the most prominent increase of CD8⁺ CTLs and (6) triggers the strongest increase in the expression of an anti-tumor cytotoxic gene signature.

In summary, this work serves as the basis for the combinatorial application of CDK7 inhibition and immunotherapy and provides evidence for combining YKL-5-124 and PD-1 blockade in future clinical trials (e.g., in SCLC).

### Experimental Model and Subject Details

### Animal Studies

All animal studies were reviewed and approved by the Institutional Animal Care and Use Committee (IACUC) at the New York University Langone Health (NYULH) and Dana-Farber Cancer Institute (DFCI). From 6 weeks of age, mice were induced with adenovirus-Cre recombinase (Ad-Cre) by intratracheal intubation to allow cre-lox-mediated recombination of floxed Rb1, p53 and p130 alleles. For syngeneic orthotopic models, ultrasound-guided transthoracic injection was performed using 200,000 RPP631, RP or RPP-MYC cells in 30 μL PBS directly into the lung of 6 to S-week-old C57BL/6 (Jackson Laboratory, Stock No: 000664). For OT-IT cell assay, OT-I mice were purchased from the Jackson Laboratory (Stock No: 003S31). Both males and females mice were used and all mice were maintained in accordance with the respective NYULH and DFCI on the care, welfare, and treatment of laboratory animals. All experiments met or exceeded the standards of the Association for the Assessment and Accreditation of Laboratory Animal Care, International (AAALAC), the United States Department of Health and Human Services, and all local and federal animal welfare laws.

### Cell Lines

Established mouse SCLC Rbr1^{-/-}p53^{-/-}p130^{-/-} (RPP)631, Rbr1^{-/-}p53^{-/-} (RP) and RPP-MYC cells were maintained in HITES medium and confirmed by sequencing and western blotting. Human cell lines (GLC16, DMS79, NCI-H69, NCI-H82) were maintained in RPMI 1640 (Thermo Fisher Scientific). The HAP1 CDK7 WT and C312S cell lines were cultured in IMDM media (Thermo Fisher Scientific). All cell line media were supplemented with 10% Fetal Bovine Serum (FBS, Sigma-Aldrich) and 1% penicillin/streptomycin (Thermo Fisher Scientific) and all cell lines were cultured in a humidified chamber with 5% CO₂.

### Method Details

### Chemicals

YKL-5-124 was synthesized according to Olson, et al., Cell Chem Biol. 2019, 26(6), 792-803. THZ531 was synthesized according to Zhang, et al., Nat. Chem. Biol. 2016, 12, 876-884. THZ1 and Bio-THZ1 was synthesized according to Kwiatkowski, et al., Nature, 2014, 511, 616-620. Anti-PD-1 (clone 29F.1A12) was a kind gift from Dr. Gordon Freeman (DFCI).

### In Vivo Toxicity Evaluation and Treatment Studies

For evaluation of in vivo toxicity of YKL-5-124, a dose-escalating study from 2.5 mg/kg to 15 mg/kg q.d. five times/week via intraperitoneal injection (i.p.) was tested in C57BL/6 mice. In vivo toxicities including body weight and blood cell counts including platelet, red blood cells and white blood cells were monitored. Similarly, a separate cohort was used to evaluate in vivo toxicity of combining YKL-5-124 and anti-PD-1 in C57BL/6 mice. For treatment studies, mice were evaluated by MRI imaging (Preclinical Imaging Laboratory, NYULH) to quantify lung tumor burden before for randomization and after drug treatment for efficacy evaluation. Mice were treated with either vehicle and isotype IgG (Control), anti-PD-1 200 μg/mouse (clone 29F.1A12), YKL-5-124 10 mg/kg, or combined anti-PD-1 and YKL-5-124 (Combo). YKL-5-124 was administered daily from Monday to Friday, and PD-1 antibody was administered three times a week (Monday, Wednesday, and Friday). For chemotherapy, cisplatin (5 mg/kg) was given on Day 1 and etoposide (10 mg/kg) on Day 1, 2, 3 every seven days per cycle for three cycles. For CD4 or CDS neutralization study, mice were injected intraperitoneally with either a.CDS antibody (400 μg, Bio X Cell, clone 2.43), or αCD4 (400 μg, Bio X Cell, clone GK1.5), or IgG2b isotype control (400 μg, Bio X Cell, clone LTF-2) 48 and 24 h before beginning anti-PD-1 and YKL-5-124 (Combo) treatment, and every 4 days thereafter.

### Cell Viability Assay

Cells were seeded in 96-well plates (0.01-0.02×10⁶ cells/well) in media and treated with YKL-5-124 at indicated concentrations and time points. Cell viability was measured using the MTS-based CCK-8 assay (Dojindo, Cat #CK04). Absorption at 450 nm was measured 3 hr after addition of CCK-8 reagent to cells.

### Cell Cycle Analysis

Cells were seeded in 6-well plates (0.5-1×10⁶ cells/well) in media and treated with YKL-5-124 at indicated concentrations and time points. Cells were collected by centrifugation, washed in cold PBS and resuspended in 1 ml of 80% EtOH in PBS and stored overnight at −20° C. Cells were again collected by centrifugation and washed 3× with cold PBS. Hereafter cells were resuspended in PBS containing: 0.1% Triton X-100, 25 μg/ml Propidium Iodide (PI) and 0.2 mg/ml RNase A and incubated for 45 min at 37° C. After incubation, cells were placed at 4° C. until ready for analysis with flow cytometry. Cells were gated for PI staining and cell accumulation in G1, S and G2/M were calculated using ModFit LT software.

### BrdU Analysis

Cells were seeded in 6-well plates (0.5-1×10⁶ cells/well) in media and treated with YKL-5-124 at indicated concentrations and time points. Prior to collection, cells were pulsed with 1 mM BrdU (BD Biosciences, #559619) for 2-4 hr (depending on cell line doubling time) and collected by centrifugation. Hereafter cells were permeabilized, fixated and stained according to BrdU kit instruction (BD Biosciences, Cat #559619). Cells were gated for BrdU incorporation (FITC) and 7-aminoactinomycin D (7-AAD) to quantify cells in G1, S (BrdU positive) and G2/M.

### RNA Extraction and RT-qPCR

Cell pellets were collected and then subjected to total RNA extraction using RNeasy Plus Mini Kit (QIAGEN, Cat #74136) according to the manufacturer's instructions, or using TRIzol:Chloroform phase-separation by centrifugation followed by RNA precipitation using isopropanol. The extracted RNA was reversely transcribed into cDNA using the High-Capacity RNA-to-cDNA™ Kit (Thermo Fisher Scientific, Cat #4387406) according to the manufacturer's instructions. The obtained cDNA samples were diluted and used for RT-qPCR. PowerUp™ SYBR™ Green Master Mix (Thermo Fisher Scientific, Cat #A25742) or TaqMan Gene Expression Master Mix (Life Technologies, Cat #4369514). Gene specific primers with sequences listed in Table 3 were used for PCR amplification and detection on the StepOnePlus™ Real-Time PCR System (Thermo Fisher Scientific). RT-qPCR data were normalized to Actb (mouse cells) or GUSB (human cells) and presented as fold changes of gene expression in the test sample compared to the control.

### Protein Extraction and Western Blotting Analysis

Cells in culture were collected by centrifuging at 1,500 rpm and then washed with ice-cold PBS twice to completely remove residual medium. RIPA lysis buffer (Thermo Fisher Scientific, Cat #89900) supplemented with Halt™ Protease and Phosphatase Inhibitor Cocktail (Thermo Fisher Scientific, Cat #78440) was added to cell pellets to extract protein. Protein concentrations in lysates were measured by Pierce™ BCA Protein Assay Kit (Thermo Fisher Scientific, Cat #23225) and followed by the addition of SDS loading buffer (6×) and heated at 95° C. for 5 min. Equal amount of protein samples was subjected to 4-20% gradient gel SDS-PAGE and transferred to a nitrocellulose membrane (Bio-Rad, Cat #1704271). The membrane was blocked in Odyssey® Blocking Buffer (LI-COR, Cat #927-50003) at room temperature for 1 hour and incubated with appropriate antibodies at 4° C. overnight (Key Resources Table). Antibodies were diluted in TBST (TBS with 0.1% Tween) with 20% LI-COR Odyssey Blocking Buffer. On the next day, the membrane was washed with TBST (TBS with 0.1% Tween) four times and incubated with appropriate secondary antibodies (LI-COR, anti-Rabbit, Cat #925-32213; anti-Mouse, Cat #925-68072) at room temperature for 1 hour. Membranes were imaged using the LI-COR Odyssey® Imaging System.

### Competitive Pulldown Assay for YKL-5-124 Target Engagement

Bio-THZ1 pulldown experiments followed by western blotting of enriched proteins was performed as described in Kwiatkowski, et al., Nature, 2014, 511, 616-620. Briefly, cells were treated with YKL-5-124 or DMSO for 6 hr (in vitro) or 72 hr (in vivo). Following treatment, cells were washed twice with cold PBS and then lysed in the following lysis buffer: 50 mM TrisHCI pH 8.0, 150 mM NaCl, 1% NP-40, 5 mM EDTA, 1 mM DTT, and protease/phosphatase cocktails. Following clearance, lysates were treated with biotinylated THZ1 for pulldown overnight at 4° C. Lysates were further incubated at room temperature for 3 hr to increase the efficiency of covalent bond formation. Lysates were then incubated with streptavidin agarose for pulldown for an additional 2-3 hr at 4° C. Agarose beads were washed 5 times with lysis buffer and then boiled in 2×SDS at 95° C. SDS-page resolved precipitated proteins were probed for the indicated proteins.

### Immunofluorescence Staining and Imaging

Before fixation, cells in PBS were seeded for 30 min on 0.01% Poly-lysine coated coverslip. Cells were then fixed in 4% paraformaldehyde (Diluted the 32% paraformaldehyde in PBS, Electron Microscopy Sciences 15714) for 10 min at room temperature. Cells were washed three times for 5 min with 200 mM glycine containing PBS, followed by permeabilization with 0.3% Triton X-100 in PBS for 15 min. After blocking with 5% bovine serum albumin (BSA) in PBS for 1 hour, cells were incubated with primary antibody (γH2A.X, Cell signaling 9718, 1:200) diluted in a 5% BSA in PBS solution overnight at 4° C. After washing four times with PBS, cells were incubated with Alexa Fluor Plus 555 (Invitrogen A32732, 1: 500) secondary antibody for 1 hour and washed three times with PBS. Cell nuclei were counterstained with DAPI (Biolengend 422801, diluted to 600 nM in PBS) for 5 min. Cells were washed two more times in PBS before mounting with Fluorescence Mounting Medium (Dako, S3023). Images were acquired using Zeiss 880 Laser Scanning Confocal Microscope and were processed by FIJI (NIH). Micronucleus were identified manually by distinct DAPI staining outside the main nucleus.

### EdU and MCM2 Imaging by the Stochastic Reconstruction Microscopy (STORM) Sample Preparation

RPP631 cells were incubated in the presence of 100 nM YKL-5-124 for 48-72 hr. EdU was pulsed during the last 30 minutes of YKL-5-124 treatment. Treated cells were pre-extracted using 0.5% Triton in CSK buffer (10 mM Hepes, 300 mM Sucrose, 100 mM NaCl, and 3 mM MgCl2, pH=7.6) for 10 minutes, and fixed with paraformaldehyde (4%) for 30 minutes. Cells were then rinsed 3 times with PBS and blocked (2% glycine, 2% BSA, 0.2% gelatin, and 50 mM NH₄Cl in PBS) overnight at 4° C. for further staining. EdU was tagged with Alexa Fluor 647 picolyl azide through Click reaction (Thermo Fisher, C₁₀₆₄₀) before immunofluorescent labeling of target proteins, for which the antibodies are mouse anti-PCNA (Santa Cruz Biotech., sc56, 1:1000), goat anti-mouse (Alexa Fluor 750 conjugated, Thermo Fisher, A-21039, 1:5000), and rabbit anti-MCM2 (Alexa Fluor 647 conjugated, Abcam ab223403, 1:500). Fixed cells were mounted onto microscope glass for STORM imaging in freshly mixed imaging buffer (1 mg/ml glucose oxidase (Sigma-Aldrich, G2133), 0.02 mg/ml catalase (Sigma-Aldrich, C3155), 10% glucose (Sigma-Aldrich, G8270), and 100 mM cysteamine (MEA, Fisher Scientific, BP2664100).

### Optics

STORM imaging was performed on a customized inverted microscope. In brief, the 639 nm laser (UltraLaser, MRL-FN-639-800) was collimated and reflected into the TIRF Objective (HCX PL APO 63×NA=1.47, Zeiss), and the adjusted illumination power is ˜1.5 kW/cm2. A 405 nm Laser line (MDL-III-405-150, CNI) was equipped to reactivate Alexa Fluor 647 fluorophores. A 750 nm laser (UltraLaser, MDL-III-750-500) was also applied to illuminate Alexa Fluor 750 labeled PCNA for S-phase cell selection. Cell samples were sequentially illuminated with 639 and 750 laser lines and the emitted fluorescence was collected by the same objective with a 2× magnification tube lens (Diagnostic Instruments). The fluorescence was then filtered by single band filters (Semrock FF01-676/37 and FF02-809/81) switched in a filter wheel accordingly. Photons were eventually fed to a sCMOS camera (Photometrics, Prime95B) and collected at 33 Hz, 2000 frames for an image stack.

### Single-Molecule Localization and Pair-Correlation Analysis

Each 2000-frame single-molecule image stack was submitted to a home-built software for precise single-molecule localization. Briefly, each frame from an image stack was box filtered, roughly-local-maxima-localized, segmented, and submitted to GPU for Point-Spread-Function (PSF) fitting using the Maximum Likelihood Estimation (MLE). The fitting accuracy was evaluated using Cramer-Rao Lower Bound (CRLB). Note that the patterned read-out noise of the sCMOS camera was calibrated before imaging. Such read-out noise for each pixel was approximated into a Gaussian distribution and convolved with the Poisson shot noise for MLE fitting. The generated coordinates were then submitted for Auto-Pair-Correlation analysis to estimate the nuclear density of the fluorophores within a nucleus, as well as how many fluorophores are on average in each EdU/MCM2 focus. The number of fluorophores is proportional to the quantity of EdU and MCM2. This method empowered us to map the precise molecular coordinates of EdU and MCM2 molecules within a cell with a resolution of ˜10 nm, and to extract robust metrics such as the amount of EdU and MCM incorporated per nucleus as well as their quantity within each focus.

### Histology and Immunohistochemistry

Lungs were perfused with 10% formalin, stored in fixative overnight, and embedded in paraffin. Four-micron thick sections of formalin fixed tissue were used for immunoperoxidase analysis after baking at 60° C. for 1 hour, deparaffinization and rehydration (100% xylene ×4 for 3 minutes each, 100% ethanol ×4 for 3 minutes each and running water for 5 minutes). The sections were blocked for peroxidase activity with 3% hydrogen peroxide in methanol for 10 minutes and washed under the running water for 5 minutes. The sections with pressure cooked (Biocare Medical) antigen retrieval were at 120° C. in Citrate Buffer (Dako Target Retrieval Solution, S1699). The slides were cooled for 15 minutes, and transferred to Tris buffer saline (TBS). The sections were incubated with rabbit monoclonal anti-MYC (Abcam Cat #ab32072; 1:900) and or rabbit monoclonal anti-ASCL1 antibody (Abcam Cat #ab2l1327; 1:100) was incubated 40 min room temperature. The secondary antibody was used Leica Novolink Polymer (Cat #RE7161) 30 min incubation. All the incubations were carried out in a humid chamber at room temperature. The slides were rinsed with TBS in between incubation. The sections were developed using 3,3′-diaminobenzidine (DAB) as substrate and counter-stained with Mayer's Hematoxylin.

### Luminex Analysis of Murine BAL Fluid

Mouse lung bronchoalveolar lavage (BAL) was performed by intratracheal injection of 2 ml of sterile PBS followed by collection by aspiration. TNFα, CXCL9 and CXCL10 levels were measured using mouse Cytokine/Chemokine 32-plex Assay (MILLIPLEX, Millipore) on Luminex® SD system (Luminex). Concentrations (pg/ml) of each protein were derived from 5-parameter curve fitting models according to the manufacturer's instructions.

### MRI Quantification

Animals were anesthetized with isoflurane to perform MRI of the lung field using BioSpec USR70/30 horizontal bore system (Bruker) to scan 24 consecutive sections. Tumor volumes within the whole lung were quantified using 3-D slicer software to reconstruct MRI volumetric measurements as described previously. Acquisition of the MRI signal was adapted according to cardiac and respiratory cycles to minimize motion effects during imaging.

### Immune Profiling Flow Cytometry

Mice were humanely euthanized, and mouse lungs were perfused using sterile PBS through heart perfusion from the left ventricle after BAL fluid collection. The whole lung was cut and minced into small pieces followed by digestion in collagenase D (Sigma-Aldrich) and DNase I (Sigma-Aldrich) in Hank's Balanced Salt Solution (HBSS) at 37° C. for 30 minutes. After incubation, the digested tissue was subjected to a 70 μm cell strainer (Thermo Fisher Scientific) to obtain single-cell suspensions. Separated cells were treated with 1×red blood cell (RBC) lysis buffer (BioLegend). Live cells were determined by LIVE/DEAD fixable aqua dead cell stain kit (Molecular Probes). The cell pellets were resuspended in PBS with 2% FBS for FAGS analysis. Cells were stained with cell surface markers as indicated followed by fixation/permeabilization (eBioscience). Cells were imaged on BD LSRFortessa (BD Biosciences) and analyzed using FlowJo software (Tree Star).

### Ex Vivo OT-I T Cell Assay

The spleen of OT-I mice was minced with a razor and mashed through a 40 m strainer to form a single-cell suspension. Separated cells were then treated with RBC lysis buffer and a number of 5×10⁵ cells were seeded in a 96-well U-bottom plate. For conditioned medium culture assay, RPP631 cells were treated with either DMSO or YKL-5-124 for 48 hr and drug was washed off after first 6-hour treatment. Subsequently, DMSO-conditioned medium or YKL-5-124-conditioned medium were collected and added to above single-cell suspension in a 96-well U-bottom plate in the presence of Ova₂₅₇₋₂₆₄ peptide (10 μg/ml, GenScript, Cat #RP10611) for 4 days. Alternatively, DMSO or YKL-5-124 was added directly to single-cell suspension in the presence of Ova257-264 peptide for 4 days in the T cell assay medium (complete RPMI with HEPES, sodium pyruvate, MEM nonessential amino acids and 2-mercaptethonal). T cell activation markers CD69, TNFα. and IFNγ were analyzed by flow cytometry.

Generation of cGAS CRISPR/Cas9 Knockout

Single guide RNA (sgRNA) oligonucleotides (Sigma-Aldrich) targeting cGAS (Table 3) were cloned into lentiviral expression vector lentiCRISPR v2 (Add gene #52961). Lentivirus was generated by transfection of HEK-293T cells with lentiCRISPR v2, or lentiCRISPR v2-sgcGAS and the packaging plasmids psPAX2 (Addgene #12260) and pMD2.G (Addgene #12259) using Lipofectamine 3000 (Thermo Fisher Scientific). Viral particles released into the cell culture supernatant were filtered with 0.45-μm filters and added to target cells.

### Bulk-RNA Sequencing Analysis

Paired-end reads were aligned to mouse mm10 genome using STAR (v2.5.2b). Reads with good mapping quality (MAPQ>30) that aligned to genomic exons were counted using featureCounts (mm10 Ensembl 93) to generate a table with counts for each gene. Differential gene expression analysis was performed using the R package DESeq2 using the lfcShrink function. Genes with false discovery rate (FDR) lower than 0.05 were considered significantly differentially expressed. Gene set enrichment analysis (GSEA) was performed on a list of genes ranked from high to low DESeq2 estimated fold-change using the GSEAPreRanked function with enrichment statistic classic and 1000 permutations.

### Single-Cell RNA Sequencing

### Experimental Protocol and Library Generation

To account for interindividual variability, we harvested pooled fresh tumor-bearing lungs from two mice in two independent cohorts of RPP orthotopic mice which were treated with control, YKL-5-124, anti-PD-1 and combination treatment for seven days. Single cell suspensions were achieved as described above and were sorted using DAPI staining. Cells were then resuspended into single cells with 0.4% BSA for 10× genomics processing. Cell suspensions were loaded onto a 10× Genomics Chromium instrument to generate single-cell gel beads in emulsion (GEMs). Approximately 5,000 to 10,000 cells were loaded per channel. scRNA-seq libraries were prepared using the following Single Cell 3′ Reagent Kits: Chromium™ Single Cell 3′ Library & Gel Bead Kit v2 (PN-120237), Single Cell 3′ Chip Kit v2 (PN-120236) and i7 Multiplex Kit (PN-120262) (10× Genomics, Pleasanton, Calif., USA), and following the Single Cell 3′ Reagent Kits v2 User Guide (Manual Part #CG00052 Rev A). Libraries were run on an Illumina HiSeq 4000 system (SY-401-4001, Illumina) as 2×150 paired-end reads, one full lane per sample, for approximately >90% sequencing saturation.

### Data (Pre-)Processing

The Cell Ranger Single Cell Software Suite, version 1.3 was used to perform sample de-multiplexing, barcode and UMI processing, and single-cell 3′ gene counting. A detailed description of the pipeline and specific instructions to run it can be found at: https://Support.10×genomics.com/single-cell-gene-expression/software/pipelines/latest/what-is-cell-ranger). A high quality gene expression matrix was created in sequential preprocessing steps as provided in the Seurat (v2.3.4) pipeline. First only genes that were detected in both independent scRNAseq datasets were retained and cells were excluded if respectively more than 0.2% or 0.3% of detected genes were from mitochondrial or ribosomal origin or if less than 500 genes were detected. The count matrix was subsequently log-normalized with a scale-factor of 10000 and the obtained normalized matrix was further corrected by retaining the residuals after regressing out systematic changes due to number of detected UMIs, percent genes from mitochondrial or ribosomal origin.

### Clustering and Annotation

Cells from both independent datasets were merged together in canonical correlation (CC) space using the intersection of highly variable genes, identified as genes with higher-than-expected variability in consecutive ranked expression bins, from both datasets. The implementation of CC alignment and selection of downstream dimensions to use was performed as described in the Seurat package. Cells were clustered using the Louvain algorithm based on a shared nearest-neighbor network. The final resolution of all subsequent clustering analyses was determined by the biological questions and the need for coarse or fine-grained detail as explored in the subsequent cluster annotation. Clusters were annotated using input from multiple sources, we i) calculated a stromal, cancer and immune score using the estimate (v1.0.13) package in R, ii) identified cluster specific gene expression markers based on gini-scores, iii) created a hierarchical tree based on Pearson correlation scores between clusters to visualize lineage relationships and iv) compared clusters to those of the Immgen database using the SingleR (v0.2.1) package in R. UMAP as implemented by the uwot (v0.0.0.9010) package in R was used to visualize clusters in 2D CC space. Overall this strategy was hierarchically used to assess all cells, cancer cells only, immune cells only and finally T-cells only.

### Quality Control

In the first clustering step, several additional analyses were performed to remove dubious or non-reproducible clusters from downstream analyses. To identify contamination of immune cell clusters with cancer cells we performed k-means (2 centers) clustering on all cells on identified and known canonical markers (Epcam, Calca, Stmn1, Ascl1, Krt7, Nfib, Krt8, Krt18) specific to cancer cells. Single-cell outliers with high expression of the aforementioned cancer genes were marked using a density based clustering algorithm using the dbscan (v1.1-3) R package. Similarly, at the cluster level, immune cell clusters with high expression of aforementioned cancer genes were split and labeled as cancer-contaminated and cancer-free cells. Furthermore, clusters that represent doublets were identified using the doubletCluster function of the scran (v1.10.1) and scater (v1.10.0) package in R and concomitantly observing a significant distribution shift for total number of detected UMIs per cell. To detect non-reproducible clusters between datasets we compared changes in distribution and only removed a cluster if this cluster was only observed in all treatment conditions of only one dataset with all individual z-scores greater than 3. Together all marked single-cells identified in any of the previous quality analyses were removed from further analyses.

### Cell-Cycle Analysis

Cells were first classified in G1, G2/M or S categories using the Cyclone function in the scran package in R, which provides a discrete grouping based on genes known to be specific to the specific cell-cycle phases. To infer more continuous cell-cycle states we first identified the top 50 genes that are associated with each cell-cycle phase by performing pairwise comparisons using the limma (v3.38.2) package in R. Based on those cell-cycle phase specific genes we calculated a G1, G2/M and S-score for each cell by summarizing the normalized expression values. All cells were subsequently clustered by k-means in eight groups using the three cell-cycle scores. We selected eight groups since there are at least three cell-cycle states and each cell could be considered inside (high score) or outside (low score) a state (2\hat{ } 3=8). To visualize the continuous cell-cycle progression states the median of each group was visualized in a 30 plots and nearest neighbors were connected. The 30 visualization was project in 20 by rescaling the average of each k-means group between 0 and 1. To assess changes in cell-cycle progression state distribution upon treatment we calculated the percentage of single-cells within each group for each treatment condition and compared that to vehicle-treated samples.

### Gene Set Enrichment Analysis

GSEA was performed on a list of genes ranked according to limma PI-values (−log 10 (adjusted p-value)×absolute log FC), using the GSEAPreRanked function with enrichment statistic classic and 1000 permutations. For specific enrichment of superenhancer-associated genes found in Christensen, et al., Cancer Cell, 2014, 26, 909-922, a custom gene set for those genes was first created and merged with in the GO BP category of the MSigDB database to obtain the enrichment ranking. Specific gene enrichment within a cluster was visualized by averaging the expression of each gene per cluster and subsequently rescaling z-scores for each gene between −1 and 1.

CD4⁺ and CD8⁺ T-Cell Identification

To identify CD4⁺ and CD8⁺ subgroups within the annotated T-cell cluster, to mitigate known dropout effects in single-cell data, the T-cell population was over-clustered and all subclusters divided in CD4⁺ or CD8⁺ using k-means clustering (groups=2) based on the expression of Cd4 and Cd8a. This provided a clear separation between predominant CD4⁺ and CD8⁺ cells. scRNAseq uncovered five distinctive clusters of CD4⁺ T cells (c1, c4, c5, c6 and c7) and seven clusters of CD8⁺ T cells (c0, c3, c5, c7, c8, c9 and c10). Among these, functionally defined clusters c5 and c7 are comprised of both CD4⁺ T cells and CD8⁺ T cells.

### Illustration Tool

The graphical abstract image was created with BioRender.

### Quantification and Statistical Analysis

Statistical analyses were performed using GraphPad Prism 7 software and statistical significance was determined by p<0.05. Data are presented as mean with SD unless otherwise specified. Statistical comparisons were performed using unpaired Student t test for two-tailed p value unless otherwise specified (*p<0.05, **p<0.01, ***p<0.001, ****p<0.0001).

Reagents and resources for the above examples were sourced as shown in Table 2.

TABLE 2

| | | |
|---|---|---|
| REAGENT or RESOURCE | SOURCE | IDENTIFIER |

Antibodies

| | | |
|---|---|---|
| Goat IRDye 680RD anti-Mouse IgG | LI-COR Biosciences | Cat#926-68070 |
| Goat IRDye 800CW anti-Rabbit IgG | LI-COR Biosciences | Cat#926-32211 |
| CDK7 | Cell Signaling | Cat#2916 |
| Cyclin H | Bethyl Labs | Cat#A301-674A |
| Cyclin K | Bethyl Labs | Cat#A301-939A |
| pCDK2 T160 | Cell Signaling | Cat#2561 |
| pCDKl T161 | Cell Signaling | Cat#9114 |
| CDK1 | Cell Signaling | Cat#77055 |
| CDK2 | Cell Signaling | Cat#2546 |
| CDK1/2 | Santa Cruz | Cat#sc-135400 |
| Tubulin | Cell Signaling | Cat#3873 |
| pCTD Ser2 | Millipore | Cat#04-1571 |
| pCTD Ser5 | Millipore | Cat#04-1572 |
| Total RNA Polymerase II | Bethyl Labs | Cat#3A300-653A |
| Cyclin E D7T3U | Cell Signaling | Cat#20808 |
| Cyclin E HE 12 | Cell Signaling | Cat#4129 |
| Anti-MCM2 antibody [EPR4120] | Abeam | Cat#ab223403 |
| Anti-PCNA | Santa Cruz | Cat#sc-56 |
| yH2AX | Cell Signaling | Cat#9718S |
| ASCL1 | Abeam | Cat#ab211327 |
| MYC | Abeam | Cat#ab32072 |
| STING (22P2F) | Cell Signaling | Cat#13647S |
| pSTING (S365) (D8F4W) | Cell Signaling | Cat#72971S |
| IRF3 (D83B9) | Cell Signaling | Cat#4302S |
| pIRF3 (S396) (4D4G) | Cell Signaling | Cat#4947S |
| pTBK1 (S172) (D52C2) | Cell Signaling | Cat#5483S |
| Actin Clone AC-15 | Sigma-Aldrich | Cat#A5441 |
| STING | Cell Signaling | Cat#13647 |
| Phospho-STING | Cell Signaling | Cat#50907 |
| IRF3 | Cell Signaling | Cat#4302 |
| pIRF3 | Cell Signaling | Cat#37829 |
| TBK1 | Cell Signaling | Cat#3504 |
| pTBK1 | Cell Signaling | Cat#5403 |
| IKKE | Cell Signaling | Cat#2905 |
| pIKKE | Cell Signaling | Cat#8766 |
| InVivoMAb anti-mouse CD8a (clone | Bio X Cell | BE0061 |
| InVivoMAb anti-mouse CD4 Iclone | Bio X Cell | BE0003-1 |
| InVivoMAb rat lgG2b isotype | Bio X Cell | BE0090 |
| control | Gordon J. | N/A |
| anti-mouse PD-1 (CD279) (Clone: | Freeman, DFCI | |
| CD45 (clone 30-F11) | BioLegend | 103155 |
| CD3 (clone 17A2) | BioLegend | 100216 |
| CD4 (clone GK1.5) | BioLegend | 100453 |
| CD8 (clone 53-6.7) | BioLegend | 100759 |
| CD11b (clone M1/70) | BioLegend | 101242 |
| CD 11c (clone N418) | BioLegend | 117336 |
| CD 103 (clone 2E7) | BioLegend | 121425 |
| Ki67 (clone 16A8) | BioLegend | 652411 |
| CD44 (clone IM7) | BioLegend | 103032 |
| CD62L (clone MEL-14) | BioLegend | 104417 |
| TNfo (MP6-XT22) | BioLegend | 506304 |
| IFNy (XMG1.2) | BioLegend | 505826 |

Chemicals , Peptides, and Recombinant Proteins

| | | |
|---|---|---|
| YKL-5-124 | Olson, et al., Cell Chem | N/A |
| | Biol. 2019, 26(6), 792- | |
| | 803 | |
| THZ531 | Zhang, et al., Nat. Chem. | N/A |
| | Biol. 2016,12, 876-884 | |
| THZI | Kwiatkowski, et al., | N/A |
| | Nature, 2014, 511, 616- | |
| | 620 | |
| Cisplatin | TEVA | Cat#18E01 LA |
| | PHARMACEUTICALS | |
| Etoposide | Accord Healthcare, Inc. | Cat#X15146 |
| Biotinylated-THZl (Bio-THZ) | Kwiatkowski, et al., | N/A |
| | Nature, 2014, 511, 616- | |
| | 620 | |
| CCK8 reagent | Dojindo | Cat#CK04 |
| Odyssey Blocking Buffer | LI-COR | Cat#927-50003 |
| Propidium Iodide | Sigma-Aldrich | Cat#P4864 |
| Halt ™ Protease and Phosphatase | Thermo Fisher Scientific | Cat#78440 |
| TaqMan Gene Expression Master | Life Technologies | Cat#4369514 |
| RIPA lysis buffer | Thermo Fisher Scientific | Cat#89900) |
| IDIM media | Thermo Fisher Scientific | Cat#12440061 |
| RPMI media | Thermo Fisher Scientific | Cat#l 1875093 |
| Ovalbumin peptide residues | GenScript | Cat#RP10611 |
| 257-264 | | |
| Collagenase D | Sigma-Aldrich | Cat#11088866001 |
| Dnase 1 | Sigma-Aldrich | Cat#10104159001 |
| ABC Lysis Buffer | BioLegend | Cat#420301 |
| BD Cytofix/Cytoperm | BD Biosciences | Cat#554722 |
| SYBR Green Master Mix | Thermo Fisher Scientific | Cat#A25742 |
| Lipofectamine 3000 | Thermo Fisher Scientific | Cat#L3000015 |
| Glucose Oxidase | Sigma-Aldrich | Cat#2133 |
| Catalase | Sigma-Aldrich | Cat#C3155 |
| Glucose | Sigma-Aldrich | Cat#G8270 |
| Cysteamine | Fisher Scientific | BP2664100 |
| Citrate Buffer (Dako Target Retrieval | Dako | Cat#S1699 |
| Leica Novolink Polymer | Leica | Cat#RE7161 |

Critical Commercial Assays

| | | |
|---|---|---|
| Rneasy Plus Mini Kit | QIAGEN | Cat#74136 |
| BrdU assay | BO Biosciences | Cat#559619 |
| High-Capacity RNA-to-cDNA ™ Kit | Thermo Fisher Scientific | Cat#4387406 |
| UltraComp eBeads'M Compensation | Thermo Fisher Scientific | 01-2222-42 |
| ArC ™ Amine Reactive | Thermo Fisher Scientific | A10628 |
| Cytokine/Chemokine 32-plex Assay | Millipore | MCYTMAG-70K-PX32 |
| Pierce ™ BCA Protein Assay Kit | Thermo Fisher Scientific | Cat#23225 |
| Chromium ™ Single Cell 3 +40 Library | 10x Genomics | PN-120237 |
| & Gel Bead Kit v2 | | |
| Chromium ™ Single Cell Chip Kit | 10x Genomics | PN-120236 |
| Chromium ™ i7 Multiplex Kit | 10x Genomics | PN-120262 |

Deposited Data

| | | |
|---|---|---|
| RNA-seq (bulk) | This paper | Accession Number GEO: |
| | | GSE129299 |
| scRNA-seq | This paper | Accession Number GEO: |
| | | GSE129299 |

Experimental Models: Cell Lines

| | | |
|---|---|---|
| Human: HAPI | Horizon Discovery | C859 |
| Human: GLC16 | Christensen et al., Cancer | N/A |
| | Cell, 2014, 26, 909-922 | |
| Human: NC1-H69 | Christensen et al., Cancer | N/A |
| | Cell, 2014, 26, 909-922 | |
| Human: DMS79 | Christensen et al., Cancer | N/A |
| | Cell, 2014, 26, 909-922 | |
| Human: NC1-H82 | Christensen et al., Cancer | N/A |
| | Cell, 2014, 26, 909-922 | |
| Human: HEK-293T | ATCC | CRL-3216 ™ |
| Mouse: RPP631 | This paper | N/A |
| Mouse: RPP-MYC | This paper | N/A |
| Mouse: RP | This paper | N/A |

Experimental Models: Organisms/Strains

| | | |
|---|---|---|
| C57BU6J | The Jackson Laboratory | Stock No: 000664 |
| RPP631 Orthotopic Model | This paper | N/A |
| RPP-MYC Orthotopic Model | This paper | N/A |
| RP Orthotopic Model | This paper | N/A |
| RPP GEMM | Schaffer, et al., Cancer | N/A |
| | Res. 2010, 70, 3877-3883 | |
| OT-I model | The Jackson Laboratory | Stock No: 003831 |

Oligonucleotides-See Table 3
Recombinant DNA

| | | |
|---|---|---|
| lentiCRISPR v2 | Addgene | Cat#52961 |
| lentiCRISPR v2-sgcGAS | This paper | N/A |
| psPAX2 | Addgene | Cat#12260 |
| pMD2 .G | Addgene | Cat#12259 |

Software and Algorithms

| | | |
|---|---|---|
| GraphPad Prism 7 | GraphPad Software Inc. | http://www .graphpad.com |
| ImageStudio Light | ImageStudio Software | https://www.licor.com/bio/image- |
| | | studio-lite/ |
| R v3 .5.1 | R Core Team (2016) The | http://www.r-project.org |
| | R Project for Statistical | |
| | Computing | |
| Gene Set Enrichment Analysis | Broad Institute | http://software.broadinstitute.org/gsea/ |
| (GSEA) | | index .jsp |
| ModFit LT | Verity Software House | https://www.vsh .com/products/mflV |
| Fiji NIH | ImageJ | https://imagej.nih.gov/ |
| | | ii/docs/guide/146-2 .html |
| Flowjo | FlowJo Software | https://www.flowjo .com/ |
| STAR (v2.5.2b) | STAR Software | https://github.com/alexdobin/STAR |
| featureCounts | featureCounts Software | http ://bioinf.wehi.edu.au/featureCounts/ |
| Cell Ranger (vl.3) | Cell Ranger Single Cell | https://support.10xgenomics.com/ |
| | Software Suite | single-cell-gene- |
| | | expression/software/pipelines/latesV |
| | | what-is -cell-ranger |
| Seurat (v2.3.4) | Seurat R package | https://satij alab.org/seuraV |
| Estimate (v 1.0.13) | Estimate R package | https://bioinformatics.mdanderson.org/ |
| | | estimate/rpackage.html |
| SingleR (vO.2.1) | SingleR R package | https://github.com/dviraran/SingleR |
| uwot (v0.0.0.9010) | Uwot R package | https://github.com/j imelville/uwot |
| dbscan (vl.1-3) | dbscan R package | https://cran.r- |
| | | project.org/web/packages/ dbscan/ |
| | | index.html |
| scran (vl.10.1) | scran R package | https://bioconductor.org/packages/release/ |
| | | hioe/html/cran html |
| seater (vl.10.0) | seater R package | https://bioconductor.org/packages/release/ |
| | | hioc/html/seater html |
| limma (v3.38.2) | limma R package | https://bioconductor.org/packages/release/ |
| | | bioc/html/limma.html |
| BioRender | BioRender Software | https://biorender.com/ |

TABLE 3
RT-qPCR Primer Sequences and sgRNAs,
related to STAR Methods

| RT-qPCR Primers | | |
|---|---|---|
| Gene | | |
| Mouse | Forward | Reverse |
| Tnf | ACTCCAGGC | GAGCGTG |
| | GGTGCCTAT | GTGGCCC |
| | G | CT |
| Cxcl9 | GAACGGAGA | TCTTTTC |
| | TCAAACCTG | CCATTCT |
| | CC | TTCATCA |
| | | GC |
| Cxcl10 | GAATCCGGA | GTGCGTG |
| | ATCTAAGAC | GCTTCAC |
| | CATCAA | TCCAGT |
| Actb | GTGACGTTG | GCCGGAC |
| | ACATCCGTA | TCATCGT |
| | AAGA | ACTCC |
| Tapman Probe | SOURCE | IDENTIFIER |
| Ccne1 (FAM) | Thermo | Cat#4331182, |
| | Fisher | MmO1266311_m1 |
| | Scientific | |
| Ccna1 (FAM) | Thermo | Cat#4331182, |
| | Fisher | Mm00432337_m1 |
| | Scientific | |
| Ccnb1 (FAM) | Thermo | Cat#4331182, |
| | Fisher | Mm03053893_m1 |
| | Scientific | |
| Ccnd1 (FAM) | Thermo | Cat#4331182, |
| | Fisher | Mm00432359_m1 |
| | Scientific | |
| Actb (VIC) | Thermo | Cat#4448489, |
| | Fisher | Mm02619580_g1 |
| | Scientific | |
| Human | | |
| Taqman Probe | SOURCE | IDENTIFIER |
| ASCL1 (FAM) | Thermo | Cat#4331182, |
| | Fisher | Hs00269932_m1 |
| | Scientific | |
| INSM1 (FAM) | Thermo | Cat#4331182, |
| | Fisher | Hs00357871_s1 |
| | Scientific | |
| NFIB (FAM) | Thermo | Cat#4331182, |
| | Fisher | Hs01029175_m1 |
| | Scientific | |
| MYC (FAM) | Thermo | Cat#4331182, |
| | Fisher | Hs00153408_m1 |
| | Scientific | |
| TNF (FAM) | Thermo | Cat#4331182, |
| | Fisher | Hs00174128_m1 |
| | Scientific | |
| CXCL10 (FAM) | Thermo | Cat#4331182, |
| | Fisher | Hs00171042_m1 |
| | Scientific | |
| CCNE1 (FAM) | Thermo | Cat#4331182, |
| | Fisher | Hs01026536_m1 |
| | Scientific | |
| CCNA1 (FAM) | Thermo | Cat#4331182, |
| | Fisher | Hs00171105_m1 |
| | Scientific | |
| CCNB1 (FAM) | Thermo | Cat#4331182, |
| | Fisher | Hs01030099_m1 |
| | Scientific | |
| CCND1 (FAM) | Thermo | Cat#4331182, |
| | Fisher | Hs00765553_m1 |
| | Scientific | |
| GUSB (VIC) | Thermo | Cat#4448490, |
| | Fisher | Hs00939627_m1 |
| | Scientific | |
| ACTB (VIC) | Thermo | Cat#4448489, |
| | Fisher | Hs00157387_m1 |
| | Scientific | |
| Mouse | Target | |
| sgRNA | Sequence | |
| cGAS sgRNA 1 | GAAACGCAAA | |
| | GATATCTCGG | |
| cGAS sgRNA 2 | GGAGCGTGAC | |
| | GGGGACACCA | |

### EQUIVALENTS AND SCOPE

In the claims articles such as “a,” “an,” ˜ and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A method of treating cancer in a subject in need thereof, the method comprising administering a cyclin-dependent kinase 7 (CDK7) inhibitor and an immunotherapy.

2. The method of claim 1, wherein the CDK7 inhibitor is a compound of Formula (I): 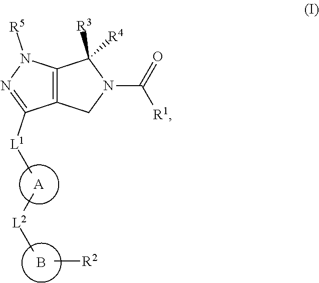
or a pharmaceutically acceptable salt, solvate, hydrate, polymorph, co-crystal, tautomer, stereoisomer, isotopically labeled derivative, or prodrug thereof, wherein:
R¹ is —NR^{a}R^{b}, —CHR^{a}R^{b} or —OR^{a}, wherein each of R^{a} and R^{b} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group when attached to a nitrogen atom, or an oxygen protecting group when attached to an oxygen atom, or R^{a} and R^{b} are joined to form an optionally substituted carbocyclic, optionally substituted heterocyclic, optionally substituted aryl, or optionally substituted heteroaryl ring;
each of R³ and R⁴ is independently hydrogen, halogen, optionally substituted C₁-C₆ alkyl, or optionally substituted aryl, or R³ and R⁴ are joined to form an optionally substituted C₃-C₆ carbocyclyl ring;
R⁵ is hydrogen, optionally substituted C₁-C₆ alkyl, or a nitrogen protecting group;
L¹ is —NR^{L1}—, —NR^{L1}C(═O)—, —C(═O)NR^{L1}—, —O—, or —S—, wherein R^{L1} is hydrogen, optionally substituted C₁-C₆ alkyl, or a nitrogen protecting group;
Ring A is optionally substituted carbocyclyl, optionally substituted heterocyclyl, optionally substituted aryl, or optionally substituted heteroaryl;
L² is a bond, —C(═O)—, —NR^{L2}—, —C(═O)NR^{L2}—, —NR^{L2}C(═O)—, —O—, or —S—, wherein R^{L2} is hydrogen, optionally substituted C₁-C₆ alkyl, or a nitrogen protection group;
Ring B is absent, optionally substituted carbocyclyl, optionally substituted heterocyclyl, optionally substituted aryl, or optionally substituted heteroaryl; and
R² is of the formula: 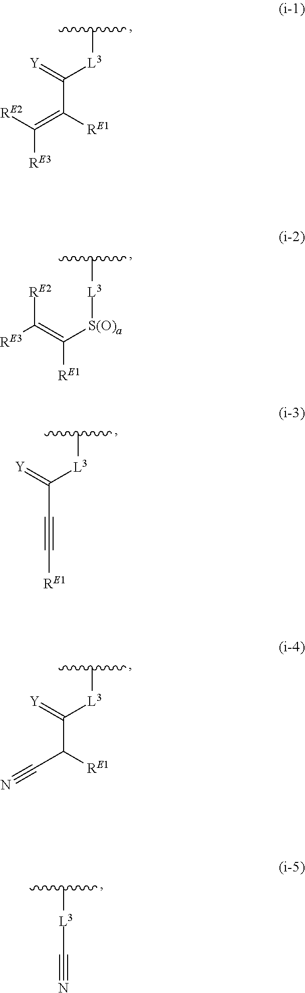 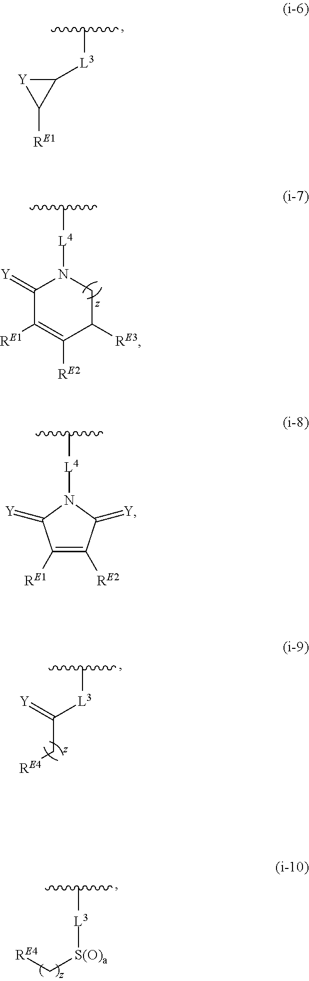 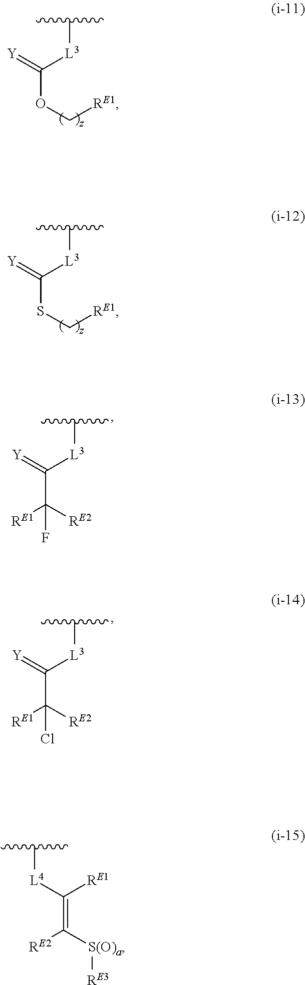 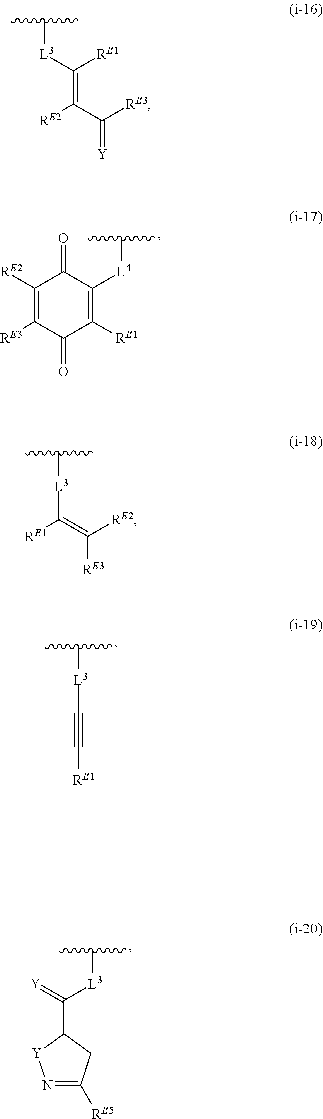 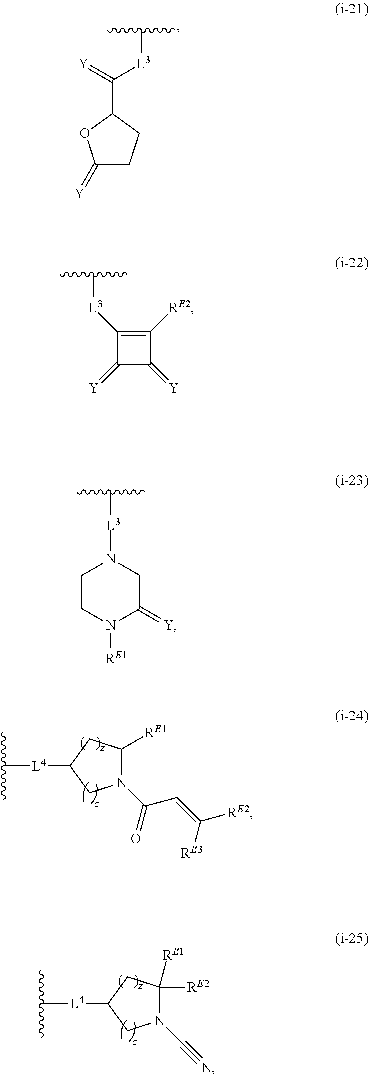 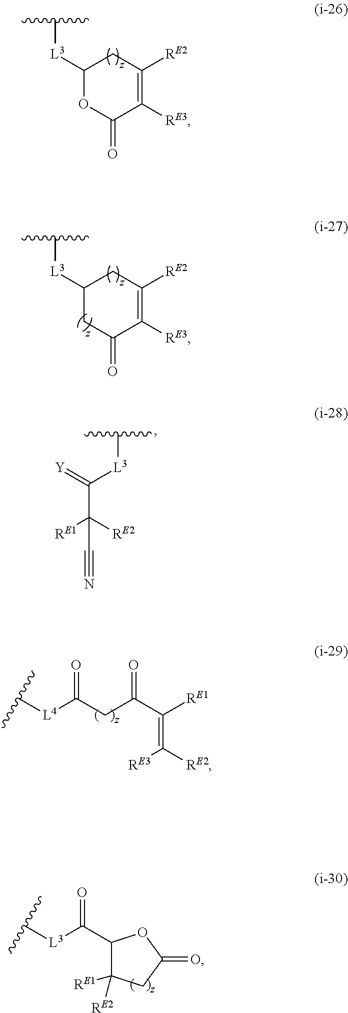 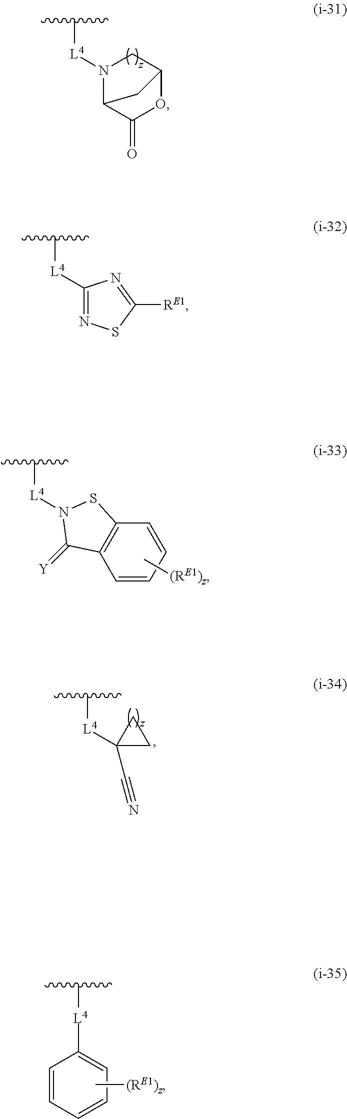 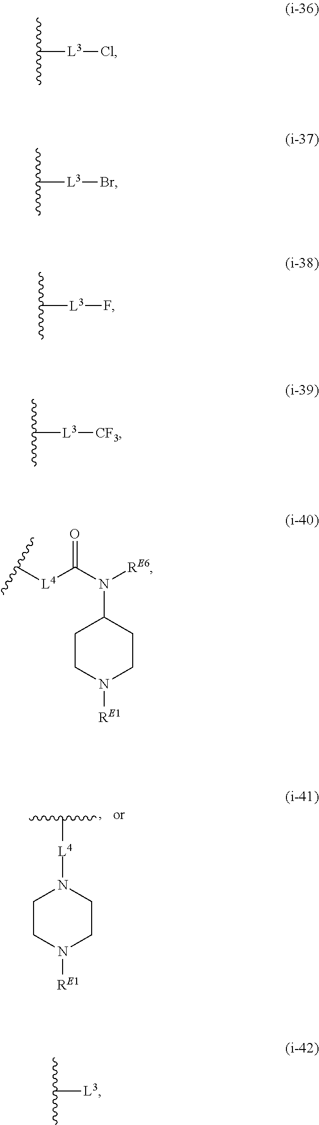
wherein:
L³ is a bond or an optionally substituted C₁₋₄ hydrocarbon chain, optionally wherein one or more carbon units of the hydrocarbon chain are independently replaced with —C═O—, —O—, —S—, —NR^{L3a}—, —NR^{L3a}C(═O)—, —C(═O)NR^{L3a}—, —SC(═O)—, —C(═O)S—, —OC(═O)—, —C(═O)O—, —NR^{L3a}C(═S)—, —C(═S)NR^{L3a}—, trans-CR^{L3b}═CR^{L3b}—, cis-CR^{L3b}═CR^{L3b}—, —C≡C—, —S(═O)—, —S(═O)O—, —OS(═O)—, —S(═O)NR^{L3a}—, —NR^{L3a}S(═O)—, —S(═O)₂—, —S(═O)₂O—, —OS(═O)₂—, —S(═O)₂NR^{L3a}—, or —NR^{L3a}S(═O)₂—, wherein R^{L3a} is hydrogen, substituted or unsubstituted C₁₋₆ alkyl, or a nitrogen protecting group, and wherein each occurrence of R^{L3b} is independently hydrogen, halogen, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R^{L3b} groups are joined to form an optionally substituted carbocyclic or optionally substituted heterocyclic ring;
L⁴ is a bond or an optionally substituted, branched or unbranched C₁₋₆ hydrocarbon chain;
each of R^{E1}, R^{E2}, and R^{E3} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{EE}, —CH₂N(R^{EE})₂, —CH₂SR^{EE}, —OR^{EE}, —N(R^{EE})₂, —Si(R^{EE})₃, and —SR^{EE}, wherein each occurrence of R^{EE} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl, or two R^{EE} groups are joined to form an optionally substituted heterocyclic ring;
or R^{E1} and R^{E3}, or R^{E2} and R^{E3}, or R^{E1} and R^{E2} are joined to form an optionally substituted carbocyclic or optionally substituted heterocyclic ring;
R^{E4} is a leaving group;
R^{E5} is halogen;
R^{E6} is hydrogen, substituted or unsubstituted C₁₋₆ alkyl, or a nitrogen protecting group;
each instance of Y is independently O, S, or NR^{E7}, wherein R^{E7} is hydrogen, substituted or unsubstituted C₁₋₆ alkyl, or a nitrogen protecting group;
a is 1 or 2; and
each instance of z is independently 0, 1, 2, 3, 4, 5, or 6, as valency permits.

3. The method of claim 2, wherein the CDK7 inhibitor is a compound of Formula (III): 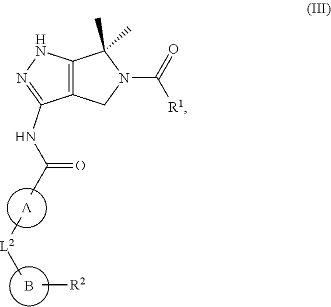
or a pharmaceutically acceptable salt, solvate, hydrate, polymorph, co-crystal, tautomer, stereoisomer, isotopically labeled derivative, or prodrug thereof.

4. The method of claim 2, wherein the CDK7 inhibitor is a compound of Formula (IV-b): 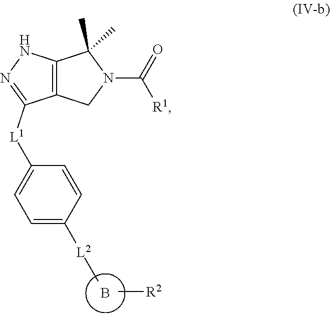
or a pharmaceutically acceptable salt, solvate, hydrate, polymorph, co-crystal, tautomer, stereoisomer, isotopically labeled derivative, or prodrug thereof.

5. The method of claim 2, wherein the CDK7 inhibitor is a compound of Formula (V-d): 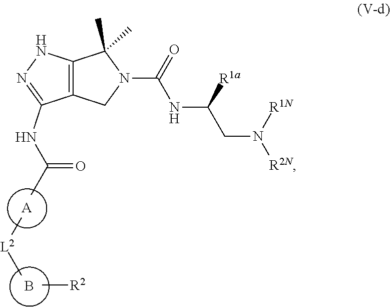
or a pharmaceutically acceptable salt, solvate, hydrate, polymorph, co-crystal, tautomer, stereoisomer, isotopically labeled derivative, or prodrug thereof.

6. The method of claim 2, wherein the CDK7 inhibitor is a compound of Formula (VI-c): 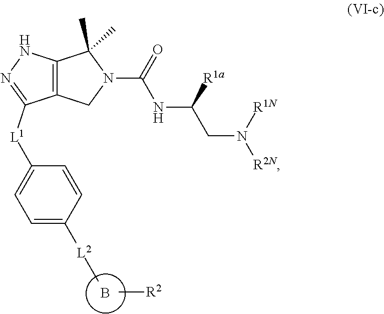
or a pharmaceutically acceptable salt, solvate, hydrate, polymorph, co-crystal, tautomer, stereoisomer, isotopically labeled derivative, or prodrug thereof.

7. The method of claim 2, wherein the CDK7 inhibitor is a compound of Formula (VII-c): 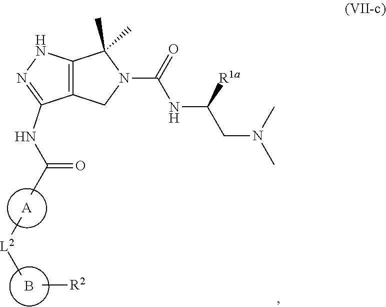
or a pharmaceutically acceptable salt, solvate, hydrate, polymorph, co-crystal, tautomer, stereoisomer, isotopically labeled derivative, or prodrug thereof.

8. The method of claim 2, wherein the CDK7 inhibitor is a compound of Formula (VIII-c): 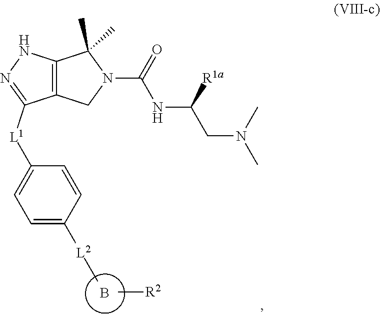
or a pharmaceutically acceptable salt, solvate, hydrate, polymorph, co-crystal, tautomer, stereoisomer, isotopically labeled derivative, or prodrug thereof.

9. The method of claim 2, wherein the CDK7 inhibitor is a compound of Formula (IX-c): 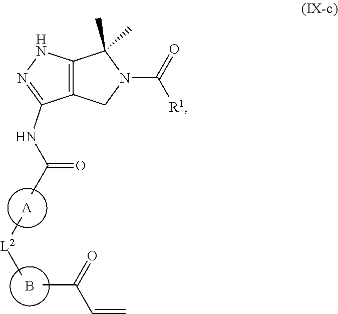
or a pharmaceutically acceptable salt, solvate, hydrate, polymorph, co-crystal, tautomer, stereoisomer, isotopically labeled derivative, or prodrug thereof.

10. The method of claim 2, wherein the CDK7 inhibitor is a compound of Formula (X-c): 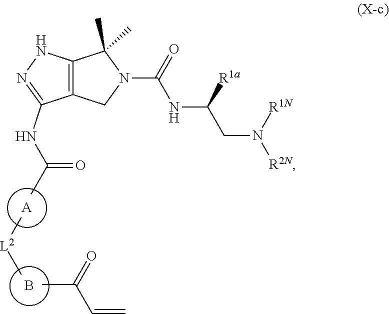
or a pharmaceutically acceptable salt, solvate, hydrate, polymorph, co-crystal, tautomer, stereoisomer, isotopically labeled derivative, or prodrug thereof.

11. The method of claim 2, wherein the CDK7 inhibitor is a compound of Formula (XI-c): 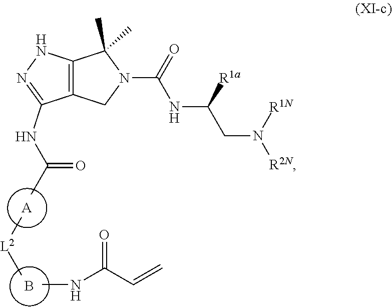
or a pharmaceutically acceptable salt, solvate, hydrate, polymorph, co-crystal, tautomer, stereoisomer, isotopically labeled derivative, or prodrug thereof.

12. The method of claim 2, wherein the CDK7 inhibitor is a compound of Formula (XII-c): 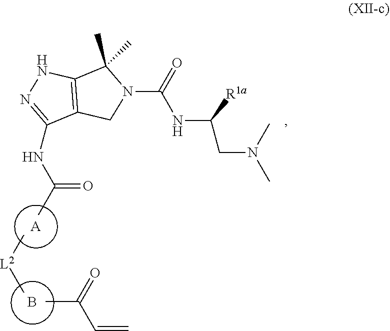
or a pharmaceutically acceptable salt, solvate, hydrate, polymorph, co-crystal, tautomer, stereoisomer, isotopically labeled derivative, or prodrug thereof.

13. The method of claim 2, wherein the CDK7 inhibitor is a compound of Formula (XIII-c): 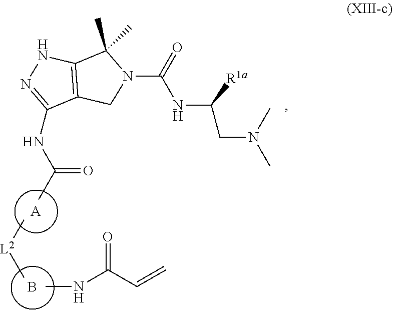
or a pharmaceutically acceptable salt, solvate, hydrate, polymorph, co-crystal, tautomer, stereoisomer, isotopically labeled derivative, or prodrug thereof.

14. The method of any of claims 2-13, wherein Ring A comprises an optionally substituted phenyl ring.

15. The method of any of claims 2-14, wherein Ring A is 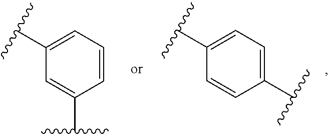 [SMILES: *c1cccc(*)c1 ; *c1ccc(*)cc1]
wherein each ring atom is optionally substituted.

16. The method of any of claims 2-15, wherein L¹ is —NR^{L1}(C═O)—.

17. The method of any of claims 2-16, wherein L¹ is —NH(C═O)—.

18. The method of any of claims 2-17, wherein L² is a bond.

19. The method of any of claims 2-18, wherein Ring B is absent.

20. The method of any of claims 2-19, wherein L² is a bond; and Ring B is absent.

21. The method of any of claims 2-20, wherein R¹ comprises an optionally substituted alkylamino group.

22. The method of any of claims 2-21, wherein R¹ comprises a dimethylamino group.

23. The method of any of claims 2-22, wherein R¹ is of Formula (ii-1): 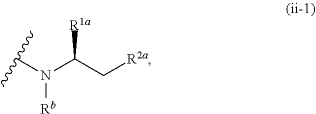
wherein:
R^{b} is hydrogen, optionally substituted C₁-C₆ alkyl or a nitrogen protecting group;
R^{1a} is hydrogen, C₁-C₆ alkyl, or optionally substituted aryl; and
R^{2a} is hydrogen, —OR^{1N}, or —NR^{1N}R^{2N}, wherein each of R^{1N} and R^{2N} is independently hydrogen, C₁-C₆ alkyl, a nitrogen protecting group when attached to a nitrogen atom, or an oxygen protecting group when attached to an oxygen atom, or R^{1N} and R^{2N} are joined to form an optionally substituted carbocyclic, optionally substituted heterocyclic, optionally substituted aryl, or optionally substituted heteroaryl ring.

24. The method of claim 23, wherein R^{2a} is —N(CH₃)₂.

25. The method of any of claims 2-24, wherein R¹ is:

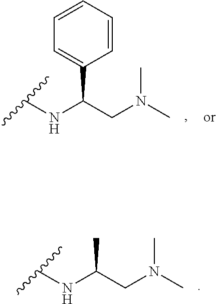

26. The method of any of claims 2-25, wherein R² is of Formula (i-1):

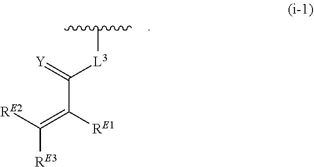

27. The method of any of claims 2-26, wherein R² is:

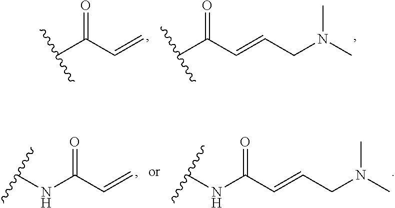

28. The method of any of claims 2-27, wherein R² is

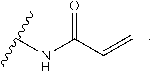 [SMILES: *NC(=O)C=C]

29. The method of any of claims 2-28, wherein R³ and R⁴ are each independently optionally substituted C₁₋₆ alkyl or optionally substituted aryl, or R³ and R⁴ are joined to form an optionally substituted C₃₋₆ carbocyclic ring.

30. The method of any of claims 2-29, wherein R³ and R⁴ are each independently methyl, isopropyl, or phenyl.

31. The method of any of claims 2-30, wherein R³ and R⁴ are each methyl.

32. The method of any of claims 2-31, wherein R⁵ is hydrogen or optionally substituted methyl.

33. The method of any of claims 2-32, wherein R⁵ is hydrogen.

34. The method of claim 1 or 2, wherein the CDK7 inhibitor is of formula: 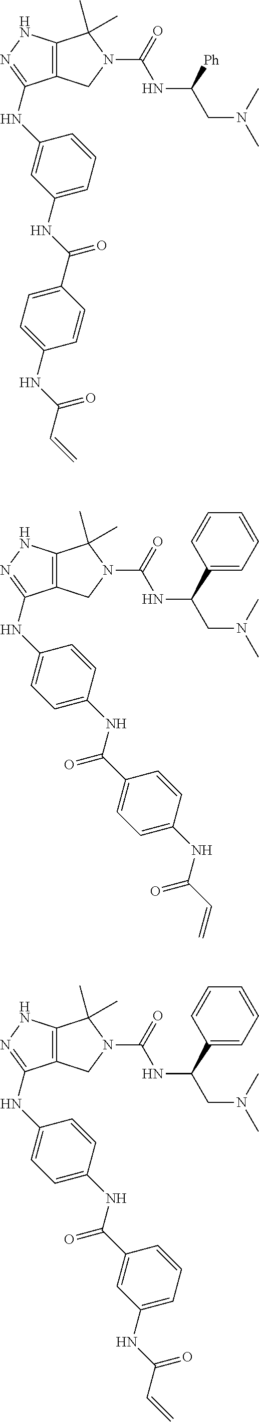 [SMILES: C=CC(=O)Nc1ccc(C(=O)Nc2cccc(Nc3n[nH]c4c3CN(C(=O)N[C@H](CN(C)C)c3ccccc3)C4(C)C)c2)cc1 ; C=CC(=O)Nc1ccc(C(=O)Nc2ccc(Nc3n[nH]c4c3CN(C(=O)N[C@H](CN(C)C)c3ccccc3)C4(C)C)cc2)cc1 ; C=CC(=O)Nc1cccc(C(=O)Nc2ccc(Nc3n[nH]c4c3CN(C(=O)N[C@H](CN(C)C)c3ccccc3)C4(C)C)cc2)c1] 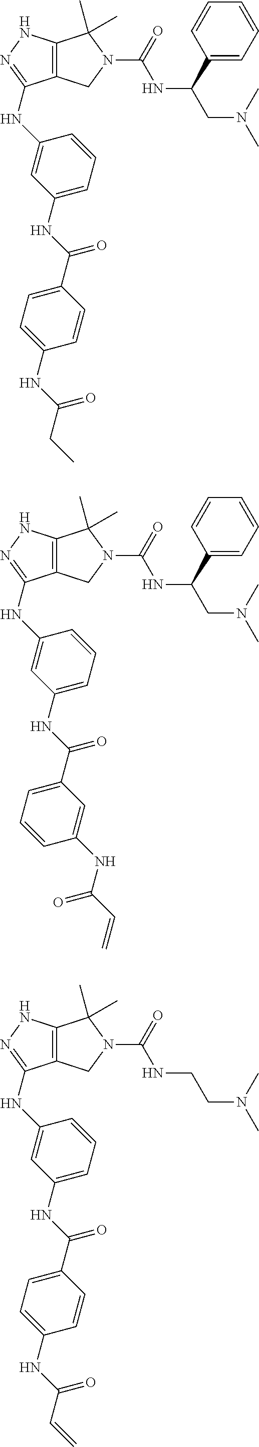 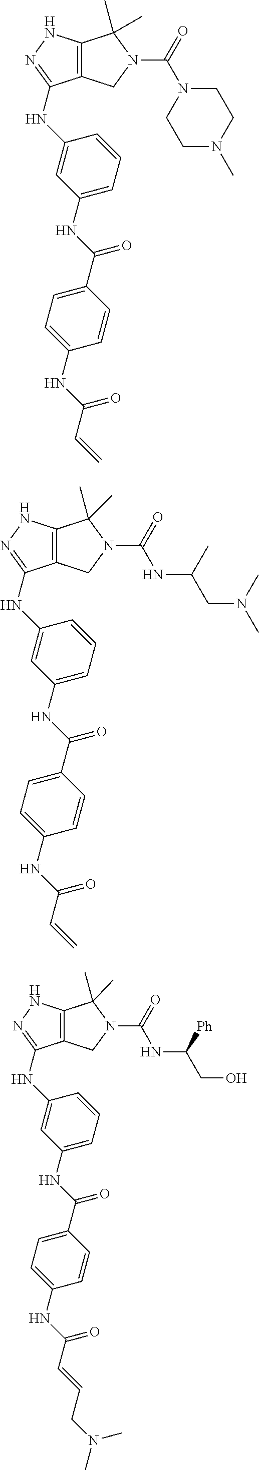 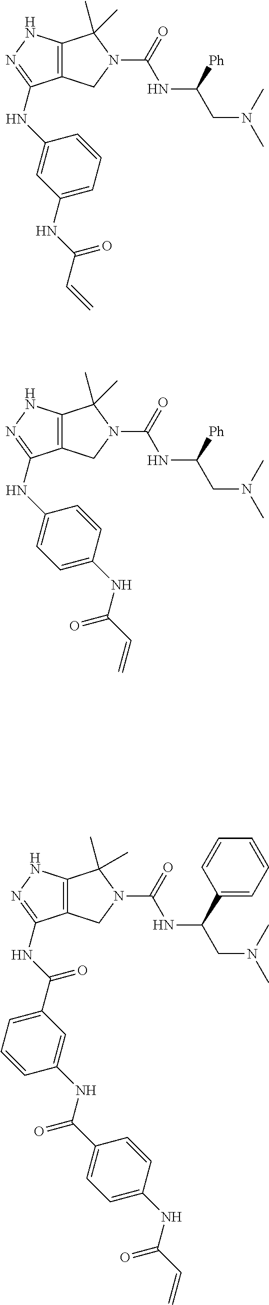 [SMILES: C=CC(=O)Nc1cccc(Nc2n[nH]c3c2CN(C(=O)N[C@H](CN(C)C)c2ccccc2)C3(C)C)c1 ; C=CC(=O)Nc1ccc(Nc2n[nH]c3c2CN(C(=O)N[C@H](CN(C)C)c2ccccc2)C3(C)C)cc1 ; C=CC(=O)Nc1ccc(C(=O)Nc2cccc(C(=O)Nc3n[nH]c4c3CN(C(=O)N[C@H](CN(C)C)c3ccccc3)C4(C)C)c2)cc1] 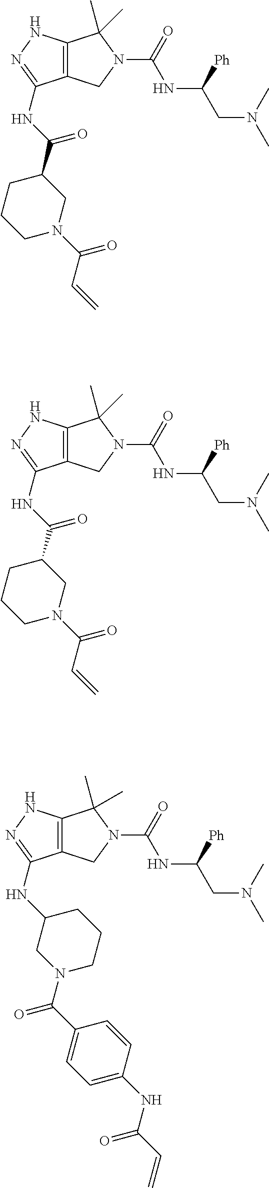 [SMILES: C=CC(=O)N1CCC[C@@H](C(=O)Nc2n[nH]c3c2CN(C(=O)N[C@H](CN(C)C)c2ccccc2)C3(C)C)C1 ; C=CC(=O)N1CCC[C@H](C(=O)Nc2n[nH]c3c2CN(C(=O)N[C@H](CN(C)C)c2ccccc2)C3(C)C)C1 ; C=CC(=O)Nc1ccc(C(=O)N2CCCC(Nc3n[nH]c4c3CN(C(=O)N[C@H](CN(C)C)c3ccccc3)C4(C)C)C2)cc1] 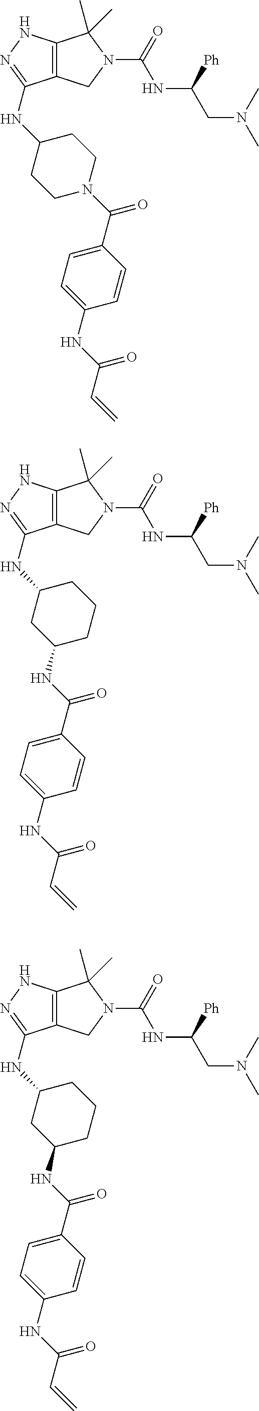 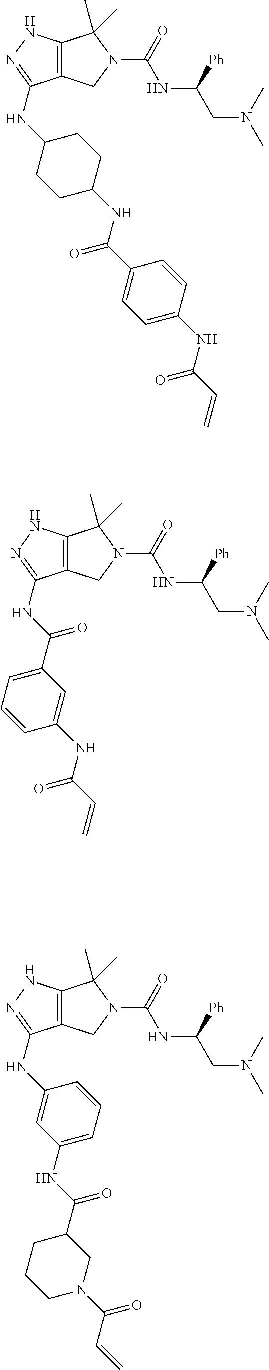 [SMILES: C=CC(=O)Nc1ccc(C(=O)NC2CCC(Nc3n[nH]c4c3CN(C(=O)N[C@H](CN(C)C)c3ccccc3)C4(C)C)CC2)cc1 ; C=CC(=O)Nc1cccc(C(=O)Nc2n[nH]c3c2CN(C(=O)N[C@H](CN(C)C)c2ccccc2)C3(C)C)c1 ; C=CC(=O)N1CCCC(C(=O)Nc2cccc(Nc3n[nH]c4c3CN(C(=O)N[C@H](CN(C)C)c3ccccc3)C4(C)C)c2)C1] 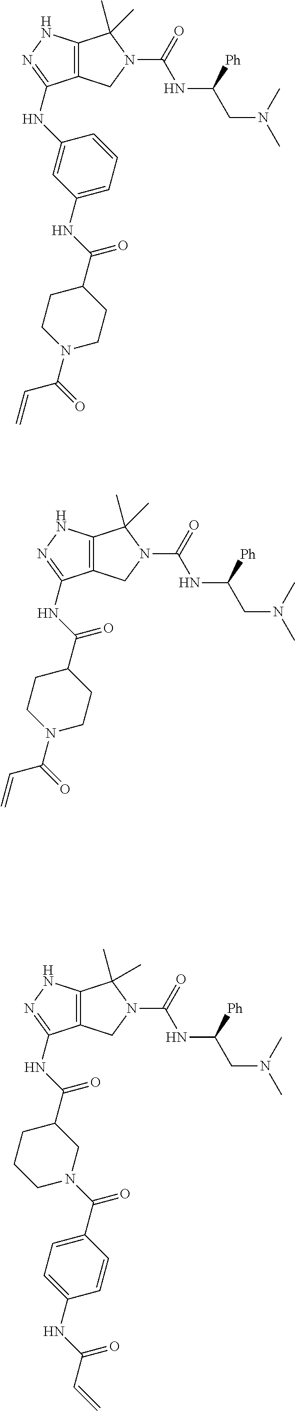 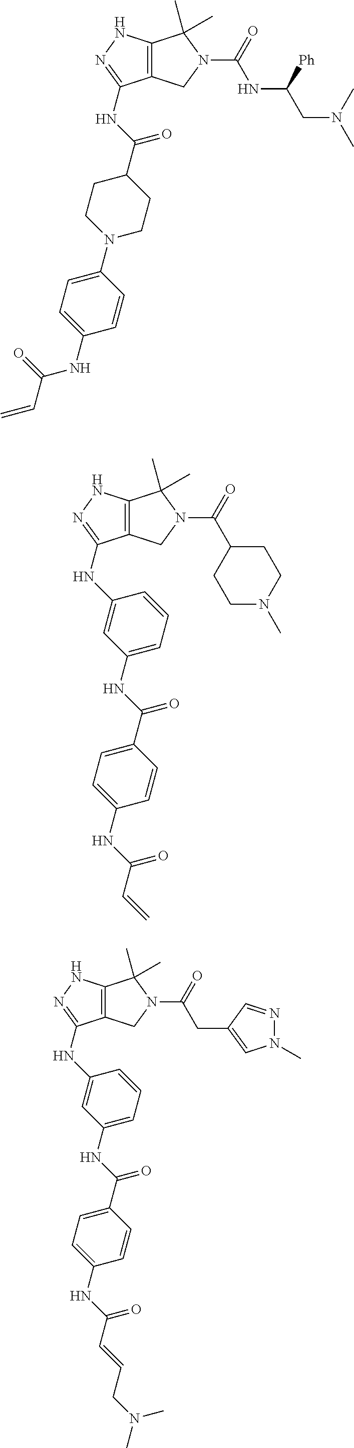 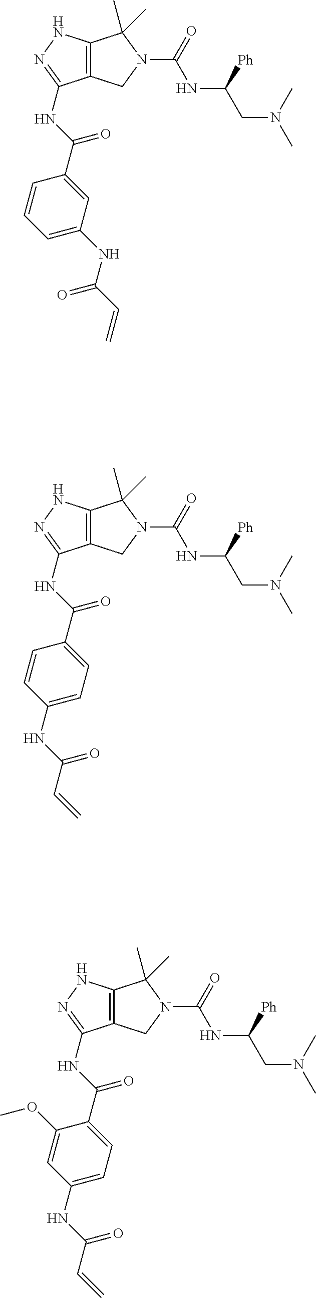 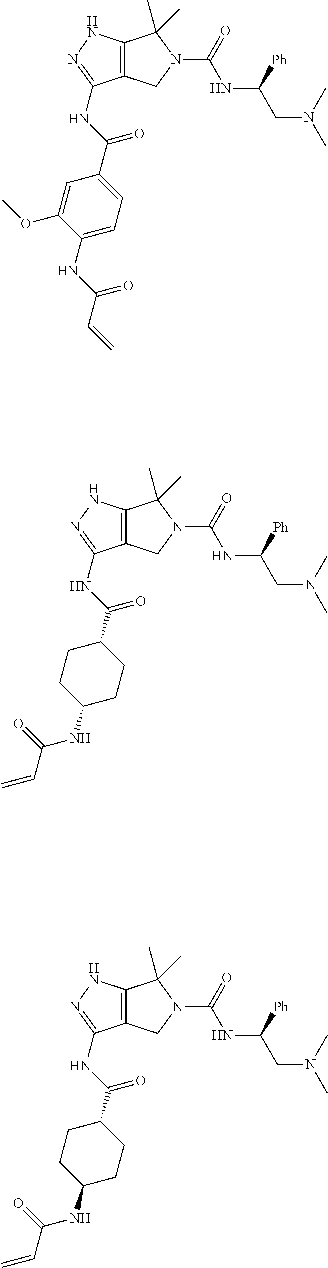 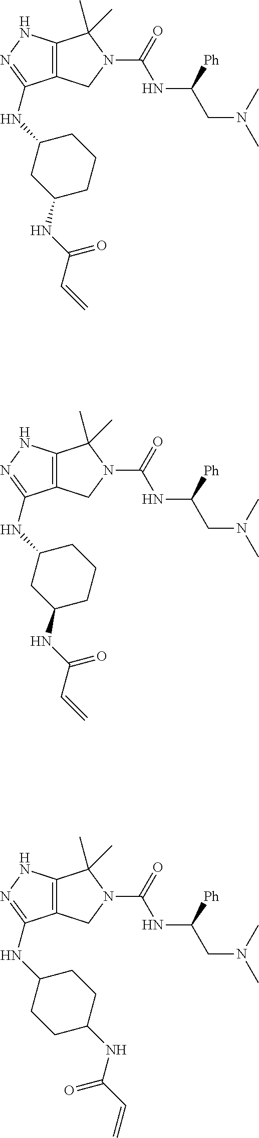 [SMILES: C=CC(=O)N[C@H]1CCC[C@@H](Nc2n[nH]c3c2CN(C(=O)N[C@H](CN(C)C)c2ccccc2)C3(C)C)C1 ; C=CC(=O)N[C@@H]1CCC[C@@H](Nc2n[nH]c3c2CN(C(=O)N[C@H](CN(C)C)c2ccccc2)C3(C)C)C1 ; C=CC(=O)NC1CCC(Nc2n[nH]c3c2CN(C(=O)N[C@H](CN(C)C)c2ccccc2)C3(C)C)CC1] 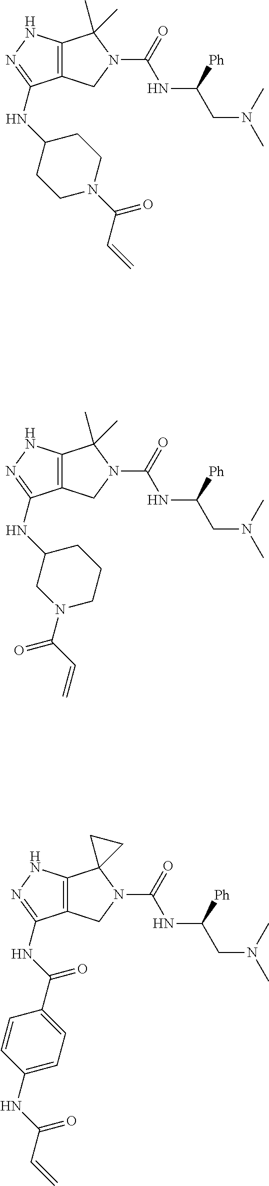 [SMILES: C=CC(=O)N1CCC(Nc2n[nH]c3c2CN(C(=O)N[C@H](CN(C)C)c2ccccc2)C3(C)C)CC1 ; C=CC(=O)N1CCCC(Nc2n[nH]c3c2CN(C(=O)N[C@H](CN(C)C)c2ccccc2)C3(C)C)C1 ; C=CC(=O)Nc1ccc(C(=O)Nc2n[nH]c3c2CN(C(=O)N[C@H](CN(C)C)c2ccccc2)C32CC2)cc1] 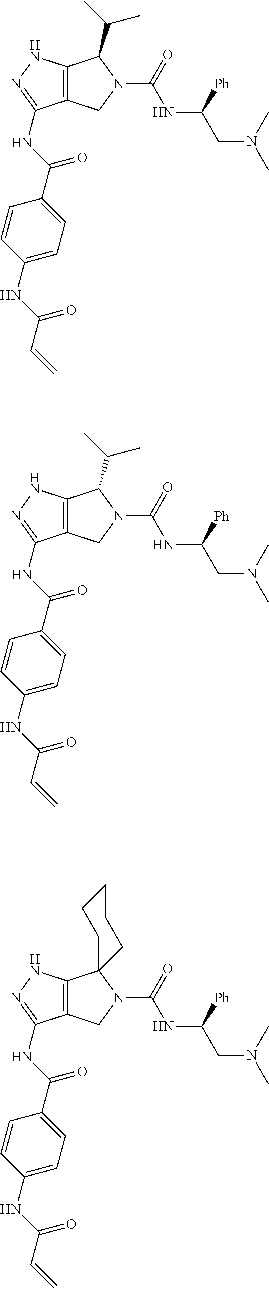 [SMILES: C=CC(=O)Nc1ccc(C(=O)Nc2n[nH]c3c2CN(C(=O)N[C@H](CN(C)C)c2ccccc2)[C@@H]3C(C)C)cc1 ; C=CC(=O)Nc1ccc(C(=O)Nc2n[nH]c3c2CN(C(=O)N[C@H](CN(C)C)c2ccccc2)[C@H]3C(C)C)cc1 ; C=CC(=O)Nc1ccc(C(=O)Nc2n[nH]c3c2CN(C(=O)N[C@H](CN(C)C)c2ccccc2)C32CCCCC2)cc1] 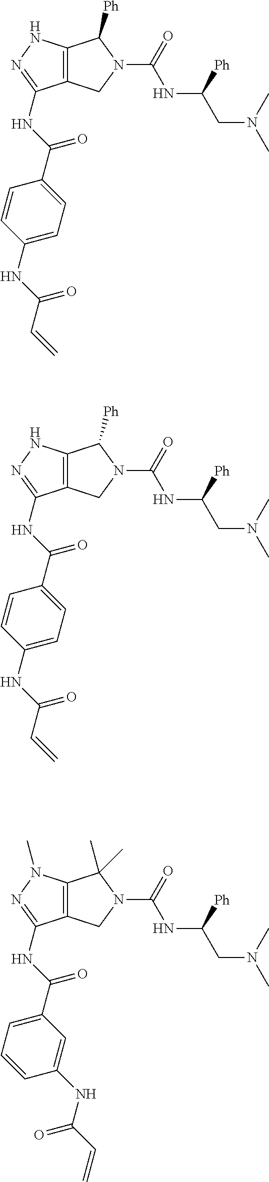 [SMILES: C=CC(=O)Nc1ccc(C(=O)Nc2n[nH]c3c2CN(C(=O)N[C@H](CN(C)C)c2ccccc2)[C@@H]3c2ccccc2)cc1 ; C=CC(=O)Nc1ccc(C(=O)Nc2n[nH]c3c2CN(C(=O)N[C@H](CN(C)C)c2ccccc2)[C@H]3c2ccccc2)cc1 ; C=CC(=O)Nc1cccc(C(=O)Nc2nn(C)c3c2CN(C(=O)N[C@H](CN(C)C)c2ccccc2)C3(C)C)c1] 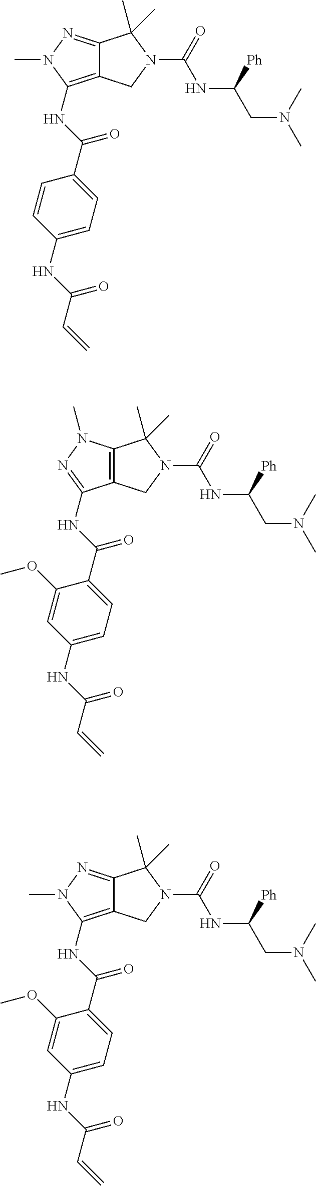 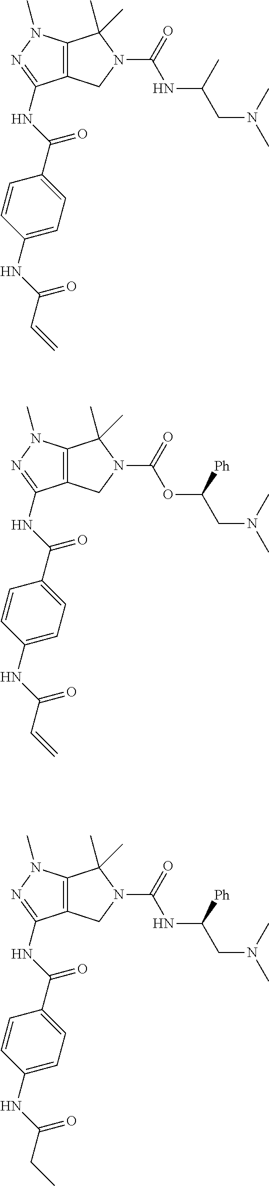 [SMILES: C=CC(=O)Nc1ccc(C(=O)Nc2nn(C)c3c2CN(C(=O)NC(C)CN(C)C)C3(C)C)cc1 ; C=CC(=O)Nc1ccc(C(=O)Nc2nn(C)c3c2CN(C(=O)O[C@H](CN(C)C)c2ccccc2)C3(C)C)cc1 ; CCC(=O)Nc1ccc(C(=O)Nc2nn(C)c3c2CN(C(=O)N[C@H](CN(C)C)c2ccccc2)C3(C)C)cc1]
or a pharmaceutically acceptable salt, solvate, hydrate, polymorph, co-crystal, stereoisomer, tautomer, isotopically labeled derivative, or prodrug thereof.

35. The method of claim 1 or 2, wherein the CDK7 inhibitor is of formula: 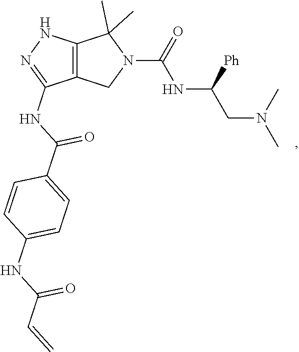 [SMILES: C=CC(=O)Nc1ccc(C(=O)Nc2n[nH]c3c2CN(C(=O)N[C@H](CN(C)C)c2ccccc2)C3(C)C)cc1]
or a pharmaceutically acceptable salt, solvate, hydrate, polymorph, co-crystal, stereoisomer, tautomer, isotopically labeled derivative, or prodrug thereof.

36. The method of any of claims 1-35, wherein the immunotherapy is an immunotherapeutic agent.

37. The method of any of claims 1-36, wherein the immunotherapy is an activator of adaptive immune response.

38. The method of any of claims 1-37, wherein the immunotherapy is an immune checkpoint inhibitor.

39. The method of any of claims 1-38, wherein the immunotherapy is an inhibitor of PD-1, PD-L1, or CTLA-4.

40. The method of any of claims 1-39, wherein the immunotherapy is an inhibitor of PD-1.

41. The method of any of claims 1-40, wherein the immunotherapy is an anti-PD-1 antibody.

42. The method of any of claims 1-39, wherein the immunotherapy is ipilimumab, nivolumab, pembrolizumab, spartalizumab, atezolizumab, tislelizumab, tremelimumab, durvalumab, avelumab, or cemiplimab.

43. The method of any of claims 1-42, wherein the CDK7 inhibitor is of formula: 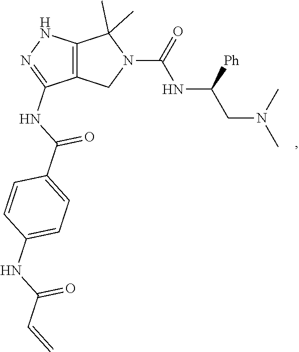 [SMILES: C=CC(=O)Nc1ccc(C(=O)Nc2n[nH]c3c2CN(C(=O)N[C@H](CN(C)C)c2ccccc2)C3(C)C)cc1]
or a pharmaceutically acceptable salt, solvate, hydrate, polymorph, co-crystal, stereoisomer, tautomer, isotopically labeled derivative, or prodrug thereof; and the immunotherapy is an anti-PD-1 antibody.

44. The method of any of claims 1-43, wherein the combination of the CDK7 inhibitor and the immunotherapy are synergistic in treating the cancer, compared to treatment with the CDK7 inhibitor alone or treatment with the immunotherapy alone.

45. The method of any of claims 1-44 further comprising administering a chemotherapeutic agent.

46. The method of any of claims 1-45 further comprising administering at least one of cisplatin and etoposide.

47. The method of any of claims 1-46 further comprising administering a targeted agent.

48. The method of any of claims 1-47, wherein the cancer is bladder cancer, esophageal cancer, stomach cancer, skin cancer, throat cancer, or lung cancer.

49. The method of any of claims 1-48, wherein the cancer is small cell lung cancer.

50. A pharmaceutical composition comprising a CDK7 inhibitor and an immunotherapeutic agent, and optionally a pharmaceutically acceptable excipient.

51. The pharmaceutical composition of claim 50, wherein the CDK7 inhibitor is of the formula: 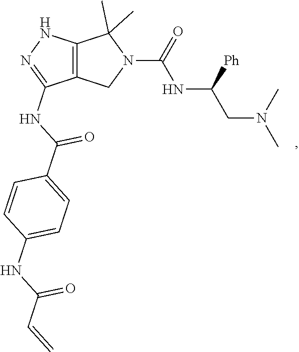 [SMILES: C=CC(=O)Nc1ccc(C(=O)Nc2n[nH]c3c2CN(C(=O)N[C@H](CN(C)C)c2ccccc2)C3(C)C)cc1]
or a pharmaceutically acceptable salt, solvate, hydrate, polymorph, co-crystal, stereoisomer, tautomer, isotopically labeled derivative, or prodrug thereof; and the immunotherapy is an anti-PD-1 antibody.

52. The pharmaceutical composition of claim 50 or 51 further comprising a chemotherapeutic agent.

53. The pharmaceutical composition of claim 52, wherein the chemotherapeutic agent is cisplatin or etoposide.

54. A kit comprising a CDK7 inhibitor and an immunotherapeutic agent and instructions for using the kit.


SEQUENCE LISTING (11 sequences)

SEQ ID NO: 1 - protein, 346 aa, Homo sapiens
MALDVKSRAKRYEKLDFLGEGQFATVYKARDKNTNQIVAIKKIKLGHRSEAKDGINRTALREIKLLQELSHPNIIGLLDAFGHKSNISLVFDFMETDLEVIIKDNSLVLTPSHIKAYMLMTLQGLEYLHQHWILHRDLKPNNLLLDENGVLKLADFGLAKSFGSPNRAYTHQVVTRWYRAPELLFGARMYGVGVDMWAVGCILAELLLRVPFLPGDSDLDQLTRIFETLGTPTEEQWPDMCSLPDYVTFKSFPGIPLHHIFSAAGDDLLDLIQGLFLFNPCARITATQALKMKYFSNRPGPTPGCQLPRPNCPVETLKEQSNPALAIKRKRTEALEQGGLPKKLIF

SEQ ID NO: 2 - DNA, 19 bp, Artificial Sequence
  Synthetic
actccaggcggtgcctatg

SEQ ID NO: 3 - DNA, 16 bp, Artificial Sequence
  Synthetic
gagcgtggtggcccct

SEQ ID NO: 4 - DNA, 20 bp, Artificial Sequence
  Synthetic
gaacggagatcaaacctgcc

SEQ ID NO: 5 - DNA, 23 bp, Artificial Sequence
  Synthetic
tcttttcccattctttcatcagc

SEQ ID NO: 6 - DNA, 24 bp, Artificial Sequence
  Synthetic
gaatccggaatctaagaccatcaa

SEQ ID NO: 7 - DNA, 20 bp, Artificial Sequence
  Synthetic
gtgcgtggcttcactccagt

SEQ ID NO: 8 - DNA, 22 bp, Artificial Sequence
  Synthetic
gtgacgttgacatccgtaaaga

SEQ ID NO: 9 - DNA, 19 bp, Artificial Sequence
  Synthetic
gccggactcatcgtactcc

SEQ ID NO: 10 - DNA, 20 bp, Artificial Sequence
  Synthetic
gaaacgcaaagatatctcgg

SEQ ID NO: 11 - DNA, 20 bp, Artificial Sequence
  Synthetic
ggagcgtgacggggacacca